(19) United States
(12) Patent Application Publication
(10) Pub. No.: US 2024/0287172 A1
(43) Pub. Date: Aug. 29, 2024

(54) PROTEIN BINDERS TO IRHOM2 EPITOPES

(71) Applicants: SCIRHOM GMBH, Planegg (DE); NEW YORK SOCIETY FOR THE RELIEF OF THE RUPTURED AND CRIPPLED, MAINTAINING THE HOSPITAL FOR SPECIAL, New York, NY (US)

(72) Inventors: Matthias SCHNEIDER, Neufarn (DE); Kerstin SELLE, Planegg (DE); Jens RUHE, Planegg (DE); Gisela WESKAMP, Eastchester, NY (US); Carl BLOBEL, Eastchester, NY (US)

(21) Appl. No.: 17/764,819

(22) PCT Filed: Sep. 30, 2020
(86) PCT No.: PCT/EP2020/077346

§ 371 Date: Sep. 7, 2022

(30) Foreign Application Priority Data
Sep. 30, 2019 (EP) 19200589.0

(51) Int. Cl. C07K 16/28; A61K 39/00

(52) U.S. Cl. CPC C07K 16/28; A61K 2039/505; C07K 2317/24; C07K 2317/33; C07K 2317/34; C07K 2317/76; C07K 2317/92

(57) ABSTRACT

The present invention relates to a protein binder which, when bound to human iRhom2, binds at least within a region of Loop 1 thereof.

### SEQUENCE LISTING

The instant application contains a Sequence Listing which has been submitted electronically in ASCII format and is hereby incorporated by reference in its entirety. Said ASCII copy, created on Sep. 2, 2022, is named 51656-003001_Sequence_Listing_9_2_22_ST25 and is 213,140 bytes in size.

### FIELD OF THE INVENTION

The present application relates to protein binders against iRhom2.

### BACKGROUND

ADAM metallopeptidase domain 17 (ADAM17) (NCBI reference of human ADAM17: NP_003174), also called TACE (tumor necrosis factor-α-converting enzyme), is an enzyme that belongs to the ADAM protein family of disintegrins and metalloproteases. It is an 824-amino acid polypeptide.

ADAM17 is understood to be involved in the processing of tumor necrosis factor alpha (TNF-α) at the surface of the cell, and from within the intracellular membranes of the trans-Golgi network. This process, which is also known as ‘shedding’, involves the cleavage and release of α soluble ectodomain from membrane-bound pro-proteins (such as pro-TNF-α), and is of known physiological importance. ADAM17 was the first ‘sheddase’ to be identified, and is also understood to play a role in the release of a variety of membrane-anchored cytokines, cell adhesion molecules, receptors, ligands, and enzymes.

Cloning of the TNF-α gene revealed it to encode a 26 kDa type II transmembrane pro-polypeptide that becomes inserted into the cell membrane during its translocation in the endoplasmic reticulum. At the cell surface, pro-TNF-α is biologically active and is able to induce immune responses via juxtacrine intercellular signaling. However, pro-TNF-α can undergo proteolytic cleavage at its Ala76-Val77 amide bond, which releases α soluble 17 kDa extracellular domain (ectodomain) from the pro-TNF-α molecule. This soluble ectodomain is the cytokine commonly known as TNF-α, which is of pivotal importance in paracrine signaling of this molecule. This proteolytic liberation of soluble TNF-α is catalyzed by ADAM17.

ADAM17 also modulates the MAP kinase signaling pathway by regulating the cleavage of the EGFR ligand amphiregulin in the mammary gland. ADAM17 is important for activating several ligands of the EGFR, TGFα, AREG, EREG, HB-EGF, Epigen. Moreover, ADAM17 has a role in shedding of L-selectin, a cellular adhesion molecule.

Recently, ADAM17 was discovered as a crucial mediator of resistance formation to radiotherapy. It was also shown that radiotherapy activates ADAM17 in non-small cell lung cancer, which results in shedding of multiple survival factors, growth factor pathway activation, and radiotherapy-induced treatment resistance.

Since ADAM17 seems to be a crucial factor for the release of different pathogenic and non-pathogenic factors, including TNFα, it has come into the focus as therapeutic target molecule. For that reason, different attempts have been made to develop inhibitors of ADAM17.

However, so far, no such inhibitor has proven clinically successful.

It is hence one object of the present invention to provide a new approach which allows the control, regulation, reduction or inhibition of ADAM17 activity.

It is another object of the present invention to provide a new approach that allows the treatment of inflammatory diseases.

These and other objects are solved by the features of the independent claims. The dependent claims disclose embodiments of the invention which may be preferred under particular circumstances. Likewise, the specification discloses further embodiments of the invention which may be preferred under particular circumstances.

### SUMMARY OF THE INVENTION

The present invention provides, among others, a protein binder that binds to human iRhom2, and inhibits and/or reduces TACE/ADAM17 activity when bound to human iRhom2.

### BRIEF DESCRIPTION OF THE FIGURES

FIG. 1 provides an overview of the target sequences encoded by the expression vectors that were used for DNA immunization of iRhom2 knockout mice.

FIG. 2 shows results from TNFα release assays (shedding assays) for functional screening of hybridoma supernatants, demonstrating that the supernatant of the hybridoma cell pool 14C2 (the primary material leading to the antibody 3 of the invention) as a representative example of selected candidates effectively interferes with LPS-induced shedding of TNFα in THP-1 cells.

FIG. 3 depicts results from fluorescence activated cell sorting (FACS) analyses on genetically engineered murine L929 cell populations, demonstrating that two variants of human iRhom2-a T7-tagged deletion mutant lacking amino acids 1-242 (A242) and a T7-tagged full length (FL) wild type (WT) form—ectopically expressed by L929-2041-hiR2-Δ242-T7 and L929-2041-hiR2-FL-WT-T7 cells, respectively, are localized on the surface of these cells. Stainings: gray=secondary antibody only; black=anti-T7-antibody

FIG. 4 shows results from FACS analyses for target recognition-based screening of hybridoma supernatants, demonstrating that the supernatant of the hybridoma cell pool 14C2 (the primary material leading to the antibody 3 of the invention) as a representative example of selected candidates clearly recognizes both human iRhom2 variants ectopically expressed by L929-2041-hiR2-Δ242-T7 and L929-2041-hiR2-FL-WT-T7 cells. Stainings: gray=secondary antibody only; black=14C2 supernatant

FIG. 5 depicts results from ELISA analyses for antibody isotype determination, demonstrating the purified antibodies 3, 5, 16, 22, 34, 42, 43, 44, 46, 47, 48, 49, 50, 51, 52, 54, 56, and 57 of the invention to be of mouse IgG isotype.

FIG. 6 provides results from FACS scatchard analyses for antibody affinity determination, demonstrating that the K_{D} values for binding of the purified antibodies 3, 5, 16, 22, 34, 42, 43, 44, 48, and 50 to THP-1 cells are in the subnanomolar to low nanomolar range.

FIG. 7a depicts results from FACS analyses on genetically engineered mouse embryonic fibroblast (MEF) populations, demonstrating that T7-tagged variants of human and mouse iRhom2 full length wild type ectopically expressed by MEF-DKO-hiR2-FL-WT-T7 (SEQ ID NO 190) and MEF-DKO-miR2-FL-WT-T7 SEQ ID NO 193) cells, respectively, are localized on the surface of these cells. Stainings: gray=secondary antibody only; black=anti-T7-antibody

FIG. 7b shows results from FACS analyses for the determination of mouse cross-reactivity of the antibodies of the invention, demonstrating that the purified antibody 3 as a representative example of the antibodies of the invention (except for antibody 52) clearly recognizes the human iRhom2 variant ectopically expressed by MEF-DKO-hiR2-FL-WT-T7, but not the mouse iRhom2 variant ectopically expressed by MEF-DKO-miR2-FL-WT-T7 cells and, thus, is not cross-reactive with mouse iRhom2. Stainings: gray=secondary antibody only; black=antibody 3

FIG. 8a depicts results from FACS analyses on genetically engineered MEF populations, demonstrating that also a T7-tagged version of human iRhom1 full length wild type ectopically expressed by MEF-DKO-hiR1-FL-WT-T7 cells is localized on the surface of these cells. Stainings: gray=secondary antibody only; black=anti-T7-antibody

FIG. 8b shows results from FACS analyses for the determination of specificity of the antibodies of the invention, demonstrating that the purified antibody 3 as a representative example of the antibodies of the invention—in contrast to the human iRhom2 variant ectopically expressed by MEF-DKO-hiR2-FL-WT-T7 cells—does not recognize the closely related human iRhom1 variant ectopically expressed by MEF-DKO-hiR1-FL-WT-T7 cells and, thus, is specific for human iRhom2. Stainings: gray=secondary antibody only; black=antibody 3

FIG. 9a shows results from TNFα release assays, demonstrating the purified antibodies 3, 5, 16, 22, 34, 42, 43, and 44 of the invention to interfere with LPS-induced shedding of TNFα in THP-1 cells, whereas the purified antibodies 48 and 50 have no inhibitory effect on TNFα release. The data illustrate the effects of test articles in absolute numbers of released TNFα. The analyzed antibodies were purified from hybridoma supernatants.

FIG. 9b refers to the results depicted in FIG. 9a and illustrates the effects of test articles on TNFα release in percent inhibition.

FIG. 10a depicts results from FACS analyses on one of the MEF populations with mouse iRhom2-related single amino acid substitutions or the deletion that were genetically engineered for epitope determination. The data demonstrate that—similarly to the T7-tagged variants of human and mouse iRhom2 full length wild type ectopically expressed by MEF-DKO-hiR2-FL-WT-T7 and MEF-DKO-miR2-FL-WT-T7 cells, respectively—also the T7-tagged human iRhom2 variant hiR2-FL-P533—(deletion of P533) ectopically expressed by MEF-DKO-hiR2-FL-P533-T7 cells is localized on the surface of these cells. Stainings: gray=secondary antibody only; black=anti-T7-antibody

FIG. 10b shows results from TGFα release assays (shedding assays), demonstrating that all human iRhom2 variants with mouse iRhom2-related single amino acid substitutions including the single amino acid deletion hiR2-FL-P533—are functionally active and can support PMA-stimulated shedding of TGFα to varying degrees, indicating that these variants are most likely properly folded.

FIG. 11a depicts results from FACS analyses for epitope determination of the antibodies of the invention. Exemplarily for the entire panel of 25 human iRhom2 variants with mouse iRhom2-related single amino acid substitutions or the deletion, data for the analysis of MEF-DKO-hiR2-FL-P533-T7 cells ectopically expressing the human iRhom2 variant hiR2-FL-P533—are shown. The data demonstrate that the deletion of the single amino acid proline 533 in human iRhom2 strongly impairs and, thus, contributes to binding of the purified antibody 3 as a representative example of the antibodies of the invention with inhibitory effects on TNFα release. In contrast, this deletion does not affect and, thus, does not contribute to binding of the purified antibody 50 as a representative example of the antibodies of the invention without inhibitory effects on TNFα release. Stainings: gray=secondary antibody only; black=antibody 3/antibody 50

FIG. 11b summarizes the results of FACS analyses of all purified antibodies of the invention on the entire panel of 25 engineered MEF populations ectopically expressing human iRhom2 variants with mouse iRhom2-related single amino acid substitutions (including the deletion variant hiR2-FL-P533-). The data reveal related (except for antibody 16) patterns of amino acid positions relevant for iRhom2 binding of the antibodies 3, 5, 16, 22, 34, 42, 43, and 44 of the invention with inhibitory effects on TNFα release, which are different from patterns of amino acid positions contributing to binding of the antibodies 48 and 50 without inhibitory effects on TNFα release.

FIG. 12a depicts results from FACS analyses on one of the MEF populations with human iRhom1-related single amino acid substitutions or deletions within the central region of the large extracellular loop (AA498 to AA562 of human iRhom2) that were genetically engineered for epitope determination. The data demonstrate that—similarly to the T7-tagged variants of human iRhom2 and iRhom1 full length wild type ectopically expressed by MEF-DKO-hiR2-FL-WT-T7 and MEF-DKO-hiR1-FL-WT-T7 cells, respectively—also the T7-tagged human iRhom2 variant hiR2-FL-L539A ectopically expressed by MEF-DKO-hiR2-FL-L539A-T7 cells is localized on the surface of these cells. Stainings: gray=secondary antibody only; black =anti-T7-antibody

FIG. 12b shows results from TGFα release assays (shedding assays), demonstrating that all 30 human iRhom2 variants with human iRhom1-related single amino acid substitutions or single amino acid deletions within the central region of the large extracellular loop (AA498 to AA562 of human iRhom2) as in the case of hiR2-FL-M534-, hiR2-FL-D535—and hiR2-FL-K536—are functionally active and can support PMA-stimulated shedding of TGFα to varying degrees, indicating that these variants are most likely properly folded.

FIG. 13a depicts results from FACS analyses for epitope determination of the antibodies of the invention. Exemplarily for the entire panel of 30 human iRhom2 variants with human iRhom1-related single amino acid substitutions or deletions within the central region of the large extracellular loop (AA498 to AA562 of human iRhom2), data for the analysis of MEF-DKO-hiR2-FL-L539A-T7 cells ectopically expressing the human iRhom2 variant hiR2-FL-L539A are shown. The data demonstrate that the substitution of the single amino acid leucine 539 in human iRhom2 by alanine at the corresponding position in human iRhom1 strongly impairs and, thus, contributes to binding of the purified antibody 3 as a representative example of the antibodies of the invention with inhibitory effects on TNFα release. In contrast, this substitution does not affect and, thus, does not contribute to binding of the purified antibody 50 as a representative example of the antibodies of the invention without inhibitory effects on TNFα release. Stainings: gray=secondary antibody only; black=antibody 3/antibody 50

FIG. 13b summarizes the results of FACS analyses of all purified antibodies of the invention on the entire panel of 30 engineered MEF populations ectopically expressing human iRhom2 variants with human iRhom1-related single amino acid substitutions within the central region of the large extracellular loop (AA498 to AA562 of human iRhom2), including the deletion variants hiR2-FL-M534-, hiR2-FL-D535-, and hiR2-FL-K536-. The data again reveal related patterns of amino acid positions relevant for iRhom2 binding of the antibodies 3, 5, 16, 22, 34, 42, 43, and 44 of the invention with inhibitory effects on TNFα release, which are different from patterns of amino acid positions contributing to binding of the antibodies 48 and 50 of the invention without inhibitory effects on TNFα release.

FIG. 14a shows results from TNFα release assays, demonstrating the antibodies 3, 5, 16, 22, 34, 42, 43, 44, 46, 49, 54, 56, and 57 of the invention to interfere with LPS-induced shedding of TNFα in THP-1 cells, whereas the antibodies 47, 48, 50, 51, and 52 have no inhibitory effect on TNFα release. The data illustrate the effects of test articles in absolute numbers of released TNFα. The analyzed antibodies result from transient expression of the respective 18 heavy chain/kappa light chain pairs in Expi293F cells.

FIG. 14b refers to the results depicted in FIG. 14a and illustrates the effects of test articles on TNFα release in percent inhibition.

FIGS. 15a and 15b show a schematic representation of iRhom2 with the positions of the juxtamembrane domain (JMD) (A) adjacent to the transmembrane domain 1 (TMD1), loop 1 (B) and the C-terminus (C) being illustrated. The table in FIG. 15b shows the amino acid positions as set forth in SEQ ID NO 181.

FIG. 16 depicts the amino acid sequence of human iRhom2 according to SEQ ID NO 181, with the preferred binding regions marked.

FIG. 17a shows an alignment of human iRhom2 (>NP_078875.4 human iRhom2 isoform 1) according to SEQ ID NO 181 and human iRhom1 (>NP_071895.3 human iRhom1) according to SEQ ID NO 182.

FIG. 17b shows an alignment of human iRhom2 (>NP_078875.4 human iRhom2 isoform 1) according to SEQ ID NO 181 and mouse iRhom2 (>NP_766160 mouse iRhom2) according to SEQ ID NO 183.

FIG. 18a shows results from FACS analyses for the determination of the cross-reactivity of the antibodies of the invention with rhesus monkey, demonstrating that both the murine (upper panel) and the chimeric (lower panel) versions of antibody 16 as a representative example of antibodies 3, 5, 16, 22, 34, 42, 43, 44, 46, 47, 48, 49, 50, 51, 54, 56 and 57 of the invention clearly recognizes the rhesus monkey iRhom2 variant (UniProt Identifier: F6Y4X6) ectopically expressed by MEF-DKO-Rhesus-iR2-FL-WT-T7, but not the rhesus monkey iRhom1 variant (UniProt Identifier: F6ZPC8) ectopically expressed by MEF-DKO- Rhesus-iR1-FL-WT-T7 cells and, thus, is cross-reactive with rhesus monkey iRhom2 but does not bind to rhesus monkey iRhom1. The analyzed antibodies result from transient expression of the respective heavy chain/kappa light chain pairs in Expi293F (for the murine versions) or CHO (for the chimeric versions) cells. Stainings: gray=secondary antibody only; black=antibody FIG. 18b shows results from FACS analyses for the determination of the cross-reactivity of the antibodies of the invention with cynomolgus monkey, demonstrating that both the murine (upper panel) and the chimeric (lower panel) versions of antibody 16 as a representative example of antibodies 3, 5, 16, 22, 34, 42, 43, 44, 46, 47, 48, 49, 50, 51, 54, 56 and 57 of the invention clearly recognizes the cynomolgus monkey iRhom2 variant (UniProt Identifier: AOA2K5TX07) ectopically expressed by MEF-DKO-Cyno-iR2-FL-WT-T7, but not the cynomolgus monkey iRhom1 variant (UniProt Identifier: AOA2K5TUM2) ectopically expressed by MEF-DKO-Cyno-iR1-FL-WT-T7 cells and, thus, is cross-reactive with cynomolgus monkey iRhom2 but does not bind to cynomolgus monkey iRhom1. The analyzed antibodies result from transient expression of the respective heavy chain/kappa light chain pairs in Expi293F (for the murine versions) or CHO (for the chimeric versions) cells. Stainings: gray=secondary antibody only; black=antibody 16

FIG. 18c shows results from FACS analyses for the determination of the cross-reactivity of the antibodies of the invention with dog, demonstrating that both the murine (upper panel) and the chimeric (lower panel) versions of antibody 16 as a representative example of antibodies 3, 5, 16, 22, 34, 42, 43, 44, 46, 47, 48, 49, 50, 51, 54, 56 and 57 of the invention clearly recognizes the dog iRhom2 variant (UniProt Identifier: Q00M95) ectopically expressed by MEF-DKO-Dog-iR2-FL-WT-T7, but not the dog iRhom1 variant (UniProt Identifier: AOA5F4CNN3) ectopically expressed by MEF-DKO-Dog-iR1-FL-WT-T7 cells and, thus, is cross-reactive with dog iRhom2 but does not bind to dog iRhom1. The analyzed antibodies result from transient expression of the respective heavy chain/kappa light chain pairs in Expi293F (for the murine versions) or CHO (for the chimeric versions) cells. Stainings: gray =secondary antibody only; black=antibody 16

FIG. 18d shows results from FACS analyses for the determination of the cross-reactivity of the antibodies of the invention with rabbit, demonstrating that both the murine (upper panel) and the chimeric (lower panel) versions of antibody 16 as a representative example of antibodies 3, 5, 16, 22, 34, 42, 43, 44, 49, 51, 54 and 56 of the invention clearly recognizes the rabbit iRhom2 variant (UniProt Identifier: G1T7M2) ectopically expressed by MEF-DKO-Rabbit-iR2-FL-WT-T7, but not the rabbit iRhom1 variant (UniProt Identifier: B8K128) ectopically expressed by MEF-DKO-Rabbit-iR1-FL-WT-T7 cells and, thus, is cross-reactive with rabbit iRhom2 but does not bind to rabbit iRhom1. The analyzed antibodies result from transient expression of the respective heavy chain/kappa light chain pairs in Expi293F (for the murine versions) or CHO (for the chimeric versions) cells. Stainings: gray=secondary antibody only; black=antibody 16.

FIG. 19a depicts results from FACS analyses on genetically engineered mouse embryonic fibroblast (MEF) populations, demonstrating that FLAG-tagged variants of human and mouse iRhom2 full length wild type ectopically expressed by MEF-DKO-hiR2-FL-WT-FLAG (SEQ ID NO 198) and MEF-DKO-miR2-FL-WT-FLAG (SEQ ID NO 199) cells, respectively, are localized on the surface of these cells. Stainings: gray=secondary antibody only; black=anti-FLAG-antibody

FIG. 19b shows results from FACS analyses for the determination of mouse cross-reactivity of the antibodies of the invention, demonstrating that the murine antibody 3 as a representative example of the antibodies of the invention, except for antibody 52, clearly recognizes the human iRhom2 variant ectopically expressed by MEF-DKO-hiR2-FL-WT-FLAG, but not the mouse iRhom2 variant ectopically expressed by MEF-DKO-miR2-FL-WT-FLAG cells and, thus, is not cross-reactive with mouse iRhom2. The analyzed murine antibodies result from transient expression of the respective heavy chain/kappa light chain pairs in Expi293F cells. Stainings: gray=secondary antibody only; black=antibody 3

FIG. 20a shows results from FACS analyses for the determination of specificity of the antibodies of the invention, demonstrating that the primary material leading to the antibody 16 of the invention as a representative example of antibodies 3, 16, 22 and 42 of the invention binds to RPMI-8226 (left panel) and THP-1 cells (middle panel), both of which express iRhom2 endogenously, but does not bind to RH-30 cells (right panel), which do not express iRhom2 endogenously and, thus, is specifically recognizing endogenous human iRhom2. Stainings: gray=secondary antibody only; black=supernatant leading to antibody 16

FIG. 20b shows results from FACS analyses for the determination of specificity of the antibodies of the invention, demonstrating that both the murine (upper panel) and the chimeric (lower panel) versions of the antibody 16 as a representative example of antibodies 16, 22 and 42 of the invention bind to RPMI-8226 (left panel) and THP-1 cells (middle panel), both of which express iRhom2 endogenously, but do not bind to RH-30 cells (right panel), which do not express iRhom2 endogenously and, thus, are specifically recognizing endogenous human iRhom2. The analyzed antibodies result from transient expression of the respective heavy chain/kappa light chain pairs in Expi293F (for the murine versions) or CHO (for the chimeric versions) cells. Stainings: gray=secondary antibody only; black=antibody 16

FIG. 21a depicts results from FACS analyses on one of the MEF populations with human iRhom1-related single amino acid substitutions N-terminal of the central region of the large extracellular loop (AA431 to AA496 of human iRhom2) that were genetically engineered for epitope determination. The data demonstrate that—similar to the T7-tagged variant of human iRhom2 full length wild type ectopically expressed by MEF-DKO-hiR2-FL-WT-T7 cells—the T7-tagged human iRhom2 variant hiR2-FL-S448N ectopically expressed by MEF-DKO-hiR2-FL-S448N-T7 cells is also localized on the surface of these cells. Stainings: gray=secondary antibody only; black=anti-T7-antibody

FIG. 21b shows results from TGFα release assays (shedding assays), demonstrating that all 23 human iRhom2 variants with human iRhom1-related single amino acid substitutions N-terminal of the central region of the large extracellular loop (AA431 to AA496 of human iRhom2) are functionally active and can support PMA-stimulated shedding of TGFα to varying degrees, indicating that these variants are most likely properly folded.

FIG. 22a depicts results from FACS analyses for epitope determination of the antibodies of the invention. Exemplarily for the entire panel of 23 human iRhom2 variants with human iRhom1-related single amino acid substitutions N-terminal of the central region of the large extracellular loop (AA431 to AA496 of human iRhom2), data for the analysis of MEF-DKO-hiR2-FL-S448N-T7 cells ectopically expressing the human iRhom2 variant hiR2-FL-S448N are shown. The data demonstrate that the substitution of the single amino acid serine 448 in human iRhom2 by asparagine at the corresponding position in human iRhom1 does not affect and, thus, does not contribute to binding of both antibody 5 as a representative example of the antibodies of the invention with inhibitory effects on TNFα release and antibody 50 as a representative example of the antibodies of the invention without inhibitory effects on TNFα release. Stainings: gray=secondary antibody only; black=antibody 5/antibody 50, respectively FIG. 22b summarizes the results of FACS analyses of all antibodies of the invention on the entire panel of 23 engineered MEF populations ectopically expressing human iRhom2 variants with human iRhom1-related single amino acid substitutions N-terminal of the central region of the large extracellular loop (AA431 to AA496 of human iRhom2). The data reveal no amino acid positions relevant for iRhom2 binding of the antibodies 3, 5, 16, 22, 34, 42, 43, and 44 of the invention with inhibitory effects on TNFα release. In contrast, some of them contribute to the binding of the antibodies 48 and 50 without inhibitory effects on TNFα release.

FIG. 23a depicts results from FACS analyses on one of the MEF populations with human iRhom1-related single amino acid substitutions C-terminal of the central region of the large extracellular loop (AA563 to AA638 of human iRhom2), in loop5 (AA771 of human iRhom2) or in the C-terminus (AA825 to AA844 of human iRhom2) that were genetically engineered for epitope determination. The data demonstrate that—similar to the T7-tagged variant of human iRhom2 full length wild type ectopically expressed by MEF-DKO-hiR2-FL-WT-T7 cells the T7-tagged human iRhom2 variant hiR2-FL-1566E ectopically expressed by MEF-DKO-hiR2-FL-I566E-T7 cells is also localized on the surface of these cells. Stainings: gray=secondary antibody only; black=anti-T7-antibody

FIG. 23b shows results from TGFα release assays (shedding assays), demonstrating that all 33 human iRhom2 variants with human iRhom1-related single amino acid substitutions C-terminal of the central region of the large extracellular loop (AA563 to AA638 of human iRhom2), in loop5 (AA771 of human iRhom2) or in the C-terminus (AA825 to AA844 of human iRhom2) are functionally active and can support PMA-stimulated shedding of TGFα to varying degrees, indicating that these variants are most likely properly folded.

FIG. 24a depicts results from FACS analyses for epitope determination of the antibodies of the invention. Exemplarily for the entire panel of 33 human iRhom2 variants with human iRhom1-related single amino acid substitutions C-terminal of the central region of the large extracellular loop (AA563 to AA638 of human iRhom2), in loop5 (AA771 of human iRhom2) or in the C-terminus (AA825 to AA844 of human iRhom2), data for the analysis of MEF-DKO-hiR2-FL-I566E-T7 cells ectopically expressing the human iRhom2 variant hiR2-FL-I566E are shown. The data demonstrate that the substitution of the single amino acid isoleucine 566 in human iRhom2 by glutamic acid at the corresponding position in human iRhom1 strongly impairs and, thus, contributes to binding of antibody 5 as a representative example of the antibodies of the invention with inhibitory effects on TNFα release. In contrast, this substitution does not affect and, thus, does not contribute to binding of antibody 50 as a representative example of the antibodies of the invention without inhibitory effects on TNFα release. Stainings: gray=secondary antibody only; black=antibody 5/antibody 50, respectively

FIG. 24b summarizes the results of FACS analyses of all antibodies of the invention on the entire panel of 33 engineered MEF populations ectopically expressing human iRhom2 variants with human iRhom1-related single amino acid substitutions C-terminal of the central region of the large extracellular loop (AA563 to AA638 of human iRhom2), in loop5 (AA771 of human iRhom2) or in the C-terminus (AA825 to AA844 of human iRhom2). The data again reveal related patterns of amino acid positions relevant for iRhom2 binding of the antibodies 3, 5, 22, 34, 42, 43, and 44 of the invention with inhibitory effects on TNFα release, which are different from patterns of amino acid positions contributing to binding of the antibodies 48 and 50 without inhibitory effects on TNFα release.

FIG. 25a depicts results from FACS analyses on one of the MEF populations with alanine single amino acid substitutions within the central region of the large extracellular loop (AA503 to AA593 of human iRhom2) that were genetically engineered for epitope determination. The data demonstrate that—similarly to the T7-tagged variant of human iRhom2 full length wild type ectopically expressed by MEF-DKO-hiR2-FL-WT-T7 cells the T7-tagged human iRhom2 variant hiR2-FL-K536A ectopically expressed by MEF-DKO-hiR2-FL-K536A-T7 cells is also localized on the surface of these cells. Stainings: gray=secondary antibody only; black=anti-T7-antibody

FIG. 25b shows results from TGFα release assays (shedding assays), demonstrating that all 91 human iRhom2 variants with single amino acid substitutions to alanine within the central region of the large extracellular loop (AA503 to AA593 of human iRhom2), except for the human iRhom2 variants hiR2-FL-C516A, hiR2-FL-F523A, hiR2-FL-C549A, hiR2-FL-D552A, hiR2-FL-C556A, hiR2-FL-W567A, hiR2-FL-W574A and hiR2-FL-C577A, are functionally active and can support PMA-stimulated shedding of TGFα to varying degrees, indicating that these variants are most likely properly folded.

FIG. 26a depicts results from FACS analyses for epitope determination of the antibodies of the invention. Exemplary for the entire panel of 83 functional human iRhom2 variants with single amino acid substitutions to alanine within the central region of the large extracellular loop (AA503 to AA593 of human iRhom2), data for the analysis of MEF-DKO-hiR2-FL-K536A-T7 cells ectopically expressing the human iRhom2 variant hiR2-FL-K536A are shown. The data demonstrate that the substitution of the single amino acid leucine 536 in human iRhom2 by alanine strongly impairs and, thus, contributes to binding of antibody 5 as a representative example of the antibodies of the invention with inhibitory effects on TNFα release. In contrast, this substitution does not affect and, thus, does not contribute to binding of antibody 50 as a representative example of the antibodies of the invention without inhibitory effects on TNFα release. Stainings: gray=secondary antibody only; black=antibody 5 /antibody 50, respectively

FIG. 26b summarizes the results of FACS analyses of all antibodies of the invention on the entire panel of 83 engineered functional MEF populations ectopically expressing human iRhom2 variants with single amino acid substitutions to alanine within the central region of the large extracellular loop (AA503 to AA593 of human iRhom2). The data again reveal related patterns of amino acid positions relevant for iRhom2 binding of the antibodies 3, 5, 16, 22, 34, 42, 43, and 44 of the invention with inhibitory effects on TNFα release, which are different from patterns of amino acid positions contributing to binding of the antibodies 48 and 50 without inhibitory effects on TNFα release.

FIG. 27a shows results from TNFα release assays, demonstrating the antibodies 3, 5, 16, 22, 34, 42, 43 and 44 of the invention to interfere with PMA-induced shedding of TNFα in U937 cells. The data illustrate the effects of test articles in absolute numbers of released TNFα. The analyzed murine antibodies result from transient expression of the respective heavy chain/kappa light chain pairs in Expi293F cells.

FIG. 27b refers to the results depicted in FIG. 27a and illustrates the effects of test articles on TNFα release in percent inhibition.

FIG. 28a shows results from TNFα release assays, demonstrating both the murine (indicated with m followed by the antibody number) and the chimeric (indicated with ch followed by the antibody number) version of the antibodies 16, 22, 34, 42, and 44 of the invention to interfere with PMA-induced shedding of TNFα in U937 cells. The data illustrate the effects of test articles in absolute numbers of released TNFα. The analyzed antibodies result from transient expression of the respective heavy chain/kappa light chain pairs in Expi293F (for the murine versions) or CHO (for the chimeric versions) cells.

FIG. 28b refers to the results depicted in FIG. 28a and illustrates the effects of test articles on TNFα release in percent inhibition.

FIG. 29a shows results from IL-6R release assays, demonstrating the antibodies 3, 5, 16, 22, 34, 42, 43 and 44 of the invention to interfere with PMA-induced shedding of IL-6R in THP-1 cells. The data illustrate the effects of test articles in absolute numbers of released IL-6R. The analyzed murine antibodies result from transient expression of the respective heavy chain/kappa light chain pairs in Expi293F cells.

FIG. 29b refers to the results depicted in FIG. 29c and illustrates the effects of test articles on IL-6R release in percent inhibition.

FIG. 30a shows results from IL-6R release assays, demonstrating the antibodies 3, 5, 16, 22, 34, 42, 43 and 44 of the invention to interfere with PMA-induced shedding of IL-6R in U937 cells. The data illustrate the effects of test articles in absolute numbers of released IL-6R. The analyzed murine antibodies result from transient expression of the respective heavy chain/kappa light chain pairs in Expi293F cells.

FIG. 30b refers to the results depicted in FIG. 30a and illustrates the effects of test articles on IL-6R release in percent inhibition.

FIG. 31a shows results from IL-6R release assays, demonstrating both the murine (indicated with m followed by the antibody number) and the chimeric (indicated with ch followed by the antibody number) version of the antibodies 16, 22, 34, 42, and 44 of the invention to interfere with PMA-induced shedding of IL-6R in U937 cells. The data illustrate the effects of test articles in absolute numbers of released IL-6R. The analyzed antibodies result from transient expression of the respective heavy chain/kappa light chain pairs in Expi293F (for the murine versions) or CHO (for the chimeric versions) cells.

FIG. 31b refers to the results depicted in FIG. 31a and illustrates the effects of test articles on IL-6R release in percent inhibition.

FIG. 32a shows results from HB-EGF release assays, demonstrating both the murine (indicated with m followed by the antibody number) and the chimeric (indicated with ch followed by the antibody number) version of the antibodies 16, 22, 34, 42, and 44 of the invention to interfere with PMA-induced shedding of HB-EGF in THP-1 cells. The data illustrate the effects of test articles in absolute numbers of released HB-EGF. The analyzed antibodies result from transient expression of the respective heavy chain/kappa light chain pairs in Expi293F (for the murine versions) or CHO (for the chimeric versions) cells.

FIG. 32b refers to the results depicted in FIG. 32a and illustrates the effects of test articles on HB-EGF release in percent inhibition.

FIG. 33a shows results from HB-EGF release assays, demonstrating both the murine (indicated with m followed by the antibody number) and the chimeric (indicated with ch followed by the antibody number) version of the antibodies 16, 22, 34, 42, and 44 of the invention to interfere with PMA-induced shedding of HB-EGF in U937 cells. The data illustrate the effects of test articles in absolute numbers of released HB-EGF. The analyzed antibodies result from transient expression of the respective heavy chain/kappa light chain pairs in Expi293F (for the murine versions) or CHO (for the chimeric versions) cells.

FIG. 33b refers to the results depicted in FIG. 33a and illustrates the effects of test articles on HB-EGF release in percent inhibition.

FIG. 34a shows results from TGFα release assays, demonstrating the antibodies 16, 22, 42, 43 and 44 of the invention to weakly interfere with PMA-induced shedding of TGFα in PC3 cells, whereas the antibodies 3, 5 and 34 have no inhibitory effect on TGFα release. The data illustrate the effects of test articles in absolute numbers of released TGFα. The analyzed murine antibodies result from transient expression of the respective heavy chain/kappa light chain pairs in Expi293F cells.

FIG. 34b refers to the results depicted in FIG. 34a and illustrates the effects of test articles on TGFα release in percent inhibition.

FIG. 35a shows results from TNFα release assays, demonstrating the antibodies 16, 22 and 42 of the invention to interfere with LPS-induced shedding of TNFα in human peripheral blood mononuclear cells (PBMCs) isolated from healthy donors. The data illustrate the effects of test articles in absolute numbers of released TNFα. The analyzed murine antibodies result from transient expression of the respective heavy chain/kappa light chain pairs in Expi293F cells.

FIG. 35b refers to the results depicted in FIG. 35a and illustrates the effects of test articles on TNFα release in percent inhibition.

FIG. 36a shows results from TNFα release assays, demonstrating the antibodies 16, 22 and 42 of the invention to interfere with LPS-induced shedding of TNFα in human macrophages isolated from peripheral blood mononuclear cells (PBMCs) of healthy donors. The data illustrate the effects of test articles in absolute numbers of released TNFα. The analyzed murine antibodies result from transient expression of the respective heavy chain/kappa light chain pairs in Expi293F cells.

FIG. 36b refers to the results depicted in FIG. 36a and illustrates the effects of test articles on TNFα release in percent inhibition.

FIG. 37a shows results from IL-6R release assays, demonstrating the antibodies 16, 22 and 42 of the invention to interfere with PMA-induced shedding of IL-6R in human peripheral blood mononuclear cells (PBMCs) isolated from healthy donors. The data illustrate the effects of test articles in absolute numbers of released IL-6R. The analyzed murine antibodies result from transient expression of the respective heavy chain/kappa light chain pairs in Expi293F cells.

FIG. 37b refers to the results depicted in FIG. 37a and illustrates the effects of test articles on IL-6R release in percent inhibition.

FIG. 38a shows results from HB-EGF release assays, demonstrating the antibodies 16, 22 and 42 of the invention to interfere with PMA-induced shedding of HB-EGF in human peripheral blood mononuclear cells (PBMCs) isolated from healthy donors. The data illustrate the effects of test articles in absolute numbers of released HB-EGF. The analyzed murine antibodies result from transient expression of the respective heavy chain/kappa light chain pairs in Expi293F cells.

FIG. 38b refers to the results depicted in FIG. 38a and illustrates the effects of test articles on HB-EGF release in percent inhibition.

FIG. 39a shows results from in vivo septic shock models in humanized hsNOG-EXL mice (human cd34+), demonstrating that the antibodies 16, 22 and 42 of the invention interfere with LPS-induced shedding of TNFα in humanized hsNOG-EXL mice. The data illustrate the effects of test articles in absolute numbers of released TNFα. The analyzed murine antibodies result from transient expression of the respective heavy chain/kappa light chain pairs in Expi293F cells.

FIG. 39b refers to the results depicted in FIG. 39a and illustrates the effects of test articles on TNFα release in percent compared to the buffer treated control animals, which were set to 100%.

FIG. 40a shows results from TNFα release assays, demonstrating that the antibodies 16, 22, 34, 42 and 44 of the invention interfere with LPS-induced shedding of TNFα in human peripheral blood mononuclear cells (PBMCs) isolated from patients suffering from rheumatoid arthritis. The data illustrate the effects of test articles in absolute numbers of released TNFα. The analyzed chimeric antibodies result from transient expression of the respective heavy chain/kappa light chain pairs in CHO cells.

FIG. 40b refers to the results depicted in FIG. 40a and illustrates the effects of test articles on TNFα release in percent inhibition.

FIG. 41a shows results from IL-6R release assays, demonstrating that the antibodies 16, 22, 34, 42 and 44 of the invention interfere with PMA-induced shedding of IL-6R in human peripheral blood mononuclear cells (PBMCs) isolated from patients suffering from rheumatoid arthritis. The data illustrate the effects of test articles in absolute numbers of released IL-6R. The analyzed chimeric antibodies result from transient expression of the respective heavy chain/kappa light chain pairs in CHO cells.

FIG. 41b refers to the results depicted in FIG. 41a and illustrates the effects of test articles on IL-6R release in percent inhibition.

FIG. 42a shows results from HB-EGF release assays, demonstrating that the antibodies 16, 22, 34, 42 and 44 of the invention interfere with PMA-induced shedding of HB-EGF in human peripheral blood mononuclear cells (PBMCs) isolated from patients suffering from rheumatoid arthritis. The data illustrate the effects of test articles in absolute numbers of released HB-EGF. The analyzed chimeric antibodies result from transient expression of the respective heavy chain/kappa light chain pairs in CHO cells.

FIG. 42b refers to the results depicted in FIG. 42a and illustrates the effects of test articles on HB-EGF release in percent inhibition.

### DETAILED DESCRIPTION

According to one aspect of the invention, a protein binder is provided which, when bound to human iRhom2, binds at least within a region of Loop 1 thereof.

According to another aspect of the invention, a protein binder is provided which binds to the extracellular domain of human iRhom2, which protein binder is an IgG antibody.

Inactive Rhomboid family member 2 (iRhom2) is a protein that in humans is encoded by the RHBDF2 gene. It is a transmembrane protein consisting of about 850 amino acids, having seven transmembrane domains. The inventors of the present invention have for the first time demonstrated that iRhom2 can act as a target for protein binders to inhibit TACE/ADAM17 activity. iRhom2 comes in different isoforms. The experiments made herein have been established with the isoform defined as NCBI reference NP_078875.4. However, the teachings are transferable, without limitation, to other isoforms of iRhom2, as shown in the following table:

| mRNA | protein | name |
|---|---|---|
| NM_024599.5 | NP_078875.4 | inactive rhomboid protein 2 |
| | | transcript variant 1/isoform 1 |
| NM_001005498.3 | NP_001005498.2 | inactive rhomboid protein 2 |
| | | transcript variant 2/isoform 2 |

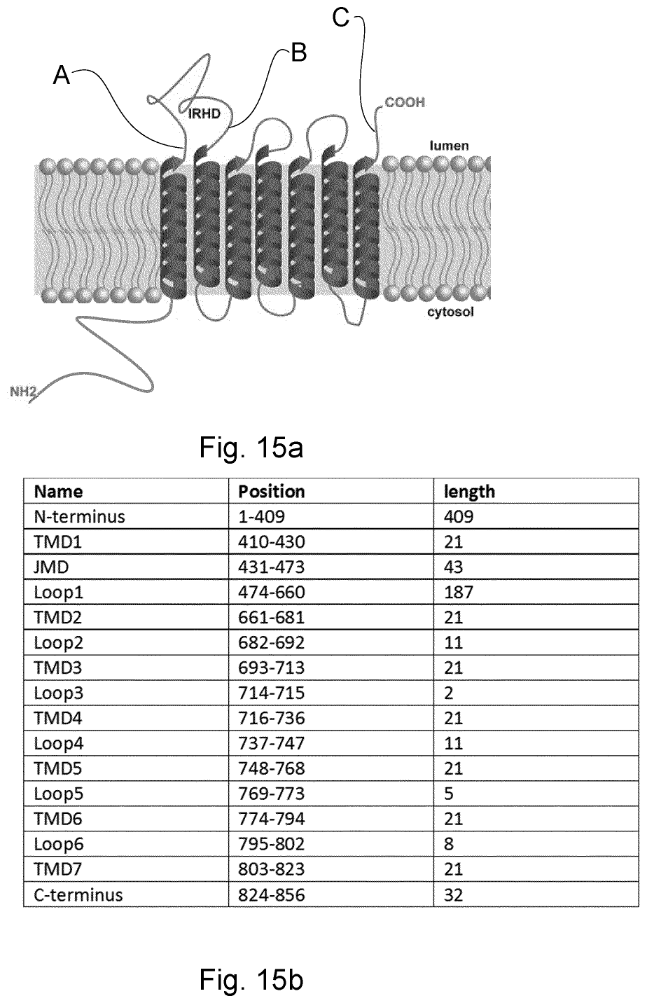

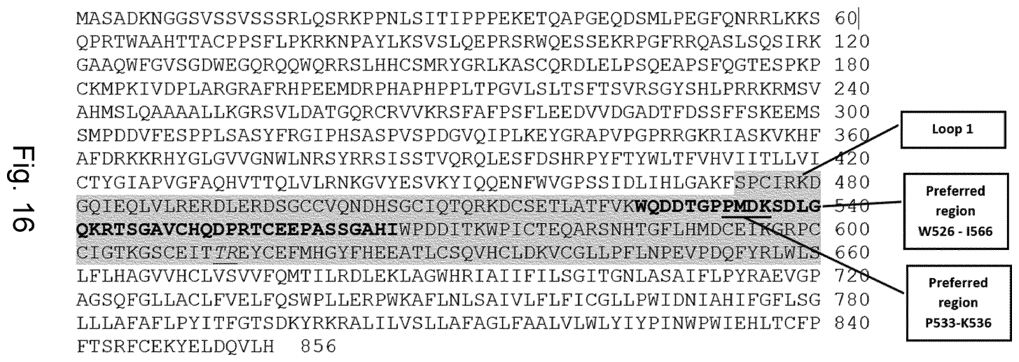

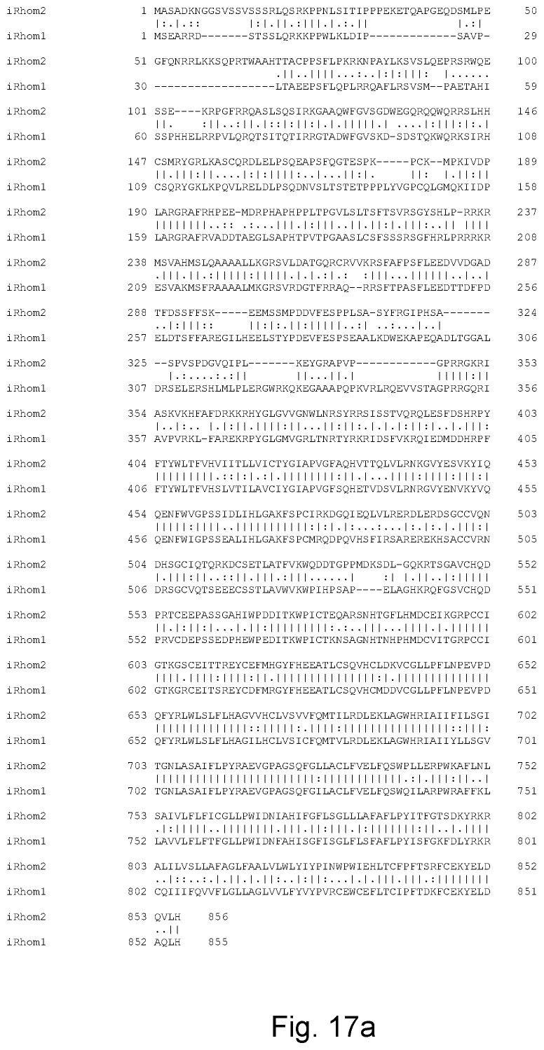

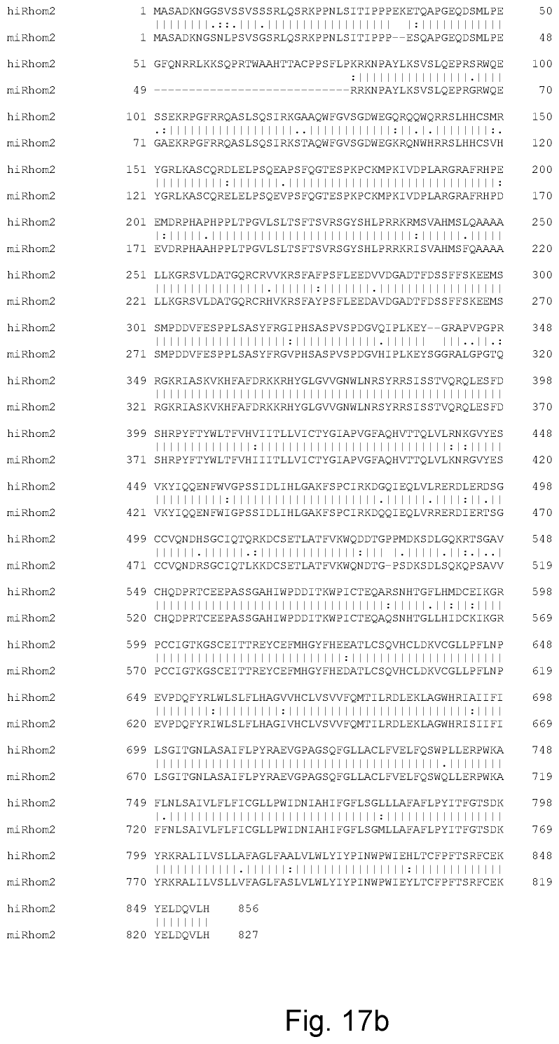

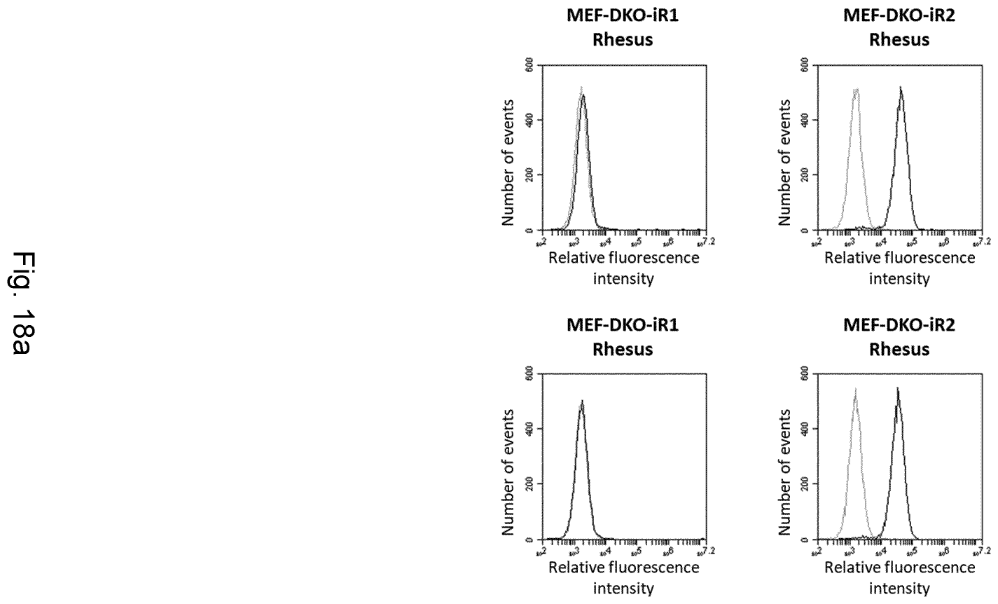

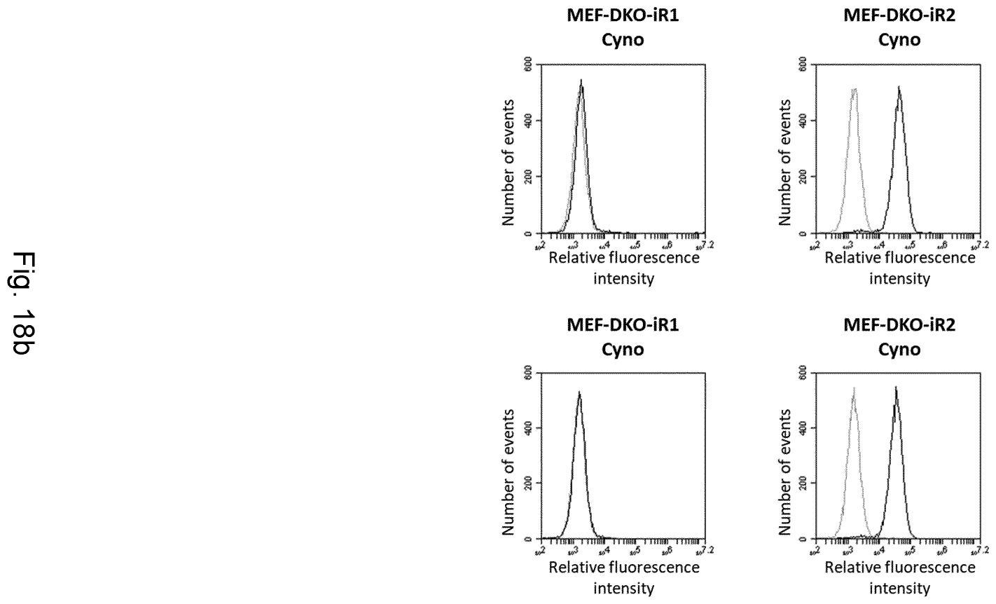

Loop 1 of Rhom2 comprises amino acid residues 474-660 of SEQ ID NO 181. See item “B” in FIG. 15 for an explanation.

In one embodiment, of the invention, the protein binder binds also to one or more other regions of human iRhom2, like e.g. the juxtamembrane domain (JMD) located N-terminally of Loop 1 (shown as item “A” in FIG. 15), or to a region near the C.-terminus. The JMD comprises amino acid residues 431-473 of SEQ ID NO 181.

In another embodiment, the protein binder does not bind to the juxtamembrane domain (JMD) located on the N-terminal side of Loop 1.

According to one embodiment of the invention, the protein binder binds within at least a region of human iRhom2 spanning from (and including) W526 to (and including) 1566, according to the numbering set forth in SEQ ID NO 181.

Preferably, the protein binder binds within a region which has at least 3 amino acids in length.

In one or more embodiments. the protein binder binds to ≥2, ≥3, ≥4, ≥5, ≥6, ≥7, ≥8, ≥9, ≥10, ≥11, ≥12, 13, ≥14, ≥15, ≥16, ≥17, ≥18, ≥19, ≥20, ≥21, ≥22, ≥23, ≥24, or ≥25 amino acids within the above region. The respective amino acid residues can be present in a discrete, consecutive sequence, or in two or more clusters, each of which comprising one or more amino acid residues.

Preferably, the protein binder binds within a region of human iRhom2 spanning from (and including) P533 to (and including) K536, according to the numbering set forth in SEQ ID NO 181.

According to one embodiment of the invention, the protein binder binds a stretch of human iRhom2 comprising at least one residue selected from the group comprising W526; Q527; P532; P533; M534; D535; K536; S537; L539; K542; R543; T544; G546; R554; E557; S561; and/or 1566, according to the numbering set forth in SEQ ID NO 181.

In one or more embodiments, the protein binder binds to ≥2, ≥3, ≥4, ≥5, ≥6, ≥7, ≥8, ≥9, ≥10, ≥11, or ≥12 amino acid residues from the above list. The respective amino acid residues can be present in a discrete, consecutive sequence, or in two or more clusters, each of which comprising one or more amino acid residues.

Preferably, the protein binder binds a stretch of iRhom2 comprising at least one residue selected from the group comprising P533; M534; D535; K536; and/or L539, according to the numbering set forth in SEQ ID NO 181.

In one or more embodiments. the protein binder binds to ≥2, ≥3 or ≥4 amino acid residues from the above list. The respective amino acid residues can be present in a discrete, consecutive sequence, or in two or more clusters, each of which comprising one or more amino acid residues.

According to one embodiment of the invention, the protein binder inhibits and/or reduces TACE/ADAM17 activity when bound to human iRhom2.

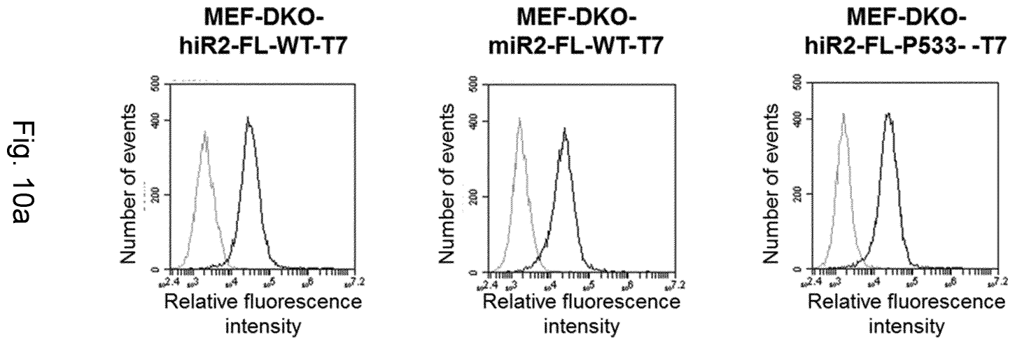

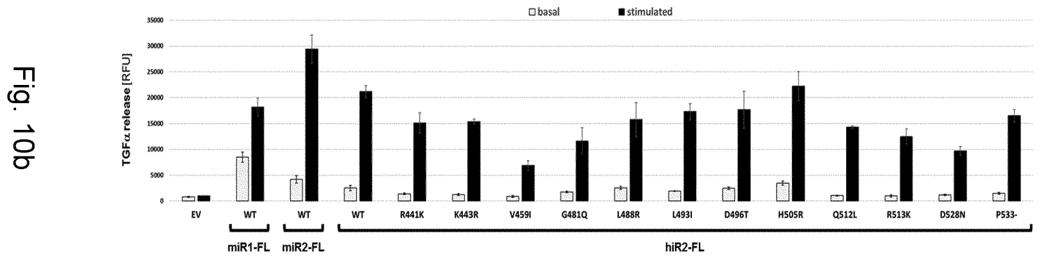

As used herein, the term “inhibits and/or reduces TACE/ADAM17 activity is meant to describe an effect caused by a protein binder that blocks or reduces the activity of TACE/ADAM17, as measured e.g. in a respective shedding assay (see., e.g., FIG. 9 and example 14).

ADAM metallopeptidase domain 17 (ADAM17), also called TACE (tumor necrosis factor-α-converting enzyme), is an enzyme that belongs to the ADAM protein family of disintegrins and metalloproteases. ADAM17 is understood to be involved in the processing of tumor necrosis factor alpha (TNF-α) at the surface of the cell, and from within the intracellular membranes of the trans-Golgi network. This process, which is also known as ‘shedding’, involves the cleavage and release of α soluble ectodomain from membrane-bound pro-proteins (such as pro-TNF-α), and is of known physiological importance. ADAM17 was the first ‘sheddase’ to be identified, and it is also understood to play a role in the release of a diverse variety of membrane-anchored cytokines, cell adhesion molecules, receptors, ligands, and enzymes.

Cloning of the TNF-α gene revealed it to encode a 26 kDa type II transmembrane pro-polypeptide that becomes inserted into the cell membrane during its maturation. At the cell surface, pro-TNF-α is biologically active, and is able to induce immune responses via juxtacrine intercellular signaling. However, pro-TNF-α can undergo a proteolytic cleavage at its Ala76-Val77 amide bond, which releases α soluble 17 kDa extracellular domain (ectodomain) from the pro-TNF-α molecule. This soluble ectodomain is the cytokine commonly known as TNF-α, which is of pivotal importance in paracrine signaling. This proteolytic liberation of soluble TNF-α is catalyzed by ADAM17.

Recently, ADAM17 was discovered as a crucial mediator of resistance to radiotherapy. It was also shown that radiotherapy activates ADAM17 in non-small cell lung cancer, which results in shedding of multiple survival factors, growth factor pathway activation, and radiotherapy-induced treatment resistance.

ADAM17 also regulates the MAP kinase signaling pathway by regulating shedding of the EGFR ligand amphiregulin in the mammary gland. ADAM17 also has a role in the shedding of L-selectin, a cellular adhesion molecule.

According to one embodiment of the invention, the inhibition or reduction of TACE/ADAM17 activity is caused by interference of the protein binder with iRhom2-mediated TACE/ADAM17 activation or TACE/ADAM17 interaction with other proteins including substrate molecules.

According to one embodiment of the invention, the protein binder, when bound to human iRhom2, inhibits or reduces induced TNFα shedding.

According to one embodiment of the invention, the protein binder, when bound to human iRhom2, inhibits or reduces induced IL-6R shedding.

According to one embodiment of the invention, the protein binder, when bound to human iRhom2, inhibits or reduces induced HB-EGF shedding.

Tumor necrosis factor alpha (TNFα) shedding or release, as used herein, refers to a process in which membrane-anchored tumor necrosis factor alpha (mTNFα/pro-TNFα) upon cleavage is released into the environment to become soluble TNFα (sTNFα or simply TNFα). This process is, inter alia, triggered by TACE/ADAM17.

Release or shedding of Interleukin 6 receptor (IL-6R) refers to a process in which soluble IL-6R is produced by proteolytic cleavage of the membrane-bound IL-6R on the cell surface at a proteolytic site close to its transmembrane domain by TACE/ADAM17 Release or shedding of Heparin-binding EGF-like growth factor (HB-EGF) refers to a cleavage process in which the soluble form of HB-EGF is generated and set free from the cell surface. Heparin-binding EGF-like growth factor, an epidermal growth factor with an affinity for heparin, is synthesized as a membrane-anchored mitogenic and chemotactic glycoprotein. . First identified in the conditioned media of human macrophage-like cells, HB-EGF is an 87-amino acid glycoprotein that displays highly regulated gene expression.

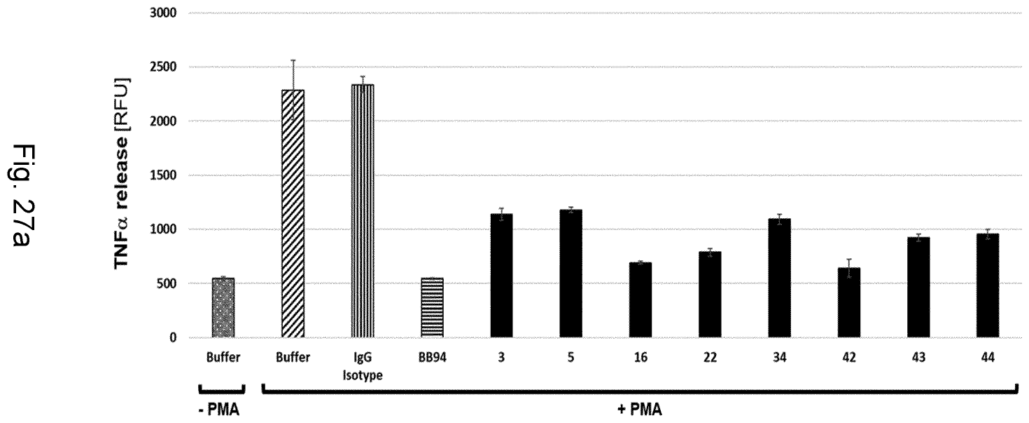

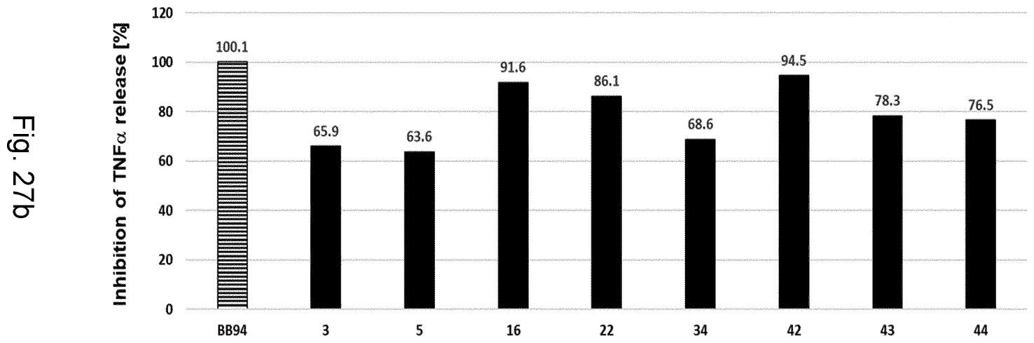

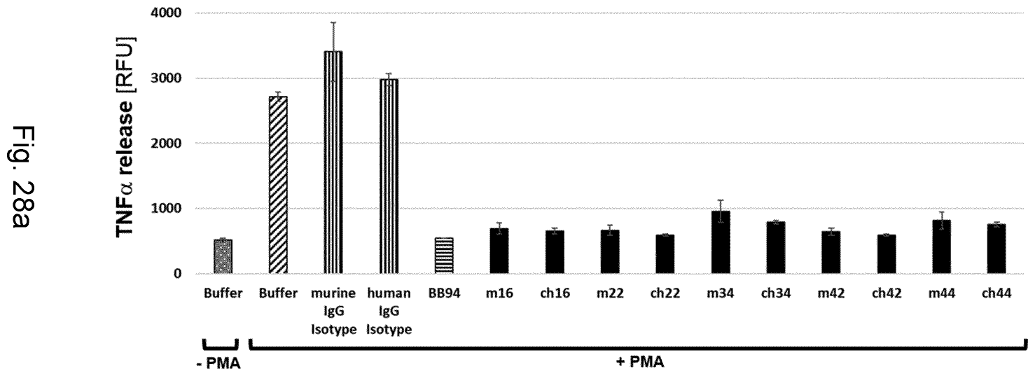

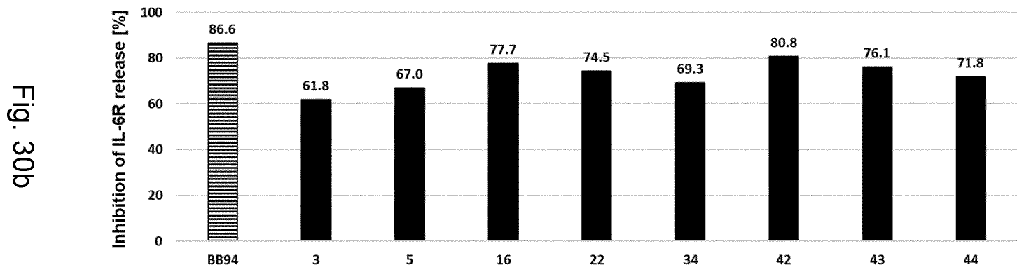

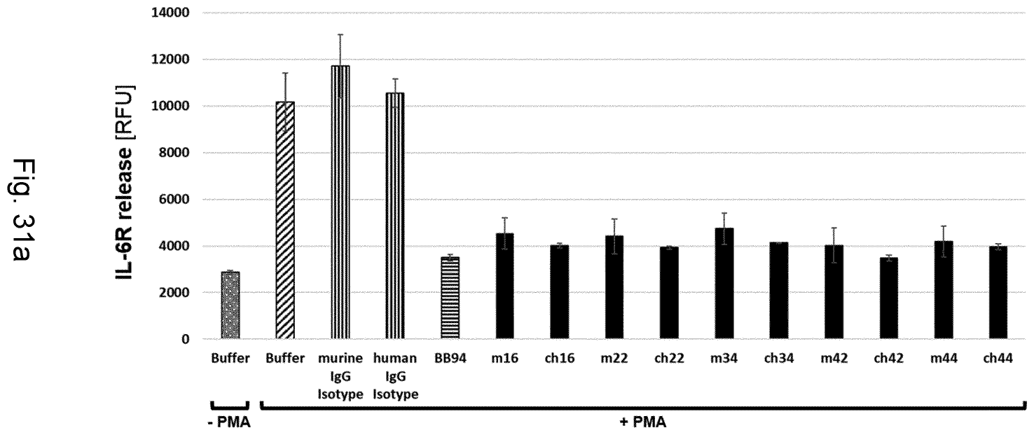

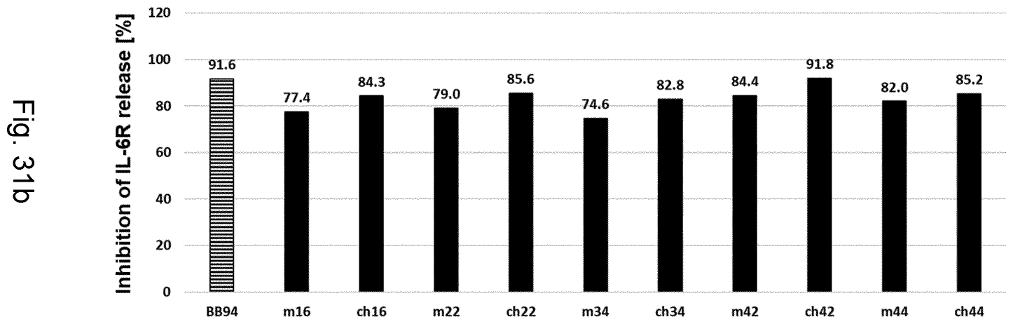

Suitable Assays to determine the TNFα shedding effect are described, e.g., in FIG. 9 and example 14. Suitable Assays to determine the release or shedding of IL-6R and/or HB-EGF are described, e.g., in FIG. 29 and example 26 or in FIG. 32 and example 29, respectively.

According to one embodiment of the invention, the human iRhom2 to which the protein binder binds comprises
a) the amino acid sequence set forth in SEQ ID NO 181, or
b) an amino acid sequence that has at least 80% sequence identity with SEQ ID NO 181, with the proviso that said sequence maintains iRhom2 activity.

In some embodiments, human iRhom2 comprises an amino acid sequence that has ≥81%, preferably 82%, more preferably 83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98 or most preferably ≥99% sequence identity with SEQ ID NO 181.

SEQ ID NO 181 represents the amino acid sequence of inactive rhomboid protein 2 (iRhom2) isoform 1 [Homo sapiens], accessible under NCBI reference NP_078875.4. Generally, different variants and isoforms of iRhom2 exist. Likewise, mutants comprising conservative or silent amino acid substitutions exist, or may exist, which maintain full or at least substantial iRhom2 activity. These isoforms, variants and mutants are encompassed by the identity range specified above, meaning however that dysfunctional, non-active variants and mutants are excluded.

According to one embodiment of the invention, the protein binder is a monoclonal antibody, or a target-binding fragment or derivative thereof retaining target binding capacities, or an antibody mimetic.

As used herein, the term “monoclonal antibody (mAb)” shall refer to an antibody composition having a homogenous antibody population, i.e., a homogeneous population consisting of a whole immunoglobulin, or a fragment or derivative thereof retaining target binding capacities.

Particularly preferred, such antibody is an IgG antibody, or a fragment or derivative thereof retaining target binding capacities. Immunoglobulin G (IgG) is a type of antibody. Representing approximately 75% of serum antibodies in humans, IgG is the most common type of antibody found in blood circulation. IgG molecules are created and released by plasma B cells. Each IgG has two antigen binding sites.

IgG antibodies are large molecules with a molecular weight of about 150 kDa made of four peptide chains. It contains two identical class 7 heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. The Fc regions of IgGs bear a highly conserved N-glycosylation site. The N-glycans attached to this site are predominantly core-fucosylated diantennary structures of the complex type. In addition, small amounts of these N-glycans also bear bisecting GlcNAc and α-2,6-linked sialic acid residues.

There are four IgG subclasses (IgG1, 2, 3, and 4) in humans, named in order of their abundance in serum (IgG1 being the most abundant).

As used herein, the term “fragment” shall refer to fragments of such antibody retaining target binding capacities, e.g.
a CDR (complementarity determining region)
a hypervariable region,
a variable domain (Fv)
an IgG or IgM heavy chain (consisting of VH, CH1, hinge, CH2 and CH3 regions)
an IgG or IgM light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs, and further retaining target binding capacities. All these items are explained below.

Other antibody derivatives known to the skilled person are Diabodies, Camelid Antibodies, Nanobodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerized constructs comprising CH3+VL+VH, and antibody conjugates (e.g. antibody or fragments or derivatives linked to a toxin, a cytokine, a radioisotope or a label). These types are well described in the literature and can be used by the skilled person on the basis of the present disclosure, without adding further inventive activity.

Methods for the production of a hybridoma cell are disclosed in Köhler & Milstein (1975).

Methods for the production and/or selection of chimeric or humanised mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies. Methods for the production and/or selection of fully human mAbs are known in the art. These can involve the use of a transgenic animal which is immunized with the respective protein or peptide, or the use of a suitable display technique, like yeast display, phage display, B-cell display or ribosome display, where antibodies from a library are screened against human iRhom2 in a stationary phase.

In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by TaconicArtemis.

IgG, IgM, scFv, Fab and/or F(ab)₂ are antibody formats well known to the skilled person. Related enabling techniques are available from the respective textbooks.

As used herein, the term “Fab” relates to an IgG/IgM fragment comprising the antigen binding region, said fragment being composed of one constant and one variable domain from each heavy and light chain of the antibody

As used herein, the term “F(ab)₂” relates to an IgG/IgM fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

Modified antibody formats are for example bi- or trispecific antibody constructs, antibody-based fusion proteins, immunoconjugates and the like. These types are well described in the literature and can be used by the skilled person on the basis of the present disclosure, with adding further inventive activity.

As used herein, the term “antibody mimetic” relates to an organic molecule, most often a protein that specifically binds to a target protein, similar to an antibody, but is not structurally related to antibodies. Antibody mimetics are usually artificial peptides or proteins with a molar mass of about 3 to 20 kDa. The definition encompasses, inter alia, Affibody molecules, Affilins, Affimers, Affitins, Alphabodies, Anticalins, Avimers, DARPins, Fynomers, Kunitz domain peptides, Monobodies, and nanoCLAMPs.

In one or more embodiments, the protein binder is an isolated antibody, or a target-binding fragment or derivative thereof retaining target binding capacities, or an isolated antibody mimetic

In one or more embodiments, the antibody is an engineered or recombinant antibody, or a target binding fragment or derivative thereof retaining target binding capacities, or an engineered or recombinant antibody mimetic.

According to one embodiment of the invention, the protein binder is an antibody in at least one of the formats selected from the group consisting of: IgG, scFv, Fab, or (Fab)2.

According to one embodiment of the invention, the protein binder is not cross-reactive with human iRhom1.

According to one embodiment of the invention, the protein binder is a murine, chimerized, humanized, or human antibody.

According to one embodiment of the invention, the protein binder is an antibody that
a) comprises a set of heavy chain/light chain complementarity determining regions (CDR) comprised in the heavy chain/light variable domain sequence pair set forth in the following pairs of SEQ ID NOs: 2and 7; 12and 17;22and 27; 32and 37;42and 47; 52and 57;62and 67;72and 77; 82 and 87; 112 and 117; 152 and 157; 162 and 167; and/or 172 and 177;
b) comprises a set of heavy chain/light chain complementarity determining regions (CDR) comprising the following SEQ ID NOs, in the order (HCDR1; HCDR2; HCDR3; LCDR1; LCDR2 and LCDR3)
   3, 4, 5, 8, 9, 10;
   13, 14, 15, 18, 19, 20;
   23,24,25,28,29,30;
   33,34,35,38,39,40;
   43,44,45,48,49,50;
   53,54,55,58,59,60;
   63,64,65,68,69,70;
   73,74,75,78,79,80;
   83,84,85,88,89,90;
   113,114,115,118,119,120;
   153, 154, 155, 158, 159, 160;
   163, 164, 165, 168, 169, 170; and/or
   173, 174, 175, 178, 179, 180;
c) comprises the heavy chain/light chain complementarity determining regions (CDR) of b), with the proviso that at least one of the CDRs has up to 3 amino acid substitutions relative to the respective SEQ ID NOs, and/or
d) comprises the heavy chain/light chain complementarity determining regions (CDR) of b) or c), with the proviso that at least one of the CDRs has a sequence identity of ≥66% to the respective SEQ ID NOs,

wherein the CDRs are embedded in a suitable protein framework so as to be capable to bind to human iRhom2 with sufficient binding affinity and to inhibit or reduce TACE/ADAM17 activity.

As used herein, the term “CDR” or “complementarity determining region” is intended to mean the non-contiguous antigen combining sites found within the variable region of both heavy and light chain polypeptides. These particular regions have been described by Kabat et al. (1977), Kabat et al. (1991), Chothia et al. (1987) and MacCallum et al., (1996) where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. Note that this numbering may differ from the CDRs that are actually disclosed in the enclosed sequence listing, because CDR definitions vary from case to case.

TABLE 1
CDR definitions

| | Kabat | Chothia | MacCallum |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the term “framework” when used in reference to an antibody variable region is entered to mean all amino acid residues outside the CDR regions within the variable region of an antibody. Therefore, a variable region framework is between about 100-120 amino acids in length but is intended to reference only those amino acids outside of the CDRs.

As used herein, the term “capable to bind to target X with sufficient binding affinity” has to be understood as meaning that respective binding domain binds the target with a K_{D} of 10-4 or smaller. K_{D} is the equilibrium dissociation constant, a ratio of k_{off}/kₒₙ, between the protein binder and its antigen. K_{D} and affinity are inversely related. The K_{D} value relates to the concentration of protein binder (the amount of protein binder needed for a particular experiment) and so the lower the K_{D} value (lower concentration) and thus the higher the affinity of the binding domain. The following table shows typical K_{D} ranges of monoclonal antibodies

TABLE 2
K_{D} and Molar Values

| K_{D} value | Molar range |
|---|---|
| 10⁻⁴ to 10⁻⁶ | Micromolar (μM) |
| 10⁻⁷ to 10⁻⁹ | Nanomolar (nM) |
| 10⁻¹⁰ to 10⁻¹² | Picomolar (pM) |
| 10⁻¹³ to 10⁻¹⁵ | Femtomolar (fM) |

Preferably, the protein binder has up to 2 amino acid substitutions, and more preferably up to 1 amino acid substitution.

Preferably, at least one of the CDRs of the protein binder has a sequence identity of ≥67%; ≥68%; ≥69%; ≥70%; ≥71%; ≥72%; ≥73%; ≥74%; ≥75%; ≥76%; ≥77%; ≥78%; ≥79%; ≥80%; ≥81%; ≥82%; ≥83%; ≥84%; ≥85%; ≥86%; ≥87%; ≥88%; ≥89%; ≥90%;≥91%;≥92%;≥93%;≥94%;≥95%;≥96%;≥97%;≥98%;≥99%, and most preferably 100% to the respective SEQ ID NO.

“Percentage of sequence identity” as used herein, is determined by comparing two optimally aligned biosequences (amino acid sequences or polynucleotide sequences) over a comparison window, wherein the portion of the corresponding sequence in the comparison window may comprise additions or deletions (i.e., gaps) as compared to the reference sequence, which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides that are substantially identical to the polypeptides exemplified herein. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

Preferably, at least one of the CDRs has been subject to CDR sequence modification, including
affinity maturation
reduction of immunogenicity

Affinity maturation in the process by which the affinity of a given antibody is increased in vitro. Like the natural counterpart, in vitro affinity maturation is based on the principles of mutation and selection. It has successfully been used to optimize antibodies, antibody fragments or other peptide molecules like antibody mimetics. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range. For principles see Eylenstein et al. (2016) or US20050169925A1, the content of which is incorporated herein by reference for enablement purposes.

Engineered antibodies contain murine-sequence derived CDR regions that have been engrafted, along with any necessary framework back-mutations, into sequence-derived V regions. Hence, the CDRs themselves can cause immunogenic reactions when the humanized antibody is administered to a patient. Methods of reducing immunogenicity caused by CDRs are disclosed in Harding et al. (2010), or US2014227251A1, the content of which is incorporated herein by reference for enablement purposes.

According to one embodiment of the invention, the protein binder is an antibody that
a) the heavy chain/light chain variable domain (HCVD/LCVD) pairs set forth in the following pairs of SEQ ID NOs:

2and 7; 12and 17;22 and 27;32 and 37;42and 47;52 and 57; 62and 67;72 and 77; 82 and 87; 112 and 117; 152 and 157; 162 and 167; and/or 172 and 177;
b) the heavy chain/light chain variable domains (HCVD/LCVD) pairs of α), with the proviso that
   the HCVD has a sequence identity of ≥80% to the respective SEQ ID NO, and/or
   the LCVD has a sequence identity of ≥80% to the respective SEQ ID NO,
c) the heavy chain/light chain variable domains (VD) pairs of α) or b), with the proviso that at least one of the HCVD or LCVD has up to 10 amino acid substitutions relative to the respective SEQ ID NO,

said protein binder still being capable to bind to human iRhom2 with sufficient binding affinity and to inhibit or reduce TACE/ADAM17 activity.

A “variable domain” when used in reference to an antibody or a heavy or light chain thereof is intended to mean the portion of an antibody which confers antigen binding onto the molecule and which is not the constant region. The term is intended to include functional fragments thereof which maintain some of all of the binding function of the whole variable region.

Variable region binding fragments include, for example, functional fragments such as Fab, F(ab)₂, Fv, single chain Fv (scfv) and the like. Such functional fragments are well known to those skilled in the art. Accordingly, the use of these terms in describing functional fragments of a heteromeric variable region is intended to correspond to the definitions well known to those skilled in the art. Such terms are described in, for example, Huston et al., (1993) or Pluckthun and Skerra (1990).

Preferably, the HCVD and/or LCVD has a sequence identity of 81%; ≥82%; ≥83%; ≥84%; ≥85% 86%≥87%≥88%≥89%≥90%≥91%≥92%≥93%≥94%≥95%; ≥96%; ≥97%; ≥98%; ≥99%; or most preferably 100% to the respective SEQ ID NO.

According to one embodiment of the invention, at least one amino acid substitution is a conservative amino acid substitution.

A “conservative amino acid substitution”, as used herein, has a smaller effect on antibody function than a non-conservative substitution. Although there are many ways to classify amino acids, they are often sorted into six main groups on the basis of their structure and the general chemical characteristics of their R groups.

In some embodiments, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and
aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Conservative changes can further include substitution of chemically homologous non-natural amino acids (i.e. a synthetic non-natural hydrophobic amino acid in place of leucine, a synthetic non-natural aromatic amino acid in place of tryptophan).

According to one embodiment of the invention, the protein binder has at least one of
target binding affinity of ≥50% to human iRhom2 compared to that of the protein binder according to any one of the aforementioned claims, and/or
≥50% of the inhibiting or reducing effect on TACE/ADAM17 activity of the protein binder according to any one of the aforementioned claims.

As used herein the term “binding affinity” is intended to mean the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction. For example, a bivalent heteromeric variable region binding fragment can exhibit altered or optimized binding affinity due to its valency.

A suitable method for measuring the affinity of a binding agent is through surface plasmon resonance (SPR). This method is based on the phenomenon which occurs when surface plasmon waves are excited at a metal/liquid interface. Light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously and therefore allows for determination of the individual components of an affinity constant. More specifically, the method enables accurate measurements of association rates (k ₒₙ) and disassociation rates (k_{off}).

Measurements of k ₒₙ and k_{off} values can be advantageous because they can identify altered variable regions or optimized variable regions that are therapeutically more efficacious. For example, an altered variable region, or heteromeric binding fragment thereof, can be more efficacious because it has, for example, a higher kₒₙ valued compared to variable regions and heteromeric binding fragments that exhibit similar binding affinity. Increased efficacy is conferred because molecules with higher kₒₙ values can specifically bind and inhibit their target at a faster rate. Similarly, a molecule of the invention can be more efficacious because it exhibits a lower k_{off} value compared to molecules having similar binding affinity. Increased efficacy observed with molecules having lower k_{off}rates can be observed because, once bound, the molecules are slower to dissociate from their target. Although described with reference to the altered variable regions and optimized variable regions of the invention including, heteromeric variable region binding fragments thereof, the methods described above for measuring associating and disassociation rates are applicable to essentially any protein binder or fragment thereof for identifying more effective binders for therapeutic or diagnostic purposes.

Another suitable method for measuring the affinity of a binding agent is through surface is by FACS/scatchard analysis. See inter alia example 10 for a respective description.

Methods for measuring the affinity, including association and disassociation rates using surface plasmon resonance are well known in the arts and can be found described in, for example, Jonsson and Malmquist, (1992) and Wu et al. (1998). Moreover, one apparatus well known in the art for measuring binding interactions is a BIAcore 2000 instrument which is commercially available through Pharmacia Biosensor, (Uppsala, Sweden).

Preferably said target binding affinity is ≥51%, ≥52%, ≥53%, ≥54%, ≥55%, ≥56%, ≥57%, 58%, ≥59%, ≥60%, ≥61%, ≥62%, ≥63%, ≥64%, ≥65%, ≥66%, ≥67%, ≥68%, ≥69%, 70%, ≥71%, ≥72%, ≥73%, ≥74%, ≥75%, ≥76%, ≥77%, ≥78%, ≥79%, ≥80%, ≥81%, 82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, and most preferably ≥99% compared to that of the reference binding agent.

As used herein, the quantification of the inhibiting or reducing effect on TACE/ADAM17 activity, compared to a benchmark binding agent, is determined with a suitable assay to determine the TNFα shedding effect, as, e.g., described, e.g., in FIG. 9 and example 14.

According to another aspect of the invention, a protein binder is provided that binds to human iRhom2, and competes for binding to human iRhom2 with
a) an antibody according to the above description, and/or
b) an antibody selected from the group consisting of clones #3, #5, #16, #22, #34, #42, #43, #44, #46, #49, #54, #56, or #57

According to another aspect of the invention, a protein binder is provided that binds to essentially the same, or the same, region on human iRhom2 as
a) an antibody according to the above description, and/or
b) an antibody selected from the group consisting of clones #3, #5, #16, #22, #34, #42, #43, #44, #46, #49, #54, #56, or #57.

Clones #3, #5, #16, #22, #34, #42, #43, #44, #46, #47, #48, #49, #50, #51, #52, #54, #56, or #57 are identified in the sequence table herein.

As regards the format or structure of such protein binder, the same preferred embodiments as set forth above apply. In one embodiment, said protein binder is a monoclonal antibody, or a target-binding fragment or derivative thereof retaining target binding capacities, or an antibody mimetic.

As used herein, the term “competes for binding” is used in reference to one of the antibodies defined by the sequences as above, meaning that the actual protein binder as an activity which binds to the same target, or target epitope or domain or subdomain, as does said sequence defined protein binder, and is a variant of the latter. The efficiency (e.g., kinetics or thermodynamics) of binding may be the same as or greater than or less than the efficiency of the latter. For example, the equilibrium binding constant for binding to the substrate may be different for the two antibodies.

Such competition for binding can be suitably measured with a competitive binding assay. Such assays are disclosed in Finco et al. 2011, the content of which is incorporated herein by reference for enablement purposes, and their meaning for interpretation of a patent claim is disclosed in Deng et al 2018, the content of which is incorporated herein by reference for enablement purposes.

In order to test for this characteristic, suitable epitope mapping technologies are available, including, inter alia,
X-ray co-crystallography and cryogenic electron microscopy (cryo-EM)
Array-based oligo-peptide scanning
Site-directed mutagenesis mapping
High-throughput shotgun mutagenesis epitope mapping
Hydrogen-deuterium exchange
Cross-linking-coupled mass spectrometry

These methods are, inter alia, disclosed and discussed in Banik et al (2010), and DeLisser (1999), the content of which is herein incorporated by reference for enablement purposes.

According to another aspect of the invention, a nucleic acid is provided that encodes for at least one chain of the binding agent according to the above description.

In one embodiment, at least acids are provided which encode for the heavy chain and the light chain, respectively, of the binding agent, in case the later is a monoclonal antibody having a heteromeric stricture of at least one light chain and one heavy chain.

Generally, due to the degeneracy of the genetic code, there is a large number of different nucleic acids that have the capacity to encode for such chain. The skilled person is perfectly able to determine if a given nucleic acid satisfies the above criterion. On the other hand, the skilled person is perfectly able to reverse engineer, from a given amino acid sequence, based on codon usage tables, a suitable nucleic acid encoding therefore. For this purpose, software tools such as “reverse translate” provided by the online tool “sequence manipulation suite”, (https://www.bioinfonnatics.org/sms2/rev_trans.html) can be used.

Such nucleic acid can be also be used for pharmaceutic purposes. In such case, it is an RNA-derived molecule that is administered to a patient, wherein the protein expression machinery of the patient expresses the respective binding agent. The mRNA can for example be delivered in suitable liposomes and comprises either specific sequences or modified uridine nucleosides to avoid immune responses and/or improve folding and translation efficiency, sometimes comprising cap modifications at the 5′—and/or 3′ terminus to target them to specific cell types. Such nucleic acid can be used for transfecting an expression host to then express the actual binding agent. In such case, the molecule can be a cDNA that is optionally integrated into a suitable vector.

According to another aspect of the invention, the use of the protein binder or nucleic acid according to the above description is provided (for the manufacture of a medicament) in the treatment of a human or animal subject
being diagnosed for,
suffering from or
being at risk of developing

an inflammatory condition, or for the prevention of such condition.

In order to diagnose am inflammatory condition, the patient may have a physical exam and may also be asked about medical history. A practitioner may look for inflammation in the joints, joint stiffness and loss of function in the joint. In addition, the practitioner may order X-rays and/or Blood tests to detect inflammatory markers, like e.g. serum hs-CRP, IL-6, TNF-α, and IL-10, erythrocyte sedimentation rate, plasma viscosity, fibrinogen, and/or ferritin, as compared to healthy controls

According to another aspect of the invention, a pharmaceutical composition comprising the protein binder or nucleic acid according to the above description, and optionally one or more pharmaceutically acceptable excipients, is provided.

According to another aspect of the invention, a combination comprising (i) the protein binder or the nucleic acid or the pharmaceutical composition according to the above description and (ii) one or more therapeutically active compounds is provided.

According to another aspect of the invention, a method for treating or preventing an inflammatory condition is provided, which method comprises administration, to a human or animal subject, of (i) the protein binder according to the above description (ii) the nucleic acid according to the above description, (iii) the pharmaceutical composition according to the above description, or (iv) the combination according to the above description is provided in a therapeutically sufficient dose.

According to one embodiment of the invention, the inflammatory condition is Rheumatoid Arthritis (RA) According to another aspect of the invention, a therapeutic kit of parts comprising:
a) the protein binder according to the above description, the nucleic acid according to the above description, the pharmaceutical composition according to the above description, or the combination according to the above description
b) an apparatus for administering the composition, composition or combination, and
c) instructions for use.

is provided.

Further embodiments of the present invention relate to antibodies 47, 48, 50, 51 and 52, which the inventors have shown to have specific effects, as shown in the following table (+ means inhibitory effect,—means no inhibitory effect)

| | CDR sequences | | Inhibitory effect on the release of | | |
|---|---|---|---|---|---|
| | (HCDR1-3/ | Variable domain | LPS-induced | PMA-induced | PMA-induced |
| Antibody | LCDR1-3) | sequences (HC/LC) | TNFα | IL-6R | HB-EGF |
| 47 | 93-95/98-100 | 92/97 | − | + | + |
| 48 | 103-105/108-110 | 102/107 | − | + | + |
| 50 | 123-125/128-130 | 122/127 | − | + | + |
| 51 | 133-135/138-140 | 132/137 | − | + | + |
| 52 | 143-145/148-150 | 142/147 | − | − | − |

The table above compares the properties of the antibodies 47, 48, 50, 51 and 52 of the invention on LPS-induced shedding of TNFα versus PMA-induced shedding of IL-6R and HB-EGF in THP-1 cells. In contrast to the LPS-induced release of TNFα in THP-1 cells, where the antibodies 47, 48, 50, 51 and 52 of the invention had no inhibitory effect, an inhibitory effect on the release of PMA-induced IL-6R and HB-EGF in THP-1 cells was observed for the antibodies 47, 48, 50, and 51 of the invention, but not for the antibody 52 of the invention.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′-≥3′.

### Example 1: Generation of Expression Vectors for Immunization

In total 8 different expression vectors were generated for immunization, 3 of them coding for different human iRhom2 and the remaining five coding for different mouse iRhom2 variants.

Gene synthesis was performed at Thermo Fisher Scientific GeneArt GmbH, Regensburg, Germany. In brief, submitted DNA sequences were optimized using GeneOptimizer software for maximum protein production. After genes were synthesized using synthetic oligonucleotides, assembled by primer extension-based PCR, constructs were cloned into standard cloning vectors and subsequently verified by sequencing. The fragments were sub cloned into pcDNA 3.1(+) expression vector (Thermo Fisher Scientific, USA), plasmid DNA was purified from transformed bacteria and purity and concentration were determined by UV spectroscopy. The final constructs were verified by restriction mapping and sequencing.

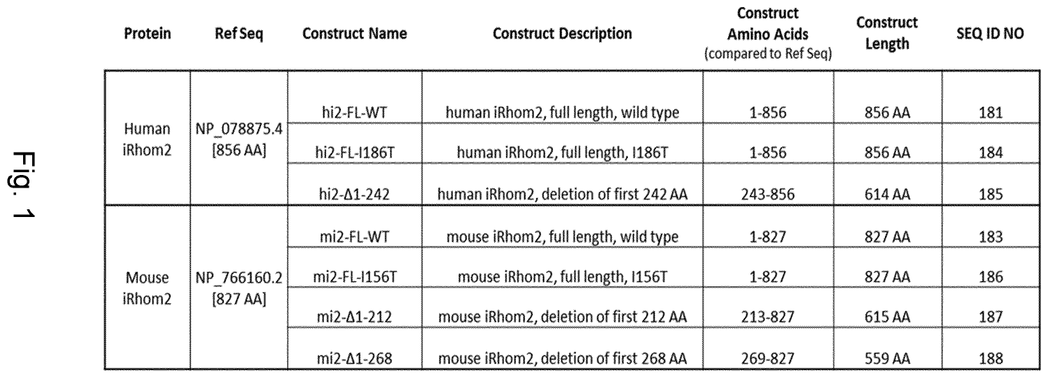

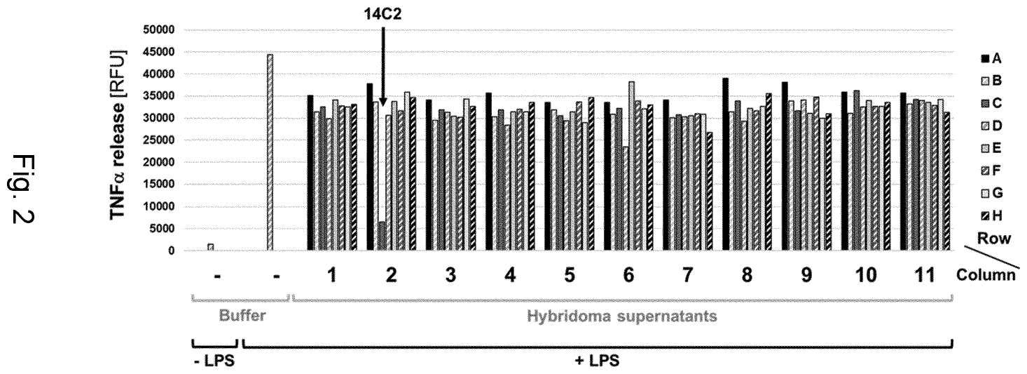

FIG. 1 depicts the expression vectors used for immunization, indicating their designation, description, amino acids with regard to NCBI reference sequence NP_078875.4 for human iRhom2 and NCBI reference sequence NP_766160.2 for mouse iRhom2 and their respective sequence identification numbers (SEQ ID NO).

### Example 2: Breeding of iRhom2 knockout mice for immunization

Due to the high sequence homology of human versus mouse iRhom2 protein (referring to the NCBI reference sequence NP_078875.4 for human iRhom2 and the NCBI reference sequence NP_766160.2 for mouse iRhom2, the amino acid sequence identity for the extracellular loops 1, 2, 3 and the C-terminal tail of human versus mouse iRhom2 are calculated as 89.96%, 100.00%, 100.00% and 96.97%, respectively), iRhom2 knockout rather than wild type mice were bred for immunization.

In brief, the Rhbdf2tmlb(KOMP)Wtsi mouse strain (Rhbdf2 is an alternative name for iRhom2) was ordered for resuscitation from the KOMP Mouse Biology Program at University of California, Davis, and resulted in the availability of three heterozygous male mice. These three animals, which were in a C57BL/6N background (C57BL/6N-Rhbdf2tmlb(KOMP)Wtsi), were mated with wild type female mice of a 129Sv/J genetic background to produce heterozygous offspring. These heterozygous mice were mated with one another to generate male and female mice with homozygous knockout of the Rhbdf2 gene. The resulting homozygous Rhbdf2 knockout mouse colony was further expanded for immunization.

### Example 3: Immunization of Mice and Serum Titer Analysis

Ten cohorts of 8 to 12 weeks old male and female iRhom2 knockout mice (as described in Example 2) were genetically immunized with pBT2-8HAX3-vectors coding for hi2-FL-WT, hi2-FL-I186T, hi2-A1-242, mi2-FL-WT & mi2-FL-I156T, mi2-Δ1-212, mi2-A1-268, hi2-FL-WT & mi2-FL-WT, hi2-A1-242 & mi2-Δ1-212, hi2-A1-242 & mi2-A1-268, hi2-A1-242 & mi2-Δ1-212 & mi2-A1-268, respectively. Using the Helios™ Gene Gun System (Biorad, USA) DNA-coated (approximately five pg DNA) nano-goldparticles were administered by nonoverlapping shots on shaved skin of the animals.

Four to ten mice per cohort were injected every 7 days for four to nine times. Ten days after the last injection, blood (serum) was collected and tested for antibody titer.

Assessment of the immune response was conducted by serum antibody titer analysis applying the FACS method. In brief, sera, diluted 1:50 in PBS containing 3% FBS, were tested on murine L929 cells stably expressing human iRhom2 using goat F(ab′)2 anti-Mouse IgG (H+L)-R-phycoerythrin (RPE) conjugate (Dianova, Germany) as secondary antibody. As a negative control, parental L929 cells were used. Tests were performed on an Accuri C6 Plus (BD Biosciences, USA) flow cytometer. Pre-immune serum taken at day 0 of the immunization protocol served as negative control.

Four days after a final booster immunization, lymph nodes of selected animals were collected, lymphocytes were isolated and either directly used or cryopreserved for subsequent fusions.

### Example 4: Recovery of Lymphocytes and Fusions for the Generation of Hybridomas

Fresh or cryopreserved lymph node-derived lymphocytes from six to fifteen selected animals were fused with Ag8 mouse myeloma cells for the generation of hybridoma cells. Fusions and subsequent growth of cells were either carried out in liquid or semi-solid media. For fusion and growth on semi-solid media plates, IgM depletion and B-cell enrichment was performed before cells were plated. Advanced imaging with integrated robotics and data tracking for automated picking of the highest value clones using the CellCelector device (ALS, Germany) was applied for further propagation and testing. For fusion and growth using liquid media, IgM depletion and B-cell enrichment was not performed.

Fused cells were plated and grown on 96-well plates in the presence of hypoxanthine-aminopterin-thymidine (HAT) medium.

### Example 5: Screens of Hybridoma Supernatants for Candidate Selection

In this example, the approaches for screening of hybridoma supernatants are described. Both functional and binding screens were conducted. Thus, in the following sections, screens for assaying the effects of hybridoma supernatants on TNFα shedding in THP-1 cells, the generation of murine L929 cells expressing different forms of human iRhom2, and the binding screens on these engineered test systems will be described.

Functional Screen of Hybridoma Supernatants for Candidate Selection After 14 days of culture, supernatants of hybridoma cells were collected and subjected to an ELISA-based functional screen for iRhom2 activity-neutralizing antibodies. Since the crucial role of iRhom2 in TACE-mediated release of tumor necrosis factor alpha (TNFα) from macrophages is very well established (McIlwain et al., 2012, Adrain et al., 2012, Siggs et al., 2012), the human TNF-alpha DuoSet ELISA (R&D Systems, USA) was employed to compare the lipopolysaccharide (LPS)-induced release of endogenous TNFα from human THP-1 monocytic cells in the presence and absence of all DNA immunization-derived hybridoma supernatants.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked for 3 hours with 300 μl per well of TBS, 1% BSA at room temperature. 20,000 THP-1 (American Type Culture Collection, USA) cells in 80 μl of normal growth medium were seeded in each well of Greiner CELLSTAR V-bottom 96-well plates (Thermo Fisher Scientific, USA) and pre-incubated with 20 μl of hybridoma supernatants at 37° C., 5% CO₂ for 30 minutes. In case of stimulation controls, 20 μl of standard growth medium instead of hybridoma supernatants were added.

Subsequently, cells (except those for unstimulated controls) were stimulated with 20 μl per well of LPS (Sigma-Aldrich, USA) at 300 ng/ml growth medium for a final concentration of 50 ng/ml at 37° C., 5% CO₂ for 2 hours. Afterwards, the 96-well plates were centrifuged to pellet cells. In parallel, blocking buffer was removed from the MaxiSorp® plates and plates were washed 4 times with 350 μl per well of TBS-T (Carl Roth, Germany) on a 96-head plate washer (Tecan Group, Switzerland). To avoid drying-up, 30 μl of TBS were added to each well of the MaxiSorp® plates immediately, followed by the transfer of 70 μl of cell-free supernatant per sample. Additionally, 100 μl of recombinant human TNFα protein (provided as part of the DuoSet ELISA kit) diluted in TBS at defined concentrations were added to the plate as standard references. Thereafter, 100 μl per well of biotinylated goat anti-human TNFα detection antibody (provided as part of the DuoSet ELISA kit) at 50 ng/ml TBS were added and, protected from direct light, plates were incubated at room temperature for 2 hours. After 4 times washing with 350 μl per well of TBS-T (Carl Roth, Germany) on a 96-head plate washer (Tecan Group, Switzerland) and careful removal of all buffer traces after the fourth cycle, 100 μl of streptavidin-AP (R&D Systems, USA) diluted 1:10,000 in TBS were added to each well and, again protected from direct light, plates were incubated at room temperature for 30 minutes. Following another round of 4 times washing with 350 μl per well of TBS-T (Carl Roth, Germany) on a 96-head plate washer (Tecan Group, Switzerland) and careful removal of all buffer traces after the fourth cycle, 100 μl of AttoPhos substrate solution (Promega, USA) was added for incubation in the dark at room temperature for 1 hour. Using an infinite M1000 PRO (Tecan Group, Switzerland) microplate reader, the fluorescence of each well was collected at an excitation wavelength of 435 nm and an emission wavelength of 555 nm.

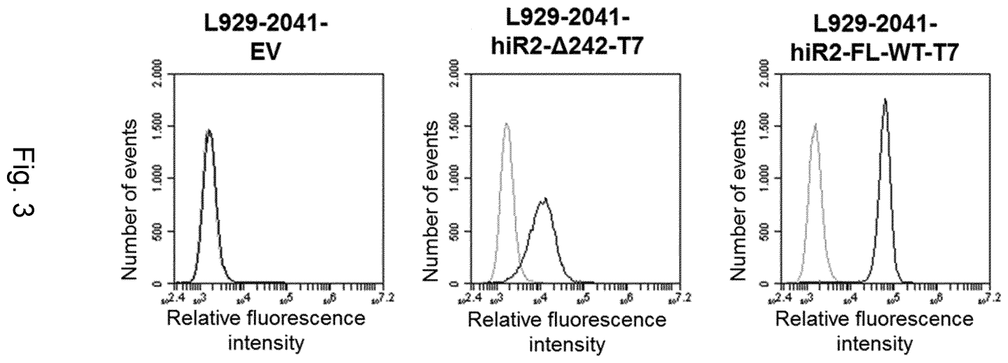

FIG. 2 shows representative results of these experiments for one 96-well plate demonstrating the effects of DNA immunization-derived hybridoma supernatants on LPS-induced release of TNFα from THP-1 cells. The supernatant collected from the hybridoma cell population of plate number 14, row C, column 2, (14C2), the originator of the later antibody 3 as a representative example of selected candidates, effectively interferes with LPS-induced shedding of TNFα in THP-1 cells.

### Generation of Cell Populations for Cell Binding FACS Analyses

In order to generate a cell system that is suited for comparable and reliable binding analyses of the antibodies, L929 (NCTC clone 929) mouse fibroblast cells (ATCC, USA) were genetically modified to knock-out the mouse iRhom2 gene. The resulting L929 mouse iRhom2 knock-out cell line was afterwards infected with different human iRhom2 constructs to obtain cell line derivatives, stably expressing different human iRhom2 proteins, that allow for binding analyses to different iRhom2 variants in the same genetic background.

In brief, mRhbdf2.3 IVT gRNA (AAGCATGCTATCCTGCTCGC) (SEQ ID NO 197) was synthesized at Thermo Fisher Scientific GeneArt GmbH, Regensburg, Germany. One day post seeding in 24 well plates, L929 parental cells were transfected according to GeneArt CRISPR Nuclease mRNA user guide (Thermo Fisher Scientific, USA) with the gRNA/GeneArt Platinium Cas9 Nucelase (Thermo Fisher Scientific, USA) mix using Lipofectamine CRISPRMAX Transfection Reagent (Thermo Fisher Scientific, USA). 3 days post transfection, cells were lysed and DNA was extracted for amplification of specific PCR products using the mRhbdf2.3 fwd (TCAATGAGCTCTTTATGGGGCA) (SEQ ID NO 195)/mRhbdf2.3 rev (AAGGTCTCCATCCCCTCAGGTC) (SEQ ID NO 196) 5primer pair (Thermo Fisher Scientific, USA). For selection of positive wells, GeneArt Genomic Cleavage Detection Kit (Thermo Fisher Scientific, USA) was applied to those samples that had a prominent single band of the correct size in an Invitrogen 2% E-Gel Size Select agarose gel (Thermo Fisher Scientific, USA). Cleavage assay PCR products were also analyzed on Invitrogen 2% E-Gel Size Select agarose gels. Two rounds of subsequent sub cloning of the identified polyclonal L929 population using limited dilution technique were performed, using the Cleavage Detection Kit for identification of positive sub clones. Thereby, the most promising positive sub clone identified in the first round, named 1029, was further sub cloned in the second round to obtain the final clone, named 2041. The monoclonal cell population derived from this sub clone is named L929-2041 and was used for subsequent infections (according to the procedure described in Example 13) with the human iRhom2 constructs hiR2-Δ242-T7 and hiR2-FL-WT-T7 for the generation of the two cell lines L929-2041-hiR2-Δ242-T7 and L929-2041-hiR2-FL-WT-T7, respectively.

### FACS Analyses for Validation of the Test Systems

Upon selection, fluorescence activated cell sorting (FACS) analyses were conducted to verify the plasma membrane localization of the human iRhom2 variants ectopically expressed by the genetically engineered murine L929 cell populations.

In brief, murine L929-2041-EV control cells stably infected with pMSCV empty vector, L929-2041-hiR2-Δ242-T7 cells expressing a human iRhom2 variant deleted for amino acids 1-242 and C-terminally tagged with 3 consecutive copies of the T7 epitope (MASMTGGQQMG), and L929-2041-hiR2-FL-T7 cells expressing human iRhom2 full length wild type also C-terminally tagged with 3 consecutive copies of the T7 epitope were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or mouse monoclonal anti-T7 IgG (Merck Millipore, USA) at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

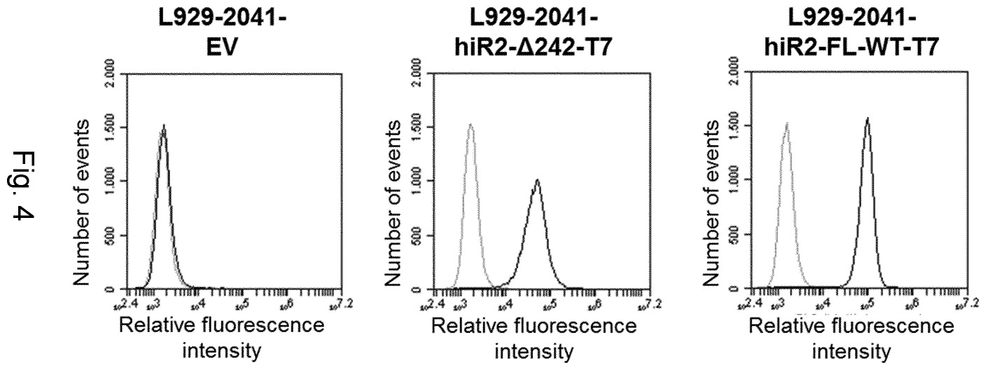

FIG. 3 shows representative results of this experiment. As compared to control samples incubated with anti-mouse IgG secondary antibody only (gray), co-incubation with anti-T7 tag antibody (black) results in no background staining of L929-2041-EV control cells at all (left). In contrast, binding analyses of the anti-T7 tag antibody on both L929-2041-hiR2-Δ242-T7 (middle) and, even more pronounced, on L929-2041-hiR2-FL-T7 (right) cells reveal a strong increase in relative fluorescence intensity, demonstrating that both T7-tagged variants of human iRhom2 —the Δ242 deletion form and the full length wild type form—are localized on the surface of these genetically engineered cell populations and, thus, validating them as suitable screening systems for binding of antibodies from hybridoma supernatants.

### Binding Screen of Hybridoma Supernatants for Candidate Selection

Next, the validated L929 cell populations were applied to systematically screen hybridoma supernatants for iRhom2 binding antibodies.

In brief, L929-2041-EV control cells, L929-2041-hiR2-Δ242-T7 cells and L929-2041-hiR2-FL-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or hybridoma supernatants pre-diluted 1:50 in FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

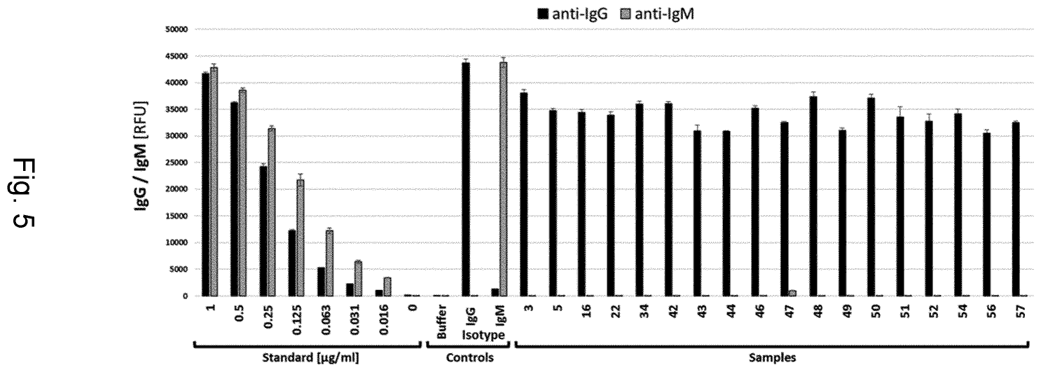

FIG. 4 shows representative results of these experiments. Incubation of the applied cell populations with supernatant of the hybridoma cell pool 14C2 (the primary material leading to the antibody 3 of the invention) as a representative example of selected candidates (black) leads to no background staining of L929-2041-EV control cells at all (left), whereas a strong shift in relative fluorescence intensity, similar to or even stronger than the one observed with the anti-T7 tag antibody, was detected on L929-2041-hiR2-Δ242-T7 (middle) and L929-2041-hiR2-FL-WT-T7 (left) cells, clearly demonstrating antibodies from the hybridoma supernatant 14C2 to recognize both forms of human iRhom2.

### Example 6: Sub-Cloning of the Hybridoma Cell Populations

Since most of the hybridoma cell populations appeared to be of oligoclonal origin, sub-cloning applying classical liquid dilution technique was performed to isolate monoclonal hybridoma cell pools.

In brief, cells of the oligoclonal hybridoma population were counted and the dilution factor to end up with an average of two cells per well of 96-well plates was calculated. Cells were diluted accordingly and wells with growth of a single cell population were identified by microscopy-based screening. After expansion of these monoclonal hybridoma populations for approximately 3 weeks, supernatants were collected and compared for inhibitory effects on LPS-induced release of TNFα from THP-1 cells as described in Example 5.1. Sub clones that turned out to significantly interfere with TNFα shedding were expanded and stocked.

### Example 7: Purification of Antibodies from the Monoclonal Hybridomas

Following the generation of monoclonal hybridoma populations, the antibodies were purified from their respective hybridoma supernatant applying affinity chromatography.

In brief, supernatants collected from the monoclonal hybridoma cells were loaded on equilibrated protein G sepharose prepacked gravity-flow columns (Protein G GraviTrap™, GE Healthcare, UK) for antibody capturing. Afterwards, columns were washed once with binding buffer and trapped antibodies were eluted with elution buffer (both buffers are provided as part of the Ab Buffer Kit; GE Healthcare, UK). Next, the eluate fractions were desalted using PD Miditrap G-25 columns (GE Healthcare, UK), and purified samples were concentrated via Amicon Ultra-4 Centrifugal Filter Units with a cutoff at 30 kDa (Sigma-Aldrich, USA).

Finally, the concentration of the purified proteins was determined applying a NanoDrop 2000/c spectrophotometer (Thermo Fisher Scientific, USA).

### Example 8: Isotype Determination of the Purified Antibodies of the Invention

As a next step, a mouse IgG/IgM ELISA was performed to determine the isotype of the purified antibodies of the invention. In brief, on day 1, Nunc black MaxiSorp® 96-well plates (Thermo Fisher Scientific, USA) were coated overnight with 100 μl per well of goat anti mouse IgG+ IgM (H+L) capture antibody (Sigma-Aldrich, USA) at 1 μg/ml TBS at 4° C. On day 2, the capture antibody solution was removed and MaxiSorp® plates were blocked with 300 μl Pierce protein-free (TBS) blocking buffer (Thermo Fisher Scientific, USA) per well at room temperature for 2 hours. The blocking buffer was then removed and plates were washed 4 times with 350 μl TBS-T (Carl Roth, Germany) per well on a 96-head plate washer (Tecan Group, Switzerland). Afterwards, 100 μl TBS per well as blank and negative control, mouse IgG (Thermo Fisher Scientific, USA) and mouse IgM (Sigma-Aldrich, USA) antibody at defined concentrations (both 1:2 titrations starting at 1 μg/ml in TBS) as standard references, mouse IgG (Thermo Fisher Scientific, USA) and mouse IgM (Sigma-Aldrich, USA) antibody at 3 μg/ml in TBS each as positive and specificity controls, and the purified antibodies of the invention at 3 μg/ml in TBS were added to the wells and incubated at room temperature for 2 hours. Subsequently, the plates were washed 4 times with 350 μl per well of TBS-T (Carl Roth, Germany) on a 96-head plate washer (Tecan Group, Switzerland). For isotype detection, one half of the sample each were, protected from direct light, incubated with 100 μl per well of AP-conjugated goat anti mouse IgM (Sigma-Aldrich, USA) or AP-conjugated goat anti mouse IgG F(ab′)2 Fragment (Dianova, Germany) detection antibodies diluted 1:5,000 in TBS for 1.5 hours at room temperature. Following another round of 4 washing steps with 350 μl per well of TBS-T (Carl Roth, Germany) on a 96-head plate washer (Tecan Group, Switzerland) and careful removal of all buffer traces after the last cycle, 100 μl of AttoPhos substrate solution (Promega, USA) were added for incubation in the dark and at room temperature for 10 minutes. Using an infinite M1000 (Tecan Group, Switzerland) microplate reader, the fluorescence of each well was collected at an excitation wavelength of 435 nm and an emission wavelength of 555 nm. FIG. 5 shows results of this experiment clearly demonstrating the antibodies 3, 5, 16, 22, 34, 42, 43, 44, 46, 47, 48, 49, 50, 51, 52, 54, 56, and 57 of the invention to be of mouse IgG isotype.

### Example 9: CDR Sequence Determination of the Purified Antibodies of the Invention

All 18 antibodies of the invention were subjected to sequence determination. In brief, total RNA was isolated from the hybridoma cells following the technical manual of Ambion's TRIzol® Reagent (Thermo Fisher Scientific, USA). Total RNA was then reverse-transcribed into cDNA using either isotype-specific anti-sense primers or universal primers following the technical manual of PrimeScript™ 1st Strand cDNA Synthesis Kit (Takara, Japan). Antibody fragments of heavy chain and light chain were amplified according to the standard operating procedure (SOP) of rapid amplification of cDNA ends (RACE) of GenScript. Amplified antibody fragments were cloned into the pCE2 TA/Blunt-zero standard cloning vector (Vazyme Biotech Co.,Ltd, China) separately. Colony PCR was performed to screen for clones with inserts of correct sizes. In total five clones of each antibody were sequenced on an Applied Biosystems 3730 DNA Analyzer (Thermo Fisher Scientific, USA).

### Example 10: Affinity Determination of the Purified Antibodies of the Invention

In this study, affinity measurements of the purified antibodies 3, 5, 16, 22, 34, 42, 43, 44, 48, and 50 of the invention were performed by indirect FACS scatchard analysis on THP-1 cells, a human monocytic cell line endogenously expressing iRhom2.

In brief, human THP-1 cells (American Type Culture Collection, USA) were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. In order to pellet cells and remove supernatants, the plates were centrifuged at 1,500 rpm and 4° C. for 3 minutes. For primary staining, cells were resuspended in 100 μl per well of either FACS buffer alone (controls) or serial two-fold dilutions (in total 22 concentrations) of the purified antibodies 3, 5, 16, 22, 34, 42, 43, 44, 48, and 50 of the invention in FACS buffer starting at 40 μg/ml and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany). Applying Prism8 software (GraphPad Sowtware, USA), the respective KD value for each of the antibodies of the invention were calculated.

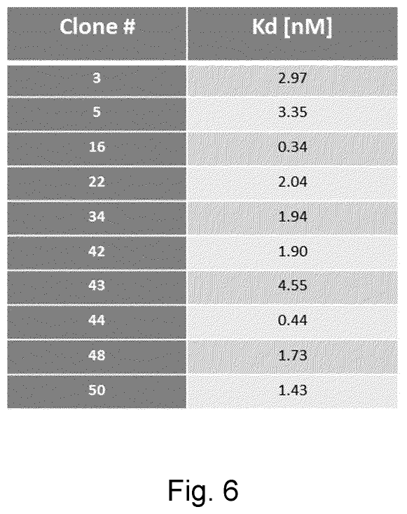

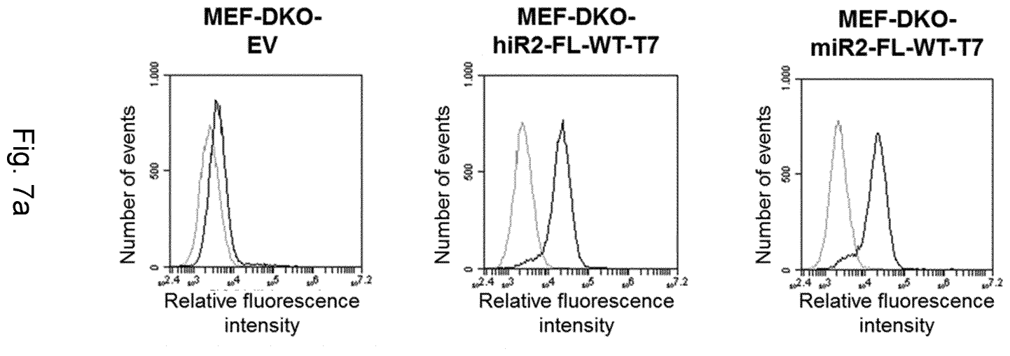

FIG. 6 shows representative results of this study, demonstrating that the KD values for binding of the purified antibodies 3, 5, 16, 22, 34, 42, 43, 44, 48, and 50 of the invention to THP-1 cells are in the subnanomolar to low nanomolar range.

### Example 11: Generation of iRhom1/2−/− Double Knockout Mouse Embryonic Fibroblasts

For various purposes, in particular binding studies, described in some of the following examples, cell systems expressing defined levels of particular iRhom variants of interest against a background lacking any endogenous iRhom1 or iRhom2 protein were required. For this purpose, mouse embryonic fibroblasts (MEFs) from double knockout (DKO) mice homozygously negative for both mouse iRhom1 and mouse iRhom2 (iRhom1/2−/−) were established. This example describes the mouse strains used for the establishment of iRhom1/2−/− DKO MEFs and the generation of an immortalized iRhom1/2−/− DKO MEF cell line.

Mouse Strains Used for the Establishment of iRhom1/2−/− DKO MEFs

In brief, the Rhbdf2tmlb(KOMP)Wtsi mouse strain on a C57BL/6N background (C57BL/6N-Rhbdf2tmlb(KOMP)Wtsi) was obtained from the Knockout Mouse Project (KOMP) Repository at the University of California, Davis, USA (Rhbdf2 is an alternative name for iRhom2). Heterozygous male Rhbdf2tmlb mice were mated with wild type female mice of a 129Sv/J genetic background to produce heterozygous offspring of mixed genetic background (129Sv/J-C57BL/6N). These heterozygous mice were mated with one another to generate male and female offspring that were homozygous for the deletion of the Rhbdf2 gene (Rhbdf2−/− mice, 129Sv/J-C57BL/6N). The resulting homozygous Rhbdf2 knockout mouse colony was further expanded by breeding of Rhbdf−/− male and female mice to generate sufficient numbers of mice. Homozygous Rhbdf2−/− mice are viable and fertile with no evident spontaneous pathological phenotypes. Rhbdf1 knockout mice were obtained from the European Conditional Mouse Mutagenesis Program (EUCOMM) of the International Knockout Mouse Consortium (IKMC). The generation of these animals is described in Li et al., PNAS, 2015, doi: 10.1073/pnas.1505649112. Homozygous Rhbdf1-/− mice are viable and fertile with no evident spontaneous pathological phenotypes.

For the generation of DKO mice for Rhbdf1 and Rhbdf2 (Rhbdf1/2−/− mice), Rhbdf1-/− mice were mated with Rhbdf2−/− mice to generate Rhbdf1+/− Rhbdf2+/− doubly heterozygous mice. These were mated with Rhbdf2−/− mice to produce Rhbdf1+/− Rhbdf2−/− animals, which were mated with one another to generate E14.5 embryos lacking both Rhbdf genes (Rhbdf1/2−/− DKO embryos) at the expected Mendelian ratios (¼ of all embryos) for production of E13.5 Rhbdf1/2−/− DKO MEFs, as described below.

Generation of an Immortalized iRhom1/2−/− DKO MEF Cell Line

In brief, pregnant Rhbdf1+/− Rhbdf2−/− females were sacrificed at E13.5. The uterine horns were removed into a dish with ice-cold PBS. Using fine tip forceps, the embryos were released from maternal tissue and each embryo was removed from placenta. Each embryo was then decapitated with a sharp scalpel and all internal organs such as liver, heart, lung and intestines were removed. A 0.5 mm section of the tail was removed and transferred to a 1.5 ml Eppendorf tube for isolation of genomic DNA and subsequent PCR genotyping to confirm the correct genotype of the embryo. Afterwards, the remaining embryonic tissue was washed once with PBS and transferred into a tissue culture dish with 2 mL of 0.25% trypsin/EDTA. The tissue was extensively minced with two sterile scalpels, and the trypsin/cell mixture was incubated at 37° C. for 15 minutes. Trypsinization was stopped by the addition of FCS-containing growth medium. To generate a single cell suspension, the mixture was pipetted up and down, first five times with a 10 mL serum pipet, then five times with a 5 mL serum pipet and finally several times with a fire-polished Pasteur pipet to further dissociate any remaining cell clusters. Subsequently, cells obtained from one embryo were plated on two 10 cm tissue culture plates. The next day, the medium was replaced by fresh medium and the cells were allowed to grow until they reached 90% confluency. Finally, cells were expanded and stocked for future usage. For immortalization of primary Rhbdf1/2−/− DKO MEFs, cells were transduced with a retroviral system using the pMSCV expression system (Clontech, USA). Briefly, a pMSCV-Zeo-SV40 was generated as follows: the sequences coding for the puromycin resistance were removed from plasmid pMSCV-puro (Clontech, USA) and replaced with the sequences conferring the Zeocin resistance from pcDNA3.1(+) Zeo vector (Thermo Fisher Scientific, USA). The retroviral packaging cell line GP2-293 (Clontech, USA) was used in combination with the envelope vector pVSV-G (Clontech, USA) and the pMSCV-Zeo-SV40 plasmid to produce a retrovirus encoding the SV40 large T-antigen. The virus was filtered and added to primary Rhbdf1/2−/− DKO MEFs plated at 50% confluency for 24 hours. Afterwards, transduced Rhbdf1/2−/− DKO MEFs were allowed to grow in growth medium without selection pressure for 24 hours and were then shifted to growth medium containing 100 μg/ml of Zeocin. Cells were passaged when confluent and after ten passages were stocked for future usage.

### Example 12: Evaluation of Mouse Cross-Reactivity of the Purified Antibodies of the Invention

Next, immortalized iRhom1/2−/− DKO MEFs were reconstituted with a tagged form of human iRhom2 in order to confirm target recognition by hybridoma supernatants (as described in example 5) for the respective purified antibodies of the invention and thereby to verify reconstituted iRhom1/2−/− DKO MEFs as suitable test systems. Additionally, iRhom1/2−/− DKO MEFs stably expressing a tagged form of mouse iRhom2 were generated in order to determine cross-reactivity of the purified antibodies 3, 5, 16, 22, 34, 42, 43, 44, 48, and 50 of the invention with the mouse orthologue of iRhom2.

Generation of iRhom1/2−/− DKO MEFs Stably Expressing T7-Tagged Human or Mouse iRhom2 In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV (Clontech, USA) empty vector, pMSCV-hiR2-FL-WT-T7 encoding human iRhom2 full length wild type C-terminally tagged with 3 consecutive copies of the T7 epitope (MASMTGGQQMG) or pMSCV-miR2-FL-WT-T7 encoding mouse iRhom2 full length wild type C-terminally tagged with 3 consecutive copies of the T7 epitope, and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV, pMSCV-hiR2-FL-WT-T7 or pMSCV-miR2-FL-WT-T7 ecotrophic virus were collected, filtered with 0.45 pm CA filters, and supplemented with 4 μg/ml of polybrene (Sigma-Aldrich, USA). Upon removal of medium from the immortalized iRhom1/2−/− DKO MEFs, the virus containing supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO-EV control cells stably infected with pMSCV empty vector, MEF-DKO-hiR2-FL-WT-T7 cells stably expressing human iRhom2 full length wild type C-terminally tagged with 3 consecutive copies of the T7 epitope, and MEF-DKO-miR2-FL-WT-T7 cells stably expressing mouse iRhom2 full length wild type C-terminally tagged with 3 consecutive copies of the T7 epitope. Upon propagation, cells were stocked for future usage.

### FACS Analyses for Test System Validation and Antibody Characterization

In brief, immortalized MEF-DKO-EV control cells, MEF-DKO-hiR2-FL-WT-T7 cells and MEF-DKO-miR2-FL-WT-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls), mouse monoclonal anti-T7 IgG (Merck Millipore, USA) at 3 μg/ml FACS buffer or the purified antibodies 3, 5, 16, 22, 34, 42, 43, 44, 48, and 50 of the invention also at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

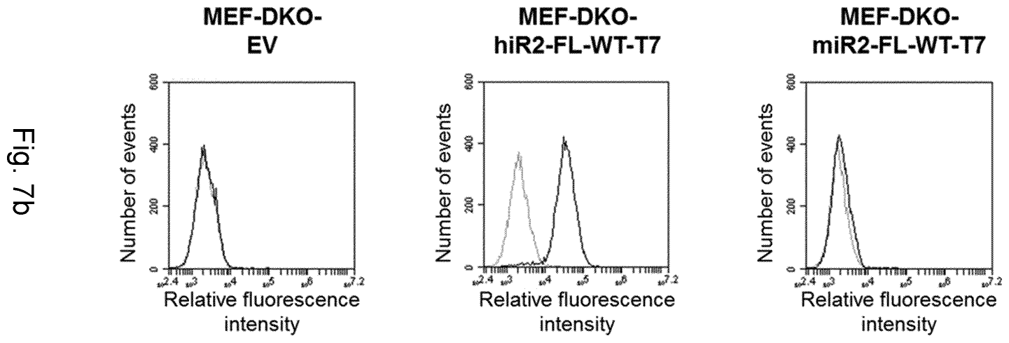

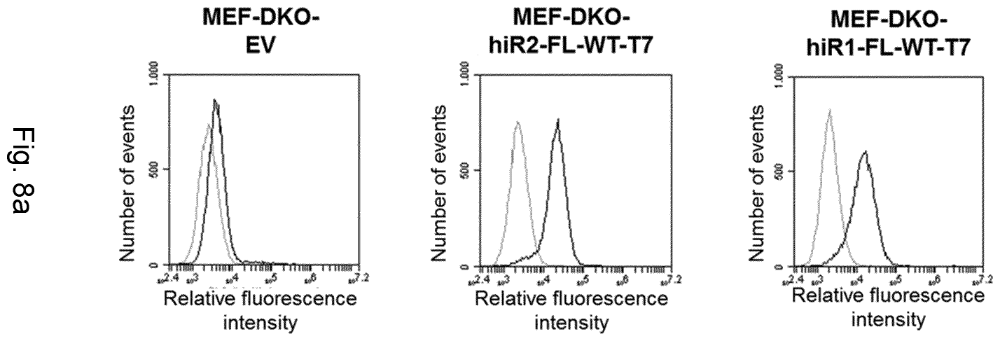

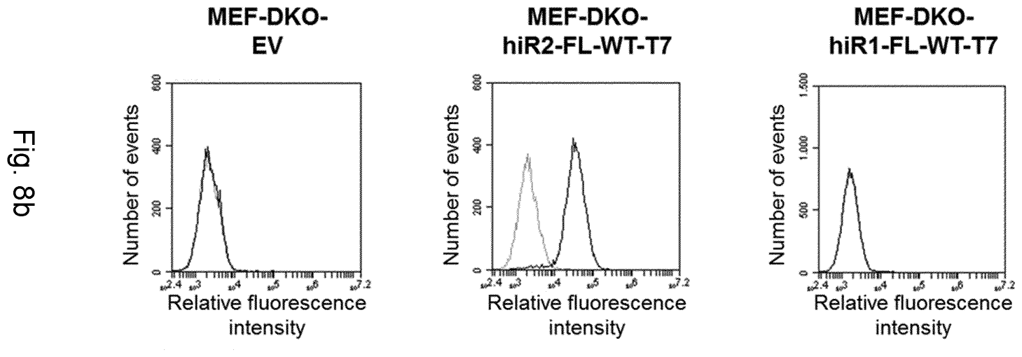

FIGS. 7a & 7b show representative results of this experiment. As compared to control samples incubated with anti-mouse IgG secondary antibody only (7a & 7b, gray), co-incubation with anti-T7 tag antibody (FIG. 7a, black) results in very little background staining of MEF-DKO-EV control cells (FIG. 7a, left). In contrast, binding analyses of the anti-T7 tag antibody on both MEF-DKO-hiR2-FL-WT-T7 (FIG. 7a, middle) and MEF-DKO-miR2-FL-WT-T7 (FIG. 7a, right) cells reveal a strong increase in relative fluorescence intensity, demonstrating both the ectopically expressed human and the mouse iRhom2 variant to be localized on the surface of these genetically engineered cell populations and, thus, validating them as suitable test systems for characterizing the antibodies of the invention. Co-incubation of these cell populations with purified antibody 3 as a representative example of the purified antibodies of the invention (FIG. 7b, black) leads to no background staining of MEF-DKO-EV control cells at all (FIG. 7b, left), while the strong shift in relative fluorescence intensity, similar to the one observed with the anti-T7 tag antibody, on MEF-DKO-hiR2-FL-WT-T7 cells demonstrates strong binding of the purified antibody 3 of the invention to the human iRhom2 variant (FIG. 7b, middle), thereby confirming the results described in example 5 for the supernatant of the corresponding hybridoma pool. In contrast, no significant binding of the purified antibody 3 of the invention to MEF-DKO-miR2-FL-WT-T7 cells is detectable (FIG. 7b, right), providing evidence that the mouse iRhom2 variant, whose presence on the cell surface is verified with the anti-T7 tag antibody (FIG. 7a, right), is not being recognized by the purified antibody 3 of the invention. Similar results were obtained with the purified antibodies 5, 16, 22, 34, 42, 43, 44, 48, and 50 of the invention, demonstrating that none of these purified antibodies of the invention are cross-reactive with mouse iRhom2.

### Example 13: Assessment of Binding Specificity of the Purified Antibodies of the Invention

Due to the sequence homology of the human iRhom2 protein versus its closely related family member human iRhom1 (referring to the NCBI reference sequence NP_078875.4. for human iRhom2 and the NCBI reference sequence NP_071895.3 for human iRhom1, the amino acid sequence identity for the extracellular loops 1, 2, 3 and the C-terminal tail of human iRhom2 versus human iRhom1 are calculated as 67.4%, 100.00%, 80.00% and 63.64%, respectively), the binding specificity of the purified antibodies 3, 5, 16, 22, 34, 42, 43, 44, 48, and 50 of the invention for human iRhom2 versus human iRhom1 was assessed as a next step. For this purpose, iRhom1/2−/− DKO MEFs stably expressing a tagged form of human iRhom1 were generated.

Generation of iRhom1/2−/− DKO MEFs Stably Expressing T7-Tagged Human iRhom1

In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV-hiR1-FL-WT-T7 (SEQ ID NO 189) encoding human iRhom1 full length wild type C-terminally tagged with 3 consecutive copies of the T7 epitope, and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV-hiR1-FL-WT-T7 ecotrophic virus were collected, filtered with 0.45 pm CA filters, and supplemented with 4 μg/ml of polybrene (Sigma-Aldrich, USA). Upon removal of medium from the immortalized iRhom1/2−/− DKO MEFs, these supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO-hiR1-FL-WT-T7 cells stably expressing human iRhom1 full length wild type C-terminally tagged with 3 consecutive copies of the T7 epitope. Upon propagation, cells were stocked for future usage.

### FACS Analyses for Antibody Characterization

In brief, in addition to immortalized MEF-DKO-EV control cells and MEF-DKO-hiR2-FL-WT-T7 cells (as already described in example 12), MEF-DKO-miR2-FL-WT-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls), mouse monoclonal anti-T7 IgG (Merck Millipore, USA) at 3 μg/ml FACS buffer or the purified antibodies 3, 5, 16, 22, 34, 42, 43, 44, 48, and 50 of the invention also at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

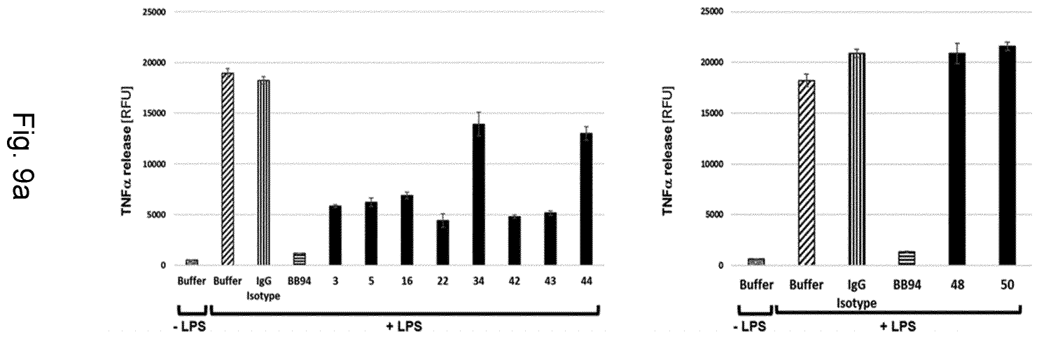

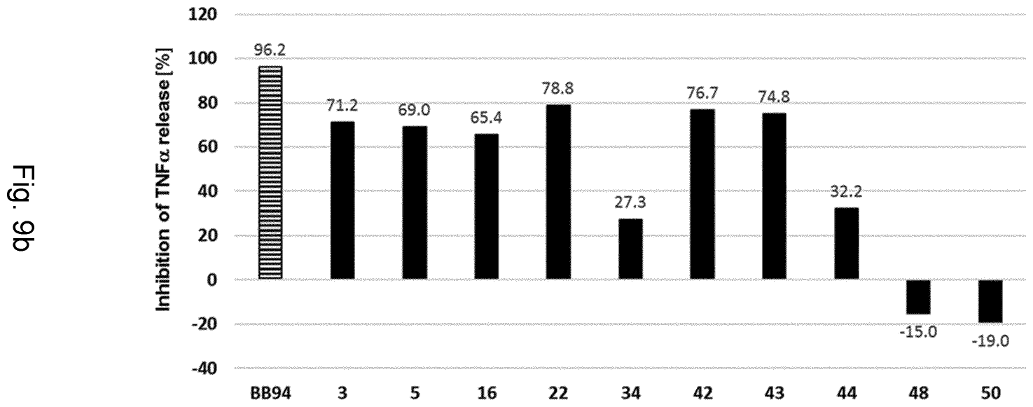

FIGS. 8a & 8b show representative results of these analyses. When compared to the stainings of MEF-DKO-EV control cells (FIG. 8a, left; identical to FIG. 7a, left) and MEF-DKO-hiR2-FL-WT-T7 (FIG. 8a, middle; identical to FIG. 7a, middle), the strong increase in relative fluorescence intensity obtained on MEF-DKO-hiR1-FL-WT-T7 with the anti-T7 tag antibody (FIG. 8a, left) demonstrates that, similarly to the human iRhom2 variant, the human iRhom1 variant is also located on the surface of this genetically engineered cell population and, thus, validates it as a suitable test systems for characterizing the antibodies of the invention. In this context, while binding of the antibody 3 as a representative example of the purified antibodies of the invention to the human iRhom2 variant expressed on MEF-DKO-hiR2-FL-WT-T7 cells (FIG. 8b, middle; identical to FIG. 7b, middle) was already shown in example 12, no significant binding of the purified antibody 3 of the invention to MEF-DKO-hiR1-FL-WT-T7 cells is detectable (FIG. 8b, right), providing evidence that the human iRhom1 variant, whose presence on the cell surface is verified with the anti-T7 tag antibody (FIG. 8a, right), is not being recognized by the purified antibody 3 of the invention. Similar results were obtained with the purified antibodies 5, 16, 22, 34, 42, 43, 44, 48, and 50 of the invention, demonstrating that none of these purified antibodies of the invention recognizes human iRhom1.

### Example 14: Analysis of Inhibitory Effects of the Antibodies of the Invention on LPS-Induced TNFα Shedding In Vitro

In the following study, ELISA-based TNFα release assays were performed to verify the inhibitory effects of the purified antibodies of the invention on LPS-induced release of endogenous TNFα from human THP-1 monocytic cells.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 20,000 THP-1 (American Type Culture Collection, USA) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 50 μg/ml as isotype control (for a final concentration of 10 μg/ml in the resulting 100 μl sample volume) or purified antibodies of the invention at 50 μg/ml (for a final concentration of 10 μg/ml in the resulting 100 μl sample volume) at 37° C., 5% CO₂ for 30 minutes. In case of stimulation controls, 20 μl of standard growth medium without test articles were added.

Subsequently, cells (except those for unstimulated controls) were stimulated with 20 μl per well of LPS (Sigma-Aldrich, USA) at 300 ng/ml in growth medium for a final concentration of 50 ng/ml at 37° C., 5% CO₂ for 2 hours. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human TNFα protein (provided as part of the DuoSet ELISA kit) diluted in TBS at defined concentrations were added to the plate as standard references. Thereafter, 100 μl biotinylated goat anti-human TNFα detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes.

Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

FIG. 9 shows representative results of this experiment demonstrating the effects of test articles on LPS-induced release of TNFα from THP-1 cells in absolute numbers (FIG. 9A) and percent inhibition (FIG. 9B). While Batimastat (BB94) as a small molecule inhibitor of metalloproteinases serves as positive control and results in 96.2% inhibition of LPS-induced release of TNFα, the presence of IgG isotype control has no significant effect on TNFα shedding. In contrast, an equal concentration of the purified antibodies 3, 5, 16, 22, 34, 42, 43 and 44 of the invention inhibits LPS-induced release of TNFα from THP-1 cells by 71.2%, 69.0%, 65.4%, 78.8%, 27.3%, 76.7%, 74.8% and 32.2%, respectively. Again in contrast and therefore comparable to the IgG isotype control, the presence of the purified antibodies 48 and 50 of the invention has no significant effect on TNFα shedding.

### Example 15: Epitope Mapping of the Purified Antibodies of the Invention Based on Species-Related Sequence Variations in Human iRhom2

Nowadays, several methods to map epitopes recognized by antibodies are available, including X-ray co-crystallography, array-based oligo-peptide scanning, hydrogen-deuterium exchange or cross-linking-coupled mass spectrometry. Genetic approaches such as site-directed mutagenesis or high-throughput shotgun mutagenesis allow epitope mapping at single amino acid resolution. However, amino acid substitutions at random positions of the protein or substitutions by non-related amino acids bear the risk of causing conformational changes and/or functional loss of the protein and, thus, may result in misinterpretations as to whether the substituted amino acid contributes to an antibody epitope. An elegant and generally accepted way to circumvent these risks is to replace individual amino acids of a given protein by the homologous amino acids of a structurally related protein, i.e. an orthologue or a closely related family member, provided these related proteins are not being recognized by the antibodies of interest. As described earlier, both is true for the purified anti-human iRhom2 antibodies 3, 5, 16, 22, 34, 42, 43, 44, 48, and 50 of the invention, since they were demonstrated to be neither cross-reactive with the mouse orthologue (example 12) nor to bind to the closely related family member human iRhom1 (example 13).

Thus, in a first approach to identify single amino acids that contribute to binding of the antibodies of the invention, plasmids for a set of 25 human iRhom2 variants with mouse iRhom2-related single amino acid substitutions were designed. These 25 substitutions reflect all amino acids in the extracellular parts, i.e. the juxtamembrane domain (JMD) and the large extracellular loop 1 as well as the C-terminus, that are non-identical in human versus mouse iRhom2. Instead of the amino acid of human iRhom2, the amino acid at the corresponding position of mouse iRhom2 was introduced, resulting in the variants hiR2-FL-R441K-T7, hiR2-FL-K443R-T7, hiR2-FL-V4591-T7, hiR2-FL-G481Q-T7, hiR2-FL-L488R-T7, hiR2-FL-L493I-T7, hiR2-FL-D496T-T7, hiR2-FL-H505R-T7, hiR2-FL-Q512L-T7, hiR2-FL- R513K-T7, hiR2-FL-D528N-T7, hiR2-FL-M534S-T7, hiR2-FL-G540S-T7, hiR2-FL-R543Q-T7, hiR2-FL-T544P-T7, hiR2-FL-G546A-T7, hiR2-FL-A547V-T7, hiR2-FL-R582Q-T7, hiR2-FL-F588L-T7, hiR2-FL-M591I-T7, hiR2-FL-E594K-T7, hiR2-FL-E626D-T7, hiR2-FL-L657I-T7, and hiR2-FL-H835Y-T7. In case no corresponding amino acid exists in mouse iRhom2, the respective amino acid of human iRhom2 was deleted, resulting in the variant hiR2-FL-P533-T7.

This example describes the generation of iRhom1/2−/− DKO MEF populations expressing the 25 mouse iRhom2-related single amino acid substitution variants as well as their characterization in terms of cell surface localization and functional activity as indicators of proper protein conformation. Subsequently, binding analyses of the purified antibodies 3, 5, 16, 22, 34, 42, 43, 44, 48, and 50 of the invention on the entire panel of 25 engineered MEF populations expressing human iRhom2 variants with mouse iRhom2-related single amino acid substitutions (including the variant deleted for P533) are described.

Generation of iRhom1/2−/− DKO MEFs Stably Expressing 25 T7-Tagged Human iRhom2 Variants with Mouse iRhom2-Related Single Amino Acid Substitutions

In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV-hiR2-FL-R441K-T7, pMSCV-hiR2-FL-K443R-T7, pMSCV-hiR2-FL-V459I-T7, pMSCV-hiR2-FL-G481Q-T7, pMSCV-hiR2-FL-L488R-T7, pMSCV-hiR2-FL-L493I-T7, pMSCV-hiR2-FL-D496T-T7, pMSCV-hiR2-FL-H505R-T7, pMSCV-hiR2-FL-Q512L-T7, pMSCV-hiR2-FL- R513K-T7, pMSCV-hiR2-FL-D528N-T7, pMSCV-hiR2-FL-P533-T7, pMSCV-hiR2-FL-M534S-T7, pMSCV-hiR2-FL-G540S-T7, pMSCV-hiR2-FL-R543Q-T7, pMSCV-hiR2-FL-T544P-T7, pMSCV-hiR2-FL-G546A-T7, pMSCV-hiR2-FL-A547V-T7, pMSCV-hiR2-FL-R582Q-T7, pMSCV-hiR2-FL-F588L-T7, pMSCV-hiR2-FL-M591I-T7, pMSCV-hiR2-FL-E594K-T7, pMSCV-hiR2-FL-E626D-T7, pMSCV-hiR2-FL-L657I-T7, and pMSCV-hiR2-FL-H835Y-T7 encoding human iRhom2 full length single amino acid substitutions C-terminally tagged with 3 consecutive copies of the T7 epitope (MASMTGGQQMG), and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV-hiR2-FL-R441K-T7, pMSCV-hiR2-FL-K443R-T7, pMSCV-hiR2-FL-V459I-T7, pMSCV-hiR2-FL-G481Q-T7, pMSCV-hiR2-FL-L488R-T7, pMSCV-hiR2-FL-L493I-T7, pMSCV-hiR2-FL-D496T-T7, pMSCV-hiR2-FL-H505R-T7, pMSCV-hiR2-FL-Q512L-T7, pMSCV-hiR2-FL- R513K-T7, pMSCV-hiR2-FL-D528N-T7, pMSCV-hiR2-FL-P533-T7, pMSCV-hiR2-FL-M534S-T7, pMSCV-hiR2-FL-G540S-T7, pMSCV-hiR2-FL-R543Q-T7, pMSCV-hiR2-FL-T544P-T7, pMSCV-hiR2-FL-G546A-T7, pMSCV-hiR2-FL-A547V-T7, pMSCV-hiR2-FL-R582Q-T7, pMSCV-hiR2-FL-F588L-T7, pMSCV-hiR2-FL-M591I-T7, pMSCV-hiR2-FL-E594K-T7, pMSCV-hiR2-FL-E626D-T7, pMSCV-hiR2-FL-L657I-T7, and pMSCV-hiR2-FL-H835Y-T7 ecotrophic virus were collected, filtered with 0.45 pm CA filters, and supplemented with 4 μg/ml of polybrene (Sigma-Aldrich, USA).

Upon removal of medium from the immortalized iRhom1/2−/− DKO MEFs, these supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO-hiR2-FL-R441K-T7, MEF-DKO-hiR2-FL-K443R-T7, MEF-DKO-hiR2-FL-V459I-T7, MEF-DKO-hiR2-FL-G481Q-T7, MEF-DKO-hiR2-FL-L488R-T7, MEF-DKO-hiR2-FL-L493I-T7, MEF-DKO-hiR2-FL-D496T-T7, MEF-DKO-hiR2-FL-H505R-T7, MEF-DKO-hiR2-FL-Q512L-T7, MEF-DKO-hiR2-FL- R513K-T7, MEF-DKO-hiR2-FL-D528N-T7, MEF-DKO-hiR2-FL-P533-T7, MEF-DKO-hiR2-FL-M534S-T7, MEF-DKO-hiR2-FL-G540S-T7, MEF-DKO-hiR2-FL-R543Q-T7, MEF-DKO-hiR2-FL-T544P-T7, MEF-DKO-hiR2-FL-G546A-T7, MEF-DKO-hiR2-FL-A547V-T7, MEF-DKO-hiR2-FL-R582Q-T7, MEF-DKO-hiR2-FL-F588L-T7, MEF-DKO-hiR2-FL-M591I-T7, MEF-DKO-hiR2-FL-E594K-T7, MEF-DKO-hiR2-FL-E626D-T7, MEF-DKO-hiR2-FL-L657I-T7, and MEF-DKO-hiR2-FL-H835Y-T7 cells stably expressing human iRhom2 full length single amino acid substitutions C-terminally tagged with 3 consecutive copies of the T7 epitope. Upon propagation, cells were stocked for future usage.

### FACS Analyses for Test System Validation

In brief, immortalized MEF-DKO-hiR2-FL-WT-T7 cells, MEF-DKO-miR2-FL-WT-T7 cells and MEF-DKO-hiR2-FL-R441K-T7, MEF-DKO-hiR2-FL-K443R-T7, MEF-DKO-hiR2-FL-V459I-T7, MEF-DKO-hiR2-FL-G481Q-T7, MEF-DKO-hiR2-FL-L488R-T7, MEF-DKO-hiR2-FL-L493I-T7, MEF-DKO-hiR2-FL-D496T-T7, MEF-DKO-hiR2-FL-H505R-T7, MEF-DKO-hiR2-FL-Q512L-T7, MEF-DKO-hiR2-FL- R513K-T7, MEF-DKO-hiR2-FL-D528N-T7, MEF-DKO-hiR2-FL-P533-T7, MEF-DKO-hiR2-FL-M534S-T7, MEF-DKO-hiR2-FL-G540S-T7, MEF-DKO-hiR2-FL-R543Q-T7, MEF-DKO-hiR2-FL-T544P-T7, MEF-DKO-hiR2-FL-G546A-T7, MEF-DKO-hiR2-FL-A547V-T7, MEF-DKO-hiR2-FL-R582Q-T7, MEF-DKO-hiR2-FL-F588L-T7, MEF-DKO-hiR2-FL-M591I-T7, MEF-DKO-hiR2-FL-E594K-T7, MEF-DKO-hiR2-FL-E626D-T7, MEF-DKO-hiR2-FL-L657I-T7, and MEF-DKO-hiR2-FL-H835Y-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well.

To pellet cells and remove supernatants, the plates were centrifuged at 1,500 rpm and 4° C. for 3 minutes. For primary staining, cells were resuspended in 100 μl per well of either FACS buffer alone (controls) or mouse monoclonal anti-T7 IgG (Merck Millipore, USA) at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer.

Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

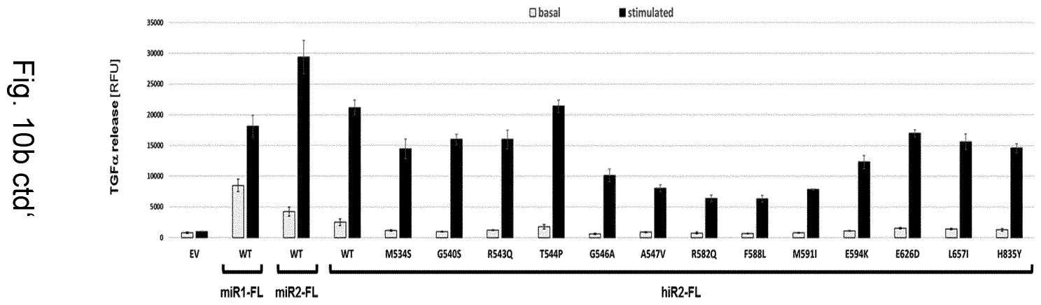

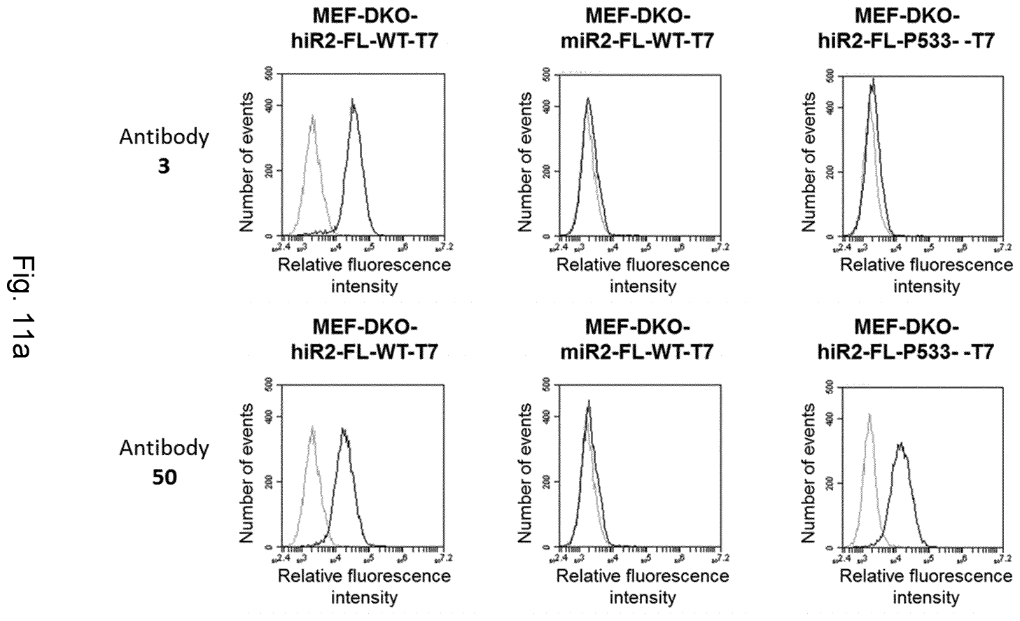

FIG. 10a shows representative results of this experiment exemplarily for the human iRhom2 variant hiR2-FL-P533-T7. Binding analyses of anti-T7 tag antibody (black) and anti-mouse IgG secondary antibody (gray) on MEF-DKO-hiR2-FL-WT-T7 (left), MEF-DKO-miR2-FL-WT-T7 (middle) and MEF-DKO-hiR2-FL-P533-T7 cells (right) reveal a comparably strong increase in relative fluorescence intensity. This demonstrates that, similarly to human and mouse iRhom2 wild type (left and middle), the human iRhom2 variant hiR2-FL-P533-T7 is equally well expressed and localized on the surface of these cells (right). Similar results were obtained for the expression and localization of the other human iRhom2 full length single amino acid substitutions expressed on MEF-DKO-hiR2-FL-R441K-T7, MEF-DKO-hiR2-FL-K443R-T7, MEF-DKO-hiR2-FL-V459I-T7, MEF-DKO-hiR2-FL-G481Q-T7, MEF-DKO-hiR2-FL-L488R-T7, MEF-DKO-hiR2-FL-L493I-T7, MEF-DKO-hiR2-FL-D496T-T7, MEF-DKO-hiR2-FL-H505R-T7, MEF-DKO-hiR2-FL-Q512L-T7, MEF-DKO-hiR2-FL- R513K-T7, MEF-DKO-hiR2-FL-D528N-T7, MEF-DKO-hiR2-FL-M534S-T7, MEF-DKO-hiR2-FL-G540S-T7, MEF-DKO-hiR2-FL-R543Q-T7, MEF-DKO-hiR2-FL-T544P-T7, MEF-DKO-hiR2-FL-G546A-T7, MEF-DKO-hiR2-FL-A547V-T7, MEF-DKO-hiR2-FL-R582Q-T7, MEF-DKO-hiR2-FL-F588L-T7, MEF-DKO-hiR2-FL-M591I-T7, MEF-DKO-hiR2-FL-E594K-T7, MEF-DKO-hiR2-FL-E626D-T7, MEF-DKO-hiR2-FL-L6571-T7, and MEF-DKO-hiR2-FL-H835Y-T7 cells.

### TGFα ELISA for Test System Validation

To test all 25 human iRhom2 variants with mouse iRhom2-specific single amino acid substitutions, or single amino acid deletion as in the case of hiR2-FL-P533-, the respective MEF-DKO cell lines stably expressing these variants, generated as described in the example above, were subjected to TGFα shedding ELISA analysis. In order to demonstrate the functionality of all variants as an indicator that these variants are properly folded, PMA-induced release of nucleofected TGFα was assessed. As the cells used in this analysis are rescue variants of iRhom1/2−/− double knockout mouse embryonic fibroblasts (described in Example 11), that are rescued by the respective human iRhom2 variant with the mouse iRhom2-specific single amino acid substitution or deletion, the iRhom2 variant stably expressed is the only iRhom protein expressed in these cells at all and is therefore the only contributing iRhom to the shedding TGFα in these cells.

In brief, on day 1, Nunc black MaxiSorp® 96-well plates (Thermo Fisher Scientific, USA) were coated overnight with 100 μl per well of mouse anti-human TGFα capture antibody (provided as part of the DuoSet ELISA kit) at 400 ng/ml in TBS at 4° C. After MEF-DKO-hiR2-FL-R441K-T7, MEF-DKO-hiR2-FL-K443R-T7, MEF-DKO-hiR2-FL-V459I-T7, MEF-DKO-hiR2-FL-G481Q-T7, MEF-DKO-hiR2-FL-L488R-T7, MEF-DKO-hiR2-FL-L493I-T7, MEF-DKO-hiR2-FL-D496T-T7, MEF-DKO-hiR2-FL-H505R-T7, MEF-DKO-hiR2-FL-Q512L-T7, MEF-DKO-hiR2-FL- R513K-T7, MEF-DKO-hiR2-FL-D528N-T7, MEF-DKO-hiR2-FL-P533-T7, MEF-DKO-hiR2-FL-M534S-T7, MEF-DKO-hiR2-FL-G540S-T7, MEF-DKO-hiR2-FL-R543Q-T7, MEF-DKO-hiR2-FL-T544P-T7, MEF-DKO-hiR2-FL-G546A-T7, MEF-DKO-hiR2-FL-A547V-T7, MEF-DKO-hiR2-FL-R582Q-T7, MEF-DKO-hiR2-FL-F588L-T7, MEF-DKO-hiR2-FL-M591I-T7, MEF-DKO-hiR2-FL-E594K-T7, MEF-DKO-hiR2-FL-E626D-T7, MEF-DKO-hiR2-FL-L657I-T7, and MEF-DKO-hiR2-FL-H835Y-T7 cells were electroporated with the hTGFα-FL-WT construct in a pcDNA3.1 vector backbone, using an 4D-Nucleofector System (Lonza, Switzerland), approximately 35,000 MEF-DKO cells carrying the human iRhom2 variant with the mouse iRhom2-specific single amino acid substitution or deletion were seeded in 100 μl of normal growth medium in each well of F-bottom 96-well cell culture plates (Thermo Fisher Scientific, USA). On day 2, the capture antibody solution was removed and MaxiSorp® plates were blocked with 300 μl per well of TBS, 1% BSA at room temperature for at least 1 hour. Meanwhile, the cells were washed once with PBS and afterwards 80 μl of OptiMEM medium (Thermo Fisher Scientific, USA) was added per well.

Subsequently, cells (except those for unstimulated controls) were stimulated with 20 μl per well of PMA (Sigma-Aldrich, USA) at a final concentration of 25 ng/ml at 37° C., 5% CO₂ for 1 hour. 20 μl of OptiMEM medium was added to the unstimulated control cells. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Thereafter, 100 μl biotinylated goat anti-human TGFα detection antibody (provided as part of the DuoSet ELISA kit) at 37.5 ng/ml in TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

FIG. 10b shows results from these TGFα release assays demonstrating that all 25 human iRhom2 variants with mouse iRhom2-specific single amino acid substitutions, or single amino acid deletion as in the case of hiR2-FL-P533-, are functionally active as TGFα shedding can be induced with PMA, indicating that these variants are properly folded, in contrast to the empty vector (EV) negative control population, where no PMA-induced shedding of TGFα is detectable.

### FACS Analyses to Characterize Binding of the Purified Antibodies of the Invention for the Purpose of Epitope Mapping

In brief, immortalized MEF-DKO-hiR2-FL-WT-T7 cells, MEF-DKO-miR2-FL-WT-T7 cells and MEF-DKO-hiR2-FL-R441K-T7, MEF-DKO-hiR2-FL-K443R-T7, MEF-DKO-hiR2-FL-V459I-T7, MEF-DKO-hiR2-FL-G481Q-T7, MEF-DKO-hiR2-FL-L488R-T7, MEF-DKO-hiR2-FL-L493I-T7, MEF-DKO-hiR2-FL-D496T-T7, MEF-DKO-hiR2-FL-H505R-T7, MEF-DKO-hiR2-FL-Q512L-T7, MEF-DKO-hiR2-FL- R513K-T7, MEF-DKO-hiR2-FL-D528N-T7, MEF-DKO-hiR2-FL-P533-T7, MEF-DKO-hiR2-FL-M534S-T7, MEF-DKO-hiR2-FL-G540S-T7, MEF-DKO-hiR2-FL-R543Q-T7, MEF-DKO-hiR2-FL-T544P-T7, MEF-DKO-hiR2-FL-G546A-T7, MEF-DKO-hiR2-FL-A547V-T7, MEF-DKO-hiR2-FL-R582Q-T7, MEF-DKO-hiR2-FL-F588L-T7, MEF-DKO-hiR2-FL-M591I-T7, MEF-DKO-hiR2-FL-E594K-T7, MEF-DKO-hiR2-FL-E626D-T7, MEF-DKO-hiR2-FL-L657I-T7, and MEF-DKO-hiR2-FL-H835Y-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well.

To pellet cells and remove supernatants, the plates were centrifuged at 1,500 rpm and 4° C. for 3 minutes. For primary staining, cells were resuspended in 100 μl per well of either FACS buffer alone (controls) or the purified antibodies 3, 5, 16, 22, 34, 42, 43, 44, 48, and 50 of the invention at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

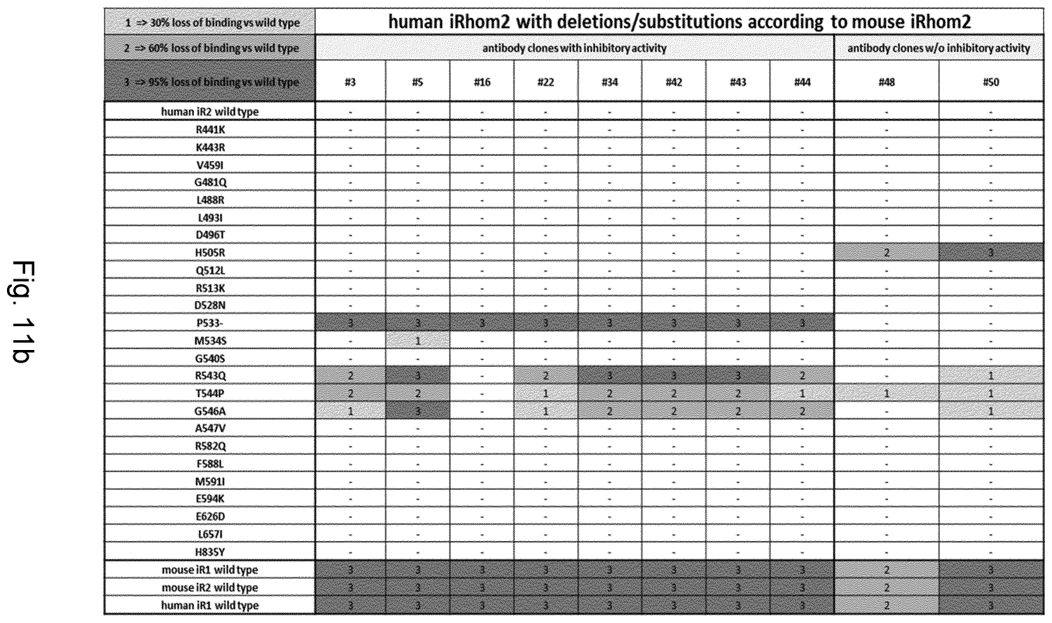

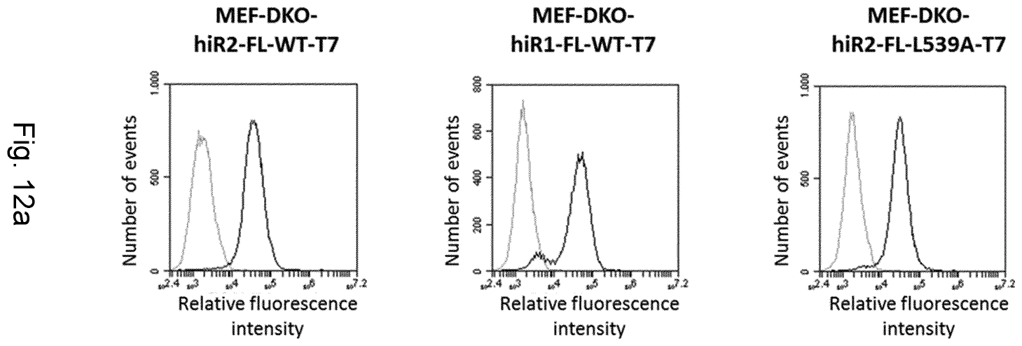

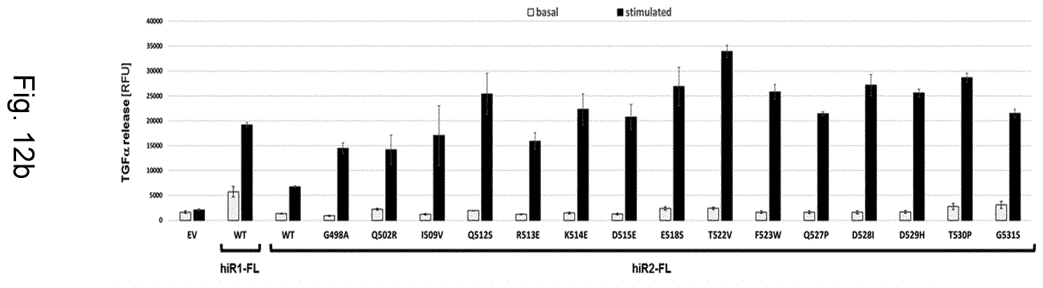

FIG. 11a shows representative results of this experiment. Exemplarily for the entire panel of 25 human iRhom2 variants with mouse iRhom2-related single amino acid substitutions or deletion, data for the analysis of cells expressing the human iRhom2 variant hiR2-FL-P533--T7 are shown. Binding analyses of the antibody 3 as a representative example of antibodies of the invention with inhibitory effects on TNFα release (black, upper panel) or the antibody 50 without inhibitory effects on TNFα release (black, lower panel) as well as anti-mouse IgG secondary antibody (gray) on MEF-DKO-hiR2-FL-WT-T7 cells (left), MEF-DKO-miR2-FL-WT-T7 (middle), and MEF-DKO-hiR2-FL-P533-T7 cells (right) demonstrate the deletion of the single amino acid proline 533 of human iRhom2 to strongly impair and, thus, to contribute to binding of the antibody 3 of the invention with inhibitory effects on TNFα release (right, upper panel). In contrast, it does not affect and, thus, does not contribute to binding of the antibody 50 without inhibitory effects on TNFα release (right, lower panel). For both antibodies, binding to MEF-DKO-hiR2-FL-WT-T7 cells (left) and MEF-DKO-miR2-FL-WT-T7 (middle) serves as positive and negative control, respectively.

FIG. 11b summarizes—in extension of FIG. 11a —the results of FACS analyses of the antibodies 3, 5, 16, 22, 34, 42, 43, and 44 of the invention with inhibitory effects on TNFα release versus the antibodies 48 and 50 without inhibitory effects on TNFα release on the entire panel of 25 engineered MEF populations expressing human iRhom2 variants with mouse iRhom2-specific single amino acid substitutions (including the variant deleted for P533).

Binding of each antibody to human iRhom2 wild type is considered 100 percent. A respective drop of antibody binding to any variant by 30-59% is indicated by cells held in light gray (and marked with “1”), an impaired binding by 60-95% is illustrated by cells colored in gray (and marked with “2”), and a loss of binding by ≥95% is highlighted by dark gray cells (marked with “3”). These data reveal related (except for antibody 16 of the invention) patterns of amino acid positions relevant for binding of the antibodies 3, 5, 16, 22, 34, 42, 43, and 44 of the invention with inhibitory effects on TNFα release to human iRhom2, which are different from patterns of amino acid positions contributing to binding of the antibodies 48 and 50 without inhibitory effects on TNFα release.

### Example 16: Epitope Mapping of the Antibodies of the Invention Based on Family Member-Specific Sequence Variations of iRhom2 in the Central Region of the Large Extracellular Loop

Complementary to Example 15, plasmids for a set of 30 human iRhom2 variants with human iRhom1-related single amino acid substitutions to identify single amino acids that contribute to binding of the antibodies of the invention, were designed in a second approach. These 30 substitutions reflect amino acids in the central region of the large extracellular loop 1 that are non-identical in human iRhom2 versus human iRhom1. Instead of the amino acid of human iRhom2, the amino acid at the corresponding position of human iRhom1 was introduced, resulting in the variants hiR2-FL-G498A-T7, hiR2-FL-Q502R-T7, hiR2-FL-1509V-T7, hiR2-FL-Q512S-T7, hiR2-FL-R513E-T7, hiR2-FL-K514E-T7, hiR2-FL-D515E-T7, hiR2-FL-E518S-T7, hiR2-FL-T522V-T7, hiR2-FL-F523W-T7, hiR2-FL-Q527P-T7, hiR2-FL-D528I-T7, hiR2-FL-D529H-T7, hiR2-FL-T530P-T7, hiR2-FL-G531S-T7, hiR2-FL-P532A-T7, hiR2-FL-S537E-T7, hiR2-FL-D538L-T7, hiR2-FL-L539A-T7, hiR2-FL-Q541H-T7, hiR2-FL-T544Q-T7, hiR2-FL-S545F-T7, hiR2-FL-A547S-T7, hiR2-FL-T555V-T7, hiR2-FL-E557D-T7, hiR2-FL-A560S-T7 and hiR2-FL-S562E-T7. In case no corresponding amino acid exists in human iRhom1, the respective amino acid of human iRhom2 was deleted, resulting in the variants hiR2-FL-M534-T7, hiR2-FL-D535-T7 and hiR2-FL-K536-T7.

This example describes the generation of iRhom1/2−/− DKO MEF populations expressing the 30 human iRhom1-related single amino acid substitution variants as well as their characterization in terms of cell surface localization and functional activity as indicators of proper protein conformation. Subsequently, binding analyses of the purified antibodies 3, 5, 16, 22, 34, 42, 43, 44, 48, and 50 of the invention on the entire panel of 30 engineered MEF populations expressing human iRhom2 variants with human iRhom1-related single amino acid substitutions (including the variants deleted for M534, D535 and K536) are described.

Generation of iRhom1/2−/− DKO MEFs Stably Expressing 30 T7-Tagged Human iRhom2 Variants with Human iRhom1-Related Single Amino Acid Substitutions

In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV-hiR2-FL-G498A-T7, pMSCV-hiR2-FL-Q502R-T7, pMSCV-hiR2-FL-1509V-T7, pMSCV-hiR2-FL-Q512S-T7, pMSCV-hiR2-FL-R513E-T7, pMSCV-hiR2-FL-K514E-T7, pMSCV-hiR2-FL-D515E-T7, pMSCV-hiR2-FL-E518S-T7, pMSCV-hiR2-FL-T522V-T7, pMSCV-hiR2-FL-F523W-T7, pMSCV-hiR2-FL-Q527P-T7, pMSCV-hiR2-FL-D528I-T7, pMSCV-hiR2-FL-D529H-T7, pMSCV-hiR2-FL-T530P-T7, pMSCV-hiR2-FL-G531S-T7, pMSCV-hiR2-FL-P532A-T7, pMSCV-hiR2-FL-M534-T7, pMSCV-hiR2-FL-D535-T7, pMSCV-hiR2-FL-K536-T7, pMSCV-hiR2-FL-S537E-T7, pMSCV-hiR2-FL-D538L-T7, pMSCV-hiR2-FL-L539A-T7, pMSCV-hiR2-FL-Q541H-T7, pMSCV-hiR2-FL-T544Q-T7, pMSCV-hiR2-FL-S545F-T7, pMSCV-hiR2-FL-A547S-T7, pMSCV-hiR2-FL-T555V-T7, pMSCV-hiR2-FL-E557D-T7, pMSCV-hiR2-FL-A560S-T7 and pMSCV-hiR2-FL-S562E-T7 encoding human iRhom2 full length single amino acid substitutions C-terminally tagged with 3 consecutive copies of the T7 epitope (MASMTGGQQMG), and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV-hiR2-FL-G498A-T7, pMSCV-hiR2-FL-Q502R-T7, pMSCV-hiR2-FL-1509V-T7, pMSCV-hiR2-FL-Q512S-T7, pMSCV-hiR2-FL-R513E-T7, pMSCV-hiR2-FL-K514E-T7, pMSCV-hiR2-FL-D515E-T7, pMSCV-hiR2-FL-E518S-T7, pMSCV-hiR2-FL-T522V-T7, pMSCV-hiR2-FL-F523W-T7, pMSCV-hiR2-FL-Q527P-T7, pMSCV-hiR2-FL-D528I-T7, pMSCV-hiR2-FL-D529H-T7, pMSCV-hiR2-FL-T530P-T7, pMSCV-hiR2-FL-G531S-T7, pMSCV-hiR2-FL-P532A-T7, pMSCV-hiR2-FL-M534-T7, pMSCV-hiR2-FL-D535-T7, pMSCV-hiR2-FL-K536-T7, pMSCV-hiR2-FL-S537E-T7, pMSCV-hiR2-FL-D538L-T7, pMSCV-hiR2-FL-L539A-T7, pMSCV-hiR2-FL-Q541H-T7, pMSCV-hiR2-FL-T544Q-T7, pMSCV-hiR2-FL-S545F-T7, pMSCV-hiR2-FL-A547S-T7, pMSCV-hiR2-FL-T555V-T7, pMSCV-hiR2-FL-E557D-T7, pMSCV-hiR2-FL-A560S-T7 and pMSCV-hiR2-FL-S562E-T7 ecotrophic virus were collected, filtered with 0.45 μm CA filters, and supplemented with 4 μg/ml of polybrene (Sigma-Aldrich, USA). Upon removal of medium from the immortalized iRhom1/2−/− DKO MEFs, these supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO-hiR2-FL-G498A-T7, MEF-DKO-hiR2-FL-Q502R-T7, MEF-DKO-hiR2-FL-1509V-T7, MEF-DKO-hiR2-FL-Q512S-T7, MEF-DKO-hiR2-FL-R513E-T7, MEF-DKO-hiR2-FL-K514E-T7, MEF-DKO-hiR2-FL-D515E-T7, MEF-DKO-hiR2-FL-E518S-T7, MEF-DKO-hiR2-FL-T522V-T7, MEF-DKO-hiR2-FL-F523W-T7, MEF-DKO-hiR2-FL-Q527P-T7, MEF-DKO-hiR2-FL-D528I-T7, MEF-DKO-hiR2-FL-D529H-T7, MEF-DKO-hiR2-FL-T530P-T7, MEF-DKO-hiR2-FL-G531S-T7, MEF-DKO-hiR2-FL-P532A-T7, MEF-DKO-hiR2-FL-M534-T7, MEF-DKO-hiR2-FL-D535-T7, MEF-DKO-hiR2-FL-K536-T7, MEF-DKO-hiR2-FL-S537E-T7, MEF-DKO-hiR2-FL-D538L-T7, MEF-DKO-hiR2-FL-L539A-T7, MEF-DKO-hiR2-FL-Q541H-T7, MEF-DKO-hiR2-FL-T544Q-T7, MEF-DKO-hiR2-FL-S545F-T7, MEF-DKO-hiR2-FL-A547S-T7, MEF-DKO-hiR2-FL-T555V-T7, MEF-DKO-hiR2-FL-E557D-T7, MEF-DKO-hiR2-FL-A560S-T7 and MEF-DKO-hiR2-FL-S562E-T7 cells stably expressing human iRhom2 full length single amino acid substitutions C-terminally tagged with 3 consecutive copies of the T7 epitope. Upon propagation, cells were stocked for future usage.

### FACS Analyses for Test System Validation

In brief, immortalized MEF-DKO-hiR2-FL-WT-T7 cells, MEF-DKO-hiR1-FL-WT-T7 cells and MEF-DKO-hiR2-FL-G498A-T7, MEF-DKO-hiR2-FL-Q502R-T7, MEF-DKO-hiR2-FL-I509V-T7, MEF-DKO-hiR2-FL-Q512S-T7, MEF-DKO-hiR2-FL-R513E-T7, MEF-DKO-hiR2-FL-K514E-T7, MEF-DKO-hiR2-FL-D515E-T7, MEF-DKO-hiR2-FL-E518S-T7, MEF-DKO-hiR2-FL-T522V-T7, MEF-DKO-hiR2-FL-F523W-T7, MEF-DKO-hiR2-FL-Q527P-T7, MEF-DKO-hiR2-FL-D528I-T7, MEF-DKO-hiR2-FL-D529H-T7, MEF-DKO-hiR2-FL-T530P-T7, MEF-DKO-hiR2-FL-G531S-T7, MEF-DKO-hiR2-FL-P532A-T7, MEF-DKO-hiR2-FL-M534-T7, MEF-DKO-hiR2-FL-D535-T7, MEF-DKO-hiR2-FL-K536-T7, MEF-DKO-hiR2-FL-S537E-T7, MEF-DKO-hiR2-FL-D538L-T7, MEF-DKO-hiR2-FL-L539A-T7, MEF-DKO-hiR2-FL-Q541H-T7, MEF-DKO-hiR2-FL-T544Q-T7, MEF-DKO-hiR2-FL-S545F-T7, MEF-DKO-hiR2-FL-A547S-T7, MEF-DKO-hiR2-FL-T555V-T7, MEF-DKO-hiR2-FL-E557D-T7, MEF-DKO-hiR2-FL-A560S-T7 and MEF-DKO-hiR2-FL-S562E-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or mouse monoclonal anti-T7 IgG (Merck Millipore, USA) at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

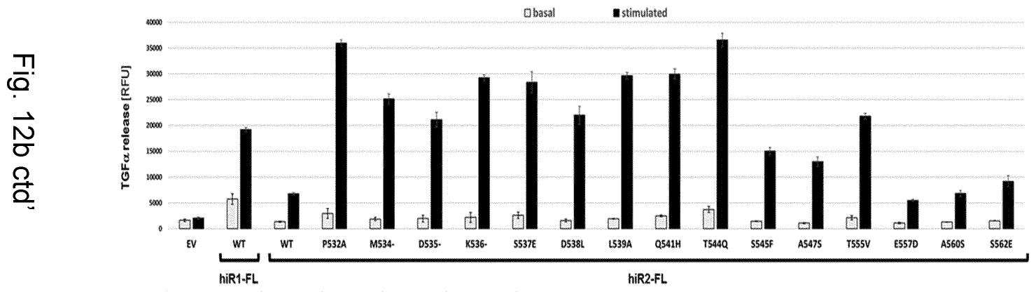

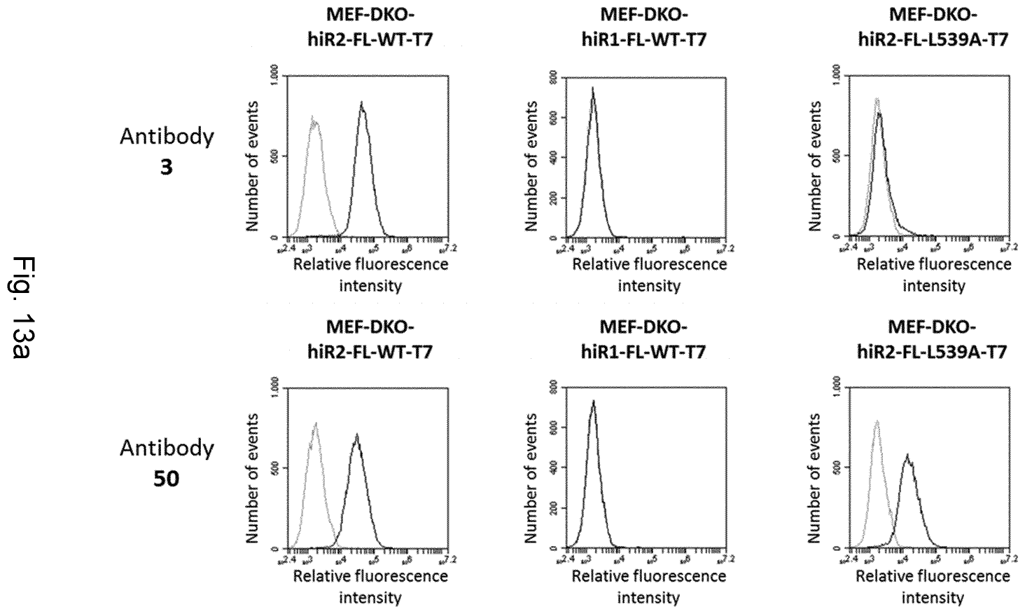

FIG. 12a shows representative results of this experiment exemplarily for the human iRhom2 variant hiR2-FL-L539A-T7. Binding analyses of anti-T7 tag antibody (black) and anti-mouse IgG secondary antibody (gray) on MEF-DKO-hiR2-FL-WT-T7 (left), MEF-DKO-hiR1-FL-WT-T7 (middle) and MEF-DKO-hiR2-FL-L539A-T7 cells (right) reveal a comparably strong increase in relative fluorescence intensity. This demonstrates that, similarly to human iRhom2 and human iRhom1 wild type (left and middle), the human iRhom2 variant hiR2-FL-L539A-T7 is equally well expressed and localized on the surface of these cells (right). Similar results were obtained for the expression and localization of the other human iRhom2 full length single amino acid substitutions expressed on MEF-DKO-hiR2-FL-G498A-T7, MEF-DKO-hiR2-FL-Q502R-T7, MEF-DKO-hiR2-FL-I509V-T7, MEF-DKO-hiR2-FL-Q512S-T7, MEF-DKO-hiR2-FL-R513E-T7, MEF-DKO-hiR2-FL-K514E-T7, MEF-DKO-hiR2-FL-D515E-T7, MEF-DKO-hiR2-FL-E518S-T7, MEF-DKO-hiR2-FL-T522V-T7, MEF-DKO-hiR2-FL-F523W-T7, MEF-DKO-hiR2-FL-Q527P-T7, MEF-DKO-hiR2-FL-D528I-T7, MEF-DKO-hiR2-FL-D529H-T7, MEF-DKO-hiR2-FL-T530P-T7, MEF-DKO-hiR2-FL-G531S-T7, MEF-DKO-hiR2-FL-P532A-T7, MEF-DKO-hiR2-FL-S537E-T7, MEF-DKO-hiR2-FL-D538L-T7, MEF-DKO-hiR2-FL-Q541H-T7, MEF-DKO-hiR2-FL-T544Q-T7, MEF-DKO-hiR2-FL-S545F-T7, MEF-DKO-hiR2-FL-A547S-T7, MEF-DKO-hiR2-FL-T555V-T7, MEF-DKO-hiR2-FL-E557D-T7, MEF-DKO-hiR2-FL-A560S-T7 and MEF-DKO-hiR2-FL-S562E-T7 cells, as well as for the expression and localization of human iRhom2 full length single amino acid deletions expressed on MEF-DKO-hiR2-FL-M534-T7, MEF-DKO-hiR2-FL-D535-T7 and MEF-DKO-hiR2-FL-K536-T7 cells.

TGFα ELISA for Test System Validation To test all 30 human iRhom2 variants with human iRhom1-specific single amino acid substitutions, or single amino acid deletion as in the case of hiR2-FL-M534-, hiR2-FL-D535—and hiR2-FL-K536-, the respective MEF-DKO cell lines stably expressing these variants, generated as described in the example above, were subjected to TGFα shedding ELISA analysis. In order to demonstrate the functionality of all variants as an indicator that these variants are properly folded, PMA-induced release of nucleofected TGFα was assessed. As the cells used in this analysis are rescue variants of iRhom1/2−/− double knockout mouse embryonic fibroblasts (described in Example 11), that are rescued by the respective human iRhom2 variant with the human iRhom1-specific single amino acid substitution or deletion, the iRhom2 variant stably expressed is the only iRhom protein expressed in these cells at all and is therefore the only contributing iRhom to the shedding TGFα in these cells.

In brief, on day 1, Nunc black MaxiSorp® 96-well plates (Thermo Fisher Scientific, USA) were coated overnight with 100 μl per well of mouse anti-human TGFα capture antibody (provided as part of the DuoSet ELISA kit) at 400 ng/ml in TBS at 4° C. After MEF-DKO-hiR2-FL-G498A-T7, MEF-DKO-hiR2-FL-Q502R-T7, MEF-DKO-hiR2-FL-I509V-T7, MEF-DKO-hiR2-FL-Q512S-T7, MEF-DKO-hiR2-FL-R513E-T7, MEF-DKO-hiR2-FL-K514E-T7, MEF-DKO-hiR2-FL-D515E-T7, MEF-DKO-hiR2-FL-E518S-T7, MEF-DKO-hiR2-FL-T522V-T7, MEF-DKO-hiR2-FL-F523W-T7, MEF-DKO-hiR2-FL-Q527P-T7, MEF-DKO-hiR2-FL-D528I-T7, MEF-DKO-hiR2-FL-D529H-T7, MEF-DKO-hiR2-FL-T530P-T7, MEF-DKO-hiR2-FL-G531S-T7, MEF-DKO-hiR2-FL-P532A-T7, MEF-DKO-hiR2-FL-M534-T7, MEF-DKO-hiR2-FL-D535-T7, MEF-DKO-hiR2-FL-K536-T7, MEF-DKO-hiR2-FL-S537E-T7, MEF-DKO-hiR2-FL-D538L-T7, MEF-DKO-hiR2-FL-L539A-T7, MEF-DKO-hiR2-FL-Q541H-T7, MEF-DKO-hiR2-FL-T544Q-T7, MEF-DKO-hiR2-FL-S545F-T7, MEF-DKO-hiR2-FL-A547S-T7, MEF-DKO-hiR2-FL-T555V-T7, MEF-DKO-hiR2-FL-E557D-T7, MEF-DKO-hiR2-FL-A560S-T7 and MEF-DKO-hiR2-FL-S562E-T7 cells were electroporated with the hTGFα-FL-WT construct in a pcDNA3.1 vector backbone, using an 4D-Nucleofector System (Lonza, Switzerland), approximately 35,000 MEF-DKO cells carrying the human iRhom2 variant with the human iRhom1-specific single amino acid substitution or deletion were seeded in 100 μl of normal growth medium in each well of F-bottom 96-well cell culture plates (Thermo Fisher Scientific, USA). On day 2, the capture antibody solution was removed and MaxiSorp® plates were blocked with 300 μl per well of TBS, 1% BSA at room temperature for at least 1 hour. Meanwhile, the cells were washed once with PBS and afterwards 80 μl of OptiMEM medium (Thermo Fisher Scientific, USA) was added per well.

Subsequently, cells (except those for unstimulated controls) were stimulated with 20 μl per well of PMA (Sigma-Aldrich, USA) at a final concentration of 25 ng/ml at 37° C., 5% CO₂ for 1 hour. 20 μl of OptiMEM medium was added to the unstimulated control cells. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Thereafter, 100 μl biotinylated goat anti-human TGFα detection antibody (provided as part of the DuoSet ELISA kit) at 37.5 ng/ml in TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

FIG. 12b shows results from these TGFα release assays demonstrating that all 30 human iRhom2 variants with human iRhom1-specific single amino acid substitutions, or single amino acid deletions as in the case of hiR2-FL-M534-, hiR2-FL-D535—and hiR2-FL-K536-, are functionally active as TGFα shedding can be induced with PMA, indicating that these variants are properly folded, in contrast to the empty vector (EV) negative control population, where no PMA-induced shedding of TGFα is detectable.

### FACS Analyses to Characterize Binding of the Purified Antibodies of the Invention for the Purpose of Epitope Mapping

In brief, immortalized MEF-DKO-hiR2-FL-G498A-T7, MEF-DKO-hiR2-FL-Q502R-T7, MEF-DKO-hiR2-FL-I509V-T7, MEF-DKO-hiR2-FL-Q512S-T7, MEF-DKO-hiR2-FL-R513E-T7, MEF-DKO-hiR2-FL-K514E-T7, MEF-DKO-hiR2-FL-D515E-T7, MEF-DKO-hiR2-FL-E518S-T7, MEF-DKO-hiR2-FL-T522V-T7, MEF-DKO-hiR2-FL-F523W-T7, MEF-DKO-hiR2-FL-Q527P-T7, MEF-DKO-hiR2-FL-D528I-T7, MEF-DKO-hiR2-FL-D529H-T7, MEF-DKO-hiR2-FL-T530P-T7, MEF-DKO-hiR2-FL-G531S-T7, MEF-DKO-hiR2-FL-P532A-T7, MEF-DKO-hiR2-FL-M534-T7, MEF-DKO-hiR2-FL-D535-T7, MEF-DKO-hiR2-FL-K536-T7, MEF-DKO-hiR2-FL-S537E-T7, MEF-DKO-hiR2-FL-D538L-T7, MEF-DKO-hiR2-FL-L539A-T7, MEF-DKO-hiR2-FL-Q541H-T7, MEF-DKO-hiR2-FL-T544Q-T7, MEF-DKO-hiR2-FL-S545F-T7, MEF-DKO-hiR2-FL-A547S-T7, MEF-DKO-hiR2-FL-T555V-T7, MEF-DKO-hiR2-FL-E557D-T7, MEF-DKO-hiR2-FL-A560S-T7 and MEF-DKO-hiR2-FL-S562E-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or the purified antibodies 3, 5, 16, 22, 34, 42, 43, 44, 48, and 50 of the invention at 3 μg/ml in FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

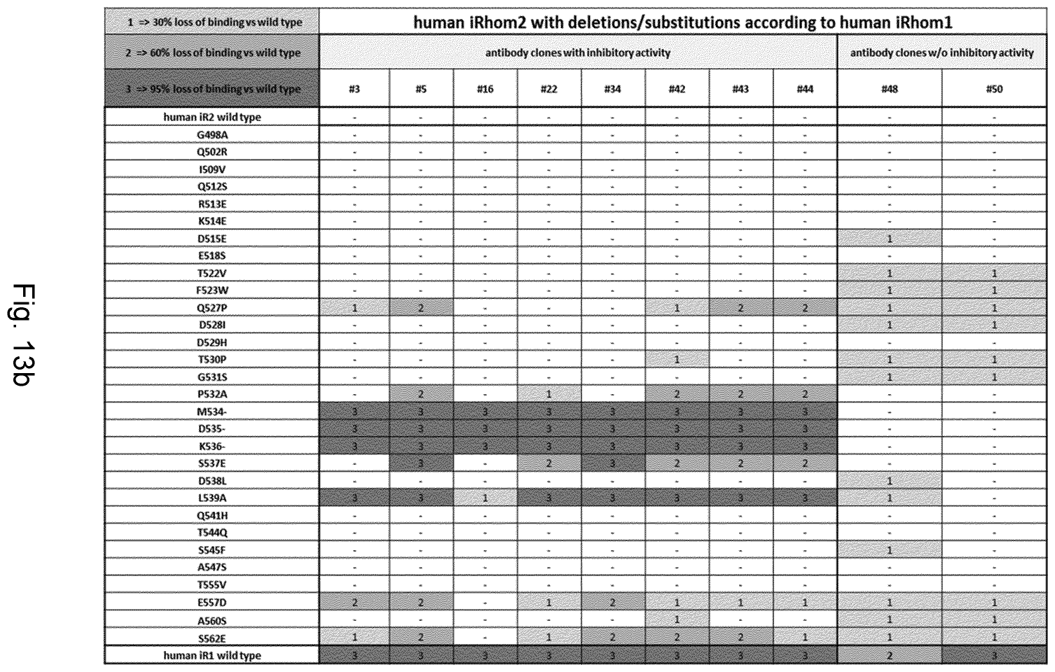

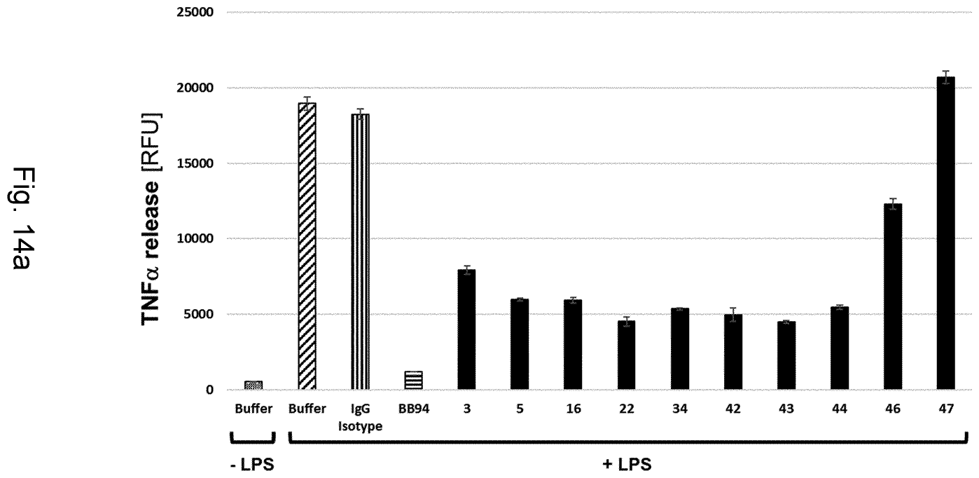

FIG. 13a shows representative results of this experiment. Exemplarily for the entire panel of 30 human iRhom2 variants with human iRhom1-related single amino acid substitutions or deletion, data for the analysis of cells expressing the human iRhom2 variant hiR2-FL-L539A-T7 are shown. Binding analyses of the antibody 3 as a representative example of the antibodies of the invention with inhibitory effects on TNFα release (black, upper panel) or the antibody 50 without inhibitory effects on TNFα release (black, lower panel) as well as anti-mouse IgG secondary antibody (gray) on MEF-DKO-hiR2-FL-WT-T7 cells (left), MEF-DKO-hiR1-FL-WT-T7 (middle), and MEF-DKO-hiR2-FL-L539A-T7 cells (right) demonstrate the substitution of the single amino acid leucine 539 of human iRhom2 by alanine to strongly impair and, thus, to contribute to binding of the antibody 3 of the invention with inhibitory effects on TNFα release (right, upper panel). In contrast, it does not affect and, thus, does not contribute to binding of the antibody 50 without inhibitory effects on TNFα release (right, lower panel). For both antibodies, binding to MEF-DKO-hiR2-FL-WT-T7 cells (left) and MEF-DKO-hiR1-FL-WT-T7 (middle) serves as positive and negative control, respectively.

FIG. 13b summarizes—in extension of FIG. 13a —the results of FACS analyses of the antibodies 3, 5, 16, 22, 34, 42, 43, and 44 of the invention with inhibitory effects on TNFα release versus the antibodies 48 and 50 without inhibitory effects on TNFα release on the entire panel of 30 engineered MEF populations expressing human iRhom2 variants with human iRhom1-specific single amino acid substitutions (including the variants deleted for M534, D535 and K536). Binding of each antibody to human iRhom2 wild type is considered 100 percent. A respective drop of antibody binding to any variant by 30-59% is indicated by cells held in light gray (and marked with “1”), an impaired binding by 60-95% is illustrated by cells colored in gray (and marked with “2”), and a loss of binding by ≥95% is highlighted by dark gray cells (marked with “3”). These data reveal related (except for antibody 16 of the invention) patterns of amino acid positions relevant for binding of the antibodies 3, 5, 16, 22, 34, 42, 43, and 44 of the invention with inhibitory effects on TNFα release to human iRhom2, which are different from patterns of amino acid positions contributing to binding of the antibodies 48 and 50 without inhibitory effects on TNFα release.

### Example 17: Analysis of Inhibitory Effects of the Antibodies of the Invention on LPS-Induced TNFα Shedding In Vitro

In contrast to Example 14, where the hybridoma supernatant-derived purified antibodies of the invention were tested in ELISA-based TNFα release assays, this analysis was conducted with recombinant produced antibodies of the invention to verify their inhibitory effects on LPS-induced release of endogenous TNFα from human THP-1 monocytic cells.

To produce the recombinant antibody material, target DNA sequence was designed, optimized and synthesized. The complete sequence was sub-cloned into pcDNA3.4 vector (Thermo Fisher Scientific, USA) and the transfection grade plasmid was maxi-prepared for Expi293F (Thermo Fisher Scientific, USA) cell expression. Expi293F cells were grown in serum-free Expi293FTM expression medium (Thermo Fisher Scientific, USA) in Erlenmeyer flasks (Coming Inc., USA) at 37° C. with 8% CO₂ on an orbital shaker (VWR Scientific, Germany). One day before transfection, the cells were seeded at an appropriate density in new Erlenmeyer flasks. On the day of transfection, DNA and transfection reagent were mixed at an optimal ratio and then added into the flask with cells ready for transfection. The recombinant plasmids encoding target protein were transiently transfected into suspension Expi293F cell cultures. The cell culture supernatant collected on day 6 post-transfection was used for purification. Cell culture broth was centrifuged and filtrated. Filtered cell culture supernatant was loaded onto either HiTrap MabSelect SuRe (GE Healthcare, UK), MabSelect SuRe™ LX (GE Healthcare, UK) or RoboColumn Eshmuno A (Merck Millipore, USA) affinity purification columns at an appropriate flowrate. After washing and elution with appropriate buffers, the eluted fractions were pooled and buffer exchanged to final formulation buffer. The purified protein was analyzed by SDS-PAGE analysis for molecular weight and purity measurements. Finally, the concentration was determined applying a NanoDrop 2000 spectrophotometer (Thermo Fisher Scientific, USA).

The ELISA-based TNFα release assay that was used in this example is identical to the one described in Example 14.

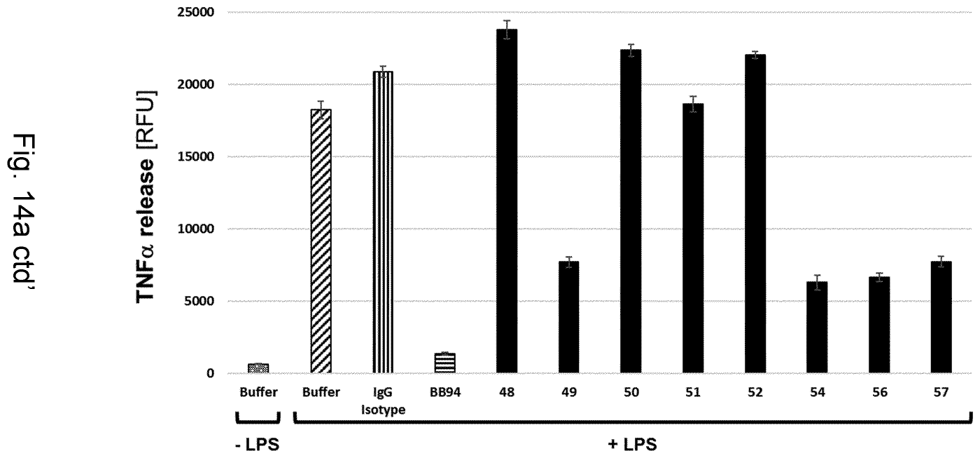

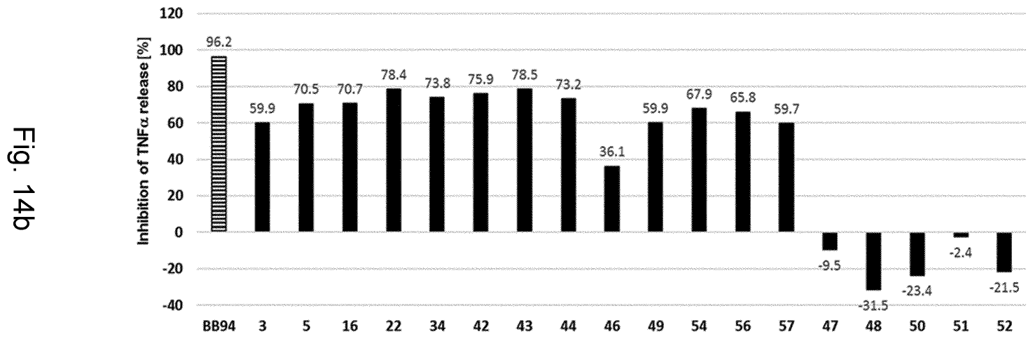

FIG. 14 shows representative results of this experiment demonstrating the effects of test articles on LPS-induced release of TNFα from THP-1 cells in absolute numbers (FIG. 14A) and percent inhibition (FIG. 14B). While Batimastat (BB94) as a small molecule inhibitor of metalloproteinases serves as positive control and results in 96.2% inhibition of LPS-induced release of TNFα, the presence of IgG isotype control has no significant effect on TNFα shedding. In contrast, an equal concentration of the purified antibodies 3, 5, 16, 22, 34, 42, 43, 44, 46, 49, 54, 56, and 57 of the invention inhibits LPS-induced release of TNFα from THP-1 cells by 59.9%, 70.5%, 70.7%, 78.4%, 73.8%, 75.9%, 78.5%, 73.2%, 36.1%, 59.9%, 67.9%, 65.8%, and 59.7%, respectively. Again in contrast and therefore comparable to the IgG isotype control, the presence of the purified antibodies 47, 48, 50, 51 and 52 of the invention has no significant effect on TNFα shedding.

### Example 18: Evaluation of Cross-Reactivity of the Antibodies of the Invention to Different Species

Next, iRhom1/2−/− DKO MEFs stably expressing a tagged form of rhesus monkey, cynomolgus monkey, dog or rabbit iRhom2 were generated in order to determine cross-reactivity of the antibodies of the invention with the respective orthologue of iRhom2. iRhom1/2−/− DKO MEFs stably expressing a tagged form of rhesus monkey, cynomolgus monkey, dog or rabbit iRhom1 were generated to confirm specificity for iRhom2 versus iRhom1 of these species.

Generation of iRhom1/2−/− DKO MEFs Stably Expressing T7-Tagged Rhesus Monkey, Cynomolgus Monkey, Dog or Rabbit iRhom2

In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV (Clontech, USA) empty vector, pMSCV-rhesus-iR2-FL-WT-T7 encoding rhesus monkey iRhom2 full length wild type C-terminally tagged with 3 consecutive copies of the T7 epitope, pMSCV-cyno-iR2-FL-WT-T7 encoding cynomolgus monkey iRhom2 full length wild type C-terminally tagged with 3 consecutive copies of the T7 epitope, pMSCV-dog-iR2-FL-WT-T7 encoding dog iRhom2 full length wild type C-terminally tagged with 3 consecutive copies of the T7 epitope or pMSCV-rabbit-iR2-FL-WT-T7 encoding rabbit iRhom2 full length wild type C-terminally tagged with 3 consecutive copies of the T7 epitope, respectively, and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV, pMSCV-rhesus-iR2-FL-WT-T7, pMSCV-cyno-iR2-FL-WT-T7, pMSCV-dog-iR2-FL-WT-T7 or pMSCV-rabbit-iR2-FL-WT-T7 ecotrophic virus, respectively were collected, filtered with 0.45 μm CA filters, and supplemented with 4 μg/ml of polybrene (Sigma-Aldrich, USA). Upon removal of medium from the immortalized iRhom1/2−/− DKO MEFs, the virus containing supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO-EV control cells stably infected with pMSCV empty vector, pMSCV-rhesus-iR2-FL-WT-T7 cells stably expressing rhesus monkey iRhom2 full length wild type C-terminally tagged with 3 consecutive copies of the T7 epitope, pMSCV-cyno-iR2-FL-WT-T7 cells stably expressing cynomolgus monkey iRhom2 full length wild type C-terminally tagged with 3 consecutive copies of the T7 epitope, pMSCV-dog-iR2-FL-WT-T7 cells stably expressing dog iRhom2 full length wild type C-terminally tagged with 3 consecutive copies of the T7 epitope or pMSCV-rabbit-iR2-FL-WT-T7 cells stably expressing rabbit iRhom2 full length wild type C-terminally tagged with 3 consecutive copies of the T7 epitope, respectively. Upon propagation, cells were stocked for future usage. In parallel, iRhom1/2−/− DKO MEFs stably expressing a tagged form of rhesus monkey, cynomolgus monkey, dog or rabbit iRhom1 were generated in an analogous manner.

### FACS Analyses for Test System Validation and Antibody Characterization

In brief, immortalized MEF-DKO-EV control cells, MEF-DKO-rhesus-iR2-FL-WT-T7 cells, MEF-DKO-cyno-iR2-FL-WT-T7 cells, MEF-DKO-dog-iR2-FL-WT-T7 cells and MEF-DKO-rabbit-iR2-FL-WT-T7 cells, as well as their respective iRhom1 counterparts, were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls), mouse monoclonal anti-T7 IgG (Merck Millipore, USA) at 3 μg/ml FACS buffer or the antibodies of the invention also at 3 μg/ml FACS buffer and incubated on ice for 1 hour.

Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

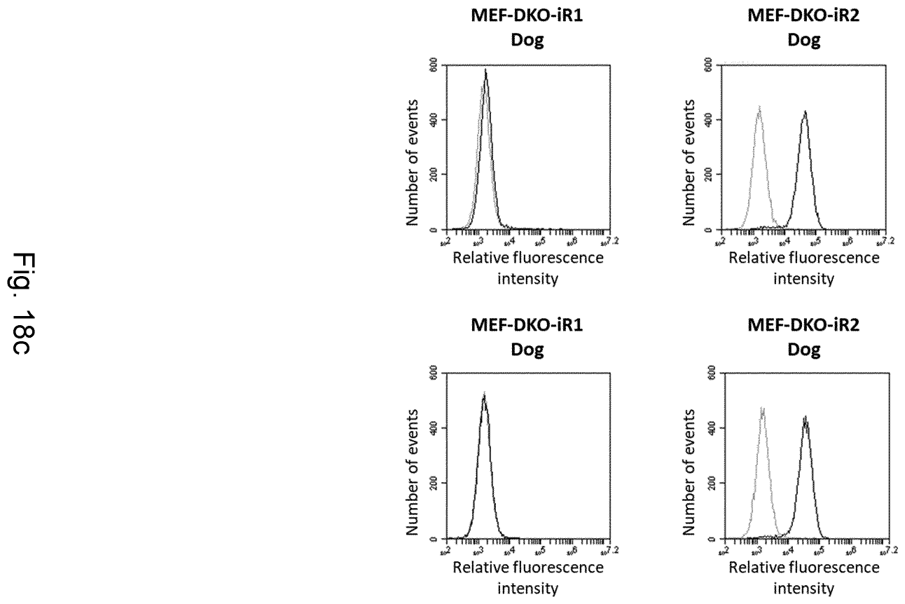

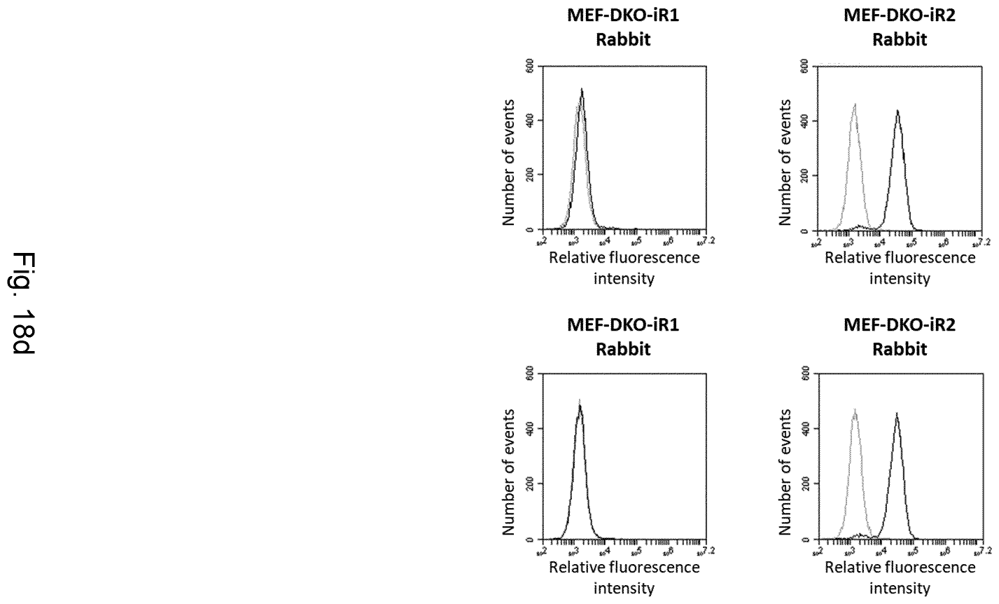

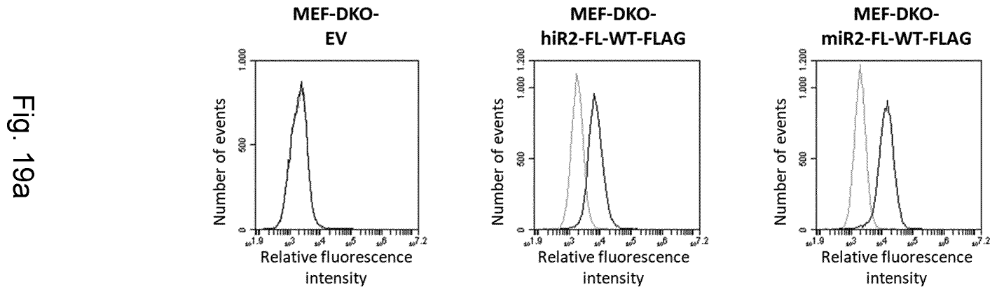

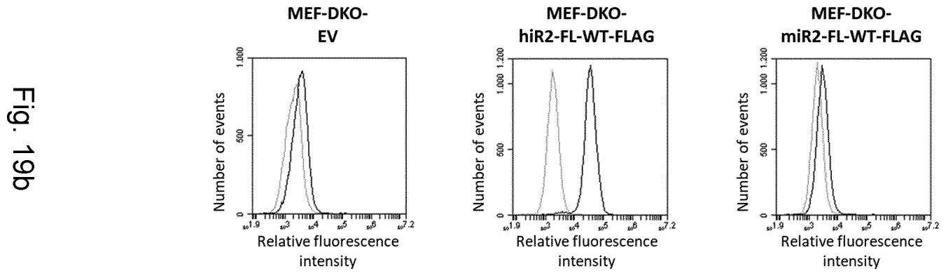

FIGS. 18a, 18b, 18c & 18d show representative results of this experiment. As compared to control samples incubated with secondary antibody only (18a, 18b, 18c & 18d, gray), the strong shift in relative fluorescence intensity on MEF-DKO-rhesus-iR2-FL-WT-T7 cells, MEF-DKO-cyno-iR2-FL-WT-T7 cells, MEF-DKO-dog-iR2-FL-WT-T7 cells and MEF-DKO-rabbit-iR2-FL-WT-T7 cells, demonstrates strong binding of the antibody 16 of the invention as a representative example of the antibodies 3, 5, 16, 22, 34, 42, 43, 44, 46, 47, 48, 49, 50, 51, 54, 56 and 57 of the invention to the rhesus monkey iRhom2 variant (FIG. 18a, black, right), cynomolgus monkey iRhom2 variant (FIG. 18b, black, right), dog iRhom2 variant (FIG. 18c, black, right) and rabbit iRhom2 variant (FIG. 18d, black, right), respectively. In contrast, no binding of the antibody 16 of the invention as a representative example of the antibodies of the invention (except for antibody 52) to MEF-DKO-rhesus-iR1-FL-WT-T7 cells, MEF-DKO-cyno-iR1-FL-WT-T7 cells, MEF-DKO-dog-iR1-FL-WT-T7 cells and MEF-DKO-rabbit-iR1-FL-WT-T7 cells is detectable, providing evidence that the rhesus monkey iRhom1 variant (FIG. 18a, black, left), cynomolgus monkey iRhom1 variant (FIG. 18b, black, left), dog iRhom1 variant (FIG. 18c, black, left) and rabbit iRhom1 variant (FIG. 18d, black, left), respectively is not being recognized by the antibody 16 of the invention. The aforementioned cross-reactivities to the different iRhom2 orthologues as well as their specificities for iRhom2 versus iRhom1 of these species are depicted both for the murine (upper panel) and for the chimeric (lower panel) version of antibody 16 in each figure.

### Example 19: Evaluation of Mouse Cross-Reactivity of the Purified Antibodies of the Invention

In addition to Example 12, where T7-tagged iRhom variants were described, immortalized iRhom1/2−/− DKO MEFs were reconstituted with a FLAG-tagged form of human iRhom2 in order to confirm target recognition for the antibodies of the invention. Additionally, iRhom1/2-/− DKO MEFs stably expressing a FLAG-tagged form of mouse iRhom2 were generated in order to determine cross-reactivity of the antibodies of the invention with the mouse orthologue of iRhom2.

Generation of iRhom1/2−/− DKO MEFs Stably Expressing FLAG-Tagged Human or Mouse iRhom2

In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV (Clontech, USA) empty vector, pMSCV-hiR2-FL-WT-FLAG encoding human iRhom2 full length wild type C-terminally tagged with the triple-FLAG epitope (DYKDHDGDYKDHDIDYKDDDDK) or pMSCV-miR2-FL-WT-FLAG encoding mouse iRhom2 full length wild type C-terminally tagged with the triple-FLAG epitope, and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV, pMSCV-hiR2-FL-WT-FLAG or pMSCV-miR2-FL-WT-FLAG ecotrophic virus were collected, filtered with 0.45 μm CA filters, and supplemented with 4 μg/ml of polybrene (Sigma-Aldrich, USA). Upon removal of medium from the immortalized iRhom1/2−/− DKO MEFs, the virus containing supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO-EV control cells stably infected with pMSCV empty vector, MEF-DKO-hiR2-FL-WT-FLAG cells stably expressing human iRhom2 full length wild type C-terminally tagged with the triple-FLAG epitope, and MEF-DKO-miR2-FL-WT-FLAG cells stably expressing mouse iRhom2 full length wild type C-terminally tagged with the triple-FLAG epitope. Upon propagation, cells were stocked for future usage.

### FACS Analyses for Test System Validation and Antibody Characterization

In brief, immortalized MEF-DKO-EV control cells, MEF-DKO-hiR2-FL-WT-FLAG cells and MEF-DKO-miR2-FL-WT-FLAG cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 3×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls), mouse monoclonal anti-FLAG IgG (Sigma-Aldrich, USA) at 3 μg/ml FACS buffer or the antibodies of the invention also at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

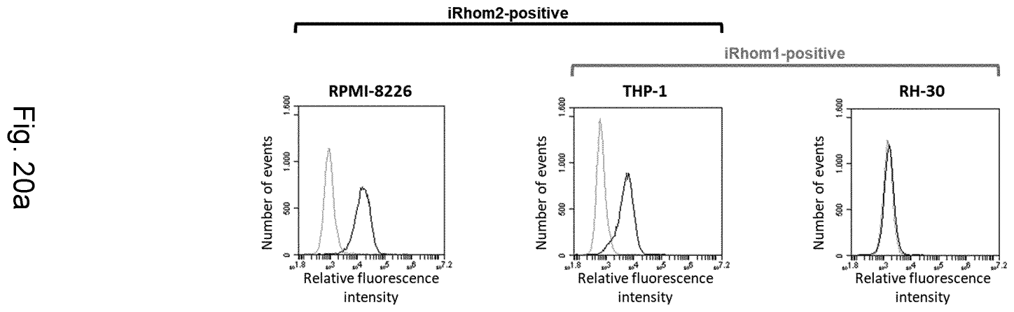

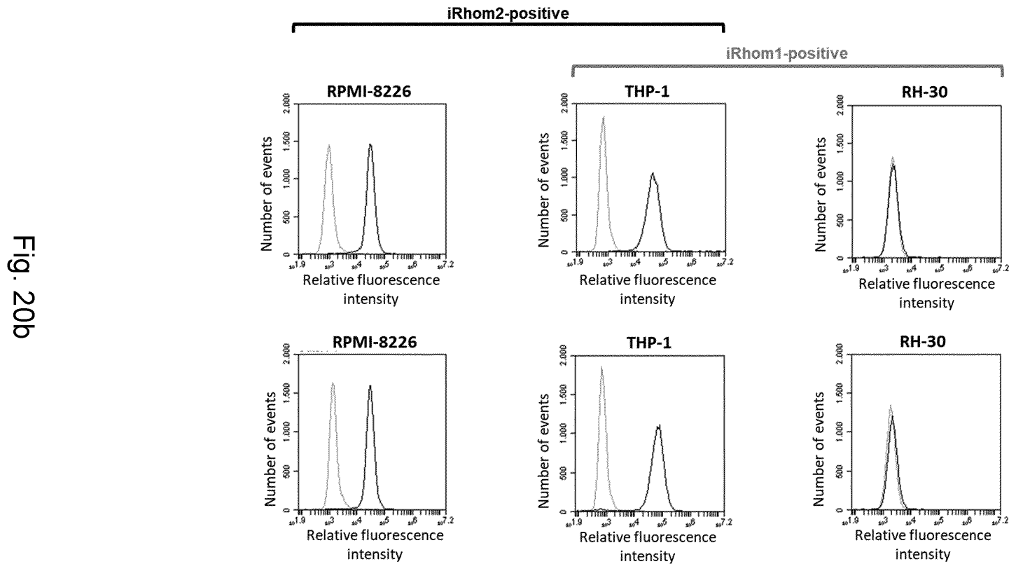

FIGS. 19a & 19b show representative results of this experiment. As compared to control samples incubated with anti-mouse IgG secondary antibody only (19a & 19b, gray), co-incubation with anti-FLAG tag antibody (FIG. 19a, black) results in no background staining of MEF-DKO-EV control cells (FIG. 19a, left). In contrast, binding analyses of the anti-FLAG tag antibody on both MEF-DKO-hiR2-FL-WT-FLAG (FIG. 19a, middle) and MEF-DKO-miR2-FL-WT-FLAG (FIG. 19a, right) cells reveal a strong increase in relative fluorescence intensity, demonstrating both the ectopically expressed human and the mouse iRhom2 orthologues to be localized on the surface of these genetically engineered cell populations and, thus, validating them as suitable test systems for characterizing the antibodies of the invention. Co-incubation of these cell populations with antibody 3 as a representative example of the antibodies of the invention (FIG. 19b, black) leads to very little background staining of MEF-DKO-EV control cells at all (FIG. 19b, left), while the strong shift in relative fluorescence intensity, similar to the one observed with the anti-FLAG tag antibody, on MEF-DKO-hiR2-FL-WT-FLAG cells demonstrates strong binding of the antibody 3 of the invention to human iRhom2 (FIG. 19b, middle). In contrast, no significant binding of the antibody 3 of the invention to MEF-DKO-miR2-FL-WT-FLAG cells is detectable (FIG. 19b, right), providing evidence that mouse iRhom2, whose presence on the cell surface is verified with the anti-FLAG tag antibody (FIG. 19a, right), is not being recognized by the antibody 3 of the invention. Similar results were obtained with the antibodies 5, 16, 22, 34, 42, 43, 44, 46, 47, 48, 49, 50, 51, 54, 56 and 57 of the invention, demonstrating that none of these antibodies of the invention are cross-reactive with mouse iRhom2.

### Example 20: Assessment of Binding Specificity of the Antibodies of the Invention in Cell Lines Endogenously Expressing iRhom2

In this study, binding specificity analyses of the hybridoma supernatant leading to the antibody 16 of the invention as a representative example of antibodies 3, 16, 22 and 42 of the invention as well as of the antibody 16 of the invention as a representative example of antibodies 16, 22 and 42 of the invention in cell lines endogenously expressing iRhom2 were performed. The studies were conducted on RPMI-8226 cells, a human B lymphocytic cell line endogenously expressing iRhom2 but being endogenously negative for iRhom1, on THP-1 cells, a human monocytic cell line endogenously expressing both iRhom2 and iRhom1 and on RH-30 cells, a human fibroblastic cell line endogenously negative for iRhom2 but endogenously expressing iRhom1.

In brief, human RPMI-8226 cells (Deutsche Sammlung von Mikroorganismen und Zellkulturen, Germany), THP-1 cells (American Type Culture Collection, USA) and RH-30 cells (Deutsche Sammlung von Mikroorganismen und Zellkulturen, Germany) were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 2×10⁵ cells per well. In order to pellet cells and remove supernatants, the plates were centrifuged at 1,500 rpm and 4° C. for 3 minutes. For primary staining, cells were resuspended in 100 μl per well of either FACS buffer alone (controls), a 1:60 dilution of the hybridoma supernatant, the primary material leading to the antibodies 3, 16, 22 and 42 of the invention in FACS buffer or 3 μg/ml of the antibodies 16, 22 and 42 of the invention in FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 or goat anti-human IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

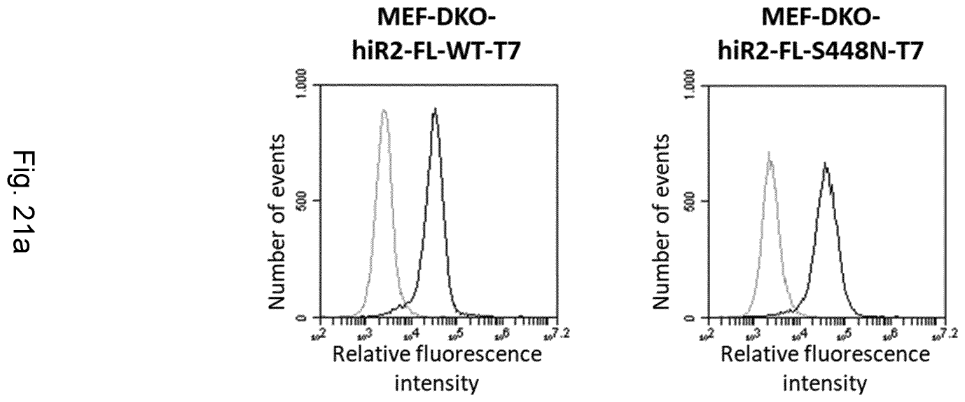

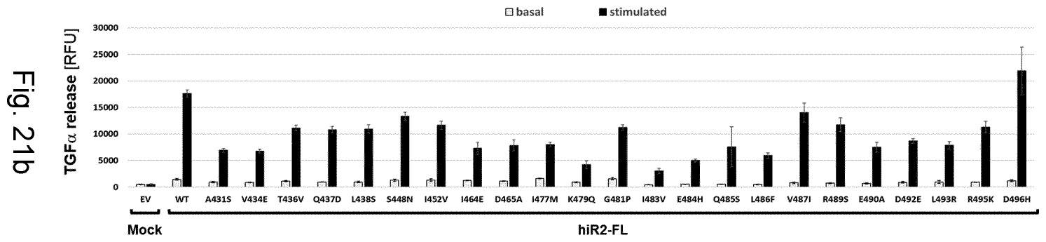

FIGS. 20a & 20b show representative results of this study. As compared to control samples incubated with secondary antibody only (20a & 20b, gray), co-incubation of both RPMI-8226 and THP-1 cells, both of which express iRhom2 endogenously, with the hybridoma supernatant leading to antibody 16 as a representative example of the antibodies 3, 16, 22 and 42 of the invention (20a, left & middle, black) or antibody 16 as a representative example of the antibodies 16, 22 and 42 of the invention (FIG. 20b, left & middle, black) leads to a strong shift in relative fluorescence intensity in both cell lines, demonstrating a strong binding of both the primary material leading to antibody 16 and the antibody 16 of the invention to the two human cell lines endogenously positive for iRhom2. In contrast, no binding of the primary material leading to antibody 16 as a representative example of the antibodies 3, 16, 22 and 42 of the invention (20a, right, black) or antibody 16 as a representative example of the antibodies 16, 22 and 42 of the invention (FIG. 20b, right, black) to RH-30 cells, which do not express iRhom2, is detectable, providing evidence that endogenously expressed iRhom2 is specifically recognized by both the primary material leading to antibody 16 of the invention and the antibody 16 of the invention. The aforementioned specificity for iRhom2 versus iRhom1 towards the endogenously expressed proteins is depicted both for the murine (upper panel) and for the chimeric (lower panel) version of antibody 16 in FIG. 20b.

### Example 21: Epitope Mapping of the Antibodies of the Invention Based on Family Member-Specific Sequence Variations of iRhom2 N-Terminal of the Central Region of the Large Extracellular Loop

Complementary to Examples 15 and 16, plasmids for a set of 23 human iRhom2 variants with human iRhom1-related single amino acid substitutions to identify single amino acids that contribute to binding of the antibodies of the invention, were designed in a third approach.

These 23 substitutions reflect amino acids N-terminal of the central region of the large extracellular loop 1 that are non-identical in human iRhom2 versus human iRhom1. Instead of the amino acid of human iRhom2, the amino acid at the corresponding position of human iRhom1 was introduced, resulting in the variants hiR2-FL-A431S-T7, hiR2-FL-V434E-T7, hiR2-FL-T436V-T7, hiR2-FL-Q437D-T7, hiR2-FL-L438S-T7, hiR2-FL-S448N-T7, hiR2-FL-I452V-T7, hiR2-FL-1464E-T7, hiR2-FL-D465A-T7, hiR2-FL-1477M-T7, hiR2-FL-K479Q-T7, hiR2-FL-G481P-T7, hiR2-FL-1483V-T7, hiR2-FL-E484H-T7, hiR2-FL-Q485S-T7, hiR2-FL-L486F-T7, hiR2-FL-V4871-T7, hiR2-FL-R489S-T7, hiR2-FL-E490A-T7, hiR2-FL-D492E-T7, hiR2-FL-L493R-T7, hiR2-FL-R495K-T7 and hiR2-FL-D496H-T7.

This example describes the generation of iRhom1/2−/− DKO MEF populations expressing the 23 human iRhom1-related single amino acid substitution variants as well as their characterization in terms of cell surface localization and functional activity as indicators of proper protein conformation. Subsequently, binding analyses of the purified antibodies 3, 5, 16, 22, 34, 42, 43, 44, 48, and 50 of the invention on the entire panel of 23 engineered MEF populations expressing human iRhom2 variants with human iRhom1-related single amino acid substitutions are described.

Generation of iRhom1/2−/− DKO MEFs Stably Expressing 23 T7-Tagged Human iRhom2 Variants with Human iRhom1-Related Single Amino Acid Substitutions

In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV-hiR2-FL-A431S-T7, pMSCV-hiR2-FL-V434E-T7, pMSCV-hiR2-FL-T436V-T7, pMSCV-hiR2-FL-Q437D-T7, pMSCV-hiR2-FL-L438S-T7, pMSCV-hiR2-FL-S448N-T7, pMSCV-hiR2-FL-I452V-T7, pMSCV-hiR2-FL-1464E-T7, pMSCV-hiR2-FL-D465A-T7, pMSCV-hiR2-FL-I477M-T7, pMSCV-hiR2-FL-K479Q-T7, pMSCV-hiR2-FL-G481P-T7, pMSCV-hiR2-FL-I483V-T7, pMSCV-hiR2-FL-E484H-T7, pMSCV-hiR2-FL-Q485S-T7, pMSCV-hiR2-FL-L486F-T7, pMSCV-hiR2-FL-V487I-T7, pMSCV-hiR2-FL-R489S-T7, pMSCV-hiR2-FL-E490A-T7, pMSCV-hiR2-FL-D492E-T7, pMSCV-hiR2-FL-L493R-T7, pMSCV-hiR2-FL-R495K-T7 and pMSCV-hiR2-FL-D496H-T7 encoding human iRhom2 full length single amino acid substitutions C-terminally tagged with 3 consecutive copies of the T7 epitope (MASMTGGQQMG), and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV-hiR2-FL-A431S-T7, pMSCV-hiR2-FL-V434E-T7, pMSCV-hiR2-FL-T436V-T7, pMSCV-hiR2-FL-Q437D-T7, pMSCV-hiR2-FL-L438S-T7, pMSCV-hiR2-FL-S448N-T7, pMSCV-hiR2-FL-1452V-T7, pMSCV-hiR2-FL-1464E-T7, pMSCV-hiR2-FL-D465A-T7, pMSCV-hiR2-FL-1477M-T7, pMSCV-hiR2-FL-K479Q-T7, pMSCV-hiR2-FL-G481P-T7, pMSCV-hiR2-FL-1483V-T7, pMSCV-hiR2-FL-E484H-T7, pMSCV-hiR2-FL-Q485S-T7, pMSCV-hiR2-FL-L486F-T7, pMSCV-hiR2-FL-V487I-T7, pMSCV-hiR2-FL-R489S-T7, pMSCV-hiR2-FL-E490A-T7, pMSCV-hiR2-FL-D492E-T7, pMSCV-hiR2-FL-L493R-T7, pMSCV-hiR2-FL-R495K-T7 and pMSCV-hiR2-FL-D496H-T7 ecotrophic virus were collected, filtered with 0.45 μm CA filters, and supplemented with 4 μg/ml of polybrene (Sigma-Aldrich, USA). Upon removal of medium from the immortalized iRhom1/2−/− DKO MEFs, these supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO-hiR2-FL-A431S-T7, MEF-DKO-hiR2-FL-V434E-T7, MEF-DKO-hiR2-FL-T436V-T7, MEF-DKO-hiR2-FL-Q437D-T7, MEF-DKO-hiR2-FL-L438S-T7, MEF-DKO-hiR2-FL-S448N-T7, MEF-DKO-hiR2-FL-I452V-T7, MEF-DKO-hiR2-FL-I464E-T7, MEF-DKO-hiR2-FL-D465A-T7, MEF-DKO-hiR2-FL-I477M-T7, MEF-DKO-hiR2-FL-K479Q-T7, MEF-DKO-hiR2-FL-G481P-T7, MEF-DKO-hiR2-FL-I483V-T7, MEF-DKO-hiR2-FL-E484H-T7, MEF-DKO-hiR2-FL-Q485S-T7, MEF-DKO-hiR2-FL-L486F-T7, MEF-DKO-hiR2-FL-V487I-T7, MEF-DKO-hiR2-FL-R489S-T7, MEF-DKO-hiR2-FL-E490A-T7, MEF-DKO-hiR2-FL-D492E-T7, MEF-DKO-hiR2-FL-L493R-T7, MEF-DKO-hiR2-FL-R495K-T7 and MEF-DKO-hiR2-FL-D496H-T7 cells stably expressing human iRhom2 full length single amino acid substitutions C-terminally tagged with 3 consecutive copies of the T7 epitope. Upon propagation, cells were stocked for future usage.

### FACS Analyses for Test System Validation

In brief, immortalized MEF-DKO-hiR2-FL-WT-T7 cells and MEF-DKO-hiR2-FL-A431S-T7, MEF-DKO-hiR2-FL-V434E-T7, MEF-DKO-hiR2-FL-T436V-T7, MEF-DKO-hiR2-FL-Q437D-T7, MEF-DKO-hiR2-FL-L438S-T7, MEF-DKO-hiR2-FL-S448N-T7, MEF-DKO-hiR2-FL-I452V-T7, MEF-DKO-hiR2-FL-I464E-T7, MEF-DKO-hiR2-FL-D465A-T7, MEF-DKO-hiR2-FL-I477M-T7, MEF-DKO-hiR2-FL-K479Q-T7, MEF-DKO-hiR2-FL-G481P-T7, MEF-DKO-hiR2-FL-I483V-T7, MEF-DKO-hiR2-FL-E484H-T7, MEF-DKO-hiR2-FL-Q485S-T7, MEF-DKO-hiR2-FL-L486F-T7, MEF-DKO-hiR2-FL-V487I-T7, MEF-DKO-hiR2-FL-R489S-T7, MEF-DKO-hiR2-FL-E490A-T7, MEF-DKO-hiR2-FL-D492E-T7, MEF-DKO-hiR2-FL-L493R-T7, MEF-DKO-hiR2-FL-R495K-T7 and MEF-DKO-hiR2-FL-D496H-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 1×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or mouse monoclonal anti-T7 IgG (Merck Millipore, USA) at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

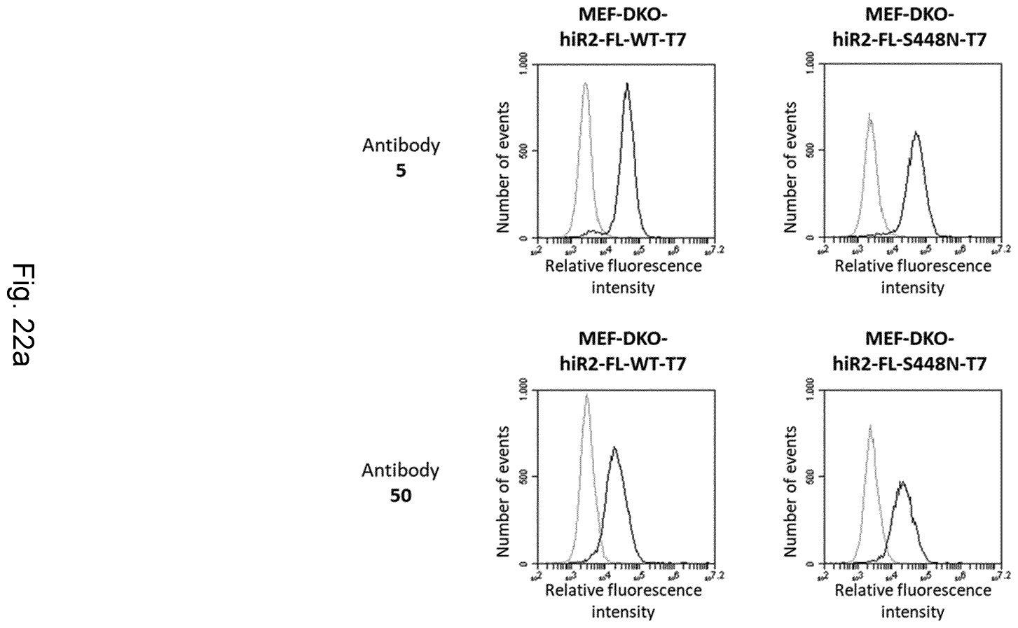

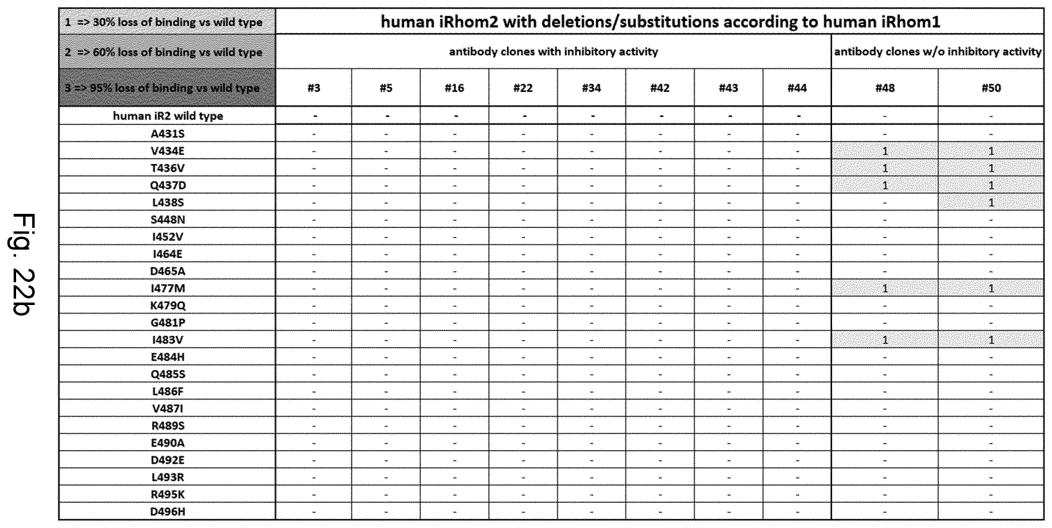

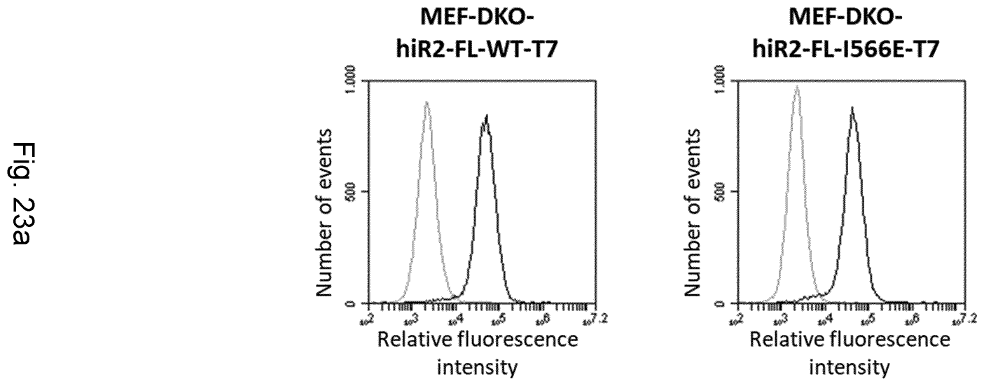

FIG. 21a shows representative results of this experiment exemplarily for the human iRhom2 variant hiR2-FL-S448N-T7. Binding analyses of anti-T7 tag antibody (black) and anti-mouse IgG secondary antibody (gray) on MEF-DKO-hiR2-FL-WT-T7 (left) and MEF-DKO-hiR2-FL-S448N-T7 cells (right) reveal a comparably strong increase in relative fluorescence intensity. This demonstrates that, similarly to human iRhom2 wild type (left), the human iRhom2 variant hiR2-FL-S448N-T7 is equally well expressed and localized on the surface of these cells (right). Similar results were obtained for the expression and localization of the other human iRhom2 full length single amino acid substitutions expressed on MEF-DKO-hiR2-FL-A431S-T7, MEF-DKO-hiR2-FL-V434E-T7, MEF-DKO-hiR2-FL-T436V-T7, MEF-DKO-hiR2-FL-Q437D-T7, MEF-DKO-hiR2-FL-L438S-T7, MEF-DKO-hiR2-FL-I452V-T7, MEF-DKO-hiR2-FL-I464E-T7, MEF-DKO-hiR2-FL-D465A-T7, MEF-DKO-hiR2-FL-I477M-T7, MEF-DKO-hiR2-FL-K479Q-T7, MEF-DKO-hiR2-FL-G481P-T7, MEF-DKO-hiR2-FL-I483V-T7, MEF-DKO-hiR2-FL-E484H-T7, MEF-DKO-hiR2-FL-Q485S-T7, MEF-DKO-hiR2-FL-L486F-T7, MEF-DKO-hiR2-FL-V487I-T7, MEF-DKO-hiR2-FL-R489S-T7, MEF-DKO-hiR2-FL-E490A-T7, MEF-DKO-hiR2-FL-D492E-T7, MEF-DKO-hiR2-FL-L493R-T7, MEF-DKO-hiR2-FL-R495K-T7 and MEF-DKO-hiR2-FL-D496H-T7 cells.

### TGFα ELISA for Test System Validation

To test all 23 human iRhom2 variants with human iRhom1-specific single amino acid substitutions, the respective MEF-DKO cell lines stably expressing these variants, generated as described in the example above, were subjected to TGFα shedding ELISA analysis. In order to demonstrate the functionality of all variants as an indicator that these variants are properly folded, PMA-induced release of nucleofected TGFα was assessed. As the cells used in this analysis are rescue variants of iRhom1/2−/− double knockout mouse embryonic fibroblasts (described in Example 11), that are rescued by the respective human iRhom2 variant with the human iRhom1-specific single amino acid substitution or deletion, the iRhom2 variant stably expressed is the only iRhom protein expressed in these cells at all and is therefore the only contributing iRhom to the shedding TGFα in these cells.

In brief, on day 1, Nunc black MaxiSorp® 96-well plates (Thermo Fisher Scientific, USA) were coated overnight with 100 μl per well of mouse anti-human TGFα capture antibody (provided as part of the DuoSet ELISA kit) at 400 ng/ml in TBS at 4° C. After MEF-DKO-hiR2-FL-A431S-T7, MEF-DKO-hiR2-FL-V434E-T7, MEF-DKO-hiR2-FL-T436V-T7, MEF-DKO-hiR2-FL-Q437D-T7, MEF-DKO-hiR2-FL-L438S-T7, MEF-DKO-hiR2-FL-S448N-T7, MEF-DKO-hiR2-FL-I452V-T7, MEF-DKO-hiR2-FL-I464E-T7, MEF-DKO-hiR2-FL-D465A-T7, MEF-DKO-hiR2-FL-I477M-T7, MEF-DKO-hiR2-FL-K479Q-T7, MEF-DKO-hiR2-FL-G481P-T7, MEF-DKO-hiR2-FL-I483V-T7, MEF-DKO-hiR2-FL-E484H-T7, MEF-DKO-hiR2-FL-Q485S-T7, MEF-DKO-hiR2-FL-L486F-T7, MEF-DKO-hiR2-FL-V487I-T7, MEF-DKO-hiR2-FL-R489S-T7, MEF-DKO-hiR2-FL-E490A-T7, MEF-DKO-hiR2-FL-D492E-T7, MEF-DKO-hiR2-FL-L493R-T7, MEF-DKO-hiR2-FL-R495K-T7 and MEF-DKO-hiR2-FL-D496H-T7 cells were electroporated with the hTGFα-FL-WT construct in a pcDNA3.1 vector backbone, using an 4D-Nucleofector System (Lonza, Switzerland), approximately 35,000 MEF-DKO cells carrying the human iRhom2 variant with the human iRhom1-specific single amino acid substitution or deletion were seeded in 100 μl of normal growth medium in each well of F-bottom 96-well cell culture plates (Thermo Fisher Scientific, USA). On day 2, the capture antibody solution was removed and MaxiSorp® plates were blocked with 300 μl per well of TBS, 1% BSA at room temperature for at least 1 hour. Meanwhile, the cells were washed once with PBS and afterwards 80 μl of OptiMEM medium (Thermo Fisher Scientific, USA) was added per well.

Subsequently, cells (except those for unstimulated controls) were stimulated with 20 μl per well of PMA (Sigma-Aldrich, USA) at a final concentration of 25 ng/ml at 37° C., 5% CO₂ for 1 hour. 20 μl of OptiMEM medium was added to the unstimulated control cells. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Thereafter, 100 μl biotinylated goat anti-human TGFα detection antibody (provided as part of the DuoSet ELISA kit) at 37.5 ng/ml in TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

FIG. 21b shows results from these TGFα release assays demonstrating that all 23 human iRhom2 variants with human iRhom1-specific single amino acid substitutions are functionally active, as TGFα shedding can be induced with PMA, indicating that these variants are properly folded, in contrast to the empty vector (EV) negative control population, where no PMA-induced shedding of TGFα is detectable.

### FACS Analyses to Characterize Binding of the Purified Antibodies of the Invention for the Purpose of Epitope Mapping

In brief, immortalized MEF-DKO-hiR2-FL-A431S-T7, MEF-DKO-hiR2-FL-V434E-T7, MEF-DKO-hiR2-FL-T436V-T7, MEF-DKO-hiR2-FL-Q437D-T7, MEF-DKO-hiR2-FL-L438S-T7, MEF-DKO-hiR2-FL-S448N-T7, MEF-DKO-hiR2-FL-I452V-T7, MEF-DKO-hiR2-FL-I464E-T7, MEF-DKO-hiR2-FL-D465A-T7, MEF-DKO-hiR2-FL-I477M-T7, MEF-DKO-hiR2-FL-K479Q-T7, MEF-DKO-hiR2-FL-G481P-T7, MEF-DKO-hiR2-FL-I483V-T7, MEF-DKO-hiR2-FL-E484H-T7, MEF-DKO-hiR2-FL-Q485S-T7, MEF-DKO-hiR2-FL-L486F-T7, MEF-DKO-hiR2-FL-V487I-T7, MEF-DKO-hiR2-FL-R489S-T7, MEF-DKO-hiR2-FL-E490A-T7, MEF-DKO-hiR2-FL-D492E-T7, MEF-DKO-hiR2-FL-L493R-T7, MEF-DKO-hiR2-FL-R495K-T7 and MEF-DKO-hiR2-FL-D496H-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 1×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or the purified antibodies 3, 5, 16, 22, 34, 42, 43, 44, 48, and 50 of the invention at 3 μg/ml in FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

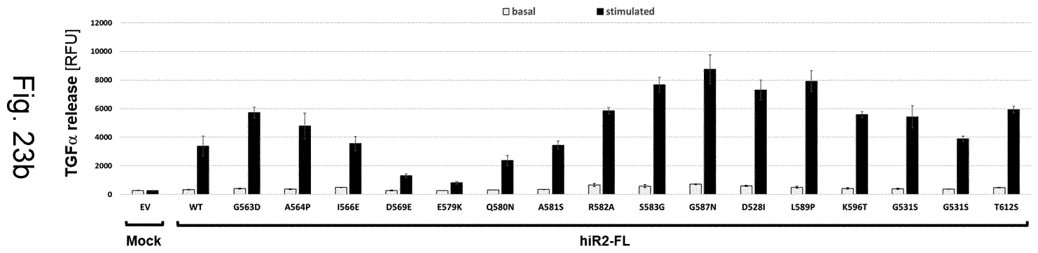

FIG. 22a shows representative results of this experiment. Exemplarily for the entire panel of 23 human iRhom2 variants with human iRhom1-related single amino acid substitutions data for the analysis of cells expressing the human iRhom2 variant hiR2-FL-S448N-T7 are shown. Binding analyses of the antibody 5 as a representative example of the antibodies of the invention with inhibitory effects on TNFα release (black, upper panel) or the antibody 50 without inhibitory effects on TNFα release (black, lower panel) as well as anti-mouse IgG secondary antibody (gray) on MEF-DKO-hiR2-FL-WT-T7 cells (left) and MEF-DKO-hiR2-FL-S448N-T7 cells (right) demonstrate that the substitution of the single amino acid serine 448 of human iRhom2 by asparagine does not impair and, thus, does not contribute to binding of the antibody 5 of the invention with inhibitory effects on TNFα release (right, upper panel). Likewise, it does not affect and, thus, does not contribute to binding of the antibody 50 without inhibitory effects on TNFα release (right, lower panel). For both antibodies, binding to MEF-DKO-hiR2-FL-WT-T7 cells (left) serves as positive control.

FIG. 22b summarizes—in extension of FIG. 22a —the results of FACS analyses of the antibodies 3, 5, 16, 22, 34, 42, 43, and 44 of the invention with inhibitory effects on TNFα release versus the antibodies 48 and 50 without inhibitory effects on TNFα release on the entire panel of 23 engineered MEF populations expressing human iRhom2 variants with human iRhom1-specific single amino acid substitutions. Binding of each antibody to human iRhom2 wild type is considered 100 percent. A respective drop of antibody binding to any variant by 30-59% is indicated by cells held in light gray (and marked with “1”), an impaired binding by 60-95% is illustrated by cells colored in gray (and marked with “2”), and a loss of binding by ≥95% is highlighted by dark gray cells (marked with “3”). These data reveal that none of the iRhom1-specific single amino acid substitutions analyzed in this approach is relevant for binding of the antibodies 3, 5, 16, 22, 34, 42, 43, and 44 of the invention with inhibitory effects on TNFα release to human iRhom2. In contrast, some of them contribute to the binding of the antibodies 48 and 50 without inhibitory effects on TNFα release.

### Example 22: Epitope Mapping of the Antibodies of the Invention Based on Family Member-Specific Sequence Variations of iRhom2 C-Terminal of the Central Region of the Large Extracellular Loop, in Loop5 and in the C-Terminus

Complementary to Examples 15 and 16 and also to Example 21 described above, plasmids for a set of 33 human iRhom2 variants with human iRhom1-related single amino acid substitutions to identify single amino acids that contribute to binding of the antibodies of the invention, were designed in a fourth approach. These 33 substitutions reflect amino acids C-terminal of the central region of the large extracellular loop 1, in loop 5 and in the C-terminus that are non-identical in human iRhom2 versus human iRhom1. Instead of the amino acid of human iRhom2, the amino acid at the corresponding position of human iRhom1 was introduced, resulting in the variants hiR2-FL-G563D-T7, hiR2-FL-A564P-T7, hiR2-FL-1566E-T7, hiR2-FL-D569E-T7, hiR2-FL-E579K-T7, hiR2-FL-Q580N-T7, hiR2-FL-A581S-T7, hiR2-FL-R582A-T7, hiR2-FL-S583G-T7, hiR2-FL-G587N-T7, hiR2-FL-F588H-T7, hiR2-FL-L589P-T7, hiR2-FL-E594V-T7, hiR2-FL-K596T-T7, hiR2-FL-S607R-T7, hiR2-FL-T612S-T7, hiR2-FL-E617D-T7, hiR2-FL-H620R-T7, hiR2-FL-L636M-T7, hiR2-FL-K638D-T7, hiR2-FL-I771F-T7, hiR2-FL-1825V-T7, hiR2-FL-1828V-T7, hiR2-FL-N829R-T7, hiR2-FL-W830C-T7, hiR2-FL-P831E-T7, hiR2-FL-1833C-T7, hiR2-FL-H835F-T7, hiR2-FL-F8391-T7, hiR2-FL-S843D-T7, hiR2-FL-Q853A-T7, hiR2-FL-V854Q-T7 and hiR2-FL-R844K-T7.

This example describes the generation of iRhom1/2−/− DKO MEF populations expressing the 33 human iRhom1-related single amino acid substitution variants as well as their characterization in terms of cell surface localization and functional activity as indicators of proper protein conformation. Subsequently, binding analyses of the purified antibodies 3, 5, 16, 22, 34, 42, 43, 44, 48, and 50 of the invention on the entire panel of 33 engineered MEF populations expressing human iRhom2 variants with human iRhom1-related single amino acid substitutions are described.

Generation of iRhom1/2−/− DKO MEFs Stably Expressing 33 T7-Tagged Human iRhom2 Variants with Human iRhom1-Related Single Amino Acid Substitutions

In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV hiR2-FL-G563D-T7, pMSCV hiR2-FL-A564P-T7, pMSCV hiR2-FL-1566E-T7, pMSCV hiR2-FL-D569E-T7, pMSCV hiR2-FL-E579K-T7, pMSCV hiR2-FL-Q580N-T7, pMSCV hiR2-FL-A581S-T7, pMSCV hiR2-FL-R582A-T7, pMSCV hiR2-FL-S583G-T7, pMSCV hiR2-FL-G587N-T7, pMSCV hiR2-FL-F588H-T7, pMSCV hiR2-FL-L589P-T7, pMSCV hiR2-FL-E594V-T7, pMSCV hiR2-FL-K596T-T7, pMSCV hiR2-FL-S607R-T7, pMSCV hiR2-FL-T612S-T7, pMSCV hiR2-FL-E617D-T7, pMSCV hiR2-FL-H620R-T7, pMSCV hiR2-FL-L636M-T7, pMSCV hiR2-FL-K638D-T7, pMSCV hiR2-FL-1771F-T7, pMSCV hiR2-FL-1825V-T7, pMSCV hiR2-FL-1828V-T7, pMSCV hiR2-FL-N829R-T7, pMSCV hiR2-FL-W830C-T7, pMSCV hiR2-FL-P831E-T7, pMSCV hiR2-FL-1833C-T7, pMSCV hiR2-FL-H835F-T7, pMSCV hiR2-FL-F8391-T7, pMSCV hiR2-FL-S843D-T7, pMSCV hiR2-FL-Q853A-T7, pMSCV hiR2-FL-V854Q-T7 and pMSCV hiR2-FL-R844K-T7 encoding human iRhom2 full length single amino acid substitutions C-terminally tagged with 3 consecutive copies of the T7 epitope (MASMTGGQQMG), and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV hiR2-FL-G563D-T7, pMSCV hiR2-FL-A564P-T7, pMSCV hiR2-FL-1566E-T7, pMSCV hiR2-FL-D569E-T7, pMSCV hiR2-FL-E579K-T7, pMSCV hiR2-FL-Q580N-T7, pMSCV hiR2-FL-A581S-T7, pMSCV hiR2-FL-R582A-T7, pMSCV hiR2-FL-S583G-T7, pMSCV hiR2-FL-G587N-T7, pMSCV hiR2-FL-F588H-T7, pMSCV hiR2-FL-L589P-T7, pMSCV hiR2-FL-E594V-T7, pMSCV hiR2-FL-K596T-T7, pMSCV hiR2-FL-S607R-T7, pMSCV hiR2-FL-T612S-T7, pMSCV hiR2-FL-E617D-T7, pMSCV hiR2-FL-H620R-T7, pMSCV hiR2-FL-L636M-T7, pMSCV hiR2-FL-K638D-T7, pMSCV hiR2-FL-1771F-T7, pMSCV hiR2-FL-1825V-T7, pMSCV hiR2-FL-1828V-T7, pMSCV hiR2-FL-N829R-T7, pMSCV hiR2-FL-W830C-T7, pMSCV hiR2-FL-P831E-T7, pMSCV hiR2-FL-1833C-T7, pMSCV hiR2-FL-H835F-T7, pMSCV hiR2-FL-F8391-T7, pMSCV hiR2-FL-S843D-T7, pMSCV hiR2-FL-Q853A-T7, pMSCV hiR2-FL-V854Q-T7 and pMSCV hiR2-FL-R844K-T7 ecotrophic virus were collected, filtered with 0.45 μm CA filters, and supplemented with 4 μg/ml of polybrene (Sigma-Aldrich, USA). Upon removal of medium from the immortalized iRhom1/2−/− DKO MEFs, these supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO hiR2-FL-G563D-T7, MEF-DKO hiR2-FL-A564P-T7, MEF-DKO hiR2-FL-1566E-T7, MEF-DKO hiR2-FL-D569E-T7, MEF-DKO hiR2-FL-E579K-T7, MEF-DKO hiR2-FL-Q580N-T7, MEF-DKO hiR2-FL-A581S-T7, MEF-DKO hiR2-FL-R582A-T7, MEF-DKO hiR2-FL-S583G-T7, MEF-DKO hiR2-FL-G587N-T7, MEF-DKO hiR2-FL-F588H-T7, MEF-DKO hiR2-FL-L589P-T7, MEF-DKO hiR2-FL-E594V-T7, MEF-DKO hiR2-FL-K596T-T7, MEF-DKO hiR2-FL-S607R-T7, MEF-DKO hiR2-FL-T612S-T7, MEF-DKO hiR2-FL-E617D-T7, MEF-DKO hiR2-FL-H620R-T7, MEF-DKO hiR2-FL-L636M-T7, MEF-DKO hiR2-FL-K638D-T7, MEF-DKO hiR2-FL-1771F-T7, MEF-DKO hiR2-FL-1825V-T7, MEF-DKO hiR2-FL-I828V-T7, MEF-DKO hiR2-FL-N829R-T7, MEF-DKO hiR2-FL-W830C-T7, MEF-DKO hiR2-FL-P831E-T7, MEF-DKO hiR2-FL-1833C-T7, MEF-DKO hiR2-FL-H835F-T7, MEF-DKO hiR2-FL-F8391-T7, MEF-DKO hiR2-FL-S843D-T7, MEF-DKO hiR2-FL-Q853A-T7, MEF-DKO hiR2-FL-V854Q-T7 and MEF-DKO hiR2-FL-R844K-T7 cells stably expressing human iRhom2 full length single amino acid substitutions C-terminally tagged with 3 consecutive copies of the T7 epitope. Upon propagation, cells were stocked for future usage.

### FACS Analyses for Test System Validation

In brief, immortalized MEF-DKO-hiR2-FL-WT-T7 cells and MEF-DKO hiR2-FL-G563D-T7, MEF-DKO hiR2-FL-A564P-T7, MEF-DKO hiR2-FL-1566E-T7, MEF-DKO hiR2-FL-D569E-T7, MEF-DKO hiR2-FL-E579K-T7, MEF-DKO hiR2-FL-Q580N-T7, MEF-DKO hiR2-FL-A581S-T7, MEF-DKO hiR2-FL-R582A-T7, MEF-DKO hiR2-FL-S583G-T7, MEF-DKO hiR2-FL-G587N-T7, MEF-DKO hiR2-FL-F588H-T7, MEF-DKO hiR2-FL-L589P-T7, MEF-DKO hiR2-FL-E594V-T7, MEF-DKO hiR2-FL-K596T-T7, MEF-DKO hiR2-FL-S607R-T7, MEF-DKO hiR2-FL-T612S-T7, MEF-DKO hiR2-FL-E617D-T7, MEF-DKO hiR2-FL-H620R-T7, MEF-DKO hiR2-FL-L636M-T7, MEF-DKO hiR2-FL-K638D-T7, MEF-DKO hiR2-FL-1771F-T7, MEF-DKO hiR2-FL-1825V-T7, MEF-DKO hiR2-FL-I828V-T7, MEF-DKO hiR2-FL-N829R-T7, MEF-DKO hiR2-FL-W830C-T7, MEF-DKO hiR2-FL-P831E-T7, MEF-DKO hiR2-FL-1833C-T7, MEF-DKO hiR2-FL-H835F-T7, MEF-DKO hiR2-FL-F8391-T7, MEF-DKO hiR2-FL-S843D-T7, MEF-DKO hiR2-FL-Q853A-T7, MEF-DKO hiR2-FL-V854Q-T7 and MEF-DKO hiR2-FL-R844K-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 1×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or mouse monoclonal anti-T7 IgG (Merck Millipore, USA) at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

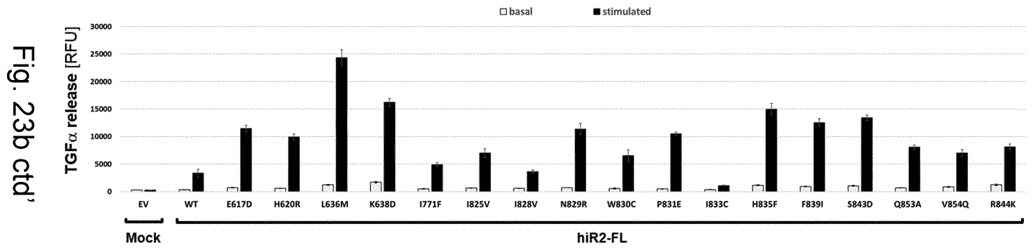

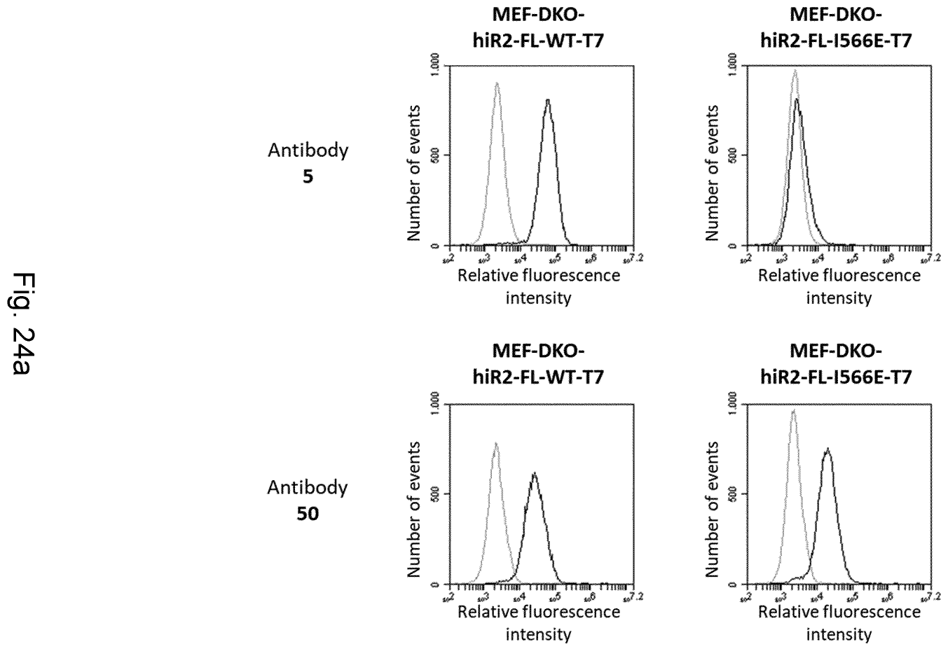

FIG. 23a shows representative results of this experiment exemplarily for the human iRhom2 variant hiR2-FL-1566E-T7. Binding analyses of anti-T7 tag antibody (black) and anti-mouse IgG secondary antibody (gray) on MEF-DKO-hiR2-FL-WT-T7 (left) and MEF-DKO-hiR2-FL-I566E-T7 cells (right) reveal a comparably strong increase in relative fluorescence intensity. This demonstrates that, similarly to human iRhom2 wild type (left), the human iRhom2 variant hiR2-FL-1566E-T7 is equally well expressed and localized on the surface of these cells (right). Similar results were obtained for the expression and localization of the other human iRhom2 full length single amino acid substitutions expressed on MEF-DKO hiR2-FL-G563D-T7, MEF-DKO hiR2-FL-A564P-T7, MEF-DKO hiR2-FL-D569E-T7, MEF-DKO hiR2-FL-E579K-T7, MEF-DKO hiR2-FL-Q580N-T7, MEF-DKO hiR2-FL-A581S-T7, MEF-DKO hiR2-FL-R582A-T7, MEF-DKO hiR2-FL-S583G-T7, MEF-DKO hiR2-FL-G587N-T7, MEF-DKO hiR2-FL-F588H-T7, MEF-DKO hiR2-FL-L589P-T7, MEF-DKO hiR2-FL-E594V-T7, MEF-DKO hiR2-FL-K596T-T7, MEF-DKO hiR2-FL-S607R-T7, MEF-DKO hiR2-FL-T612S-T7, MEF-DKO hiR2-FL-E617D-T7, MEF-DKO hiR2-FL-H620R-T7, MEF-DKO hiR2-FL-L636M-T7, MEF-DKO hiR2-FL-K638D-T7, MEF-DKO hiR2-FL-1771F-T7, MEF-DKO hiR2-FL-1825V-T7, MEF-DKO hiR2-FL-1828V-T7, MEF-DKO hiR2-FL-N829R-T7, MEF-DKO hiR2-FL-W830C-T7, MEF-DKO hiR2-FL-P831E-T7, MEF-DKO hiR2-FL-1833C-T7, MEF-DKO hiR2-FL-H835F-T7, MEF-DKO hiR2-FL-F8391-T7, MEF-DKO hiR2-FL-S843D-T7, MEF-DKO hiR2-FL-Q853A-T7, MEF-DKO hiR2-FL-V854Q-T7 and MEF-DKO hiR2-FL-R844K-T7 cells.

### TGFα ELISA for Test System Validation

To test all 33 human iRhom2 variants with human iRhom1-specific single amino acid substitutions, the respective MEF-DKO cell lines stably expressing these variants, generated as described in the example above, were subjected to TGFα shedding ELISA analysis. In order to demonstrate the functionality of all variants as an indicator that these variants are properly folded, PMA-induced release of nucleofected TGFα was assessed. As the cells used in this analysis are rescue variants of iRhom1/2−/− double knockout mouse embryonic fibroblasts (described in Example 11), that are rescued by the respective human iRhom2 variant with the human iRhom1-specific single amino acid substitution or deletion, the iRhom2 variant stably expressed is the only iRhom protein expressed in these cells and is therefore the only iRhom contributing to the shedding TGFα in these cells.

In brief, on day 1, Nunc black MaxiSorp® 96-well plates (Thermo Fisher Scientific, USA) were coated overnight with 100 μl per well of mouse anti-human TGFα capture antibody (provided as part of the DuoSet ELISA kit) at 400 ng/ml in TBS at 4° C. After MEF-DKO hiR2-FL-G563D-T7, MEF-DKO hiR2-FL-A564P-T7, MEF-DKO hiR2-FL-I566E-T7, MEF-DKO hiR2-FL-D569E-T7, MEF-DKO hiR2-FL-E579K-T7, MEF-DKO hiR2-FL-Q580N-T7, MEF-DKO hiR2-FL-A581S-T7, MEF-DKO hiR2-FL-R582A-T7, MEF-DKO hiR2-FL-S583G-T7, MEF-DKO hiR2-FL-G587N-T7, MEF-DKO hiR2-FL-F588H-T7, MEF-DKO hiR2-FL-L589P-T7, MEF-DKO hiR2-FL-E594V-T7, MEF-DKO hiR2-FL-K596T-T7, MEF-DKO hiR2-FL-S607R-T7, MEF-DKO hiR2-FL-T612S-T7, MEF-DKO hiR2-FL-E617D-T7, MEF-DKO hiR2-FL-H620R-T7, MEF-DKO hiR2-FL-L636M-T7, MEF-DKO hiR2-FL-K638D-T7, MEF-DKO hiR2-FL-I771F-T7, MEF-DKO hiR2-FL-I825V-T7, MEF-DKO hiR2-FL-I828V-T7, MEF-DKO hiR2-FL-N829R-T7, MEF-DKO hiR2-FL-W830C-T7, MEF-DKO hiR2-FL-P831E-T7, MEF-DKO hiR2-FL-I833C-T7, MEF-DKO hiR2-FL-H835F-T7, MEF-DKO hiR2-FL-F839I-T7, MEF-DKO hiR2-FL-S843D-T7, MEF-DKO hiR2-FL-Q853A-T7, MEF-DKO hiR2-FL-V854Q-T7 and MEF-DKO hiR2-FL-R844K-T7 cells were electroporated with the hTGFα-FL-WT construct in a pcDNA3.1 vector backbone, using an 4D-Nucleofector System (Lonza, Switzerland), approximately 35,000 MEF-DKO cells carrying the human iRhom2 variant with the human iRhom1-specific single amino acid substitution or deletion were seeded in 100 μl of normal growth medium in each well of F-bottom 96-well cell culture plates (Thermo Fisher Scientific, USA). On day 2, the capture antibody solution was removed and MaxiSorp® plates were blocked with 300 μl per well of TBS, 1% BSA at room temperature for at least 1 hour. Meanwhile, the cells were washed once with PBS and afterwards 80 μl of OptiMEM medium (Thermo Fisher Scientific, USA) was added per well. Subsequently, cells (except those for unstimulated controls) were stimulated with 20 μl per well of PMA (Sigma-Aldrich, USA) at a final concentration of 25 ng/ml at 37° C., 5% CO₂ for 1 hour. 20 μl of OptiMEM medium was added to the unstimulated control cells. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Thereafter, 100 μl biotinylated goat anti-human TGFα detection antibody (provided as part of the DuoSet ELISA kit) at 37.5 ng/ml in TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

FIG. 23b shows results from these TGFα release assays demonstrating that all 33 human iRhom2 variants with human iRhom1-specific single amino acid substitutions are functionally active as TGFα shedding can be induced with PMA, indicating that these variants are properly folded, in contrast to the empty vector (EV) negative control population, where no PMA-induced shedding of TGFα is detectable.

### FACS Analyses to Characterize Binding of the Purified Antibodies of the Invention for the Purpose of Epitope Mapping

In brief, immortalized MEF-DKO hiR2-FL-G563D-T7, MEF-DKO hiR2-FL-A564P-T7, MEF-DKO hiR2-FL-1566E-T7, MEF-DKO hiR2-FL-D569E-T7, MEF-DKO hiR2-FL-E579K-T7, MEF-DKO hiR2-FL-Q580N-T7, MEF-DKO hiR2-FL-A581S-T7, MEF-DKO hiR2-FL-R582A-T7, MEF-DKO hiR2-FL-S583G-T7, MEF-DKO hiR2-FL-G587N-T7, MEF-DKO hiR2-FL-F588H-T7, MEF-DKO hiR2-FL-L589P-T7, MEF-DKO hiR2-FL-E594V-T7, MEF-DKO hiR2-FL-K596T-T7, MEF-DKO hiR2-FL-S607R-T7, MEF-DKO hiR2-FL-T612S-T7, MEF-DKO hiR2-FL-E617D-T7, MEF-DKO hiR2-FL-H620R-T7, MEF-DKO hiR2-FL-L636M-T7, MEF-DKO hiR2-FL-K638D-T7, MEF-DKO hiR2-FL-1771F-T7, MEF-DKO hiR2-FL-1825V-T7, MEF-DKO hiR2-FL-1828V-T7, MEF-DKO hiR2-FL-N829R-T7, MEF-DKO hiR2-FL-W830C-T7, MEF-DKO hiR2-FL-P831E-T7, MEF-DKO hiR2-FL-I833C-T7, MEF-DKO hiR2-FL-H835F-T7, MEF-DKO hiR2-FL-F8391-T7, MEF-DKO hiR2-FL-S843D-T7, MEF-DKO hiR2-FL-Q853A-T7, MEF-DKO hiR2-FL-V854Q-T7 and MEF-DKO hiR2-FL-R844K-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 1×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or the purified antibodies 3, 5, 16, 22, 34, 42, 43, 44, 48, and 50 of the invention at 3 μg/ml in FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

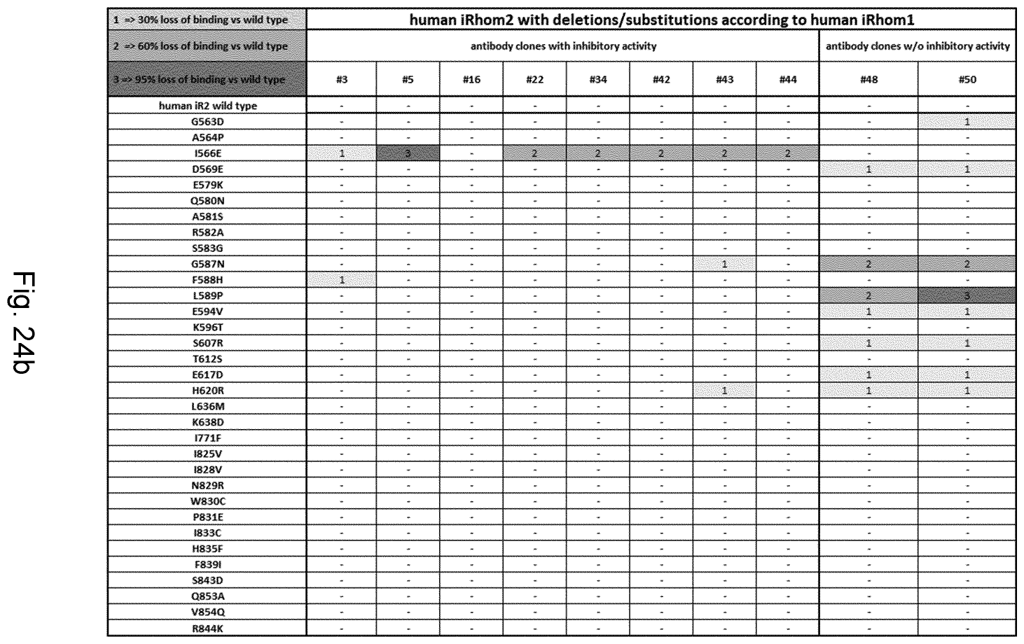

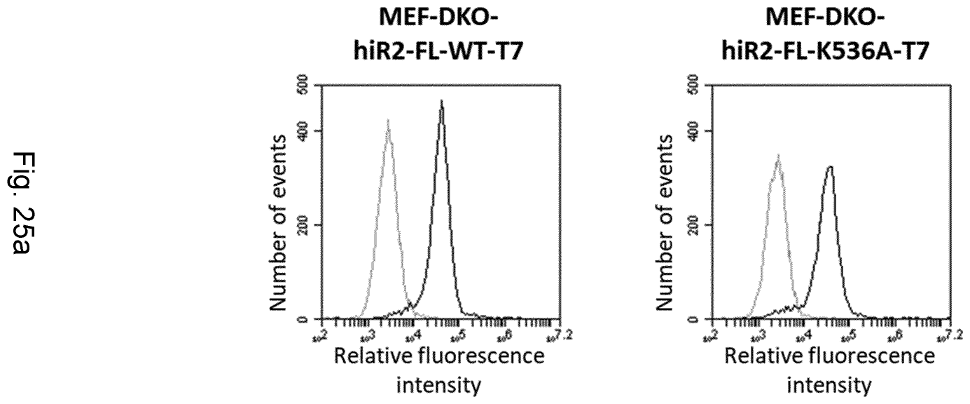

FIG. 24a shows representative results of this experiment. Exemplarily for the entire panel of 33 human iRhom2 variants with human iRhom1-related single amino acid substitutions data for the analysis of cells expressing the human iRhom2 variant hiR2-FL-1566E-T7 are shown. Binding analyses of the antibody 5 as a representative example of the antibodies of the invention with inhibitory effects on TNFα release (black, upper panel) or the antibody 50 without inhibitory effects on TNFα release (black, lower panel) as well as anti-mouse IgG secondary antibody (gray) on MEF-DKO-hiR2-FL-WT-T7 cells (left) and MEF-DKO-hiR2-FL-I566E-T7 cells (right) demonstrate the substitution of the single amino acid isoleucine 566 of human iRhom2 by glutamic acid to strongly impair and, thus, contributes to binding of the antibody 5 of the invention with inhibitory effects on TNFα release (right, upper panel). In contrast, it does not affect and, thus, does not contribute to binding of the antibody 50 without inhibitory effects on TNFα release (right, lower panel). For both antibodies, binding to MEF-DKO-hiR2-FL-WT-T7 cells (left) serves as positive control.

FIG. 24b summarizes—in extension of FIG. 24a —the results of FACS analyses of the antibodies 3, 5, 16, 22, 34, 42, 43, and 44 of the invention with inhibitory effects on TNFα release versus the antibodies 48 and 50 without inhibitory effects on TNFα release on the entire panel of 33 engineered MEF populations expressing human iRhom2 variants with human iRhom1-specific single amino acid substitutions. Binding of each antibody to human iRhom2 wild type is considered 100 percent. A respective drop of antibody binding to any variant by 30-59% is indicated by cells held in light gray (and marked with “1”), an impaired binding by 60-95% is illustrated by cells colored in gray (and marked with “2”), and a loss of binding by ≥95% is highlighted by dark gray cells (marked with “3”). These data reveal a related pattern of an amino acid position relevant for binding of the antibodies 3, 5, 16, 22, 34, 42, 43, and 44 of the invention with inhibitory effects on TNFα release to human iRhom2 (except for antibody 16 of the invention), which are different from patterns of amino acid positions contributing to binding of the antibodies 48 and 50 without inhibitory effects on TNFα release.

### Example 23: Epitope Mapping of the Antibodies of the Invention Based on Alanine Substitutions in the Central Region of the Large Extracellular Loop

Complementary to Examples 15 and 16 and also to Examples 21 & 22 described above, plasmids for a set of 91 human iRhom2 variants with single amino acid substitutions to alanine to identify single amino acids that contribute to binding of the antibodies of the invention, were designed in a fifth approach. Instead of the amino acid of human iRhom2, the amino acid alanine was introduced at the corresponding position, resulting in the variants hiR2-FL-N503A-T7, hiR2-FL-D504A-T7, hiR2-FL-H505A-T7, hiR2-FL-S506A-T7, hiR2-FL-G507A-T7, hiR2-FL-C508A-T7, hiR2-FL-1509A-T7, hiR2-FL-Q510A-T7, hiR2-FL-T511A-T7, hiR2-FL-Q512A-T7, hiR2-FL-R513A-T7, hiR2-FL-K514A-T7, hiR2-FL-D515A-T7, hiR2-FL-C516A-T7, hiR2-FL-S517A-T7, hiR2-FL-E518A-T7, hiR2-FL-T519A-T7, hiR2-FL-L520A-T7, hiR2-FL-A521S-T7, hiR2-FL-T522A-T7, hiR2-FL-F523A-T7, hiR2-FL-V524A-T7, hiR2-FL-K525A-T7, hiR2-FL-W526A-T7, hiR2-FL-Q527A-T7, hiR2-FL-D528A-T7, hiR2-FL-D529A-T7, hiR2-FL-T530A-T7, hiR2-FL-G531A-T7, hiR2-FL-P532A-T7, hiR2-FL-P533A-T7, hiR2-FL-M534A-T7, hiR2-FL-D535A-T7, hiR2-FL-K536A-T7, hiR2-FL-S537A-T7, hiR2-FL-D538A-T7, hiR2-FL-L539A-T7, hiR2-FL-G540A-T7, hiR2-FL-Q541A-T7, hiR2-FL-K542A-T7, hiR2-FL-R543A-T7, hiR2-FL-T544A-T7, hiR2-FL-S545A-T7, hiR2-FL-G546A-T7, hiR2-FL-A547S-T7, hiR2-FL-V548A-T7, hiR2-FL-C549A-T7, hiR2-FL-H550A-T7, hiR2-FL-Q551A-T7, hiR2-FL-D552A-T7, hiR2-FL-P553A-T7, hiR2-FL-R554A-T7, hiR2-FL-T555A-T7, hiR2-FL-C556A-T7, hiR2-FL-E557A-T7, hiR2-FL-E558A-T7, hiR2-FL-P559A-T7, hiR2-FL-A560S-T7, hiR2-FL-S561A-T7, hiR2-FL-S562A-T7, hiR2-FL-G563A-T7, hiR2-FL-A564S-T7, hiR2-FL-H565A-T7, hiR2-FL-1566A-T7, hiR2-FL-W567A-T7, hiR2-FL-P568A-T7, hiR2-FL-D569A-T7, hiR2-FL-D570A-T7, hiR2-FL-I571A-T7, hiR2-FL-T572A-T7, hiR2-FL-K573A-T7, hiR2-FL-W574A-T7, hiR2-FL-P575A-T7, hiR2-FL-1576A-T7, hiR2-FL-C577A-T7, hiR2-FL-T578A-T7, hiR2-FL-E579A-T7, hiR2-FL-Q580A-T7, hiR2-FL-A581S-T7, hiR2-FL-R582A-T7, hiR2-FL-S583A-T7, hiR2-FL-N584A-T7, hiR2-FL-H585A-T7, hiR2-FL-T586A-T7, hiR2-FL-G587A-T7, hiR2-FL-F588A-T7, hiR2-FL-L589A-T7, hiR2-FL-H590A-T7, hiR2-FL-M591A-T7, hiR2-FL-D592A-T7 and hiR2-FL-C593A-T7.

This example describes the generation of iRhom1/2−/− DKO MEF populations expressing the 91 single alanine amino acid substitution variants as well as their characterization in terms of cell surface localization and functional activity as indicators of proper protein conformation. Subsequently, binding analyses of the purified antibodies 3, 5, 16, 22, 34, 42, 43, 44, 48, and 50 of the invention on the entire panel of 91 engineered MEF populations expressing human iRhom2 variants with single amino acid substitutions to alanine are described.

Generation of iRhom1/2−/− DKO MEFs Stably Expressing 91 T7-Tagged Human iRhom2 Variants with Single Amino Acid Substitutions to Alanine

In brief, on day 1, Phoenix-ECO cells (American Type Culture Collection, USA) were seeded on 6-well tissue culture plates (Greiner, Germany) in standard growth medium at 8×10⁵ cells per well and kept overnight at 37° C., 5% CO₂. On day 2, the medium was replaced by fresh medium supplemented with chloroquine (Sigma-Aldrich, USA) at a final concentration of 25 μM. Applying the calcium phosphate method, cells were transfected with 2 μg/ml of pMSCV-hiR2-FL-N503A-T7, pMSCV-hiR2-FL-D504A-T7, pMSCV-hiR2-FL-H505A-T7, pMSCV-hiR2-FL-S506A-T7, pMSCV-hiR2-FL-G507A-T7, pMSCV-hiR2-FL-C508A-T7, pMSCV-hiR2-FL-I509A-T7, pMSCV-hiR2-FL-Q510A-T7, pMSCV-hiR2-FL-T511A-T7, pMSCV-hiR2-FL-Q512A-T7, pMSCV-hiR2-FL-R513A-T7, pMSCV-hiR2-FL-K514A-T7, pMSCV-hiR2-FL-D515A-T7, pMSCV-hiR2-FL-C516A-T7, pMSCV-hiR2-FL-S517A-T7, pMSCV-hiR2-FL-E518A-T7, pMSCV-hiR2-FL-T519A-T7, pMSCV-hiR2-FL-L520A-T7, pMSCV-hiR2-FL-A521S-T7, pMSCV-hiR2-FL-T522A-T7, pMSCV-hiR2-FL-F523A-T7, pMSCV-hiR2-FL-V524A-T7, pMSCV-hiR2-FL-K525A-T7, pMSCV-hiR2-FL-W526A-T7, pMSCV-hiR2-FL-Q527A-T7, pMSCV-hiR2-FL-D528A-T7, pMSCV-hiR2-FL-D529A-T7, pMSCV-hiR2-FL-T530A-T7, pMSCV-hiR2-FL-G531A-T7, pMSCV-hiR2-FL-P532A-T7, pMSCV-hiR2-FL-P533A-T7, pMSCV-hiR2-FL-M534A-T7, pMSCV-hiR2-FL-D535A-T7, pMSCV-hiR2-FL-K536A-T7, pMSCV-hiR2-FL-S537A-T7, pMSCV-hiR2-FL-D538A-T7, pMSCV-hiR2-FL-L539A-T7, pMSCV-hiR2-FL-G540A-T7, pMSCV-hiR2-FL-Q541A-T7, pMSCV-hiR2-FL-K542A-T7, pMSCV-hiR2-FL-R543A-T7, pMSCV-hiR2-FL-T544A-T7, pMSCV-hiR2-FL-S545A-T7, pMSCV-hiR2-FL-G546A-T7, pMSCV-hiR2-FL-A547S-T7, pMSCV-hiR2-FL-V548A-T7, pMSCV-hiR2-FL-C549A-T7, pMSCV-hiR2-FL-H550A-T7, pMSCV-hiR2-FL-Q551A-T7, pMSCV-hiR2-FL-D552A-T7, pMSCV-hiR2-FL-P553A-T7, pMSCV-hiR2-FL-R554A-T7, pMSCV-hiR2-FL-T555A-T7, pMSCV-hiR2-FL-C556A-T7, pMSCV-hiR2-FL-E557A-T7, pMSCV-hiR2-FL-E558A-T7, pMSCV-hiR2-FL-P559A-T7, pMSCV-hiR2-FL-A560S-T7, pMSCV-hiR2-FL-S561A-T7, pMSCV-hiR2-FL-S562A-T7, pMSCV-hiR2-FL-G563A-T7, pMSCV-hiR2-FL-A564S-T7, pMSCV-hiR2-FL-H565A-T7, pMSCV-hiR2-FL-I566A-T7, pMSCV-hiR2-FL-W567A-T7, pMSCV-hiR2-FL-P568A-T7, pMSCV-hiR2-FL-D569A-T7, pMSCV-hiR2-FL-D570A-T7, pMSCV-hiR2-FL-1571A-T7, pMSCV-hiR2-FL-T572A-T7, pMSCV-hiR2-FL-K573A-T7, pMSCV-hiR2-FL-W574A-T7, pMSCV-hiR2-FL-P575A-T7, pMSCV-hiR2-FL-1576A-T7, pMSCV-hiR2-FL-C577A-T7, pMSCV-hiR2-FL-T578A-T7, pMSCV-hiR2-FL-E579A-T7, pMSCV-hiR2-FL-Q580A-T7, pMSCV-hiR2-FL-A581S-T7, pMSCV-hiR2-FL-R582A-T7, pMSCV-hiR2-FL-S583A-T7, pMSCV-hiR2-FL-N584A-T7, pMSCV-hiR2-FL-H585A-T7, pMSCV-hiR2-FL-T586A-T7, pMSCV-hiR2-FL-G587A-T7, pMSCV-hiR2-FL-F588A-T7, pMSCV-hiR2-FL-L589A-T7, pMSCV-hiR2-FL-H590A-T7, pMSCV-hiR2-FL-M591A-T7, pMSCV-hiR2-FL-D592A-T7 and pMSCV-hiR2-FL-C593A-T7 encoding human iRhom2 full length single amino acid substitutions C-terminally tagged with 3 consecutive copies of the T7 epitope (MASMTGGQQMG), and were kept at 37° C., 5% CO₂. After 7 hours, the transfections were stopped by replacing cell supernatants with standard growth medium lacking chloroquine, and cells were incubated at 37° C., 5% CO₂ to allow virus production overnight. In parallel, immortalized iRhom1/2−/− DKO MEFs as target cells for retroviral infection were seeded on 6-well tissue culture plates (Greiner, Germany) in standard growth medium at 1×10⁵ cells per well and were also kept overnight at 37° C., 5% CO₂. On day 3, the supernatants of Phoenix-ECO cells releasing pMSCV-hiR2-FL-N503A-T7, pMSCV-hiR2-FL-D504A-T7, pMSCV-hiR2-FL-H505A-T7, pMSCV-hiR2-FL-S506A-T7, pMSCV-hiR2-FL-G507A-T7, pMSCV-hiR2-FL-C508A-T7, pMSCV-hiR2-FL-1509A-T7, pMSCV-hiR2-FL-Q510A-T7, pMSCV-hiR2-FL-T511A-T7, pMSCV-hiR2-FL-Q512A-T7, pMSCV-hiR2-FL-R513A-T7, pMSCV-hiR2-FL-K514A-T7, pMSCV-hiR2-FL-D515A-T7, pMSCV-hiR2-FL-C516A-T7, pMSCV-hiR2-FL-S517A-T7, pMSCV-hiR2-FL-E518A-T7, pMSCV-hiR2-FL-T519A-T7, pMSCV-hiR2-FL-L520A-T7, pMSCV-hiR2-FL-A521S-T7, pMSCV-hiR2-FL-T522A-T7, pMSCV-hiR2-FL-F523A-T7, pMSCV-hiR2-FL-V524A-T7, pMSCV-hiR2-FL-K525A-T7, pMSCV-hiR2-FL-W526A-T7, pMSCV-hiR2-FL-Q527A-T7, pMSCV-hiR2-FL-D528A-T7, pMSCV-hiR2-FL-D529A-T7, pMSCV-hiR2-FL-T530A-T7, pMSCV-hiR2-FL-G531A-T7, pMSCV-hiR2-FL-P532A-T7, pMSCV-hiR2-FL-P533A-T7, pMSCV-hiR2-FL-M534A-T7, pMSCV-hiR2-FL-D535A-T7, pMSCV-hiR2-FL-K536A-T7, pMSCV-hiR2-FL-S537A-T7, pMSCV-hiR2-FL-D538A-T7, pMSCV-hiR2-FL-L539A-T7, pMSCV-hiR2-FL-G540A-T7, pMSCV-hiR2-FL-Q541A-T7, pMSCV-hiR2-FL-K542A-T7, pMSCV-hiR2-FL-R543A-T7, pMSCV-hiR2-FL-T544A-T7, pMSCV-hiR2-FL-S545A-T7, pMSCV-hiR2-FL-G546A-T7, pMSCV-hiR2-FL-A547S-T7, pMSCV-hiR2-FL-V548A-T7, pMSCV-hiR2-FL-C549A-T7, pMSCV-hiR2-FL-H550A-T7, pMSCV-hiR2-FL-Q551A-T7, pMSCV-hiR2-FL-D552A-T7, pMSCV-hiR2-FL-P553A-T7, pMSCV-hiR2-FL-R554A-T7, pMSCV-hiR2-FL-T555A-T7, pMSCV-hiR2-FL-C556A-T7, pMSCV-hiR2-FL-E557A-T7, pMSCV-hiR2-FL-E558A-T7, pMSCV-hiR2-FL-P559A-T7, pMSCV-hiR2-FL-A560S-T7, pMSCV-hiR2-FL-S561A-T7, pMSCV-hiR2-FL-S562A-T7, pMSCV-hiR2-FL-G563A-T7, pMSCV-hiR2-FL-A564S-T7, pMSCV-hiR2-FL-H565A-T7, pMSCV-hiR2-FL-1566A-T7, pMSCV-hiR2-FL-W567A-T7, pMSCV-hiR2-FL-P568A-T7, pMSCV-hiR2-FL-D569A-T7, pMSCV-hiR2-FL-D570A-T7, pMSCV-hiR2-FL-I571A-T7, pMSCV-hiR2-FL-T572A-T7, pMSCV-hiR2-FL-K573A-T7, pMSCV-hiR2-FL-W574A-T7, pMSCV-hiR2-FL-P575A-T7, pMSCV-hiR2-FL-1576A-T7, pMSCV-hiR2-FL-C577A-T7, pMSCV-hiR2-FL-T578A-T7, pMSCV-hiR2-FL-E579A-T7, pMSCV-hiR2-FL-Q580A-T7, pMSCV-hiR2-FL-A581S-T7, pMSCV-hiR2-FL-R582A-T7, pMSCV-hiR2-FL-S583A-T7, pMSCV-hiR2-FL-N584A-T7, pMSCV-hiR2-FL-H585A-T7, pMSCV-hiR2-FL-T586A-T7, pMSCV-hiR2-FL-G587A-T7, pMSCV-hiR2-FL-F588A-T7, pMSCV-hiR2-FL-L589A-T7, pMSCV-hiR2-FL-H590A-T7, pMSCV-hiR2-FL-M591A-T7, pMSCV-hiR2-FL-D592A-T7 and pMSCV-hiR2-FL-C593A-T7 ecotrophic virus were collected, filtered with 0.45 μm CA filters, and supplemented with 4 μg/ml of polybrene (Sigma-Aldrich, USA). Upon removal of medium from the immortalized iRhom1/2−/− DKO MEFs, these supernatants were added to the target cells for 4 hours at 37° C., 5% CO₂ for first infection. Simultaneously, Phoenix-ECO cells were re-incubated with fresh medium, which, after another 4 hours, was filtered and used for the second infection of the respective target cell populations, again in the presence of 4 μg/ml of polybrene. Likewise, a third, but overnight infection cycle was performed. On day 4, virus containing cell supernatants were replaced by fresh standard growth medium. From day 5 onwards, cells were grown in the presence of 2 mg/ml of geneticin (G418, Thermo Fisher Scientific, USA) for the selection of immortalized MEF-DKO-hiR2-FL-N503A-T7, MEF-DKO-hiR2-FL-D504A-T7, MEF-DKO-hiR2-FL-H505A-T7, MEF-DKO-hiR2-FL-S506A-T7, MEF-DKO-hiR2-FL-G507A-T7, MEF-DKO-hiR2-FL-C508A-T7, MEF-DKO-hiR2-FL-I509A-T7, MEF-DKO-hiR2-FL-Q510A-T7, MEF-DKO-hiR2-FL-T511A-T7, MEF-DKO-hiR2-FL-Q512A-T7, MEF-DKO-hiR2-FL-R513A-T7, MEF-DKO-hiR2-FL-K514A-T7, MEF-DKO-hiR2-FL-D515A-T7, MEF-DKO-hiR2-FL-C516A-T7, MEF-DKO-hiR2-FL-S517A-T7, MEF-DKO-hiR2-FL-E518A-T7, MEF-DKO-hiR2-FL-T519A-T7, MEF-DKO-hiR2-FL-L520A-T7, MEF-DKO-hiR2-FL-A521S-T7, MEF-DKO-hiR2-FL-T522A-T7, MEF-DKO-hiR2-FL-F523A-T7, MEF-DKO-hiR2-FL-V524A-T7, MEF-DKO-hiR2-FL-K525A-T7, MEF-DKO-hiR2-FL-W526A-T7, MEF-DKO-hiR2-FL-Q527A-T7, MEF-DKO-hiR2-FL-D528A-T7, MEF-DKO-hiR2-FL-D529A-T7, MEF-DKO-hiR2-FL-T530A-T7, MEF-DKO-hiR2-FL-G531A-T7, MEF-DKO-hiR2-FL-P532A-T7, MEF-DKO-hiR2-FL-P533A-T7, MEF-DKO-hiR2-FL-M534A-T7, MEF-DKO-hiR2-FL-D535A-T7, MEF-DKO-hiR2-FL-K536A-T7, MEF-DKO-hiR2-FL-S537A-T7, MEF-DKO-hiR2-FL-D538A-T7, MEF-DKO-hiR2-FL-L539A-T7, MEF-DKO-hiR2-FL-G540A-T7, MEF-DKO-hiR2-FL-Q541A-T7, MEF-DKO-hiR2-FL-K542A-T7, MEF-DKO-hiR2-FL-R543A-T7, MEF-DKO-hiR2-FL-T544A-T7, MEF-DKO-hiR2-FL-S545A-T7, MEF-DKO-hiR2-FL-G546A-T7, MEF-DKO-hiR2-FL-A547S-T7, MEF-DKO-hiR2-FL-V548A-T7, MEF-DKO-hiR2-FL-C549A-T7, MEF-DKO-hiR2-FL-H550A-T7, MEF-DKO-hiR2-FL-Q551A-T7, MEF-DKO-hiR2-FL-D552A-T7, MEF-DKO-hiR2-FL-P553A-T7, MEF-DKO-hiR2-FL-R554A-T7, MEF-DKO-hiR2-FL-T555A-T7, MEF-DKO-hiR2-FL-C556A-T7, MEF-DKO-hiR2-FL-E557A-T7, MEF-DKO-hiR2-FL-E558A-T7, MEF-DKO-hiR2-FL-P559A-T7, MEF-DKO-hiR2-FL-A560S-T7, MEF-DKO-hiR2-FL-S561A-T7, MEF-DKO-hiR2-FL-S562A-T7, MEF-DKO-hiR2-FL-G563A-T7, MEF-DKO-hiR2-FL-A564S-T7, MEF-DKO-hiR2-FL-H565A-T7, MEF-DKO-hiR2-FL-1566A-T7, MEF-DKO-hiR2-FL-W567A-T7, MEF-DKO-hiR2-FL-P568A-T7, MEF-DKO-hiR2-FL-D569A-T7, MEF-DKO-hiR2-FL-D570A-T7, MEF-DKO-hiR2-FL-1571A-T7, MEF-DKO-hiR2-FL-T572A-T7, MEF-DKO-hiR2-FL-K573A-T7, MEF-DKO-hiR2-FL-W574A-T7, MEF-DKO-hiR2-FL-P575A-T7, MEF-DKO-hiR2-FL-1576A-T7, MEF-DKO-hiR2-FL-C577A-T7, MEF-DKO-hiR2-FL-T578A-T7, MEF-DKO-hiR2-FL-E579A-T7, MEF-DKO-hiR2-FL-Q580A-T7, MEF-DKO-hiR2-FL-A581S-T7, MEF-DKO-hiR2-FL-R582A-T7, MEF-DKO-hiR2-FL-S583A-T7, MEF-DKO-hiR2-FL-N584A-T7, MEF-DKO-hiR2-FL-H585A-T7, MEF-DKO-hiR2-FL-T586A-T7, MEF-DKO-hiR2-FL-G587A-T7, MEF-DKO-hiR2-FL-F588A-T7, MEF-DKO-hiR2-FL-L589A-T7, MEF-DKO-hiR2-FL-H590A-T7, MEF-DKO-hiR2-FL-M591A-T7, MEF-DKO-hiR2-FL-D592A-T7 and MEF-DKO-hiR2-FL-C593A-T7 cells stably expressing human iRhom2 full length single amino acid substitutions C-terminally tagged with 3 consecutive copies of the T7 epitope. Upon propagation, cells were stocked for future usage.

### FACS Analyses for Test System Validation

In brief, immortalized MEF-DKO-hiR2-FL-WT-T7 cells and MEF-DKO-hiR2-FL-N503A-T7, MEF-DKO-hiR2-FL-D504A-T7, MEF-DKO-hiR2-FL-H505A-T7, MEF-DKO-hiR2-FL-S506A-T7, MEF-DKO-hiR2-FL-G507A-T7, MEF-DKO-hiR2-FL-C508A-T7, MEF-DKO-hiR2-FL-I509A-T7, MEF-DKO-hiR2-FL-Q510A-T7, MEF-DKO-hiR2-FL-T511A-T7, MEF-DKO-hiR2-FL-Q512A-T7, MEF-DKO-hiR2-FL-R513A-T7, MEF-DKO-hiR2-FL-K514A-T7, MEF-DKO-hiR2-FL-D515A-T7, MEF-DKO-hiR2-FL-C516A-T7, MEF-DKO-hiR2-FL-S517A-T7, MEF-DKO-hiR2-FL-E518A-T7, MEF-DKO-hiR2-FL-T519A-T7, MEF-DKO-hiR2-FL-L520A-T7, MEF-DKO-hiR2-FL-A521S-T7, MEF-DKO-hiR2-FL-T522A-T7, MEF-DKO-hiR2-FL-F523A-T7, MEF-DKO-hiR2-FL-V524A-T7, MEF-DKO-hiR2-FL-K525A-T7, MEF-DKO-hiR2-FL-W526A-T7, MEF-DKO-hiR2-FL-Q527A-T7, MEF-DKO-hiR2-FL-D528A-T7, MEF-DKO-hiR2-FL-D529A-T7, MEF-DKO-hiR2-FL-T530A-T7, MEF-DKO-hiR2-FL-G531A-T7, MEF-DKO-hiR2-FL-P532A-T7, MEF-DKO-hiR2-FL-P533A-T7, MEF-DKO-hiR2-FL-M534A-T7, MEF-DKO-hiR2-FL-D535A-T7, MEF-DKO-hiR2-FL-K536A-T7, MEF-DKO-hiR2-FL-S537A-T7, MEF-DKO-hiR2-FL-D538A-T7, MEF-DKO-hiR2-FL-L539A-T7, MEF-DKO-hiR2-FL-G540A-T7, MEF-DKO-hiR2-FL-Q541A-T7, MEF-DKO-hiR2-FL-K542A-T7, MEF-DKO-hiR2-FL-R543A-T7, MEF-DKO-hiR2-FL-T544A-T7, MEF-DKO-hiR2-FL-S545A-T7, MEF-DKO-hiR2-FL-G546A-T7, MEF-DKO-hiR2-FL-A547S-T7, MEF-DKO-hiR2-FL-V548A-T7, MEF-DKO-hiR2-FL-C549A-T7, MEF-DKO-hiR2-FL-H550A-T7, MEF-DKO-hiR2-FL-Q551A-T7, MEF-DKO-hiR2-FL-D552A-T7, MEF-DKO-hiR2-FL-P553A-T7, MEF-DKO-hiR2-FL-R554A-T7, MEF-DKO-hiR2-FL-T555A-T7, MEF-DKO-hiR2-FL-C556A-T7, MEF-DKO-hiR2-FL-E557A-T7, MEF-DKO-hiR2-FL-E558A-T7, MEF-DKO-hiR2-FL-P559A-T7, MEF-DKO-hiR2-FL-A560S-T7, MEF-DKO-hiR2-FL-S561A-T7, MEF-DKO-hiR2-FL-S562A-T7, MEF-DKO-hiR2-FL-G563A-T7, MEF-DKO-hiR2-FL-A564S-T7, MEF-DKO-hiR2-FL-H565A-T7, MEF-DKO-hiR2-FL-1566A-T7, MEF-DKO-hiR2-FL-W567A-T7, MEF-DKO-hiR2-FL-P568A-T7, MEF-DKO-hiR2-FL-D569A-T7, MEF-DKO-hiR2-FL-D570A-T7, MEF-DKO-hiR2-FL-I571A-T7, MEF-DKO-hiR2-FL-T572A-T7, MEF-DKO-hiR2-FL-K573A-T7, MEF-DKO-hiR2-FL-W574A-T7, MEF-DKO-hiR2-FL-P575A-T7, MEF-DKO-hiR2-FL-1576A-T7, MEF-DKO-hiR2-FL-C577A-T7, MEF-DKO-hiR2-FL-T578A-T7, MEF-DKO-hiR2-FL-E579A-T7, MEF-DKO-hiR2-FL-Q580A-T7, MEF-DKO-hiR2-FL-A581S-T7, MEF-DKO-hiR2-FL-R582A-T7, MEF-DKO-hiR2-FL-S583A-T7, MEF-DKO-hiR2-FL-N584A-T7, MEF-DKO-hiR2-FL-H585A-T7, MEF-DKO-hiR2-FL-T586A-T7, MEF-DKO-hiR2-FL-G587A-T7, MEF-DKO-hiR2-FL-F588A-T7, MEF-DKO-hiR2-FL-L589A-T7, MEF-DKO-hiR2-FL-H590A-T7, MEF-DKO-hiR2-FL-M591A-T7, MEF-DKO-hiR2-FL-D592A-T7 and MEF-DKO-hiR2-FL-C593A-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 1×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or mouse monoclonal anti-T7 IgG (Merck Millipore, USA) at 3 μg/ml FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

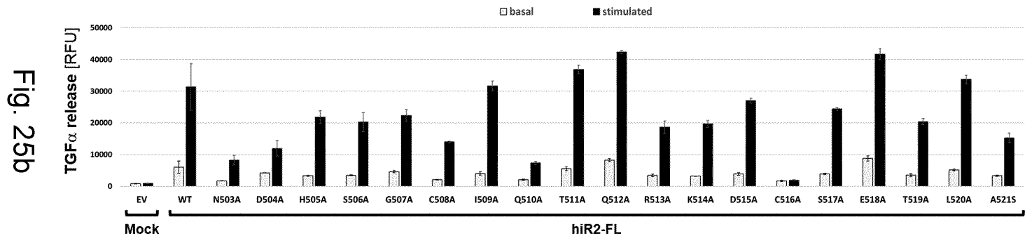

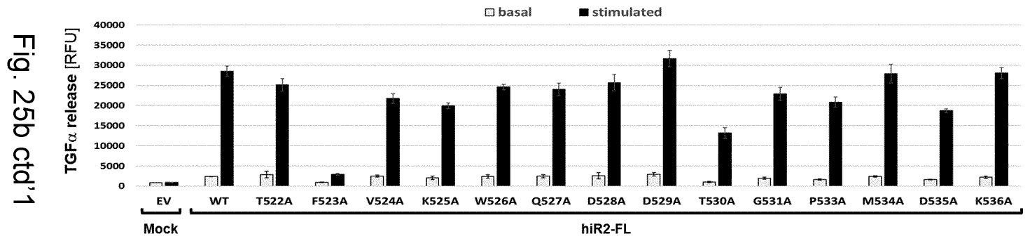

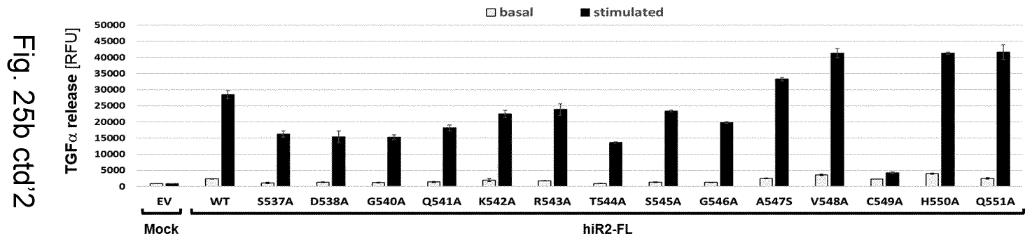

FIG. 25a shows representative results of this experiment exemplarily for the human iRhom2 variant hiR2-FL-K536A-T7. Binding analyses of anti-T7 tag antibody (black) and anti-mouse IgG secondary antibody (gray) on MEF-DKO-hiR2-FL-WT-T7 (left) and MEF-DKO-hiR2-FL-K536A-T7 cells (right) reveal a comparably strong increase in relative fluorescence intensity. This demonstrates that, similarly to human iRhom2 wild type (left), the human iRhom2 variant hiR2-FL-K536A-T7 is equally well expressed and localized on the surface of these cells (right). Similar results were obtained for the expression and localization of the human iRhom2 full length single amino acid substitutions expressed on MEF-DKO-hiR2-FL-N503A-T7, MEF-DKO-hiR2-FL-H505A-T7, MEF-DKO-hiR2-FL-S506A-T7, MEF-DKO-hiR2-FL-I509A-T7, MEF-DKO-hiR2-FL-T511A-T7, MEF-DKO-hiR2-FL-Q512A-T7, MEF-DKO-hiR2-FL-R513A-T7, MEF-DKO-hiR2-FL-K514A-T7, MEF-DKO-hiR2-FL-D515A-T7, MEF-DKO-hiR2-FL-S517A-T7, MEF-DKO-hiR2-FL-E518A-T7, MEF-DKO-hiR2-FL-T519A-T7, MEF-DKO-hiR2-FL-L520A-T7, MEF-DKO-hiR2-FL-A521S-T7, MEF-DKO-hiR2-FL-T522A-T7, MEF-DKO-hiR2-FL-V524A-T7, MEF-DKO-hiR2-FL-K525A-T7, MEF-DKO-hiR2-FL-W526A-T7, MEF-DKO-hiR2-FL-Q527A-T7, MEF-DKO-hiR2-FL-D528A-T7, MEF-DKO-hiR2-FL-D529A-T7, MEF-DKO-hiR2-FL-T530A-T7, MEF-DKO-hiR2-FL-G531A-T7, MEF-DKO-hiR2-FL-P532A-T7, MEF-DKO-hiR2-FL-P533A-T7, MEF-DKO-hiR2-FL-M534A-T7, MEF-DKO-hiR2-FL-D535A-T7, MEF-DKO-hiR2-FL-K536A-T7, MEF-DKO-hiR2-FL-S537A-T7, MEF-DKO-hiR2-FL-D538A-T7, MEF-DKO-hiR2-FL-L539A-T7, MEF-DKO-hiR2-FL-G540A-T7, MEF-DKO-hiR2-FL-Q541A-T7, MEF-DKO-hiR2-FL-K542A-T7, MEF-DKO-hiR2-FL-R543A-T7, MEF-DKO-hiR2-FL-T544A-T7, MEF-DKO-hiR2-FL-S545A-T7, MEF-DKO-hiR2-FL-G546A-T7, MEF-DKO-hiR2-FL-A547S-T7, MEF-DKO-hiR2-FL-V548A-T7, MEF-DKO-hiR2-FL-H550A-T7, MEF-DKO-hiR2-FL-Q551A-T7, MEF-DKO-hiR2-FL-P553A-T7, MEF-DKO-hiR2-FL-R554A-T7, MEF-DKO-hiR2-FL-T555A-T7, MEF-DKO-hiR2-FL-E557A-T7, MEF-DKO-hiR2-FL-E558A-T7, MEF-DKO-hiR2-FL-P559A-T7, MEF-DKO-hiR2-FL-A560S-T7, MEF-DKO-hiR2-FL-S561A-T7, MEF-DKO-hiR2-FL-S562A-T7, MEF-DKO-hiR2-FL-G563A-T7, MEF-DKO-hiR2-FL-A564S-T7, MEF-DKO-hiR2-FL-H565A-T7, MEF-DKO-hiR2-FL-I566A-T7, MEF-DKO-hiR2-FL-P568A-T7, MEF-DKO-hiR2-FL-D569A-T7, MEF-DKO-hiR2-FL-D570A-T7, MEF-DKO-hiR2-FL-1571A-T7, MEF-DKO-hiR2-FL-T572A-T7, MEF-DKO-hiR2-FL-K573A-T7, MEF-DKO-hiR2-FL-P575A-T7, MEF-DKO-hiR2-FL-I576A-T7, MEF-DKO-hiR2-FL-T578A-T7, MEF-DKO-hiR2-FL-E579A-T7, MEF-DKO-hiR2-FL-Q580A-T7, MEF-DKO-hiR2-FL-A581S-T7, MEF-DKO-hiR2-FL-R582A-T7, MEF-DKO-hiR2-FL-S583A-T7, MEF-DKO-hiR2-FL-N584A-T7, MEF-DKO-hiR2-FL-H585A-T7, MEF-DKO-hiR2-FL-T586A-T7, MEF-DKO-hiR2-FL-G587A-T7, MEF-DKO-hiR2-FL-F588A-T7, MEF-DKO-hiR2-FL-L589A-T7, MEF-DKO-hiR2-FL-H590A-T7, MEF-DKO-hiR2-FL-M591A-T7 and MEF-DKO-hiR2-FL-D592A-T7 cells. Moreover, a decrease in relative fluorescence intensity and, thus, a reduced expression on the surface of these cells was obtained for the following human iRhom2 full length single amino acid substitutions expressed on MEF-DKO-hiR2-FL-D504A-T7, MEF-DKO-hiR2-FL-G507A-T7, MEF-DKO-hiR2-FL-C508A-T7, MEF-DKO-hiR2-FL-Q510A-T7, MEF-DKO-hiR2-FL-C516A-T7, MEF-DKO-hiR2-FL-F523A-T7, MEF-DKO-hiR2-FL-C549A-T7, MEF-DKO-hiR2-FL-D552A-T7, MEF-DKO-hiR2-FL-C556A-T7, MEF-DKO-hiR2-FL-W567A-T7, MEF-DKO-hiR2-FL-W574A-T7, MEF-DKO-hiR2-FL-C577A-T7 and MEF-DKO-hiR2-FL-C593A-T7 cells.

### TGFα ELISA for Test System Validation

To test all 91 human iRhom2 variants with human iRhom1-specific single amino acid substitutions, the respective MEF-DKO cell lines stably expressing these variants, generated as described in the example above, were subjected to TGFα shedding ELISA analysis. In order to demonstrate the functionality of all variants as an indicator that these variants are properly folded, PMA-induced release of nucleofected TGFα was assessed. As the cells used in this analysis are rescue variants of iRhom1/2−/− double knockout mouse embryonic fibroblasts (described in Example 11), that are rescued by the respective human iRhom2 variant with the human iRhom1-specific single amino acid substitution or deletion, the iRhom2 variant stably expressed is the only iRhom protein expressed in these cells at all and is therefore the only contributing iRhom to the shedding TGFα in these cells.

In brief, on day 1, Nunc black MaxiSorp® 96-well plates (Thermo Fisher Scientific, USA) were coated overnight with 100 μl per well of mouse anti-human TGFα capture antibody (provided as part of the DuoSet ELISA kit) at 400 ng/ml in TBS at 4° C. After MEF-DKO-hiR2-FL-N503A-T7, MEF-DKO-hiR2-FL-D504A-T7, MEF-DKO-hiR2-FL-H505A-T7, MEF-DKO-hiR2-FL-S506A-T7, MEF-DKO-hiR2-FL-G507A-T7, MEF-DKO-hiR2-FL-C508A-T7, MEF-DKO-hiR2-FL-1509A-T7, MEF-DKO-hiR2-FL-Q510A-T7, MEF-DKO-hiR2-FL-T511A-T7, MEF-DKO-hiR2-FL-Q512A-T7, MEF-DKO-hiR2-FL-R513A-T7, MEF-DKO-hiR2-FL-K514A-T7, MEF-DKO-hiR2-FL-D515A-T7, MEF-DKO-hiR2-FL-C516A-T7, MEF-DKO-hiR2-FL-S517A-T7, MEF-DKO-hiR2-FL-E518A-T7, MEF-DKO-hiR2-FL-T519A-T7, MEF-DKO-hiR2-FL-L520A-T7, MEF-DKO-hiR2-FL-A521S-T7, MEF-DKO-hiR2-FL-T522A-T7, MEF-DKO-hiR2-FL-F523A-T7, MEF-DKO-hiR2-FL-V524A-T7, MEF-DKO-hiR2-FL-K525A-T7, MEF-DKO-hiR2-FL-W526A-T7, MEF-DKO-hiR2-FL-Q527A-T7, MEF-DKO-hiR2-FL-D528A-T7, MEF-DKO-hiR2-FL-D529A-T7, MEF-DKO-hiR2-FL-T530A-T7, MEF-DKO-hiR2-FL-G531A-T7, MEF-DKO-hiR2-FL-P532A-T7, MEF-DKO-hiR2-FL-P533A-T7, MEF-DKO-hiR2-FL-M534A-T7, MEF-DKO-hiR2-FL-D535A-T7, MEF-DKO-hiR2-FL-K536A-T7, MEF-DKO-hiR2-FL-S537A-T7, MEF-DKO-hiR2-FL-D538A-T7, MEF-DKO-hiR2-FL-L539A-T7, MEF-DKO-hiR2-FL-G540A-T7, MEF-DKO-hiR2-FL-Q541A-T7, MEF-DKO-hiR2-FL-K542A-T7, MEF-DKO-hiR2-FL-R543A-T7, MEF-DKO-hiR2-FL-T544A-T7, MEF-DKO-hiR2-FL-S545A-T7, MEF-DKO-hiR2-FL-G546A-T7, MEF-DKO-hiR2-FL-A547S-T7, MEF-DKO-hiR2-FL-V548A-T7, MEF-DKO-hiR2-FL-C549A-T7, MEF-DKO-hiR2-FL-H550A-T7, MEF-DKO-hiR2-FL-Q551A-T7, MEF-DKO-hiR2-FL-D552A-T7, MEF-DKO-hiR2-FL-P553A-T7, MEF-DKO-hiR2-FL-R554A-T7, MEF-DKO-hiR2-FL-T555A-T7, MEF-DKO-hiR2-FL-C556A-T7, MEF-DKO-hiR2-FL-E557A-T7, MEF-DKO-hiR2-FL-E558A-T7, MEF-DKO-hiR2-FL-P559A-T7, MEF-DKO-hiR2-FL-A560S-T7, MEF-DKO-hiR2-FL-S561A-T7, MEF-DKO-hiR2-FL-S562A-T7, MEF-DKO-hiR2-FL-G563A-T7, MEF-DKO-hiR2-FL-A564S-T7, MEF-DKO-hiR2-FL-H565A-T7, MEF-DKO-hiR2-FL-1566A-T7, MEF-DKO-hiR2-FL-W567A-T7, MEF-DKO-hiR2-FL-P568A-T7, MEF-DKO-hiR2-FL-D569A-T7, MEF-DKO-hiR2-FL-D570A-T7, MEF-DKO-hiR2-FL-1571A-T7, MEF-DKO-hiR2-FL-T572A-T7, MEF-DKO-hiR2-FL-K573A-T7, MEF-DKO-hiR2-FL-W574A-T7, MEF-DKO-hiR2-FL-P575A-T7, MEF-DKO-hiR2-FL-1576A-T7, MEF-DKO-hiR2-FL-C577A-T7, MEF-DKO-hiR2-FL-T578A-T7, MEF-DKO-hiR2-FL-E579A-T7, MEF-DKO-hiR2-FL-Q580A-T7, MEF-DKO-hiR2-FL-A581S-T7, MEF-DKO-hiR2-FL-R582A-T7, MEF-DKO-hiR2-FL-S583A-T7, MEF-DKO-hiR2-FL-N584A-T7, MEF-DKO-hiR2-FL-H585A-T7, MEF-DKO-hiR2-FL-T586A-T7, MEF-DKO-hiR2-FL-G587A-T7, MEF-DKO-hiR2-FL-F588A-T7, MEF-DKO-hiR2-FL-L589A-T7, MEF-DKO-hiR2-FL-H590A-T7, MEF-DKO-hiR2-FL-M591A-T7, MEF-DKO-hiR2-FL-D592A-T7 and MEF-DKO-hiR2-FL-C593A-T7 cells were electroporated with the hTGFα-FL-WT construct in a pcDNA3.1 vector backbone, using an 4D-Nucleofector System (Lonza, Switzerland), approximately 35,000 MEF-DKO cells carrying the human iRhom2 variant with the human iRhom1-specific single amino acid substitution or deletion were seeded in 100 μl of normal growth medium in each well of F-bottom 96-well cell culture plates (Thermo Fisher Scientific, USA). On day 2, the capture antibody solution was removed and MaxiSorp® plates were blocked with 300 μl per well of TBS, 1% BSA at room temperature for at least 1 hour. Meanwhile, the cells were washed once with PBS and afterwards 80 μl of OptiMEM medium (Thermo Fisher Scientific, USA) was added per well.

Subsequently, cells (except those for unstimulated controls) were stimulated with 20 μl per well of PMA (Sigma-Aldrich, USA) at a final concentration of 25 ng/ml at 37° C., 5% CO₂ for 1 hour. 20 μl of OptiMEM medium was added to the unstimulated control cells. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Thereafter, 100 μl biotinylated goat anti-human TGFα detection antibody (provided as part of the DuoSet ELISA kit) at 37.5 ng/ml in TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

FIG. 25b shows results from these TGFα release assays demonstrating that 83 of the 91 human iRhom2 variants with single amino acid substitutions to alanine are functionally active as TGFα shedding can be induced with PMA, indicating that these variants are properly folded, in contrast to the empty vector (EV) negative control population, where no PMA-induced shedding of TGFα is detectable. The human iRhom2 variants hi2-FL-WT-3xT7-C516A, hiR2-FL-F523A-T7, hiR2-FL-C549A-T7, hiR2-FL-D552A-T7, hiR2-FL-C556A-T7, hiR2-FL-W567A-T7, hiR2-FL-W574A-T7 and hiR2-FL-C577A-T7 showed no or almost no functionality and were therefore excluded from further analyses.

### FACS Analyses to Characterize Binding of the Purified Antibodies of the Invention for the Purpose of Epitope Mapping

In brief, immortalized MEF-DKO-hiR2-FL-N503A-T7, MEF-DKO-hiR2-FL-D504A-T7, MEF-DKO-hiR2-FL-H505A-T7, MEF-DKO-hiR2-FL-S506A-T7, MEF-DKO-hiR2-FL-G507A-T7, MEF-DKO-hiR2-FL-C508A-T7, MEF-DKO-hiR2-FL-1509A-T7, MEF-DKO-hiR2-FL-Q510A-T7, MEF-DKO-hiR2-FL-T511A-T7, MEF-DKO-hiR2-FL-Q512A-T7, MEF-DKO-hiR2-FL-R513A-T7, MEF-DKO-hiR2-FL-K514A-T7, MEF-DKO-hiR2-FL-D515A-T7, MEF-DKO-hiR2-FL-S517A-T7, MEF-DKO-hiR2-FL-E518A-T7, MEF-DKO-hiR2-FL-T519A-T7, MEF-DKO-hiR2-FL-L520A-T7, MEF-DKO-hiR2-FL-A521S-T7, MEF-DKO-hiR2-FL-T522A-T7, MEF-DKO-hiR2-FL-V524A-T7, MEF-DKO-hiR2-FL-K525A-T7, MEF-DKO-hiR2-FL-W526A-T7, MEF-DKO-hiR2-FL-Q527A-T7, MEF-DKO-hiR2-FL-D528A-T7, MEF-DKO-hiR2-FL-D529A-T7, MEF-DKO-hiR2-FL-T530A-T7, MEF-DKO-hiR2-FL-G531A-T7, MEF-DKO-hiR2-FL-P532A-T7, MEF-DKO-hiR2-FL-P533A-T7, MEF-DKO-hiR2-FL-M534A-T7, MEF-DKO-hiR2-FL-D535A-T7, MEF-DKO-hiR2-FL-K536A-T7, MEF-DKO-hiR2-FL-S537A-T7, MEF-DKO-hiR2-FL-D538A-T7, MEF-DKO-hiR2-FL-L539A-T7, MEF-DKO-hiR2-FL-G540A-T7, MEF-DKO-hiR2-FL-Q541A-T7, MEF-DKO-hiR2-FL-K542A-T7, MEF-DKO-hiR2-FL-R543A-T7, MEF-DKO-hiR2-FL-T544A-T7, MEF-DKO-hiR2-FL-S545A-T7, MEF-DKO-hiR2-FL-G546A-T7, MEF-DKO-hiR2-FL-A547S-T7, MEF-DKO-hiR2-FL-V548A-T7, MEF-DKO-hiR2-FL-H550A-T7, MEF-DKO-hiR2-FL-Q551A-T7, MEF-DKO-hiR2-FL-P553A-T7, MEF-DKO-hiR2-FL-R554A-T7, MEF-DKO-hiR2-FL-T555A-T7, MEF-DKO-hiR2-FL-E557A-T7, MEF-DKO-hiR2-FL-E558A-T7, MEF-DKO-hiR2-FL-P559A-T7, MEF-DKO-hiR2-FL-A560S-T7, MEF-DKO-hiR2-FL-S561A-T7, MEF-DKO-hiR2-FL-S562A-T7, MEF-DKO-hiR2-FL-G563A-T7, MEF-DKO-hiR2-FL-A564S-T7, MEF-DKO-hiR2-FL-H565A-T7, MEF-DKO-hiR2-FL-1566A-T7, MEF-DKO-hiR2-FL-P568A-T7, MEF-DKO-hiR2-FL-D569A-T7, MEF-DKO-hiR2-FL-D570A-T7, MEF-DKO-hiR2-FL-1571A-T7, MEF-DKO-hiR2-FL-T572A-T7, MEF-DKO-hiR2-FL-K573A-T7, MEF-DKO-hiR2-FL-P575A-T7, MEF-DKO-hiR2-FL-I576A-T7, MEF-DKO-hiR2-FL-T578A-T7, MEF-DKO-hiR2-FL-E579A-T7, MEF-DKO-hiR2-FL-Q580A-T7, MEF-DKO-hiR2-FL-A581S-T7, MEF-DKO-hiR2-FL-R582A-T7, MEF-DKO-hiR2-FL-S583A-T7, MEF-DKO-hiR2-FL-N584A-T7, MEF-DKO-hiR2-FL-H585A-T7, MEF-DKO-hiR2-FL-T586A-T7, MEF-DKO-hiR2-FL-G587A-T7, MEF-DKO-hiR2-FL-F588A-T7, MEF-DKO-hiR2-FL-L589A-T7, MEF-DKO-hiR2-FL-H590A-T7, MEF-DKO-hiR2-FL-M591A-T7, MEF-DKO-hiR2-FL-D592A-T7 and MEF-DKO-hiR2-FL-C593A-T7 cells were harvested with 10 mM EDTA in PBS, washed and resuspended in FACS buffer (PBS, 3% FBS, 0.05% sodium azide), and seeded in Nunc U-bottom 96-well plates (Thermo Fisher Scientific, USA) at approximately 1×10⁵ cells per well. To pellet cells and remove supernatants, the plates were centrifuged at 1,500 rpm and 4° C. for 3 minutes. For primary staining, cells were resuspended in 100 μl per well of either FACS buffer alone (controls) or the purified antibodies 3, 5, 16, 22, 34, 42, 43, 44, 48, and 50 of the invention at 3 μg/ml in FACS buffer and incubated on ice for 1 hour. Afterwards, plates were centrifuged at 1,500 rpm and 4° C. for 3 minutes and washed twice with 200 μl per well of FACS buffer. For secondary staining, cells were spun down and resuspended in 100 μl per well of PE-conjugated goat anti-mouse IgG F(ab′)2 detection fragment (Dianova, Germany) diluted 1:100 in FACS buffer. Protected from light, the cell suspensions were incubated on ice for 1 hour. Plates were then centrifuged at 1,500 rpm and 4° C. for 3 minutes and washed three times with 200 μl per well of FACS buffer. Finally, cells were resuspended in 150 μl per well of FACS buffer and analyzed using a BD Accuri™ C6 Plus flow cytometer (Becton Dickinson, Germany).

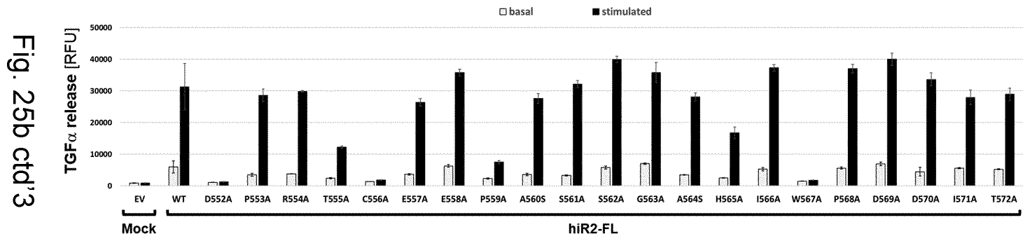

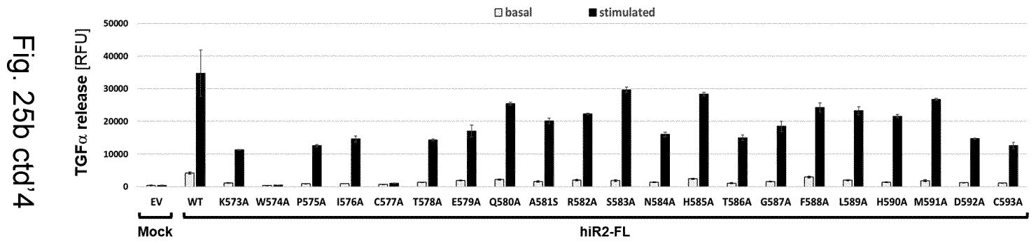

FIG. 26a shows representative results of this experiment. Exemplarily for the entire panel of 83 functional human iRhom2 variants with single amino acid substitutions to alanine, data for the analysis of cells expressing the human iRhom2 variant hiR2-FL-K536A-T7 are shown. Binding analyses of the antibody 5 as a representative example of the antibodies of the invention with inhibitory effects on TNFα release (black, upper panel) or the antibody 50 without inhibitory effects on TNFα release (black, lower panel) as well as anti-mouse IgG secondary antibody (gray) on MEF-DKO-hiR2-FL-WT-T7 cells (left) and MEF-DKO-hiR2-FL-K536A-T7 cells (right) demonstrate that the substitution of the single amino acid lysine 536 of human iRhom2 by alanine strongly impairs and, thus, contributes to binding of the antibody 5 of the invention with inhibitory effects on TNFα release (right, upper panel). In contrast, it does not affect and, thus, does not contribute to binding of the antibody 50 without inhibitory effects on TNFα release (right, lower panel). For both antibodies, binding to MEF-DKO-hiR2-FL-WT-T7 cells (left) serves as positive control.

FIG. 26b summarizes—in extension of FIG. 26a —the results of FACS analyses of the antibodies 3, 5, 16, 22, 34, 42, 43, and 44 of the invention with inhibitory effects on TNFα release versus the antibodies 48 and 50 without inhibitory effects on TNFα release on the entire panel of 83 engineered functional MEF populations expressing human iRhom2 variants with single amino acid substitutions to alanine. Binding of each antibody to human iRhom2 wild type is considered 100 percent. A respective drop of antibody binding to any variant by 30-59 % is indicated by cells held in light gray (and marked with “1”), an impaired binding by 60 -95% is illustrated by cells colored in gray (and marked with “2”), and a loss of binding by >95% is highlighted by dark gray cells (marked with “3”). These data reveal related patterns of amino acid positions relevant for binding of the antibodies 3, 5, 16, 22, 34, 42, 43, and 44 of the invention with inhibitory effects on TNFα release to human iRhom2, which are different from patterns of amino acid positions contributing to binding of the antibodies 48 and 50 without inhibitory effects on TNFα release.

### Example 24: Analysis of Inhibitory Effects of the Antibodies of the Invention on PMA-Induced TNFα Shedding In Vitro

In contrast to Example 14 and 17, where the inhibitory effects of the antibodies of the invention on LPS-induced release of endogenous TNFα from human THP-1 cells were tested, this analysis was conducted with recombinantly produced murine antibodies of the invention to verify their inhibitory effects on PMA-induced release of endogenous TNFα from human monocytic U-937 cells.

The production of the recombinant antibody material that was used in this example is identical to the one described in Example 17. The ELISA-based TNFα release assay that was used in this example is described below.

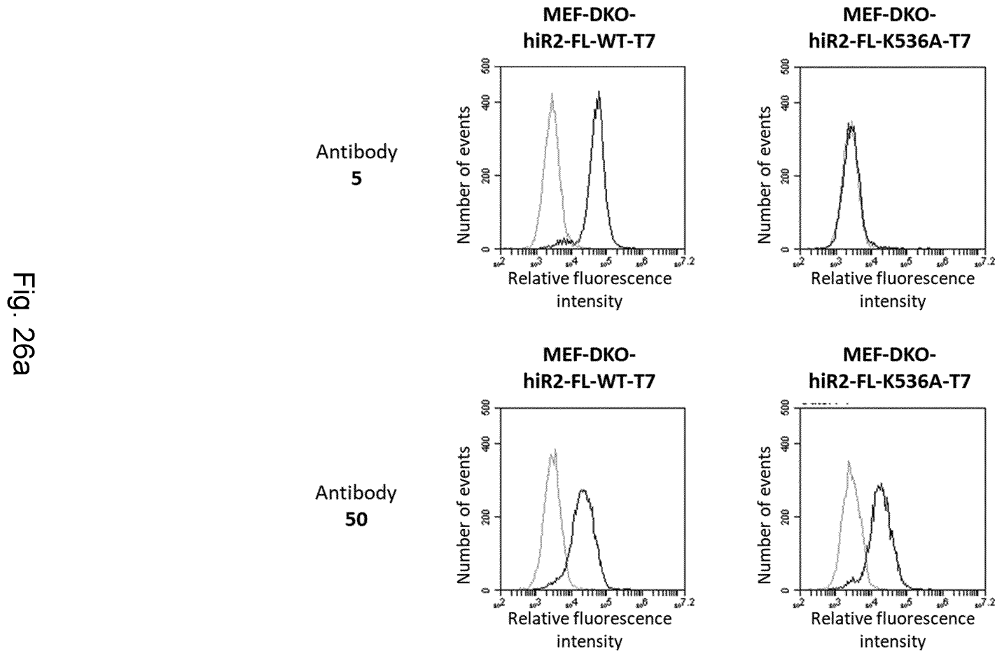

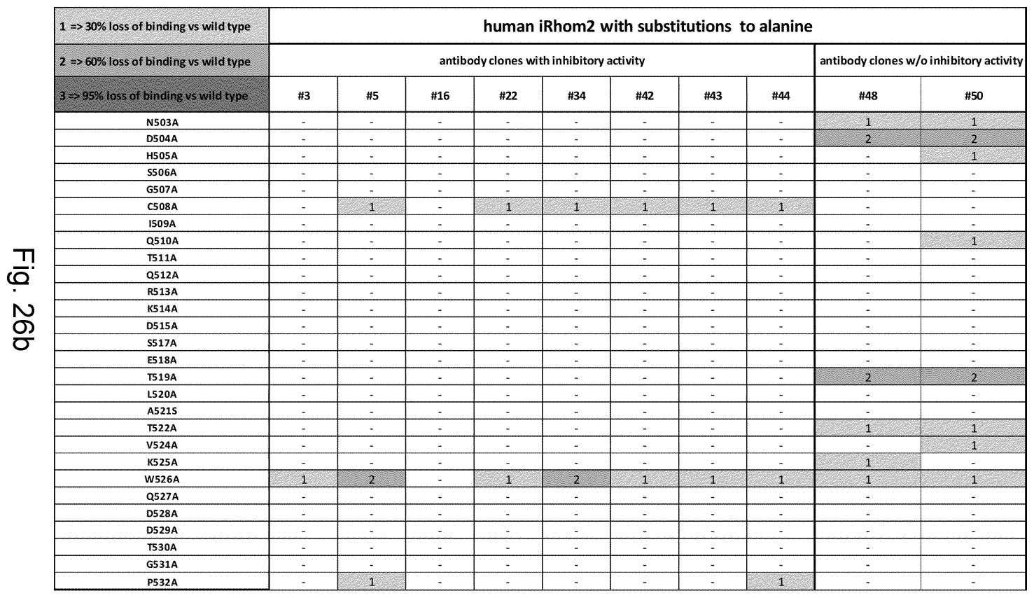

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 1-2 hours. Meanwhile, 80,000 U-937 (European Collection of Authenticated Cell Cultures, UK) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 1 hour. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human TNFα protein (provided as part of the DuoSet ELISA kit) diluted in TBS at defined concentrations were added to the plate as standard references. Thereafter, 100 μl biotinylated goat anti-human TNFα detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm. FIG. 27 shows representative results of this experiment demonstrating the effects of test articles on PMA-induced release of TNFα from U-937 cells in absolute numbers (FIG. 27a) and percent inhibition (FIG. 27b). While Batimastat (BB94) as a small molecule inhibitor of metalloproteinases serves as positive control and results in 100.1% inhibition of PMA-induced release of TNFα, the presence of IgG isotype control has no significant effect on TNFα shedding. In contrast, an equal concentration of the antibodies 3, 5, 16, 22, 34, 42, 43 and 44 of the invention inhibits PMA-induced release of TNFα from U-937 cells by 65.9%, 63.6%, 91.6%, 86.1%, 68.6%, 94.5%, 78.3% and 76.5%, respectively.

### Example 25: Analysis of Inhibitory Effects of the Antibodies of the Invention on PMA-Induced TNFα Shedding In Vitro

Complementary to Example 24 described above, ELISA-based TNFα release assays were performed to verify the inhibitory effects of the antibodies of the invention on PMA-induced release of endogenous TNFα from human U-937 cells. However, this analysis was conducted with both recombinantly produced murine and recombinantly produced chimeric antibodies of the invention.

To produce the recombinant antibody material, target DNA sequence was designed, optimized and synthesized. The complete sequence was sub-cloned into pcDNA3.4 vector (Thermo Fisher Scientific, USA), for the murine material, or into pTT5 vector (Thermo Fisher Scientific, USA), for the chimeric material and the transfection grade plasmid was maxi-prepared for Expi293F (Thermo Fisher Scientific, USA), for the murine material or for CHO-3E7 or HD CHO-S(Thermo Fisher Scientific, USA) for the chimeric material, cell expression. Expi293F cells were grown in serum-free Expi293FTM expression medium (Thermo Fisher Scientific, USA), CHO cells were grown in serum-free FreeStyle™ CHO Expression Medium (Thermo Fisher Scientific, USA) in Erlenmeyer flasks (Corning Inc., USA) at 37° C. with 5-8% CO₂ on an orbital shaker (VWR Scientific, Germany). One day before transfection, the cells were seeded at an appropriate density in new Erlenmeyer flasks. On the day of transfection, DNA and transfection reagent were mixed at an optimal ratio and then added into the flask with cells ready for transfection. The recombinant plasmids encoding target protein were transiently transfected into suspension Expi293F cell cultures, for the murine material or into suspension CHO cell cultures, for the chimeric material. The cell culture supernatant collected on day 6 post-transfection was used for purification. Cell culture broth was centrifuged and filtrated. Filtered cell culture supernatant was loaded onto either HiTrap MabSelect SuRe (GE Healthcare, UK), MabSelect SuRe™ LX (GE Healthcare, UK) or RoboColumn Eshmuno A (Merck Millipore, USA) affinity purification columns at an appropriate flowrate. After washing and elution with appropriate buffers, the eluted fractions were pooled and buffer exchanged to final formulation buffer. The purified protein was analyzed by SDS-PAGE analysis for molecular weight and purity measurements. Finally, the concentration was determined applying a NanoDrop 2000 spectrophotometer (Thermo Fisher Scientific, USA).

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 1-2 hours. Meanwhile, 80,000 U-937 (European Collection of Authenticated Cell Cultures, UK) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse or human IgG antibody (Thermo Fisher Scientific, USA) at 15 μg/ml as isotype control (for a final concentration of 3 μg/ml in the resulting 100 μl sample volume) or antibodies of the invention at 15 μg/ml (for a final concentration of 3 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 1 hour. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human TNFα protein (provided as part of the DuoSet ELISA kit) diluted in TBS at defined concentrations were added to the plate as standard references. Thereafter, 100 μl biotinylated goat anti-human TNFα detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

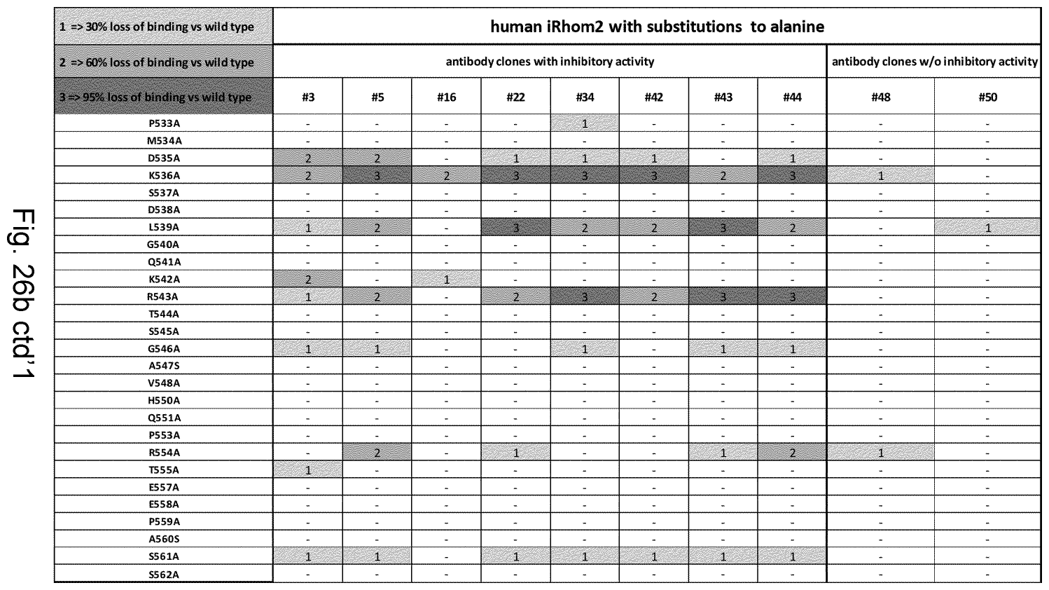

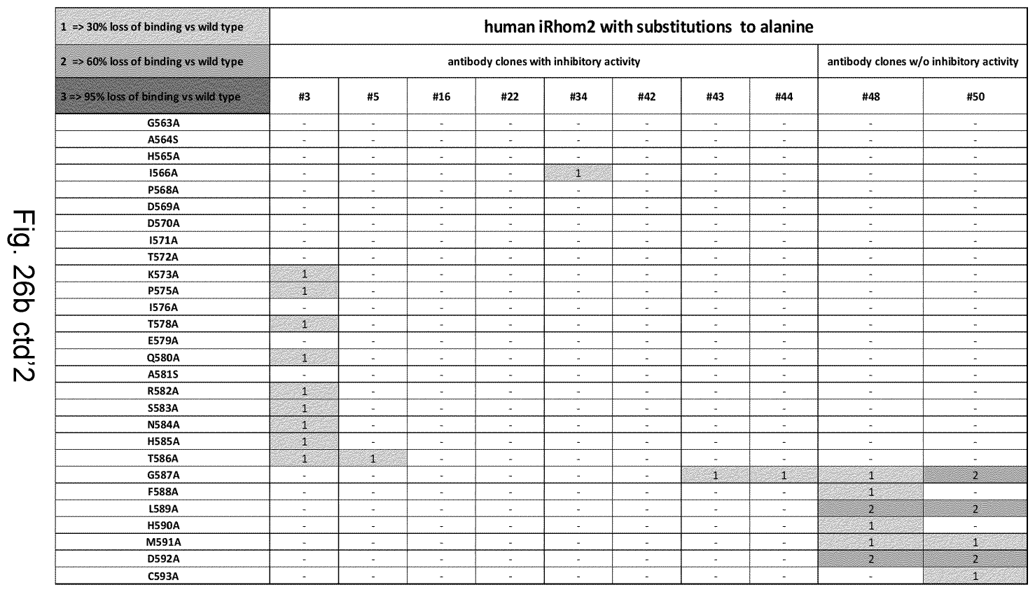

FIG. 28 shows representative results of this experiment demonstrating the effects of test articles on PMA-induced release of TNFα from U-937 cells in absolute numbers (FIG. 28a) and percent inhibition (FIG. 28b). While Batimastat (BB94) as a small molecule inhibitor of metalloproteinases serves as positive control and results in 98.7% inhibition of PMA-induced release of TNFα, the presence of mouse or human IgG isotype control has no significant effect on TNFα shedding. In contrast, an equal concentration of the murine antibodies m16, m22, m34, m42 and m44 of the invention inhibits PMA-induced release of TNFα from U-937 cells by 91.9%, 93.1%, 79.9%, 94.1% and 86.3%, respectively. Highly comparable to the results obtained with the murine antibodies of the invention, an equal concentration of the chimeric antibodies chl6, ch22, ch34, ch42 and ch44 of the invention inhibits PMA-induced release of TNFα from U-937 cells by 93.7%, 96.5%, 87.4%, 96.5% and 89.2%, respectively.

### Example 26: Analysis of Inhibitory Effects of the Antibodies of the Invention on PMA-Induced Interleukin 6 Receptor (IL-6R) Shedding In Vitro

In the following study, ELISA-based IL-6R release assays were performed to analyze the inhibitory effects of the antibodies of the invention on PMA-induced release of endogenous IL-6R from human THP-1 monocytic cells.

The production of the recombinant antibody material that was used in this example is identical to the one described in Example 17. The ELISA-based IL-6R release assay that was used in this example is described below.

In brief, on day 1, Nunc black MaxiSorp® 96-well plates (Thermo Fisher Scientific, USA) were coated for 7 hours with 100 μl per well of mouse anti-human IL-6R capture antibody (provided as part of the DuoSet ELISA kit) at 2 μg/ml TBS at room temperature. Capture antibody solution was removed and MaxiSorp® plates were blocked with 300 μl per well of TBS, 1% BSA at room temperature for 1.5 hours. Meanwhile, 40,000 THP-1 (American Type Culture Collection, USA) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 1 hour. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human IL-6R protein (provided as part of the DuoSet ELISA kit) diluted in TBS at defined concentrations were added to the plate as standard references. Thereafter, 100 μl biotinylated goat anti-human IL-6R detection antibody (provided as part of the DuoSet ELISA kit) at 100 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

FIGS. 29a & 29b show representative results of this experiment demonstrating the effects of test articles on PMA-induced release of IL-6R from THP-1 cells in absolute numbers (FIG. 29a) and percent inhibition (FIG. 29b). While Batimastat (BB94) as a small molecule inhibitor of metalloproteinases serves as positive control and results in 88.6% inhibition of PMA-induced release of IL-6R, the presence of IgG isotype control has no significant effect on IL-6R shedding. In contrast, an equal concentration of the antibodies 3, 5, 16, 22, 34, 42, 43 and 44 of the invention inhibits PMA-induced release of IL-6R from THP-1 cells by 46.7%, 64.1%, 72.5%, 67.4%, 71.1%, 85.9%, 72.9% and 73.0%, respectively.

### Example 27: Analysis of Inhibitory Effects of the Antibodies of the Invention on PMA-Induced Interleukin 6 Receptor (IL-6R) Shedding In Vitro

Complementary to Example 26 described above, ELISA-based IL-6R release assays were performed to verify the inhibitory effects of the antibodies of the invention on PMA-induced release of endogenous IL-6R from human U-937 cells.

The production of the recombinant antibody material that was used in this example is identical to the one described in Example 17. The ELISA-based IL-6R release assay that was used in this example is described below.

In brief, on day 1, Nunc black MaxiSorp® 96-well plates (Thermo Fisher Scientific, USA) were coated overnight with 100 μl per well of mouse anti-human IL-6R capture antibody (provided as part of the DuoSet ELISA kit) at 2 μg/ml TBS at 4° C. On day 2, the capture antibody solution was removed and MaxiSorp® plates were blocked with 300 μl per well of TBS, 1% BSA at room temperature for 1-2 hours. Meanwhile, 80,000 U-937 (European Collection of Authenticated Cell Cultures, UK) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 375 ng/ml in growth medium for a final concentration of 62.5 ng/ml at 37° C., 5% CO₂ for 1 hour. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human IL-6R protein (provided as part of the DuoSet ELISA kit) diluted in TBS at defined concentrations were added to the plate as standard references. Thereafter, 100 μl biotinylated goat anti-human IL-6R detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

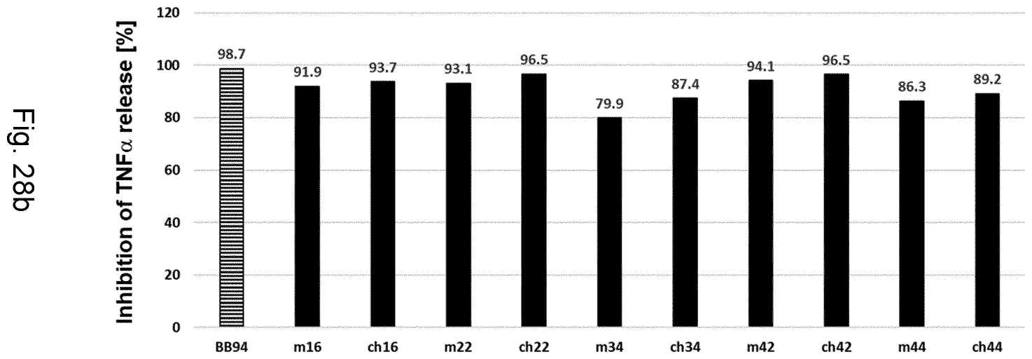

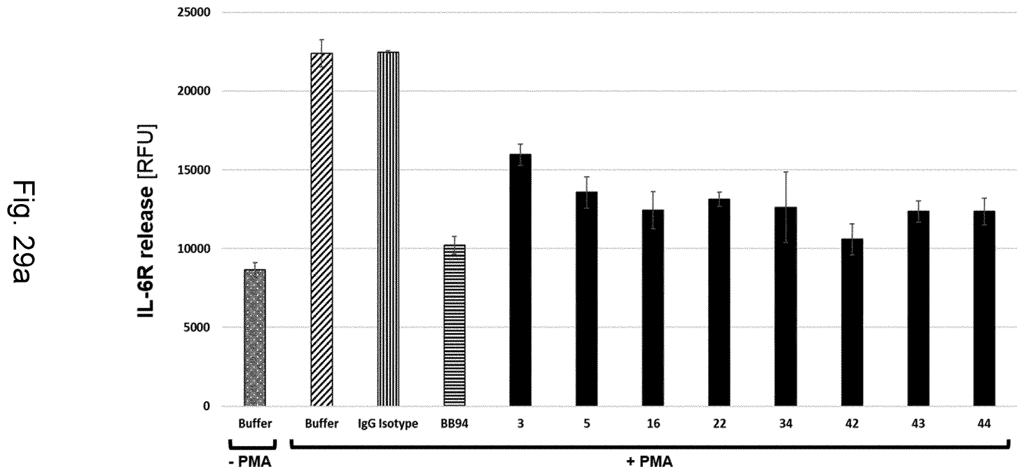

FIG. 30 shows representative results of this experiment demonstrating the effects of test articles on PMA-induced release of IL-6R from U-937 cells in absolute numbers (FIG. 30a) and percent inhibition (FIG. 30b). While Batimastat (BB94) as a small molecule inhibitor of metalloproteinases serves as positive control and results in 86.6% inhibition of PMA-induced release of IL-6R, the presence of IgG isotype control has no significant effect on IL-6R shedding. In contrast, an equal concentration of the antibodies 3, 5, 16, 22, 34, 42, 43 and 44 of the invention inhibits PMA-induced release of IL-6R from U-937 cells by 61.8%, 67.0%, 77.7%, 74.5%, 69.3%, 80.8%, 76.1% and 71.8%, respectively.

### Example 28: Analysis of Inhibitory Effects of the Antibodies of the Invention on PMA-Induced IL-6R Shedding In Vitro

Complementary to Example 27 described above, ELISA-based IL-6R release assays were performed to verify the inhibitory effects of the antibodies of the invention on PMA-induced release of endogenous IL-6R from human U-937 cells. However, this analysis was conducted with both recombinant produced murine and recombinant produced chimeric antibodies of the invention.

The production of the recombinant antibody material that was used in this example is identical to the one described in Example 25. The ELISA-based IL-6R release assay that was used in this example is described below.

In brief, on day 1, Nunc black MaxiSorp® 96-well plates (Thermo Fisher Scientific, USA) were coated for 7 hours with 100 μl per well of mouse anti-human IL-6R capture antibody (provided as part of the DuoSet ELISA kit) at 2 μg/ml TBS at room temperature. Capture antibody solution was removed and MaxiSorp® plates were blocked with 300 μl per well of TBS, 1% BSA at room temperature for 1 hour. Meanwhile, 80,000 U-937 (European Collection of Authenticated Cell Cultures, UK) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse or human IgG antibody (Thermo Fisher Scientific, USA) at 15 μg/ml as isotype control (for a final concentration of 3 μg/ml in the resulting 100 μl sample volume) or antibodies of the invention at 15 μg/ml (for a final concentration of 3 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 375 ng/ml in growth medium for a final concentration of 62.5 ng/ml at 37° C., 5% CO₂ for 1 hour. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human IL-6R protein (provided as part of the DuoSet ELISA kit) diluted in TBS at defined concentrations were added to the plate as standard references. Thereafter, 100 μl biotinylated goat anti-human IL-6R detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

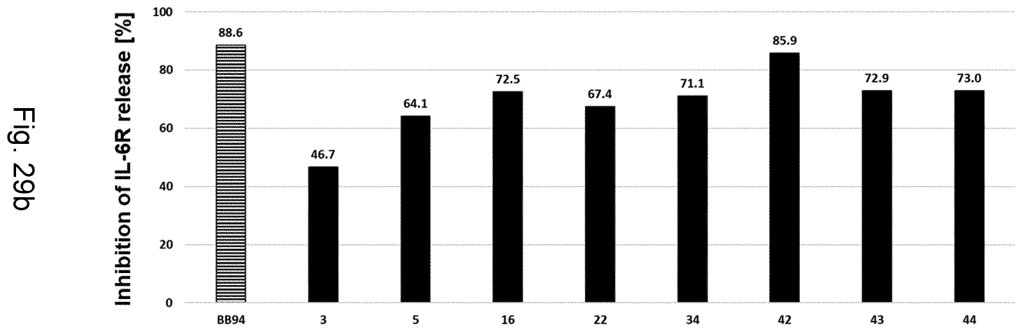

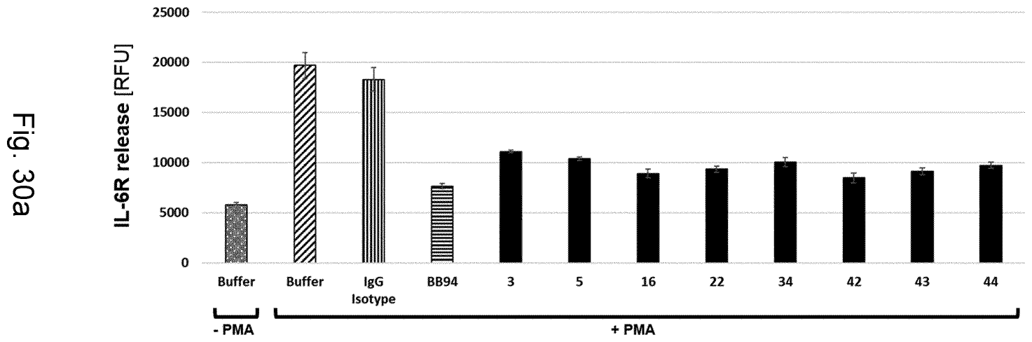

FIG. 31 shows representative results of this experiment demonstrating the effects of test articles on PMA-induced release of IL-6R from U-937 cells in absolute numbers (FIG. 31a) and percent inhibition (FIG. 31b). While Batimastat (BB94) as a small molecule inhibitor of metalloproteinases serves as positive control and results in 91.6% inhibition of PMA-induced release of IL-6R, the presence of mouse or human IgG isotype control has no significant effect on IL-6R shedding. In contrast, an equal concentration of the murine antibodies m16, m22, m34, m42 and m44 of the invention inhibits PMA-induced release of IL-6R from U-937 cells by 77.4%, 79.0%, 74.6%, 84.4% and 82.0%, respectively. Highly comparable to the results obtained with the murine antibodies of the invention, an equal concentration of the chimeric antibodies chl6, ch22, ch34, ch42 and ch44 of the invention inhibits PMA-induced release of IL-6R from U-937 cells by 84.3%, 85.6%, 82.8%, 91.8% and 85.2%, respectively.

### Example 29: Analysis of Inhibitory Effects of the Antibodies of the Invention on PMA-Induced Heparin-Binding EGF-Like Growth Factor (HB-EGF) Shedding In Vitro

In the following study, ELISA-based HB-EGF release assays were performed to analyze the inhibitory effects of the antibodies of the invention on PMA-induced release of endogenous HB-EGF from human THP-1 monocytic cells.

The production of the recombinant antibody material that was used in this example is identical to the one described in Example 25. The ELISA-based HB-EGF release assay that was used in this example is described below.

In brief, on day 1, Nunc black MaxiSorp® 96-well plates (Thermo Fisher Scientific, USA) were coated for 7 hours with 100 μl per well of rat anti-human HB-EGF capture antibody (provided as part of the DuoSet ELISA kit) at 2 μg/ml TBS at room temperature. Capture antibody solution was removed and MaxiSorp® plates were blocked with 300 μl per well of TBS, 1% BSA at room temperature for 3 hours. Meanwhile, 80,000 THP-1 (American Type Culture Collection, USA) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse or human IgG antibody (Thermo Fisher Scientific, USA) at 15 μg/ml as isotype control (for a final concentration of 3 μg/ml in the resulting 100 μl sample volume) or antibodies of the invention at 15 μg/ml (for a final concentration of 3 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 6 hours. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human HB-EGF protein (provided as part of the DuoSet ELISA kit) diluted in TBS at defined concentrations were added to the plate as standard references. Thereafter, 100 μl biotinylated goat anti-human HB-EGF detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes.

Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

FIG. 32 shows representative results of this experiment demonstrating the effects of test articles on PMA-induced release of HB-EGF from THP-1 cells in absolute numbers (FIG. 32a) and percent inhibition (FIG. 32b). While Batimastat (BB94) as a small molecule inhibitor of metalloproteinases serves as positive control and results in 98.9% inhibition of PMA-induced release of HB-EGF, the presence of mouse or human IgG isotype control has no significant effect on HB-EGF shedding. In contrast, an equal concentration of the murine antibodies m16, m22, m34, m42 and m44 of the invention inhibits PMA-induced release of HB-EGF from THP-1 cells by 71.9%, 77.7%, 64.2%, 76.6% and 67.5%, respectively. Highly comparable to the results obtained with the murine antibodies of the invention, an equal concentration of the chimeric antibodies chl6, ch22, ch34, ch42 and ch44 of the invention inhibits PMA-induced release of HB-EGF from THP-1 cells by 73.6%, 81.9%, 76.1%, 80.8% and 70.7%, respectively.

### Example 30: Analysis of Inhibitory Effects of the Antibodies of the Invention on PMA-Induced HB-EGF Shedding In Vitro

Complementary to Example 29 described above, ELISA-based HB-EGF release assays were performed to verify the inhibitory effects of the antibodies of the invention on PMA-induced release of endogenous HB-EGF from human U-937 cells.

The production of the recombinant antibody material that was used in this example is identical to the one described in Example 25. The ELISA-based HB-EGF release assay that was used in this example is identical with the one described in Example 30, with the only difference, that U-937 (European Collection of Authenticated Cell Cultures, UK) cells were used instead of THP-1 (American Type Culture Collection, USA) cells.

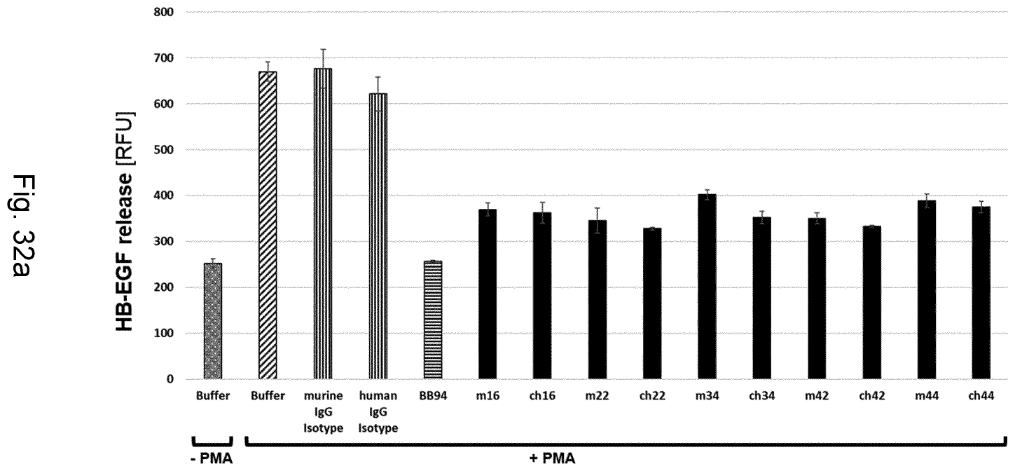

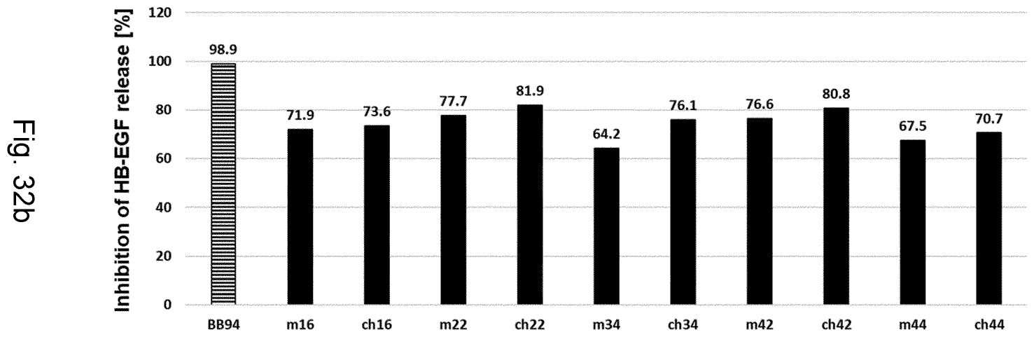

FIG. 33 shows representative results of this experiment demonstrating the effects of test articles on PMA-induced release of HB-EGF from U-937 cells in absolute numbers (FIG. 33a) and percent inhibition (FIG. 33b). While Batimastat (BB94) as a small molecule inhibitor of metalloproteinases serves as positive control and results in 100.1% inhibition of PMA-induced release of HB-EGF, the presence of mouse or human IgG isotype control has no significant effect on HB-EGF shedding. In contrast, an equal concentration of the murine antibodies m16, m22, m34, m42 and m44 of the invention inhibits PMA-induced release of HB-EGF from U-937 cells by 99.6%, 101.3%, 98.2%, 103.5% and 100.5%, respectively.

Highly comparable to the results obtained with the murine antibodies of the invention, an equal concentration of the chimeric antibodies chl6, ch22, ch34, ch42 and ch44 of the invention inhibits PMA-induced release of HB-EGF from U-937 cells by 100.8%, 103.2%, 98.1%, 103.0% and 99.2%, respectively.

### Example 31: Analysis of Inhibitory Effects of the Antibodies of the Invention on PMA-Induced Transforming Growth Factor Alpha (TGFα) Shedding In Vitro

In the following study, ELISA-based TGFα release assays were performed to analyze the inhibitory effects of the antibodies of the invention on PMA-induced release of endogenous TGFα from human PC3 prostate cancer cells.

The production of the recombinant antibody material that was used in this example is identical to the one described in Example 17. The ELISA-based TGFα release assay that was used in this example is described below.

In brief, on day 1, Nunc black MaxiSorp® 96-well plates (Thermo Fisher Scientific, USA) were coated overnight with 100 μl per well of goat anti-human TGFα capture antibody (provided as part of the DuoSet ELISA kit) at 0.4 μg/ml TBS at 4° C. On day 2, the capture antibody solution was removed and MaxiSorp® plates were blocked with 300 μl per well of TBS, 1% BSA at room temperature for 3 hours. Meanwhile, 100,000 PC3 (European Collection of Authenticated Cell Cultures, UK) cells in 80 μl of normal growth medium were seeded in each well of F-bottom 96-well cell culture plates (Coming, USA) and pre-incubated with 20 μl per well of OptiMEM medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of OptiMEM medium without test articles were added. Subsequently, cells (except those for unstimulated controls) were stimulated with 20 μl per well of PMA (Sigma-Aldrich, USA) at 150 ng/ml in OptiMEM for a final concentration of 25 ng/ml at 37° C., 5% CO₂ for 2 hours. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human TGFα protein (provided as part of the DuoSet ELISA kit) diluted in TBS at defined concentrations were added to the plate as standard references. Thereafter, 100 μl biotinylated goat anti-human TGFα detection antibody (provided as part of the DuoSet ELISA kit) at 37.5 ng/ml in TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

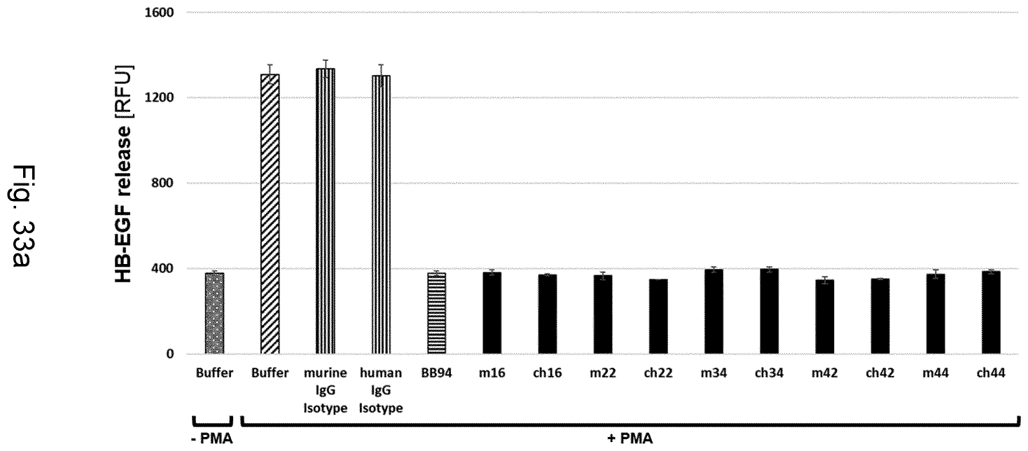

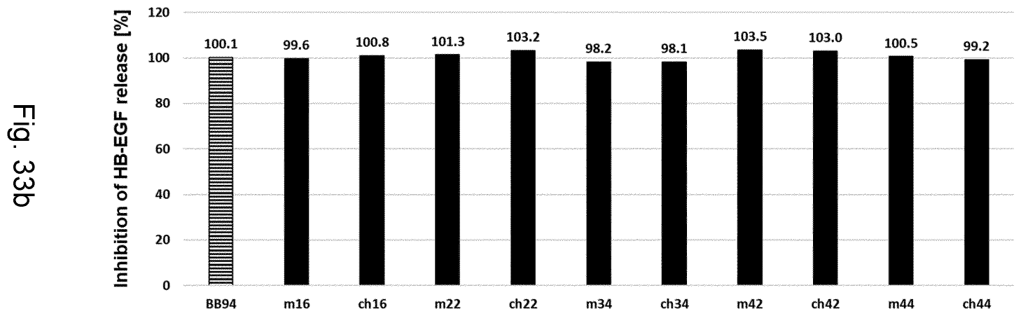

FIG. 34 shows representative results of this experiment demonstrating the effects of test articles on PMA-induced release of TGFα from PC3 cells in absolute numbers (FIG. 34a) and percent inhibition (FIG. 34b). While Batimastat (BB94) as a small molecule inhibitor of metalloproteinases serves as positive control and results in 99.1% inhibition of PMA-induced release of TGFα, the presence of IgG isotype control has no significant effect on TGFα shedding. Likewise, no significant effect on TGFα shedding was detected in the presence of equal concentrations of the antibodies 3, 5, and 34 of the invention. Moreover, only a very moderate effect on TGFα shedding was detected in the presence of equal concentrations of the antibodies 16, 22, 42, 43 and 44 of the invention which inhibit PMA-induced release of TGFα from PC3 cells by 13.9%, 12.7%, 14.3%, 12.4% and 14.1%, respectively.

### Example 32: Analysis of Inhibitory Effects of the Antibodies of the Invention on LPS-Induced TNFα Shedding in Primary Human Material from Healthy Donors In Vitro

In the following study, ELISA-based TNFα release assays were performed to analyze the inhibitory effects of the antibodies of the invention on LPS-induced release of endogenous TNFα from primary human material obtained from healthy donors using peripheral blood mononuclear cells (PBMCs).

The production of the recombinant antibody material that was used in this example is identical to the one described in Example 17. The ELISA-based TNFα release assay that was used in this example is described below.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 20,000 PBMC from healthy donors (STEMCELL Technologies, Canada) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of LPS (Sigma-Aldrich, USA) at 300 ng/ml in growth medium for a final concentration of 50 ng/ml at 37° C., 5% CO₂ for 2 hours. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human TNFα protein (provided as part of the DuoSet ELISA kit) diluted in TBS at defined concentrations were added to the plate as standard references. Thereafter, 100 μl biotinylated goat anti-human TNFα detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

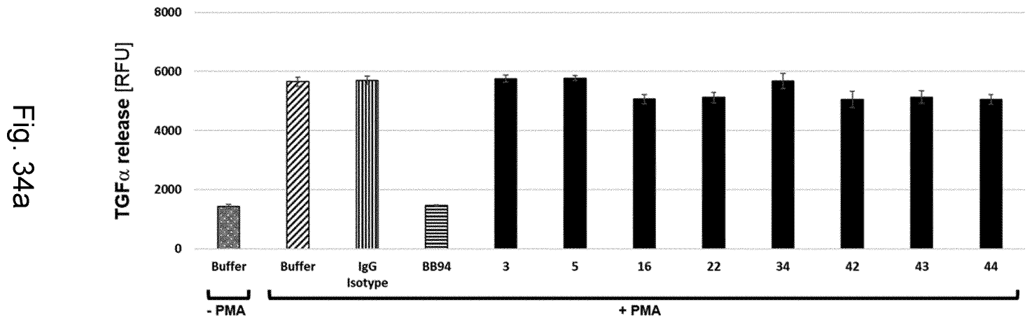

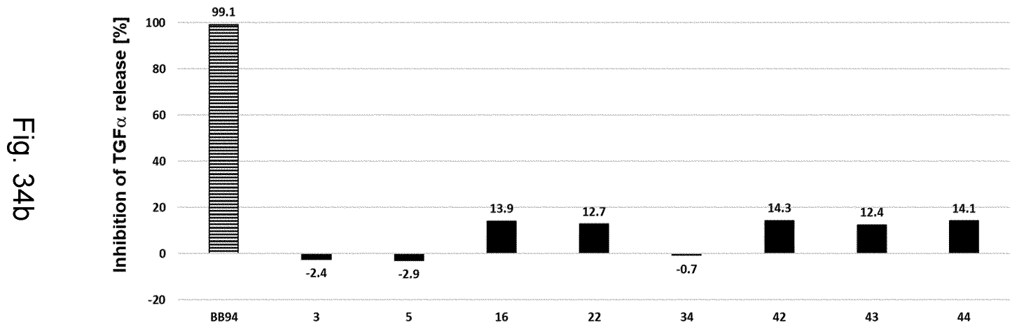

FIG. 35 shows representative results of this experiment demonstrating the effects of test articles on LPS-induced release of TNFα from PBMCs from healthy donors in absolute numbers (FIG. 35a) and percent inhibition (FIG. 35b). While Batimastat (BB94) as a small molecule inhibitor of metalloproteinases serves as positive control and results in 99.6% inhibition of LPS-induced release of TNFα, the presence of IgG isotype control has no significant effect on TNFα shedding. In contrast, an equal concentration of the antibodies 16, 22 and 42 of the invention inhibits LPS-induced release of TNFα from PBMCs from healthy donors by 81.3%, 72.8% and 77.0%, respectively.

### Example 33: Analysis of Inhibitory Effects of the Antibodies of the Invention on LPS-Induced TNFα Shedding in Primary Human Material from Healthy Donors In Vitro

Complementary to Example 32 described above, ELISA-based TNFα release assays were performed to test the inhibitory effects of the antibodies on LPS-induced release of endogenous TNFα from human macrophages from healthy donors.

The production of the recombinant antibody material that was used in this example is identical to the one described in Example 17. The isolation of macrophages and the ELISA-based TNFα release assay that was used in this example are described below.

To obtain human macrophages, peripheral blood mononuclear cells (PBMCs) from 4 healthy donors were used. Cells from 5 ml human blood were resuspended in 20 ml of DMEM medium (Corning, USA) supplemented with 10% FCS (Atlanta Biological, USA) and Penicillin/Streptomycin (Corning, USA) (DMEM/FCS) and were then incubated on two 10 cm petri dishes (Thermo Fisher, USA) at 37° C., 5% CO₂ for 3h. Non-adherent cells were subsequently removed and 6 ml of DMEM/FCS supplemented with 10 ng/ml human MCSF (Peprotech, USA) (DMEM/FCS/MCSF) was added per plate. Both plates were incubated at 37° C., 5% CO₂ for 2 days, then 2 ml of DMEM/FCS/MCSF was added to each plate on day 2 and 4. On day 5, all medium was removed and attached cells were washed once with 5 ml PBS (Corning, USA). 2 ml of Accutase (Promocell, USA) solution was added to each plate and incubated at 37° C. for 15 min. 10 ml of DMEM/FCS was added to each plate and detached cells were collected in a 15 ml Falcon tube. Cells were subsequently pelleted in a tabletop centrifuge (Eppendorf, USA) at 1,500 rpm for 5 min. The medium was removed and the cells were resuspended in 2 ml of DMEM/FCS. 10 μl from this cell suspension was manually counted in a hemocytometer (Thermo Fisher, USA). The cell number was adjusted to 2.5×10⁵ cells/ml and 200 μl of the suspension was plated in one well of a 48-well tissue culture plate (Corning, USA) resulting in a cell density of 0.5×10⁵ cells per 48-well. Cells were then incubated for 16 h at 37° C. For measuring TNFα released from PBMC cells, a OptEIA human TNFα ELISA (BD, USA) was used. Briefly, on day 1, Costar® 96-well plates (Corning, USA) were coated overnight with 100 μl per well of anti-human TNFα capture antibody (provided as part of the OptEIA® ELISA kit) at 1:250 in PBS at 4° C. On day 2, the capture antibody solution was removed, Costar® plates were washed 4 times with 350 μl PBS-T (Boston Bio, USA) per well with a Nunc Immunowash plate washer (VWR, USA) and plates were then blocked with 300 μl per well of PBS, 10% FCS at room temperature for 2 hours.

Meanwhile, on day 2, human macrophages were pre-incubated with 200 μl per well of DMEM/FCS supplemented with Batimastat (BB94, Abeam, MA, USA) at 20 μM as positive control (for a final concentration of 10 μM in the resulting 400 μl sample volume), or purified antibodies of the invention at 20 μg/ml (for a final concentration of 10 μg/ml in the resulting 400 μl sample volume) at 37° C., 5% CO₂ for 30 minutes. In case of unstimulated controls, 200 μl of standard growth medium without test articles were added. Subsequently, cells (except those for unstimulated controls) were stimulated with 200 μl per well of LPS (Sigma-Aldrich, USA) at 10 ng/ml in growth medium for a final concentration of 5 ng/ml at 37° C., 5% CO₂ for 2 hours. After 2 h, supernatants were removed and debris was spun out at 13,000 rpm at 4° C. in a tabletop centrifuge (Eppendorf, USA) and clarified supernatants were diluted 1:10 with PBS-T (Boston Bio, USA). Diluted supernatants were kept on ice until added to ELISA plates. In parallel, blocking buffer was removed from the Costar® plates and plates were washed 4 times with 350 μl PBS-T (Boston Bio, USA) per well with a Nunc Immunowash plate washer (VWR, USA). Immediately after, 100 μl clear, diluted supernatant was added to wells. Additionally, 100 μl recombinant human TNFα protein (provided as part of the OptEIA ELISA kit) diluted in PBS-BSA at defined concentrations were added to the plate as standard references. Samples and standards were incubated for 2h at room temperature. Thereafter, plates were washed 4 times with 350 μl PBS-T (Boston Bio, USA) per well with a Nunc Immunowash plate washer (VWR, USA). Then, 100 μl biotinylated anti-human TNFα detection antibody (provided as part of OptEIA ELISA kit) at a dilution of 1:250 in PBS-FCS was added per well and plates were incubated at room temperature for 2 hours. After 4 times washing with 350 μl PBS-T (Boston Bio, USA) per well with a Nunc Immunowash plate washer (VWR, USA) and careful removal of all buffer traces after the fourth cycle, 100 μl streptavidin-horseradish peroxidase conjugate (provided as part of OptEIA ELISA kit) diluted 1:40 in PBS-FCS were added to each well and plates were incubated at room temperature for 20 minutes. Following another round of 4 washes with 350 μl PBS-T (Boston Bio, USA) per well with a Nunc Immunowash plate washer (VWR, USA) and careful removal of all buffer traces after the fourth cycle, 100 μl TMB substrate solution (BD, USA) per well was added for incubation for 20 minutes. The color reaction was stopped by the addition of 50 μl 2N sulfuric acid (Boston Bio, USA) and the ELISA plate was read at the wavelength of 450 nm using a Multiskan Titertek Plate reader (VWR, USA).

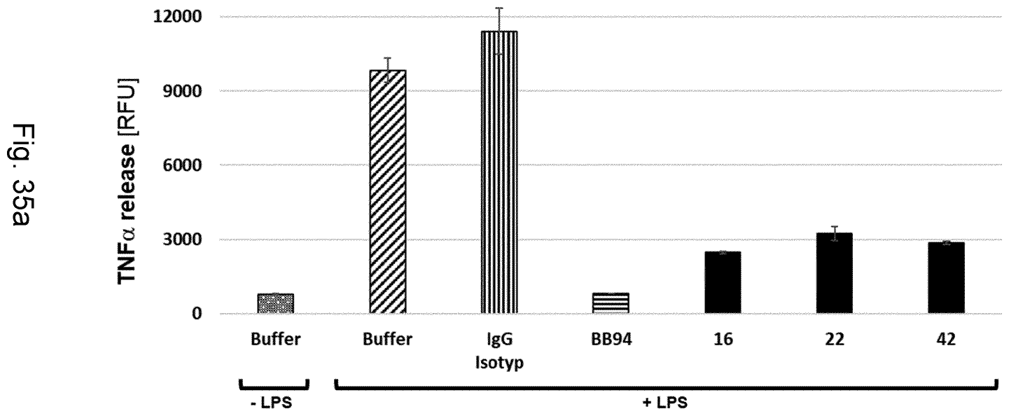

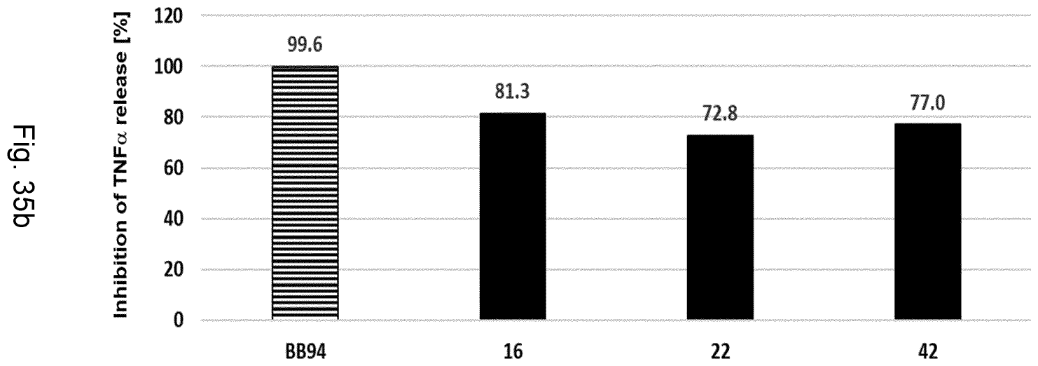

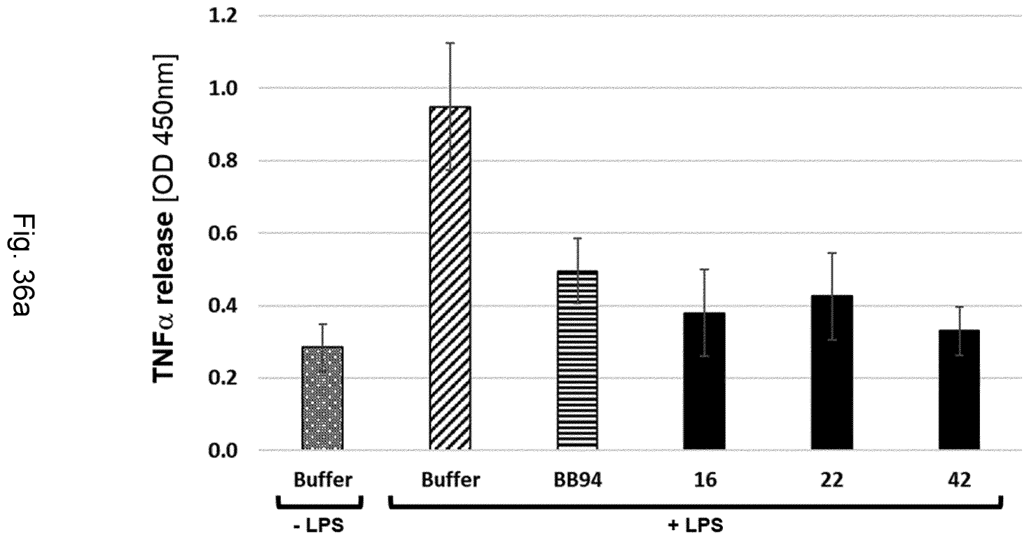

FIG. 36 shows representative results of this experiment demonstrating the effects of test articles on LPS-induced release of TNFα from human macrophages from healthy donors in absolute numbers (FIG. 36a) and percent inhibition (FIG. 36b). Batimastat (BB94) as a small molecule inhibitor of metalloproteinases serves as positive control and results in 68.3% inhibition of LPS-induced release of TNFα. Equal concentrations of the antibodies 16, 22 and 42 of the invention inhibit LPS-induced release of TNFα from human macrophages from healthy donors by 85.8%, 78.8% and 93.2%, respectively.

### Example 34: Analysis of Inhibitory Effects of the Antibodies of the Invention on PMA-Induced IL-6R Shedding in Primary Human Material from Healthy Donors In Vitro

In the following study, ELISA-based IL-6R release assays were performed to analyze the inhibitory effects of the antibodies of the invention on PMA-induced release of endogenous IL-6R from primary human material obtained from healthy donors using peripheral blood mononuclear cells (PBMCs).

The production of the recombinant antibody material that was used in this example is identical to the one described in Example 17. The ELISA-based IL-6R release assay that was used in this example is described below.

In brief, on day 1, Nunc black MaxiSorp® 96-well plates (Thermo Fisher Scientific, USA) were coated overnight with 100 μl per well of mouse anti-human IL-6R capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 40,000 PBMC from healthy donors (STEMCELL Technologies, Canada) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 6 hours. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human IL-6R protein (provided as part of the DuoSet ELISA kit) diluted in TBS at defined concentrations were added to the plate as standard references. Thereafter, 100 μl biotinylated goat anti-human IL-6R detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

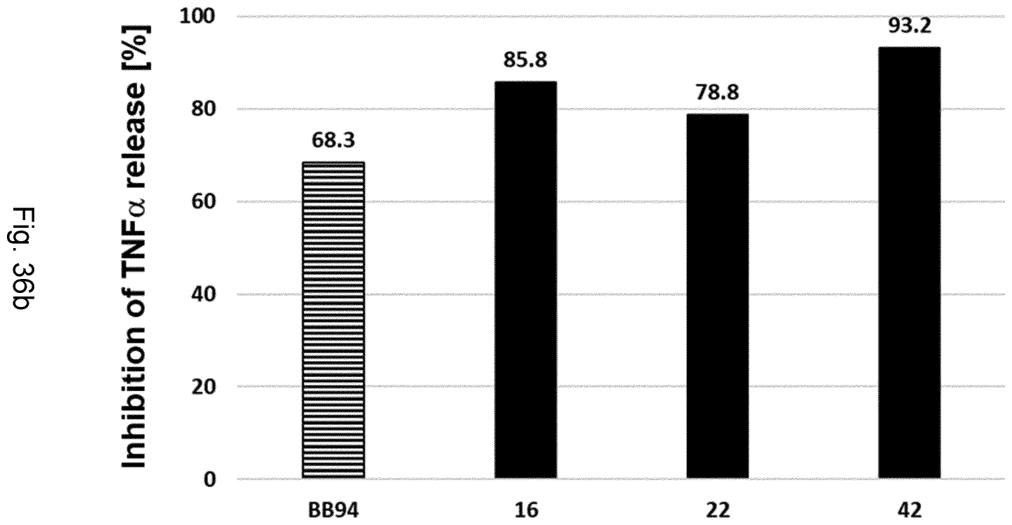

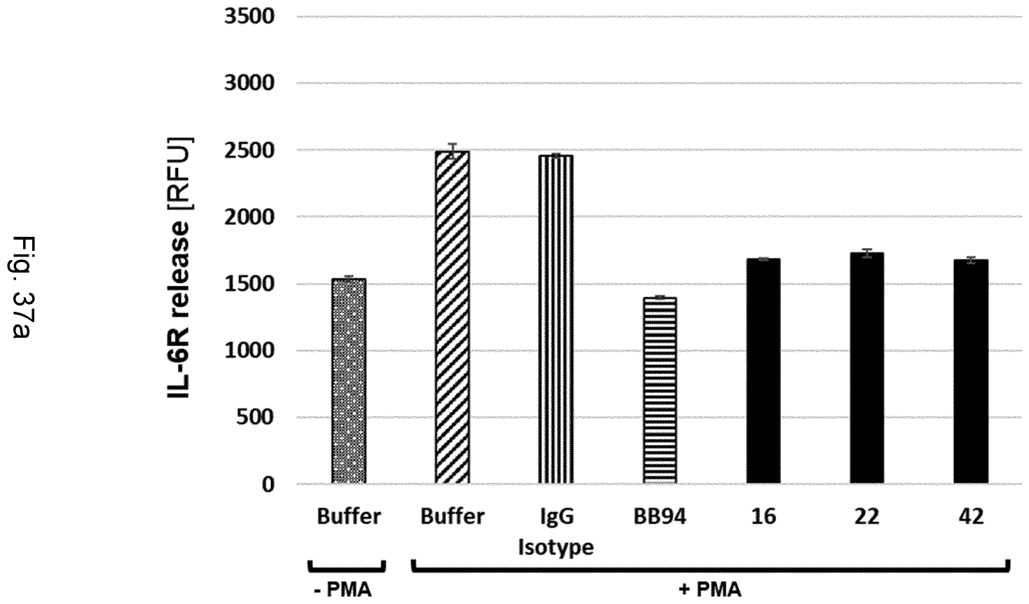

FIG. 37 shows representative results of this experiment demonstrating the effects of test articles on PMA-induced release of IL-6R from PBMCs from healthy donors in absolute numbers (FIG. 37a) and percent inhibition (FIG. 37b). While Batimastat (BB94) as a small molecule inhibitor of metalloproteinases serves as positive control and results in 114.1% inhibition of PMA-induced release of IL-6R, the presence of IgG isotype control has no significant effect on IL-6R shedding. In contrast, an equal concentration of the antibodies 16, 22 and 42 of the invention inhibits PMA-induced release of IL-6R from PBMCs from healthy donors by 84.3%, 79.3% and 85.0%, respectively.

### Example 35: Analysis of Inhibitory Effects of the Antibodies of the Invention on PMA-Induced HB-EGF Shedding in Primary Human Material from Healthy Donors In Vitro

In the following study, ELISA-based HB-EGF release assays were performed to analyze the inhibitory effects of the antibodies of the invention on PMA-induced release of endogenous HB-EGF from primary human material obtained from healthy donors using peripheral blood mononuclear cells (PBMCs).

The production of the recombinant antibody material that was used in this example is identical to the one described in Example 17. The ELISA-based HB-EGF release assay that was used in this example is described below.

In brief, on day 1, Nunc black MaxiSorp® 96-well plates (Thermo Fisher Scientific, USA) were coated overnight with 100 μl per well of mouse anti-human HB-EGF capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 80,000 PBMC from healthy donors (STEMCELL Technologies, Canada) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 6 hours. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human HB-EGF protein (provided as part of the DuoSet ELISA kit) diluted in TBS at defined concentrations were added to the plate as standard references. Thereafter, 100 μl biotinylated goat anti-human HB-EGF detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

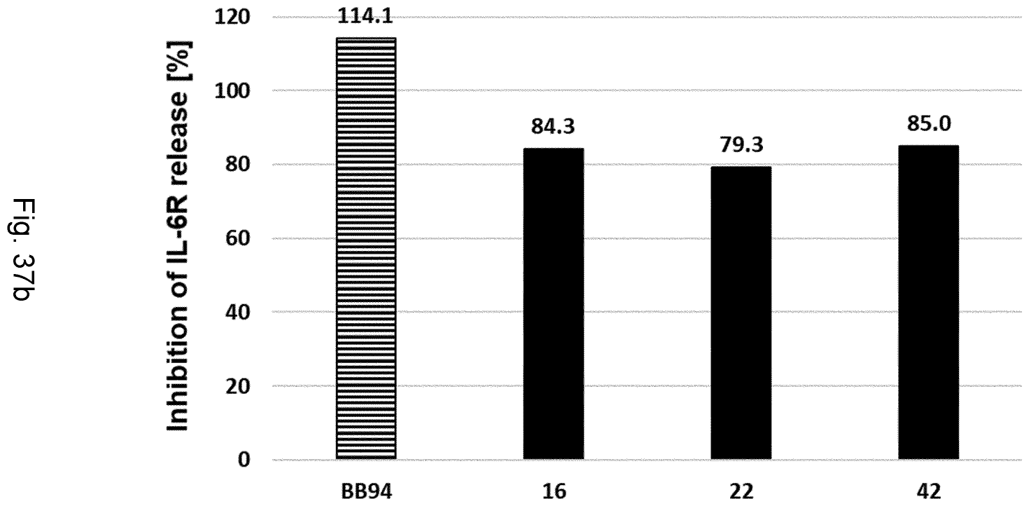

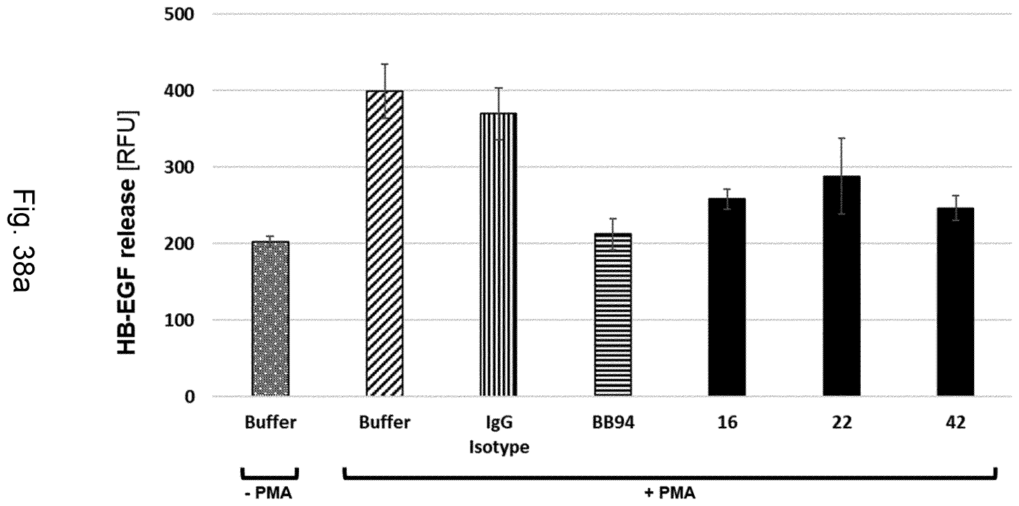

FIG. 38 shows representative results of this experiment demonstrating the effects of test articles on PMA-induced release of HB-EGF from PBMCs from healthy donors in absolute numbers (FIG. 38a) and percent inhibition (FIG. 38b). While Batimastat (BB94) as a small molecule inhibitor of metalloproteinases serves as positive control and results in 94.9% inhibition of PMA-induced release of HB-EGF, the presence of IgG isotype control has no significant effect on HB-EGF shedding. In contrast, an equal concentration of the antibodies 16, 22 and 42 of the invention inhibits PMA-induced release of HB-EGF from PBMCs from healthy donors by 71.6%, 56.5% and 77.6%, respectively.

### Example 36: Analysis of Inhibitory Effects of the Antibodies of the Invention on LPS-Induced TNFα Shedding In Vivo

In the following study, ELISA-based TNFα release assays were performed to verify the inhibitory effects of the antibodies of the invention on LPS-induced release of endogenous TNFα in a mouse model for septic shock. Humanized huNOG-EXL mice (human CD34+) were obtained from Taconic, USA. Upon arrival, mice were housed for a minimum of 12 h to acclimatize before any treatments were initiated. All mouse experiments were approved by and were in compliance with the institutional animal care and use committee (IACUC) regulations of HSS/Weill Cornell Medicine.

On day 1, one group of mice was injected with the antibodies of the invention at a concentration of 500 μg/200 μl PBS per mouse (25 mg/kg). A second group was injected with the same volume of PBS only (200 μl PBS per mouse). 12h later all mice were subjected to an injection of LPS (Sigma, USA) at a concentration of 500 ng/200 μl per mouse. All mice were closely monitored and euthanized after 2h by CO₂ inhalation. Blood was removed from the chest cavity and was centrifuged at 2000 g for 10 min at room temperature to remove cells and debris. Clear serum was transferred to a new tube and subsequently diluted 1:100 in PBS for ELISA measurements.

For measuring TNFα release, an OptEIA human TNFα ELISA (BD, USA) was used. Briefly, on day 1, Costar® 96-well plates (Coming, USA) were coated overnight with 100 μl per well of anti-human TNFα capture antibody (provided as part of the OptEIA® ELISA kit) at 1:250 in PBS at 4° C. On day 2, the capture antibody solution was removed, Costar® plates were washed 4 times with 350 μl PBS-T (Boston Bio, USA) per well with a Nunc Immunowash plate washer (VWR, USA) and plates were blocked with 300 μl per well of PBS, 10% FCS at room temperature for 2 hours. Then, blocking buffer was removed from the Costar® plates and plates were washed 4 times with 350 μl PBS-T (Boston Bio, USA) per well with a Nunc Immunowash plate washer (VWR, USA). Immediately after, 100 μl clear, diluted serum was added to wells. Additionally, 100 μl recombinant human TNFα protein (provided as part of the OptEIA ELISA kit) diluted in PBS-BSA at defined concentrations were added to the plate as standard references. Samples and standards were incubated for 2h at room temperature.

Thereafter, plates were washed 4 times with 350 μl PBS-T (Boston Bio, USA) per well with a Nunc Immunowash plate washer (VWR, USA). Then, 100 μl biotinylated anti-human TNFα detection antibody (provided as part of OptEIA ELISA kit) at a dilution of 1:250 in PBS-FCS were added per well and plates were incubated at room temperature for 2 hours. After 4 times washing with 350 μl PBS-T (Boston Bio, USA) per well with a Nunc Immunowash plate washer (VWR, USA) and careful removal of all buffer traces after the fourth cycle, 100 μl streptavidin-horseradish peroxidase conjugate (provided as part of OptEIA ELISA kit) diluted 1:40 in PBS-FCS were added to each well and plates were incubated at room temperature for 20 minutes. Following another round of 4 washes with 350 μl PBS-T (Boston Bio, USA) per well with a Nunc Immunowash plate washer (VWR, USA) and careful removal of all buffer traces after the fourth cycle, 100 μl TMB substrate solution (BD, USA) per well was added for incubation for 20 minutes. The color reaction was stopped by the addition of 50 μl 2N sulfuric acid (Boston Bio, USA) and the ELISA plate was read at the wavelength of 450 nm using a Multiskan Titertek Plate reader (VWR, USA).

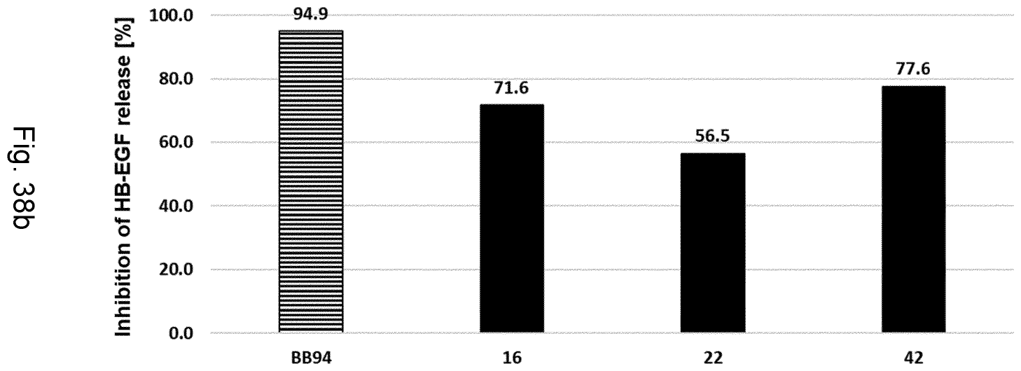

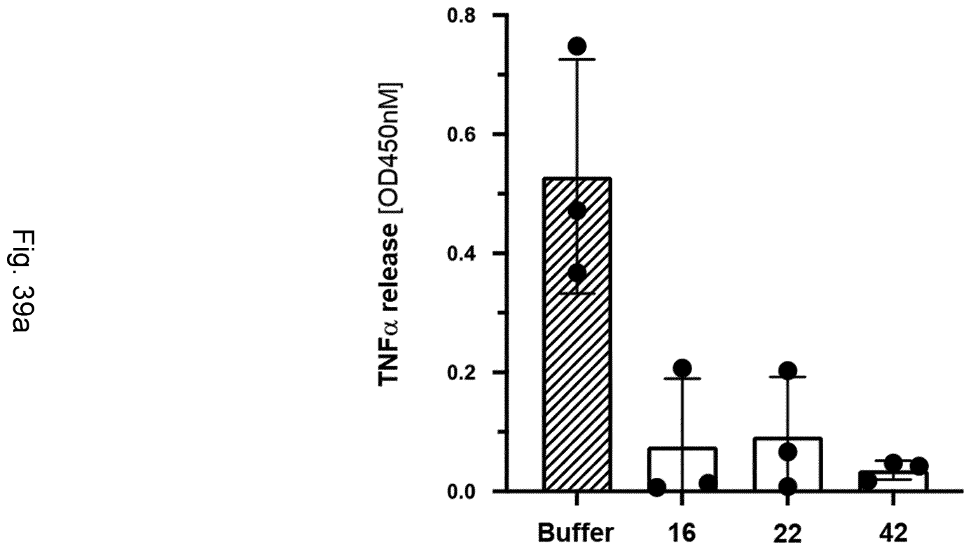

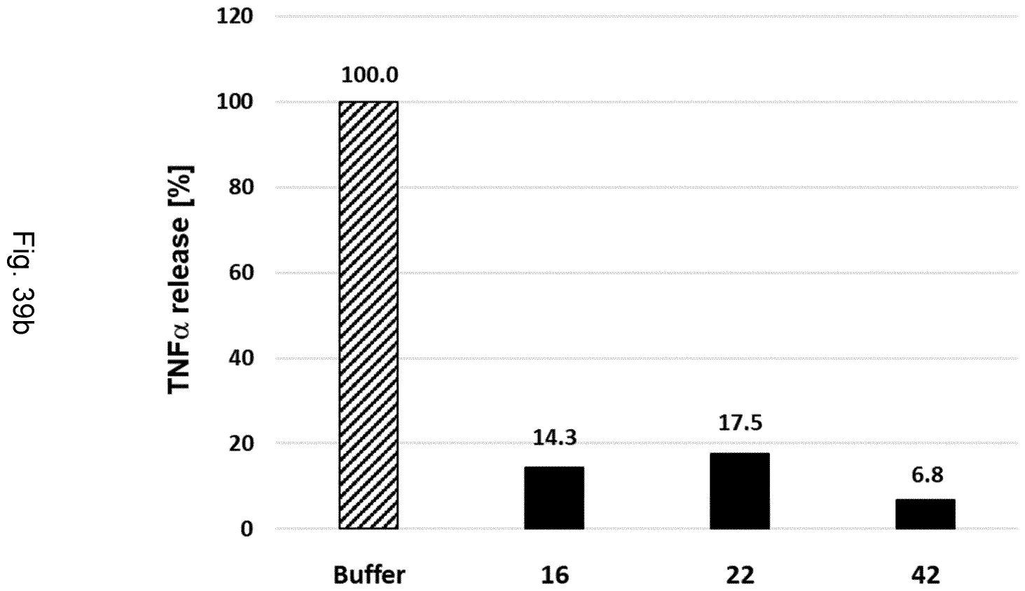

FIG. 39 shows representative results of this experiment demonstrating the effects of test articles on LPS-induced release of TNFα in serum of humanized mice in absolute numbers (FIG. 39a) and percent release (FIG. 39b). Compared to the LPS-induced release of TNFα in serum of humanized mice, which was set to 100%, the antibodies 16, 22 and 42 of the invention lead to an LPS-induced release of TNFα in serum of humanized mice of 14.3%, 17.5% and 6.8%, respectively.

### Example 37: Analysis of Inhibitory Effects of the Antibodies of the Invention on LPS-Induced TNFα Shedding in Primary Human Material from RA-Patients In Vitro

In contrast to Example 32, where the inhibitory effects of the antibodies of the invention on LPS-induced release of endogenous TNFα from primary human material obtained from healthy donors using peripheral blood mononuclear cells (PBMCs) were tested, this analysis was conducted to analyze the inhibitory effects of the antibodies of the invention on LPS-induced release of endogenous TNFα from primary human material obtained from patients suffering from rheumatoid arthritis (RA-patients).

To produce the recombinant chimeric antibody material, target DNA sequence was designed, optimized and synthesized. The complete sequence was sub-cloned into into pTT5 vector (Thermo Fisher Scientific, USA) and the transfection grade plasmid was maxi-prepared for CHO-3E7 or HD CHO-S(Thermo Fisher Scientific, USA) cell expression. CHO cells were grown in serum-free FreeStyle™ CHO Expression Medium (Thermo Fisher Scientific, USA) in Erlenmeyer flasks (Coming Inc., USA) at 37° C. with 5-8% CO₂ on an orbital shaker (VWR Scientific, Germany). One day before transfection, the cells were seeded at an appropriate density in new Erlenmeyer flasks. On the day of transfection, DNA and transfection reagent were mixed at an optimal ratio and then added into the flask with cells ready for transfection. The recombinant plasmids encoding target protein were transiently transfected into suspension CHO cell cultures. The cell culture supernatant collected on day 6 post-transfection was used for purification. Cell culture broth was centrifuged and filtrated. Filtered cell culture supernatant was loaded onto MabSelect SuRe™ LX (GE Healthcare, UK) affinity purification columns at an appropriate flowrate. After washing and elution with appropriate buffers, the eluted fractions were pooled and buffer exchanged to final formulation buffer. The purified protein was analyzed by SDS-PAGE analysis for molecular weight and purity measurements. Finally, the concentration was determined applying a NanoDrop 2000 spectrophotometer (Thermo Fisher Scientific, USA).

The ELISA-based TNFα release assay that was used in this example is described below.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 20,000 PBMC from patients suffering from rheumatoid arthritis (STEMCELL Technologies, Canada) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), human IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of LPS (Sigma-Aldrich, USA) at 6 ng/ml in growth medium for a final concentration of 1 ng/ml at 37° C., 5% CO₂ for 1.5 hours. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human TNFα protein (provided as part of the DuoSet ELISA kit) diluted in TBS at defined concentrations were added to the plate as standard references. Thereafter, 100 μl biotinylated goat anti-human TNFα detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

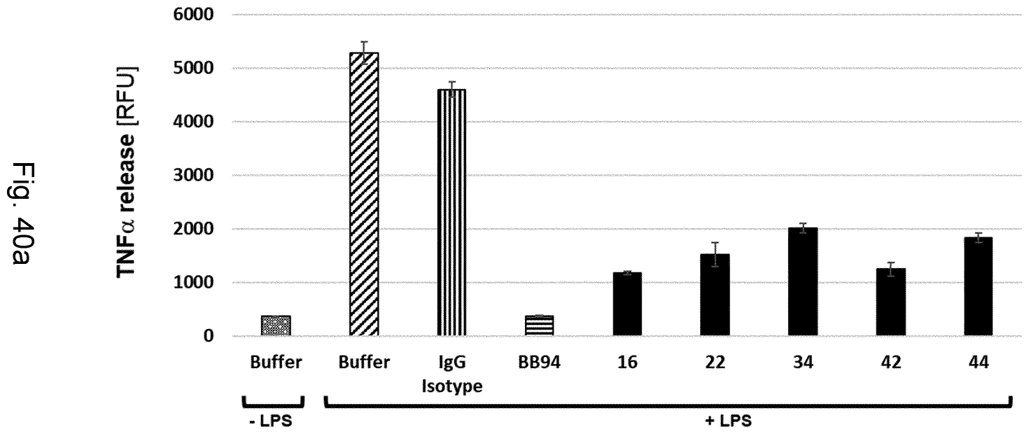

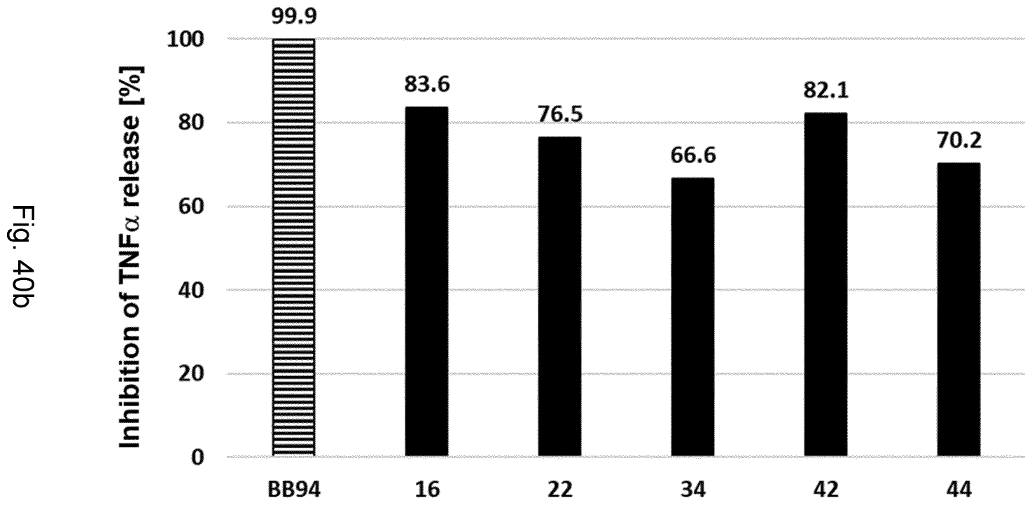

FIG. 40 shows representative results of this experiment demonstrating the effects of test articles on LPS-induced release of TNFα from PBMCs from patients suffering from rheumatoid arthritis in absolute numbers (FIG. 40a) and percent inhibition (FIG. 40b). While Batimastat (BB94) as a small molecule inhibitor of metalloproteinases serves as positive control and results in 99.9% inhibition of LPS-induced release of TNFα, the presence of IgG isotype control has no significant effect on TNFα shedding. In contrast, an equal concentration of the antibodies 16, 22, 34, 42 and 44 of the invention inhibits LPS-induced release of TNFα from PBMCs from patients suffering from rheumatoid arthritis by 83.6%, 76.5%, 66.6%, 82.1% and 70.2%, respectively.

### Example 38: Analysis of Inhibitory Effects of the Antibodies of the Invention on PMA-Induced IL-6R Shedding in Primary Human Material from RA-Patients In Vitro

In contrast to Example 34, where the inhibitory effects of the antibodies of the invention on PMA-induced release of endogenous IL-6R from primary human material obtained from healthy donors using peripheral blood mononuclear cells (PBMCs) were tested, this analysis was conducted to analyze the inhibitory effects of the antibodies of the invention on PMA-induced release of endogenous IL-6R from primary human material obtained from patients suffering from rheumatoid arthritis (RA-patients).

The production of the recombinant antibody material that was used in this example is identical to the one described in Example 37. The ELISA-based IL-6R release assay that was used in this example is described below.

In brief, on day 1, Nunc black MaxiSorp® 96-well plates (Thermo Fisher Scientific, USA) were coated overnight with 100 μl per well of mouse anti-human IL-6R capture antibody (provided as part of the DuoSet ELISA kit) at 4 μg/ml TBS at 4° C. Meanwhile, 40,000 PBMC from patients suffering from rheumatoid arthritis (STEMCELL Technologies, Canada) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), mouse IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 24 hours. On day 2, the capture antibody solution was removed and MaxiSorp® plates were blocked with 300 μl per well of TBS, 1% BSA at room temperature for 1 hour. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human IL-6R protein (provided as part of the DuoSet ELISA kit) diluted in TBS at defined concentrations were added to the plate as standard references. Thereafter, 100 μl biotinylated goat anti-human IL-6R detection antibody (provided as part of the DuoSet ELISA kit) at 100 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

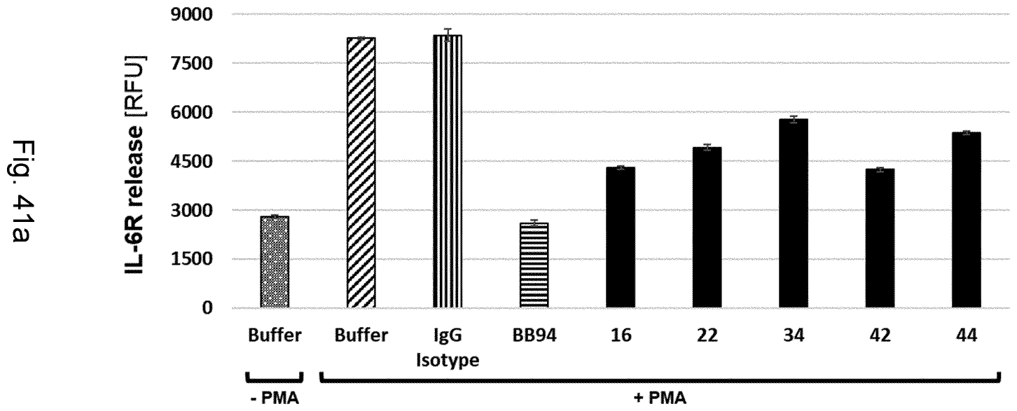

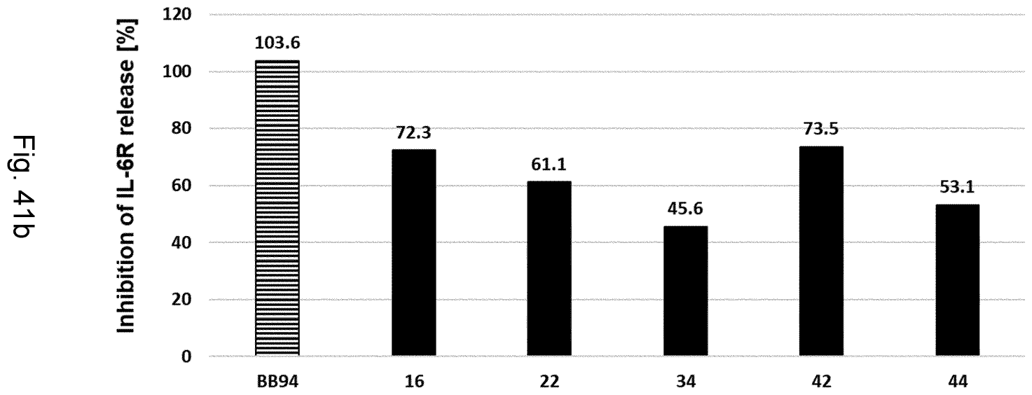

FIG. 41 shows representative results of this experiment demonstrating the effects of test articles on PMA-induced release of IL-6R from PBMCs from patients suffering from rheumatoid arthritis in absolute numbers (FIG. 41a) and percent inhibition (FIG. 41b). While Batimastat (BB94) as a small molecule inhibitor of metalloproteinases serves as positive control and results in 103.6% inhibition of PMA-induced release of IL-6R, the presence of IgG isotype control has no significant effect on IL-6R shedding. In contrast, an equal concentration of the antibodies 16, 22, 34, 42 and 44 of the invention inhibits PMA-induced release of IL-6R from PBMCs from patients suffering from rheumatoid arthritis by 72.3%, 61.1%, 45.6%, 73.5% and 53.1%, respectively.

### Example 39: Analysis of Inhibitory Effects of the Antibodies of the Invention on PMA-Induced HB-EGF Shedding in Primary Human Material from RA-Patients In Vitro

In contrast to Example 35, where the inhibitory effects of the antibodies of the invention on PMA-induced release of endogenous HB-EGF from primary human material obtained from healthy donors using peripheral blood mononuclear cells (PBMCs) were tested, this analysis was conducted to analyze the inhibitory effects of the antibodies of the invention on PMA-induced release of endogenous HB-EGF from primary human material obtained from patients suffering from rheumatoid arthritis (RA-patients).

The production of the recombinant antibody material that was used in this example is identical to the one described in Example 37. The ELISA-based HB-EGF release assay that was used in this example is described below.

In brief, on day 1, Nunc black MaxiSorp® 96-well plates (Thermo Fisher Scientific, USA) were coated overnight with 100 μl per well of rat anti-human HB-EGF capture antibody (provided as part of the DuoSet ELISA kit) at 2 μg/ml TBS at 4° C. On day 2, the capture antibody solution was removed and MaxiSorp® plates were blocked with 300 μl per well of TBS, 1% BSA at room temperature for 3 hours. Meanwhile, 80,000 PBMC from patients suffering from rheumatoid arthritis (STEMCELL Technologies, Canada) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), human IgG antibody (Thermo Fisher Scientific, USA) at 5 μg/ml as isotype control (for a final concentration of 1 μg/ml in the resulting 100 μl sample volume) or antibodies of the invention at 5 μg/ml (for a final concentration of 1 μg/ml in the resulting 100 μl sample volume) at 37° C., 5% CO₂ for 30 minutes. In case of stimulation controls, 20 μl of standard growth medium without test articles were added. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 6 hours. Afterwards, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human HB-EGF protein (provided as part of the DuoSet ELISA kit) diluted in TBS at defined concentrations were added to the plate as standard references. Thereafter, 100 μl biotinylated goat anti-human HB-EGF detection antibody (provided as part of the DuoSet ELISA kit) at 50 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

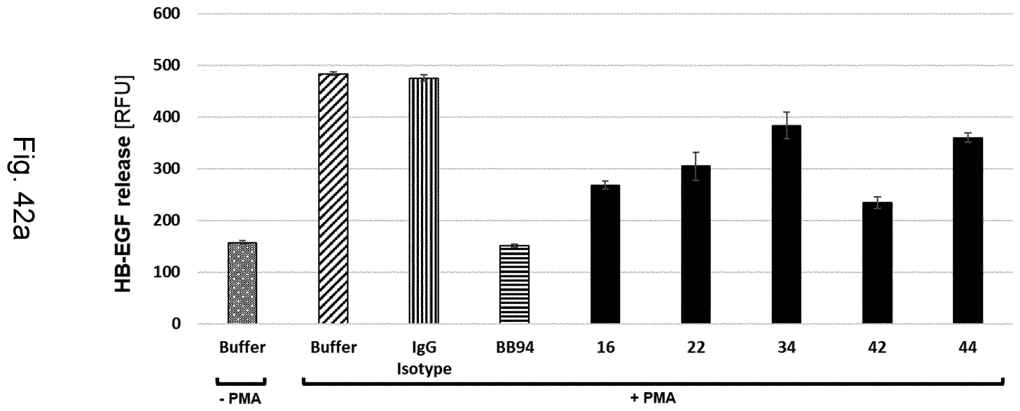

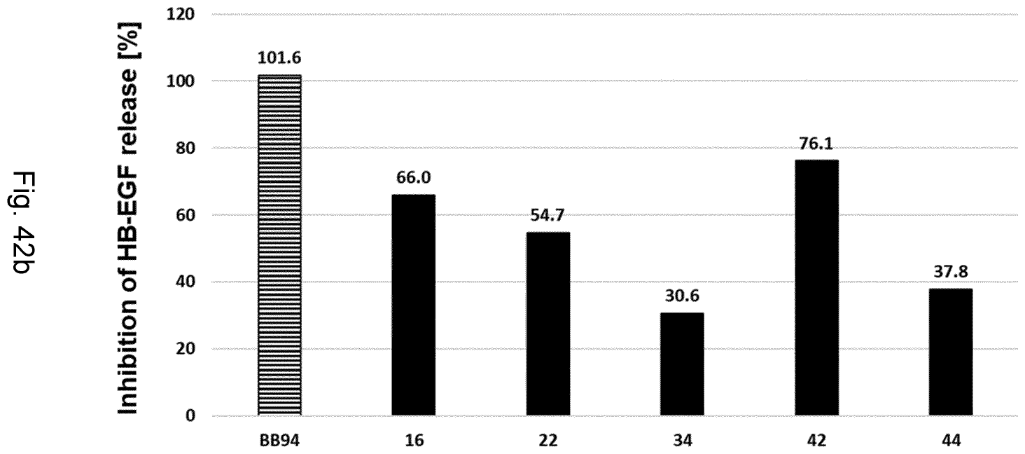

FIG. 42 shows representative results of this experiment demonstrating the effects of test articles on PMA-induced release of HB-EGF from PBMCs from patients suffering from rheumatoid arthritis in absolute numbers (FIG. 42a) and percent inhibition (FIG. 42b). While Batimastat (BB94) as a small molecule inhibitor of metalloproteinases serves as positive control and results in 101.6% inhibition of PMA-induced release of HB-EGF, the presence of IgG isotype control has no significant effect on HB-EGF shedding. In contrast, an equal concentration of the antibodies 16, 22, 34, 42 and 44 of the invention inhibits PMA-induced release of HB-EGF from PBMCs from patients suffering from rheumatoid arthritis by 66.0%, 54.7%, 30.6%, 76.1% and 37.8%, respectively.

### REFERENCES

Köhler, G. & Milstein, C. (1975): Continuous cultures of fused cells secreting antibody of predefined specificity. In: Nature. Bd. 256, S. 495-497. Jonsson and Malmquist, Advances in Biosnsors, 2:291-336 (1992)
Wu et al. Proc. Natl. Acad. Sci. USA, 95:6037-6042 (1998)
Banik, SSR; Doranz, BJ (2010). “Mapping complex antibody epitopes”. Genetic Engineering & Biotechnology News. 3 (2): 25-8
DeLisser, HM (1999). Epitope mapping. Methods Mol Biol. 96. pp. 11-20
Finco et al, Comparison of competitive ligand-binding assay and bioassay formats for the measurement of neutralizing antibodies to protein therapeutics. J Pharm Biomed Anal. 2011 Jan. 25; 54(2):351-8.
Deng et al., Enhancing antibody patent protection using epitope mapping information MAbs. 2018 Feb-Mar; 10(2): 204-209
Huston et al., Cell Biophysics, 22:189-224 (1993);
Pluckthun and Skerra, Meth. Enzymol., 178:497-515 (1989) and in Day, E. D., Advanced Immunochemistry, Second Ed., Wiley-Liss, Inc., New York, N.Y. (1990)
Harding, The immunogenicity of humanized and fully human antibodies. MAbs. 2010 May-Jun; 2(3): 256-265.
Eylenstein, et al, Molecular basis of in vitro affinity maturation and functional evolution of a neutralizing anti-human GM-CSF antibody, mAbs, 8:1, 176-186 (2016)
Kabat et al., J. Biol. Chem. 252, 6609-6616 (1977) and Kabat et al., Sequences of protein of immunological interest. (1991)
Chothia et al., J. Mol. Biol. 196:901-917 (1987)
MacCallum et al., J. Mol. Biol. 262:732-745 (1996)
Paul Baran et al, Biol Chem. 2013 May 24; 288(21): 14756-14768.

### SEQUENCES

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| | SEQ ID | | |
|---|---|---|---|
| clone | NO | qualifier | sequence |
| #3 | 1 | HC VD w signal | MGRLTSSFLLLIVPAYVLSQVTLKESGPGILQPSQTLSLTCSFSGFSLTTF |
| | | peptide | AMGIGWVRQPSGKGLEWLAHIWWDGEKYNNPVLKSRLTISKDTSKNQVFLR |
| | | | IANVDTPDTATYYCARISSIYYIIDNWGQGTSVTVSS |
| | 2 | HC VD w/o | QVTLKESGPGILQPSQTLSLTCSFSGFSLTTFAMGIGWVRQPSGKGLEWLA |
| | | signal peptide | HIWWDGEKYNNPVLKSRLTISKDTSKNQVFLRIANVDTPDTATYYCARISS |
| | | | IYYIIDNWGQGTSVTVSS |
| | 3 | HCDR1 | TFAMGIG |
| | 4 | HCDR2 | HIWWDGEKYNNPVLKS |
| | 5 | HCDR3 | ISSIYYIIDN |
| | 6 | LC VD w signal | MVFTPQILGLMLEWISASRGDIVLTQSPATLSVTPGHSVSLSCRASQSISN |
| | | peptide | NLHWYQKKSHESPRLLIKYVSQSISGIPSRFSGSGSGTDFTLSINSVETED |
| | | | FGMYFCQQSYNWPLTFGAGTKLELK |
| | 7 | LC VD w/o | DIVLTQSPATLSVTPGHSVSLSCRASQSISNNLHWYQKKSHESPRLLIKYV |
| | | signal peptide | SQSISGIPSRFSGSGSGTDFTLSINSVETEDFGMYFCQQSYNWPLTFGAGT |
| | | | KLELK |
| | 8 | LCDR1 | RASQSISNNLH |
| | 9 | LCDR2 | YVSQSIS |
| | 10 | LCDR3 | QQSYNWPLT |
| #5 | 11 | HC VD w signal | MGRLTSSFLLLIVPAYVLSQVTLKESGPGILQPSQTLSLTCSFSGFSLSTF |
| | | peptide | GMGVGWIRQPSGKGLEWLAHIWWDDEKYYNSALKSRLTISKATSKNQAFLK |
| | | | IANVDTADTATYYCARISNYGSNYWYFNVWGTGTTVTVSS |
| | 12 | HC VD w/o | QVTLKESGPGILQPSQTLSLTCSFSGFSLSTFGMGVGWIRQPSGKGLEWLA |
| | | signal peptide | HIWWDDEKYYNSALKSRLTISKATSKNQAFLKIANVDTADTATYYCARISN |
| | | | YGSNYWYFNVWGTGTTVTVSS |
| | 13 | HCDR1 | TFGMGVG |
| | 14 | HCDR2 | HIWWDDEKYYNSALKS |
| | 15 | HCDR3 | ISNYGSNYWYFNV |
| | 16 | LC VD w signal | MDFQVQIFSFLLISASVILSRGQVVLTQSPALMSASPGEKVTMTCSAGSSV |
| | | peptide | SCMYWYQQKPGSSPRVLIYDTSNLASGVPARFTGSGSGTSYSLTISRMEAE |
| | | | DAASYYCQQWNSYPLTFGAGTKLELK |
| | 17 | LC VD w/o | QVVLTQSPALMSASPGEKVTMTCSAGSSVSCMYWYQQKPGSSPRVLIYDTS |
| | | signal peptide | NLASGVPARFTGSGSGTSYSLTISRMEAEDAASYYCQQWNSYPLTFGAGTK |
| | | | LELK |
| | 18 | LCDR1 | SAGSSVSCMY |
| | 19 | LCDR2 | DTSNLAS |
| | 20 | LCDR3 | QQWNSYPLT |
| #16 | 21 | HC VD w signal | MGRLTSSFLLLIVPAYVLSQVTLKESGPGILQPSQTLSLTCSFSGFSLSTF |
| | | peptide | ALGVGWIRQPSGRGLEWLAHIWWDDDKYYNPALKSRLTISKDTSKNHVELK |
| | | | IANVDTADTATYYCARITTYYYGMDYWGQGTSVTVSS |
| | 22 | HC VD w/o | QVTLKESGPGILQPSQTLSLTCSFSGFSLSTFALGVGWIRQPSGRGLEWLA |
| | | signal peptide | HIWWDDDKYYNPALKSRLTISKDTSKNHVFLKIANVDTADTATYYCARITT |
| | | | YYYGMDYWGQGTSVTVSS |
| | 23 | HCDR1 | TFALGVG |
| | 24 | HCDR2 | HIWWDDDKYYNPALKS |
| | 25 | HCDR3 | ITTYYYGMDY |
| | 26 | LC VD w signal | MVFTPQILGLMLFWISASRGDIVLSQSPATLSVTPGDSVSLFCRASQSIGN |
| | | peptide | HLHWYQQESHASPRLLIKYASQSISGIPSRFSGSGSGTDFTLSINSVETED |
| | | | FGMYFCQQSYNWPLTFGAGTKLELK |
| | 27 | LC VD w/o | DIVLSQSPATLSVTPGDSVSLFCRASQSIGNHLHWYQQESHASPRLLIKYA |
| | | signal peptide | SQSISGIPSRFSGSGSGTDFTLSINSVETEDFGMYFCQQSYNWPLTFGAGT |
| | | | KLELK |
| | 28 | LCDR1 | RASQSIGNHLH |
| | 29 | LCDR2 | YASQSIS |
| | 30 | LCDR3 | QQSYNWPLT |
| #22 | 31 | HC VD w signal | MGRLTSSFLLLIVPAYVLSQVTLKESGPGILQPSQTLSLTCSFSGFSLSTF |
| | | peptide | GMGVGWIRQPSGKGLEWLAHIWWDDEKYYNSALKSRLTISKATSKNQVFLK |
| | | | IANVDTADTATYYCARISNYGSNYWYFNVWGTGTTVTVSS |
| | 32 | HC VD w/o | QVTLKESGPGILQPSQTLSLTCSFSGFSLSTFGMGVGWIRQPSGKGLEWLA |
| | | signal peptide | HIWWDDEKYYNSALKSRLTISKATSKNQVFLKIANVDTADTATYYCARISN |
| | | | YGSNYWYFNVWGTGTTVTVSS |
| | 33 | HCDR1 | TFGMGVG |
| | 34 | HCDR2 | HIWWDDEKYYNSALKS |
| | 35 | HCDR3 | ISNYGSNYWYFNV |
| | 36 | LC VD w signal | MDFQVQIFSFLLISASVILSRGQVVLTQSPALMSASPGEKVTMTCSAGSSV |
| | | peptide | SYMYWYQQKPGSSPRVLIYDTSNLASGVPARFTGSGSGTSYSLTISRMEAE |
| | | | DAATYYCQQWNSYPLTFGAGTKLELK |
| | 37 | LC VD w/o | QVVLTQSPALMSASPGEKVTMTCSAGSSVSYMYWYQQKPGSSPRVLIYDTS |
| | | signal peptide | NLASGVPARFTGSGSGTSYSLTISRMEAEDAATYYCQQWNSYPLTFGAGTK |
| | | | LELK |
| | 38 | LCDR1 | SAGSSVSYMY |
| | 39 | LCDR2 | DTSNLAS |
| | 40 | LCDR3 | QQWNSYPLT |
| #34 | 41 | HC VD w signal | MGRLTSSFLLLIVPAYVLSQVTLKESGPGILQPSQTLSLTCSFSGFSLSTF |
| | | peptide | SLGVGWIRQSSGKGLEWLAHIWWDDEKYYNPALKSRLTISKDTSKNQVFLK |
| | | | IANVDAADTATYYCARAYYSKSYYALDYWGQGTSVTVSS |
| | 42 | HC VD w/o | QVTLKESGPGILQPSQTLSLTCSFSGFSLSTFSLGVGWIRQSSGKGLEWLA |
| | | signal peptide | HIWWDDEKYYNPALKSRLTISKDTSKNQVFLKIANVDAADTATYYCARAYY |
| | | | SKSYYALDYWGQGTSVTVSS |
| | 43 | HCDR1 | TFSLGVG |
| | 44 | HCDR2 | HIWWDDEKYYNPALKS |
| | 45 | HCDR3 | AYYSKSYYALDY |
| | 46 | LC VD w signal | MRVPAHVFGFLLLWFPGTRCDIQMTQSPSSLSASLGERVSLTCRASQEISG |
| | | peptide | YLSWLQQKPDGTIKRLIYAASTLDSGVPKRFSGSRSGSDYSLAISSLESED |
| | | | FADYYCLQYANFPFTFGSGTKLEIK |
| | 47 | LC VD w/o | DIQMTQSPSSLSASLGERVSLTCRASQEISGYLSWLQQKPDGTIKRLIYAA |
| | | signal peptide | STLDSGVPKRFSGSRSGSDYSLAISSLESEDFADYYCLQYANFPFTFGSGT |
| | | | KLEIK |
| | 48 | LCDR1 | RASQEISGYLS |
| | 49 | LCDR2 | AASTLDS |
| | 50 | LCDR3 | LQYANFPFT |
| #42 | 51 | HC VD w signal | MGRLTSSFLLLIVPAYVLSQVTLKESGPGILQPSQTLSLTCSLSGFSLSTF |
| | | peptide | GRGVGWIRQPSGKGLEWLAHIWWDDEKYYNPALKSRLTISKDTSKNQVFLR |
| | | | IANVDTADTATYYCARIQNYGSNYWYFDVWGTGTTVTVSS |
| | 52 | HC VD w/o | QVTLKESGPGILQPSQTLSLTCSLSGFSLSTFGRGVGWIRQPSGKGLEWLA |
| | | signal peptide | HIWWDDEKYYNPALKSRLTISKDTSKNQVFLRIANVDTADTATYYCARIQN |
| | | | YGSNYWYFDVWGTGTTVTVSS |
| | 53 | HCDR1 | TFGRGVG |
| | 54 | HCDR2 | HIWWDDEKYYNPALKS |
| | 55 | HCDR3 | IQNYGSNYWYFDV |
| | 56 | LC VD w signal | MDFQVQIFSFLLISASVILSRGLIVLTQSPAIMSASPGEKVTMTCGATSRI |
| | | peptide | SYMFWYQQKPGSSPRVLIYDTSNLASGVPVRFSGSGSGTSYSLTISRVEAE |
| | | | DVATYYCQQWNSYPLTFGAGTKLELK |
| | 57 | LC VD w/o | LIVLTQSPAIMSASPGEKVTMTCGATSRISYMFWYQQKPGSSPRVLIYDTS |
| | | signal peptide | NLASGVPVRFSGSGSGTSYSLTISRVEAEDVATYYCQQWNSYPLTFGAGTK |
| | | | LELK |
| | 58 | LCDR1 | GATSRISYMF |
| | 59 | LCDR2 | DTSNLAS |
| | 60 | LCDR3 | QQWNSYPLT |
| #43 | 61 | HC VD w signal | MGRLTSSFLLLIVPAYVLSQVALKESGPGILQPSQTLSLTCSFSGFSLSTF |
| | | peptide | GMGVGWIRQSSGKGLEWLANIWWDDDKYYNPALKSRLTISKDASKNQAFLK |
| | | | IANVDTADTATYYCARIGNYGSNYWYFDVWGTGTTVTVSS |
| | 62 | HC VD w/o | QVALKESGPGILQPSQTLSLTCSFSGFSLSTFGMGVGWIRQSSGKGLEWLA |
| | | signal peptide | NIWWDDDKYYNPALKSRLTISKDASKNQAFLKIANVDTADTATYYCARIGN |
| | | | YGSNYWYFDVWGTGTTVTVSS |
| | 63 | HCDR1 | TFGMGVG |
| | 64 | HCDR2 | NIWWDDDKYYNPALKS |
| | 65 | HCDR3 | IGNYGSNYWYFDV |
| | 66 | LC VD w signal | MDFQVQIFSFLLISASVILSRGQIVLTQSPAIMSASPGERVTMTCSANSRI |
| | | peptide | SYMYWYQQKPGSSPRLLIYDTSNLASGVPVRFSGSGSGTSYSLTISRMEAE |
| | | | DAATYNCQQWSSYPLTFGAGTKLELK |
| | 67 | LC VD w/o | QIVLTQSPAIMSASPGERVTMTCSANSRISYMYWYQQKPGSSPRLLIYDTS |
| | | signal peptide | NLASGVPVRFSGSGSGTSYSLTISRMEAEDAATYNCQQWSSYPLTFGAGTK |
| | | | LELK |
| | 68 | LCDR1 | SANSRISYMY |
| | 69 | LCDR2 | DTSNLAS |
| | 70 | LCDR3 | QQWSSYPLT |
| #44 | 71 | HC VD w signal | MGRLTSSFLLLIVPAYVLSQVTLKESGPGILQPSQTLSLTCSFSGFSLSTF |
| | | peptide | GMGVGWIRQPSEKGLEWLAHIWWDDDKYYNPALKSRLTISKDTSKNQIFLK |
| | | | ITNVDTAETATYYCSRIGNYGSNYWYFDVWGTGTTVTVSS |
| | 72 | HC VD w/o | QVTLKESGPGILQPSQTLSLTCSFSGFSLSTFGMGVGWIRQPSEKGLEWLA |
| | | signal peptide | HIWWDDDKYYNPALKSRLTISKDTSKNQIFLKITNVDTAETATYYCSRIGN |
| | | | YGSNYWYFDVWGTGTTVTVSS |
| | 73 | HCDR1 | TFGMGVG |
| | 74 | HCDR2 | HIWWDDDKYYNPALKS |
| | 75 | HCDR3 | IGNYGSNYWYFDV |
| | 76 | LC VD w signal | MDFQVQIFSFLLISASVIVSRGQIVLTQSPAVMSASPGEKVTMTCTASSSV |
| | | peptide | YYMYWYQQTPGSSPRLLIYDTSNLASGVPVRFSGSGSGTSYSLTISRMEAE |
| | | | DAATYYCQQWNTYPLTFGAGTKLELK |
| | 77 | LC VD w/o | QIVLTQSPAVMSASPGEKVTMTCTASSSVYYMYWYQQTPGSSPRLLIYDTS |
| | | signal peptide | NLASGVPVRFSGSGSGTSYSLTISRMEAEDAATYYCQQWNTYPLTFGAGTK |
| | | | LELK |
| | 78 | LCDR1 | TASSSVYYMY |
| | 79 | LCDR2 | DTSNLAS |
| | 80 | LCDR3 | QQWNTYPLT |
| #46 | 81 | HC VD w signal | MGRLTSSFLLLIVPAYVLSQVTLKESGPGILQPSQTLSLTCSFSGFSLSTF |
| | | peptide | GMGVGWIRQPSGKGLEWLAHIWWDDDNYYNQALKSRLTISKDNTKNQVFLN |
| | | | IANVDTADTATYYCARIRSYDYDVRYAMDYWGQGTSVTVSS |
| | 82 | HC VD w/o | QVTLKESGPGILQPSQTLSLTCSFSGFSLSTFGMGVGWIRQPSGKGLEWLA |
| | | signal peptide | HIWWDDDNYYNQALKSRLTISKDNTKNQVFLNIANVDTADTATYYCARIRS |
| | | | YDYDVRYAMDYWGQGTSVTVSS |
| | 83 | HCDR1 | TFGMGVG |
| | 84 | HCDR2 | HIWWDDDNYYNQALKS |
| | 85 | HCDR3 | IRSYDYDVRYAMDY |
| | 86 | LC VD w signal | MRTPAQFLGILLLWFPGIKCDIKMTQSPSSMYASLRERVTITCKASQDINS |
| | | peptide | YLSWFQQKPGKSPKTLIYRANRLVDGVPSRFSGSGSGQDYSLTISSLEYED |
| | | | LGIYYCQQYYEFPLTFGAGTRLELK |
| | 87 | LC VD w/o | DIKMTQSPSSMYASLRERVTITCKASQDINSYLSWFQQKPGKSPKTLIYRA |
| | | signal peptide | NRLVDGVPSRFSGSGSGQDYSLTISSLEYEDLGIYYCQQYYEFPLTFGAGT |
| | | | RLELK |
| | 88 | LCDR1 | KASQDINSYLS |
| | 89 | LCDR2 | RANRLVD |
| | 90 | LCDR3 | QQYYEFPLT |
| #47 | 91 | HC VD w signal | MEWSRVFIFLLSVTAGVHSQVQLQQSGAELVRPGTSVKVSCKASGYAFTNY |
| | | peptide | LIEWVKQRPGQGLEWIGVINPGSGFTNYNEKFKGKAILNADKSSSTAYMQL |
| | | | ISLTSEDSAVYFCARDLKRAMDHWGQGTSVTVSS |
| | 92 | HC VD w/o | QVQLQQSGAELVRPGTSVKVSCKASGYAFTNYLIEWVKQRPGQGLEWIGVI |
| | | signal peptide | NPGSGFTNYNEKFKGKAILNADKSSSTAYMQLISLTSEDSAVYFCARDLKR |
| | | | AMDHWGQGTSVTVSS |
| | 93 | HCDR1 | NYLIE |
| | 94 | HCDR2 | VINPGSGFTNYNEKFKG |
| | 95 | HCDR3 | DLKRAMDH |
| | 96 | LC VD w signal | MLTQLLGLLLLWFAGGKCDIQMTQSPASQSASLGESVTITCLASQTIGTWL |
| | | peptide | AWYQQKPGKSPQLLIHAATSLADGVPSRFSGSGSGTKFSFKISSLQAEDFV |
| | | | SYYCQQIYSSPYTFGGGTKLEIK |
| | 97 | LC VD w/o | DIQMTQSPASQSASLGESVTITCLASQTIGTWLAWYQQKPGKSPQLLIHAA |
| | | signal peptide | TSLADGVPSRFSGSGSGTKFSFKISSLQAEDFVSYYCQQIYSSPYTFGGGT |
| | | | KLEIK |
| | 98 | LCDR1 | LASQTIGTWLA |
| | 99 | LCDR2 | AATSLAD |
| | 100 | LCDR3 | QQIYSSPYT |
| #48 | 101 | HC VD w signal | MEWSRVFIFLLSVTAGVHSQVQLQQSGTELVRPGTSVKVSCKASGYAFTNY |
| | | peptide | LIEWVKQRPGQGLEWIGVINPGSGFTKYNEKFKGKATLTADKSSSTVYMHL |
| | | | SSLTSEDSAVYFCARGLYRAMDYWGQGTSVTVSS |
| | 102 | HC VD w/o | QVQLQQSGTELVRPGTSVKVSCKASGYAFTNYLIEWVKQRPGQGLEWIGVI |
| | | signal peptide | NPGSGFTKYNEKFKGKATLTADKSSSTVYMHLSSLTSEDSAVYFCARGLYR |
| | | | AMDYWGQGTSVTVSS |
| | 103 | HCDR1 | NYLIE |
| | 104 | HCDR2 | VINPGSGFTKYNEKFKG |
| | 105 | HCDR3 | GLYRAMDY |
| | 106 | LC VD w signal | MLTQLLGLLLLWFAGGKCDIQMTQSPASQSASLGESVTITCLASQTIATWL |
| | | peptide | AWYQQKPGKSPQLLIYGATTLADGVPSRFSGGGSGTKFSFKISSLQAEDFV |
| | | | SYYCQQLYSTPYTFGGGTKLEIK |
| | 107 | LC VD w/o | DIQMTQSPASQSASLGESVTITCLASQTIATWLAWYQQKPGKSPQLLIYGA |
| | | signal peptide | TTLADGVPSRFSGGGSGTKFSFKISSLQAEDFVSYYCQQLYSTPYTFGGGT |
| | | | KLEIK |
| | 108 | LCDR1 | LASQTIATWLA |
| | 109 | LCDR2 | GATTLAD |
| | 110 | LCDR3 | QQLYSTPYT |
| #49 | 111 | HC VD w signal | MGRLTSSFLLLIVPAYVLSQVTLKESGPGILQPSQTLSLTCSFSGFSLSTF |
| | | peptide | GMGVGWIRQPSGKGLEWLAHIWWDDDKYYNPALQSRLTVSKDTAKNQVFLK |
| | | | IANVDTADTAIYYCARVGNYGSNYWYFAVWGTGTTVTVSS |
| | 112 | HC VD w/o | QVTLKESGPGILQPSQTLSLTCSFSGFSLSTFGMGVGWIRQPSGKGLEWLA |
| | | signal peptide | HIWWDDDKYYNPALQSRLTVSKDTAKNQVFLKIANVDTADTAIYYCARVGN |
| | | | YGSNYWYFAVWGTGTTVTVSS |
| | 113 | HCDR1 | TFGMGVG |
| | 114 | HCDR2 | HIWWDDDKYYNPALQS |
| | 115 | HCDR3 | VGNYGSNYWYFAV |
| | 116 | LC VD w signal | MDFQVQIFSFLLISASVILSRGQIVLTQSPAIMSASPGEKVTMTCSASSSV |
| | | peptide | SYMYWYQQKPGSSPRLLIYDTSNLASGVPVRFSGSGSGTSYSLTISRMEAE |
| | | | DAATYYCQQWNSYPLTFGAGTKLELK |
| | 117 | LC VD w/o | QIVLTQSPAIMSASPGEKVTMTCSASSSVSYMYWYQQKPGSSPRLLIYDTS |
| | | signal peptide | NLASGVPVRFSGSGSGTSYSLTISRMEAEDAATYYCQQWNSYPLTFGAGTK |
| | | | LELK |
| | 118 | LCDR1 | SASSSVSYMY |
| | 119 | LCDR2 | DTSNLAS |
| | 120 | LCDR3 | QQWNSYPLT |
| #50 | 121 | HC VD w signal | MEWSRVFIFLLSVTAGVHSQVQLQQSGAELVRPGASVKVSCKASGYAFSNY |
| | | peptide | LIEWVKQRPGQGLEWIGVINPGSGFTKYNEKFKGKATLTADKSSSTAYMQL |
| | | | SSLTSEDSAVYFCARGLYRAMDYWGQGTSVTVSS |
| | 122 | HC VD w/o | QVQLQQSGAELVRPGASVKVSCKASGYAFSNYLIEWVKQRPGQGLEWIGVI |
| | | signal peptide | NPGSGFTKYNEKFKGKATLTADKSSSTAYMQLSSLTSEDSAVYFCARGLYR |
| | | | AMDYWGQGTSVTVSS |
| | 123 | HCDR1 | NYLIE |
| | 124 | HCDR2 | VINPGSGFTKYNEKFKG |
| | 125 | HCDR3 | GLYRAMDY |
| | 126 | LC VD w signal | MLTQLLGLLLLWFAGGKCDIQMTQSPASQSASLGESVTITCLASQTIGTWL |
| | | peptide | AWYQQKPGKSPQLLIYAAASLADGVPSRFSGGGSGTKFSFKISSLQAEDFV |
| | | | NYYCQQIYSTPYTFGGGTKLEIK |
| | 127 | LC VD w/o | DIQMTQSPASQSASLGESVTITCLASQTIGTWLAWYQQKPGKSPQLLIYAA |
| | | signal peptide | ASLADGVPSRFSGGGSGTKFSFKISSLQAEDFVNYYCQQIYSTPYTFGGGT |
| | | | KLEIK |
| | 128 | LCDR1 | LASQTIGTWLA |
| | 129 | LCDR2 | AAASLAD |
| | 130 | LCDR3 | QQIYSTPYT |
| #51 | 131 | HC VD w signal | MEWPLIFLFLLSGTAGVQSQVQLQQSGAELVKPGASVKISCKASGYTFSNY |
| | | peptide | WMNWVKQRPGKGLEWIGQIYPGDGDTKYNGKFKNKATLTADKSSSTAYMQF |
| | | | SSLISEDSAVYFCARGDLVFVYWGLGTLVTVSA |
| | 132 | HC VD w/o | QVQLQQSGAELVKPGASVKISCKASGYTFSNYWMNWVKQRPGKGLEWIGQI |
| | | signal peptide | YPGDGDTKYNGKFKNKATLTADKSSSTAYMQFSSLTSEDSAVYFCARGDLV |
| | | | FVYWGLGTLVTVSA |
| | 133 | HCDR1 | NYWMN |
| | 134 | HCDR2 | QIYPGDGDTKYNGKFKN |
| | 135 | HCDR3 | GDLVFVY |
| | 136 | LC VD w signal | MRFQVQVLGLLLLWISGAQCDVQITQSPSYLAVSPGETITINCRASKNIRK |
| | | peptide | YLAWYQEKPGKTNKLLIYSGSTSQSGVPSRFSGSGSGTDFTLTISSLEPED |
| | | | FAMYYCQQHNEYPYTFGGGTKLEIK |
| | 137 | LC VD w/o | DVQITQSPSYLAVSPGETITINCRASKNIRKYLAWYQEKPGKINKLLIYSG |
| | | signal peptide | STSQSGVPSRFSGSGSGTDFTLTISSLEPEDFAMYYCQQHNEYPYTFGGGT |
| | | | KLEIK |
| | 138 | LCDR1 | RASKNIRKYLA |
| | 139 | LCDR2 | SGSTSQS |
| | 140 | LCDR3 | QQHNEYPYT |
| #52 | 141 | HC VD w signal | MEWSRVFIFLLSVTAGAHSQVQLQQSGAELVRPGTSVKVSCKASGYAFTNY |
| | | peptide | LIEWVKQRPGQGLEWIGVENPESGYINYNEKLKGKATLTADKSSSTAYMQL |
| | | | SSLISEDSAVYFCARTSRRGFDYWGHGTTLTVSS |
| | 142 | HC VD w/o | QVQLQQSGAELVRPGTSVKVSCKASGYAFTNYLIEWVKQRPGQGLEWIGVF |
| | | signal peptide | NPESGYINYNEKLKGKATLTADKSSSTAYMQLSSLTSEDSAVYFCARTSRR |
| | | | GFDYWGHGTTLTVSS |
| | 143 | HCDR1 | NYLIE |
| | 144 | HCDR2 | VENPESGYINYNEKLKG |
| | 145 | HCDR3 | TSRRGFDY |
| | 146 | LC VD w signal | MNMLTQLLGLLLLWFAGGKCDIQMTQSPASQSASLGESVTITCLASQTIGT |
| | | peptide | WLAWYQQKPGKSPQLLIYAATSLADGVPSRFSGSGSGTKFSFKISSLQAED |
| | | | FVSYYCQQLYSTPRTFGGGTKLEIK |
| | 147 | LC VD w/o | DIQMTQSPASQSASLGESVTITCLASQTIGTWLAWYQQKPGKSPQLLIYAA |
| | | signal peptide | TSLADGVPSRFSGSGSGTKFSFKISSLQAEDFVSYYCQQLYSTPRTFGGGT |
| | | | KLEIK |
| | 148 | LCDR1 | LASQTIGTWLA |
| | 149 | LCDR2 | AATSLAD |
| | 150 | LCDR3 | QQLYSTPRT |
| #54 | 151 | HC VD w signal | MGRLTSSFLLLIVPAYVLSQVTLKESGPGILQPSQTLSLTCSFSGFSLSTF |
| | | peptide | GLGVGWIRQPSGKGLECLAHIWWDDDKYSNPALKSRLTISKDTSKNQVFLK |
| | | | IANVDSADTATYFCARILNYGSNYWYFDVWGTGTTVTVSS |
| | 152 | HC VD w/o | QVTLKESGPGILQPSQTLSLTCSFSGFSLSTFGLGVGWIRQPSGKGLECLA |
| | | signal peptide | HIWWDDDKYSNPALKSRLTISKDTSKNQVFLKIANVDSADTATYFCARILN |
| | | | YGSNYWYFDVWGTGTTVTVSS |
| | 153 | HCDR1 | TFGLGVG |
| | 154 | HCDR2 | HIWWDDDKYSNPALKS |
| | 155 | HCDR3 | ILNYGSNYWYFDV |
| | 156 | LC VD w signal | MDFQVQIFSFLLISASVILSRGQIVLTQSPALMSASPGEKVTMTCSASSSI |
| | | peptide | SYMYWYQQKPGSSPRLLIYDTSNLASGVPVRFSGSGSGTSFSLTVSRMEAE |
| | | | DAATYYCQQWNSYPLTFGAGTKLELK |
| | 157 | LC VD w/o | QIVLTQSPALMSASPGEKVTMTCSASSSISYMYWYQQKPGSSPRLLIYDTS |
| | | signal peptide | NLASGVPVRFSGSGSGTSFSLTVSRMEAEDAATYYCQQWNSYPLTFGAGTK |
| | | | LELK |
| | 158 | LCDR1 | SASSSISYMY |
| | 159 | LCDR2 | DTSNLAS |
| | 160 | LCDR3 | QQWNSYPLT |
| #56 | 161 | HC VD w signal | MGRLTSSFLLLIVPAYVLSQVTLKESGPGILQPSQTLSLTCSFSGFSLSTF |
| | | peptide | GMGVGWIRQPSGKGLEWLTNIWWDDDKYYNSVLKSRLTISKDTSKNQVELK |
| | | | IANVDTADTATYYCARIAAYGSNYWYFDVWGTGTTVTVSS |
| | 162 | HC VD w/o | QVTLKESGPGILQPSQTLSLTCSFSGFSLSTFGMGVGWIRQPSGKGLEWLT |
| | | signal peptide | NIWWDDDKYYNSVLKSRLTISKDTSKNQVFLKIANVDTADTATYYCARIAA |
| | | | YGSNYWYFDVWGTGTTVTVSS |
| | 163 | HCDR1 | TFGMGVG |
| | 164 | HCDR2 | NIWWDDDKYYNSVLKS |
| | 165 | HCDR3 | IAAYGSNYWYFDV |
| | 166 | LC VD w signal | MDFQVQIFSFLLISASVKLSRGQIVLTQSPAIMSASPGEKVTMTCSASSSV |
| | | peptide | SYMYWYQQKPGSSPRVLIYDTSNLSSGVPVRFSGSGSGTSYSLTISRMEAE |
| | | | DAATYYCQQWSSYPLTFGAGTKLELK |
| | 167 | LC VD w/o | QIVLTQSPAIMSASPGEKVTMTCSASSSVSYMYWYQQKPGSSPRVLIYDTS |
| | | signal peptide | NLSSGVPVRFSGSGSGTSYSLTISRMEAEDAATYYCQQWSSYPLTFGAGTK |
| | | | LELK |
| | 168 | LCDR1 | SASSSVSYMY |
| | 169 | LCDR2 | DTSNLSS |
| | 170 | LCDR3 | QQWSSYPLT |
| #57 | 171 | HC VD w signal | MGRLTSSLLLLIVPAYVLSQVTLKESGPGILQPSQTLSLTCSFSGFSLSTF |
| | | peptide | GMGVGWIRQPSGKGLEWLAHIWWDDDKYYNPALKSRLTISKDTSKNQIFLK |
| | | | IANVDTADSATFYCARIENYGSNYWYFDVWGTGTTVTVSS |
| | 172 | HC VD w/o | QVTLKESGPGILQPSQTLSLTCSFSGFSLSTFGMGVGWIRQPSGKGLEWLA |
| | | signal peptide | HIWWDDDKYYNPALKSRLTISKDTSKNQIFLKIANVDTADSATFYCARIEN |
| | | | YGSNYWYFDVWGTGTTVTVSS |
| | 173 | HCDR1 | TFGMGVG |
| | 174 | HCDR2 | HIWWDDDKYYNPALKS |
| | 175 | HCDR3 | IENYGSNYWYFDV |
| | 176 | LC VD w signal | MDFQVQIFSFLLISASVRLSRGQIVLTQSPAIMSASPGEKVTMTCSASSSV |
| | | peptide | SYMYWYQQKPGSSPRVLIYDTSNLASGVPVRFSGSGSGTSYSLTVSRMEAE |
| | | | DAATYYCQQWNSYPLTFGAGTKLELK |
| | 177 | LC VD w/o | QIVLTQSPAIMSASPGEKVTMTCSASSSVSYMYWYQQKPGSSPRVLIYDTS |
| | | signal peptide | NLASGVPVRFSGSGSGTSYSLTVSRMEAEDAATYYCQQWNSYPLTFGAGTK |
| | | | LELK |
| | 178 | LCDR1 | SASSSVSYMY |
| | 179 | LCDR2 | DTSNLAS |
| | 180 | LCDR3 | QQWNSYPLT |

| | | |
|---|---|---|
| 181 | human iRhom2 | MASADKNGGSVSSVSSSRLQSRKPPNLSITIPPPEKETQAPGEQDSMLPEG |
| | | FQNRRLKKSQPRTWAAHTTACPPSFLPKRKNPAYLKSVSLQEPRSRWQESS |
| | | EKRPGFRRQASLSQSIRKGAAQWFGVSGDWEGQRQQWQRRSLHHCSMRYGR |
| | | LKASCQRDLELPSQEAPSFQGTESPKPCKMPKIVDPLARGRAFRHPEEMDR |
| | | PHAPHPPLTPGVLSLTSFTSVRSGYSHLPRRKRMSVAHMSLQAAAALLKGR |
| | | SVLDATGQRCRVVKRSFAFPSFLEEDVVDGADTFDSSFFSKEEMSSMPDDV |
| | | FESPPLSASYFRGIPHSASPVSPDGVQIPLKEYGRAPVPGPRRGKRIASKV |
| | | KHFAFDRKKRHYGLGVVGNWLNRSYRRSISSTVQRQLESFDSHRPYFTYWL |
| | | TFVHVIITLLVICTYGIAPVGFAQHVTTQLVLRNKGVYESVKYIQQENFWV |
| | | GPSSIDLIHLGAKFSPCIRKDGQIEQLVLRERDLERDSGCCVQNDHSGCIQ |
| | | TQRKDCSETLATFVKWQDDTGPPMDKSDLGQKRTSGAVCHQDPRTCEEPAS |
| | | SGAHIWPDDITKWPICTEQARSNHTGFLHMDCEIKGRPCCIGTKGSCEITT |
| | | REYCEFMHGYFHEEATLCSQVHCLDKVCGLLPFLNPEVPDQFYRLWLSLFL |
| | | HAGVVHCLVSVVFQMTILRDLEKLAGWHRIAIIFILSGITGNLASAIFLPY |
| | | RAEVGPAGSQFGLLACLFVELFQSWPLLERPWKAFLNLSAIVLFLFICGLL |
| | | PWIDNIAHIFGFLSGLLLAFAFLPYITFGTSDKYRKRALILVSLLAFAGLF |
| | | AALVLWLYIYPINWPWIEHLTCFPFTSRFCEKYELDQVLH |
| 182 | human iRhom1 | MSEARRDSTSSLQRKKPPWLKLDIPSAVPLTAEEPSFLQPLRRQAFLRSVS |
| | | MPAETAHISSPHHELRRPVLQRQTSITQTIRRGTADWFGVSKDSDSTQKWQ |
| | | RKSIRHCSQRYGKLKPQVLRELDLPSQDNVSLTSTETPPPLYVGPCQLGMQ |
| | | KIIDPLARGRAFRVADDTAEGLSAPHTPVTPGAASLCSFSSSRSGFHRLPR |
| | | RRKRESVAKMSFRAAAALMKGRSVRDGTFRRAQRRSFTPASFLEEDTTDFP |
| | | DELDTSFFAREGILHEELSTYPDEVFESPSEAALKDWEKAPEQADLTGGAL |
| | | DRSELERSHLMLPLERGWRKQKEGAAAPQPKVRLRQEVVSTAGPRRGQRIA |
| | | VPVRKLFAREKRPYGLGMVGRLTNRTYRKRIDSFVKRQIEDMDDHRPFFTY |
| | | WLTFVHSLVTILAVCIYGIAPVGFSQHETVDSVLRNRGVYENVKYVQQENF |
| | | WIGPSSEALIHLGAKFSPCMRQDPQVHSFIRSAREREKHSACCVRNDRSGC |
| | | VQTSEEECSSTLAVWVKWPIHPSAPELAGHKRQFGSVCHQDPRVCDEPSSE |
| | | DPHEWPEDITKWPICTKNSAGNHTNHPHMDCVITGRPCCIGTKGRCEITSR |
| | | EYCDFMRGYFHEEATLCSQVHCMDDVCGLLPFLNPEVPDQFYRLWLSLFLH |
| | | AGILHCLVSICFQMTVLRDLEKLAGWHRIAIIYLLSGVTGNLASAIFLPYR |
| | | AEVGPAGSQFGILACLFVELFQSWQILARPWRAFFKLLAVVLFLFTFGLLP |
| | | WIDNFAHISGFISGLFLSFAFLPYISFGKFDLYRKRCQIIIFQVVFLGLLA |
| | | GLVVLFYVYPVRCEWCEFLTCIPFTDKFCEKYELDAQLH |
| 183 | mouse iRhom2 | MASADKNGSNLPSVSGSRLQSRKPPNLSITIPPPESQAPGEQDSMLPERRK |
| | | NPAYLKSVSLQEPRGRWQEGAEKRPGFRRQASLSQSIRKSTAQWFGVSGDW |
| | | EGKRQNWHRRSLHHCSVHYGRLKASCQRELELPSQEVPSFQGTESPKPCKM |
| | | PKIVDPLARGRAFRHPDEVDRPHAAHPPLTPGVLSLTSFTSVRSGYSHLPR |
| | | RKRISVAHMSFQAAAALLKGRSVLDATGQRCRHVKRSFAYPSFLEEDAVDG |
| | | ADTFDSSFFSKEEMSSMPDDVFESPPLSASYFRGVPHSASPVSPDGVHIPL |
| | | KEYSGGRALGPGTQRGKRIASKVKHFAFDRKKRHYGLGVVGNWLNRSYRRS |
| | | ISSTVQRQLESFDSHRPYFTYWLTFVHIIITLLVICTYGIAPVGFAQHVIT |
| | | QLVLKNRGVYESVKYIQQENFWIGPSSIDLIHLGAKFSPCIRKDQQIEQLV |
| | | RRERDIERTSGCCVQNDRSGCIQTLKKDCSETLATFVKWQNDTGPSDKSDL |
| | | SQKQPSAVVCHQDPRICEEPASSGAHIWPDDITKWPICTEQAQSNHTGLLH |
| | | IDCKIKGRPCCIGTKGSCEITTREYCEFMHGYFHEDATLCSQVHCLDKVCG |
| | | LLPFLNPEVPDQFYRIWLSLFLHAGIVHCLVSVVFQMTILRDLEKLAGWHR |
| | | ISIIFILSGITGNLASAIFLPYRAEVGPAGSQFGLLACLFVELFQSWQLLE |
| | | RPWKAFFNLSAIVLFLFICGLLPWIDNIAHIFGELSGMLLAFAFLPYITFG |
| | | TSDKYRKRALILVSLLVFAGLFASLVLWLYIYPINWPWIEYLTCFPFTSRF |
| | | CEKYELDQVLH |

SEQ ID NO 198=Homo sapiens iRhom2, full length, wild type, FLAG-Tag
SEQ ID NO 199=Mus musculus iRhom2, full length, wild type, FLAG-Tag

1. A protein binder which, when bound to human iRhom2, binds at least within a region of Loop 1 thereof.

2. The protein binder of claim 1, which binds within at least a region of human iRhom2 spanning from (and including) W526 to (and including) 1566.

3. The protein binder of claim 1 which binds a stretch of human iRhom2 comprising at least one residue selected from the group comprising W526; Q527; P532; P533; M534; D535; K536; S537; L539; K542; R543; T544; G546; R554; E557; S561; S562 and/or 1566.

4. The protein binder of claim 1, which inhibits and/or reduces TACE/ADAM17 activity when bound to human iRhom2.

5. The protein binder of claim 1, wherein the inhibition or reduction of TACE/ADAM17 activity is caused by interference with iRhom2-mediated TACE/ADAM17 activation.

6. The protein binder of claim 1, which, when bound to human iRhom2,
inhibits or reduces induced TNFα shedding and/or
inhibits or reduces induced IL-6R shedding, and/or
inhibits or reduces induced HB-EGF shedding.

7. The protein binder of claim 1, wherein the human iRhom2 to which the protein binder binds comprises
a) the amino acid sequence set forth in SEQ ID NO; 181, or
b) an amino acid sequence that has at least 80% sequence identity with SEQ ID NO; 181, with the proviso that said sequence maintains iRhom2 activity.

8. The protein binder of claim 1, which is a monoclonal antibody, or a target-binding fragment or derivative thereof retaining target binding capacities, or an antibody mimetic.

9. (canceled)

10. The protein binder of claim 1, which is not cross-reactive with human iRhom1.

11. The protein binder of claim 8, which protein binder is an antibody that
a) comprises a set of heavy chain/light chain complementarity determining regions (CDR) comprised in the heavy chain/light variable domain sequence pair set forth in the following pairs of SEQ ID NOs:
2 and 7; 12 and 17; 22 and 27; 32 and 37; 42 and 47; 52 and 57; 62 and 67; 72 and 77; 82 and 87; 112 and 117; 152 and 157; 162 and 167; and/or 172 and 177;
b) comprises a set of heavy chain/light chain complementarity determining regions (CDR) comprising the following SEQ ID NOs, in the order (HCDR1; HCDR2; HCDR3; LCDR1; LCDR2 and LCDR3)
3,4, 5, 8, 9,10;
13,14,15,18,19,20;
23,24,25,28,29,30;
33,34,35,38,39,40;
43,44,45,48,49, 50;
53, 54, 55, 58, 59, 60;
63, 64, 65, 68, 69,70;
73,74,75,78,79, 80;
83, 84, 85, 88, 89, 90;
113,114,115,118,119,120;
153,154,155,158,159,160;
163, 164, 165, 168, 169, 170; and/or
173,174,175,178,179,180;
c) comprises the heavy chain/light chain complementarity determining regions (CDR) of b), with the proviso that at least one of the CDRs has up to 3 amino acid substitutions relative to the respective SEQ ID NOs, and/or
d) comprises the heavy chain/light chain complementarity determining regions (CDR) of b) or c), with the proviso that at least one of the CDRs has a sequence identity of 66% to the respective SEQ ID NOs,
wherein the CDRs are embedded in a suitable protein framework so as to be capable to bind to human iRhom2 with sufficient binding affinity and to inhibit or reduce TACE/ADAM17 activity.

12. The protein binder of claim 8, which comprises
a) the heavy chain/light chain variable domain (HCVD/LCVD) pairs set forth in the following pairs of SEQ ID NOs:
2 and 7; 12 and 17; 22 and 27; 32 and 37; 42 and 47; 52 and 57; 62 and 67; 72 and 77; 82 and 87; 112 and 117; 152 and 157; 162 and 167; and/or 172 and 177;
b) the heavy chain/light chain variable domains (HCVD/LCVD) pairs of α), with the proviso that
the HCVD has a sequence identity of 80% to the respective SEQ ID NO, and/or
the LCVD has a sequence identity of 80% to the respective SEQ ID NO, and/or
c) the heavy chain/light chain variable domains (VD) pairs of α) or b), with the proviso that at least one of the HCVD or LCVD has up to 10 amino acid substitutions relative to the respective SEQ ID NO,
said protein binder still being capable to bind to human iRhom2 with sufficient binding affinity and to inhibit or reduce TACE/ADAM17 activity.

13. (canceled)

14. The protein binder of claim 1, which protein binder has at least one of
target binding affinity of 50% to human iRhom2 compared to that of the protein binder according to any one of the aforementioned claims, and/or
50% of the inhibiting or reducing effect on TACE/ADAM17 activity of the protein binder according to any one of the aforementioned claims.

15. A protein binder that binds to human iRhom2, and competes for binding to human iRhom2 with
a) an antibody of claim 8, or
b) an antibody selected from the group consisting of clones #3, #5, #16, #22, #34, #42, #43, #44, #46, #49, #54, #56, or #57.

16. A protein binder that binds to essentially the same, or the same, region on human iRhom2 as
a) an antibody of claim 8, or
b) an antibody selected from the group consisting of clones #3, #5, #16, #22, #34, #42, #43, #44, #46, #49, #54, #56, or #57.

17. A nucleic acid that encodes for at least one chain of the binding agent of claim 1.

18. (canceled)

19. A pharmaceutical composition comprising the protein binder of claim 1, and one or more pharmaceutically acceptable excipients.

20. A combination comprising (i) the protein binder of claim 1, and (ii) one or more therapeutically active compounds.

21. A method for treating or preventing an inflammatory condition, which method comprises administration, to a human or animal subject, of a composition comprising the protein binder of claim 1, in a therapeutically sufficient dose.

22. The method of claim 21, wherein the inflammatory condition is Rheumatoid Arthritis (RA)

23. A therapeutic kit of parts comprising:
a) the protein binder of claim 1,
b) an apparatus for administering the protein binder, and
c) instructions for use.


SEQUENCE LISTING (199 sequences)

SEQ ID NO: 1 - protein, 139 aa, artificial sequence
  antibody sequence of fragment
MGRLTSSFLLLIVPAYVLSQVTLKESGPGILQPSQTLSLTCSFSGFSLTTFAMGIGWVRQPSGKGLEWLAHIWWDGEKYNNPVLKSRLTISKDTSKNQVFLRIANVDTPDTATYYCARISSIYYIIDNWGQGTSVTVSS

SEQ ID NO: 2 - protein, 120 aa, artificial sequence
  antibody sequence of fragment
QVTLKESGPGILQPSQTLSLTCSFSGFSLTTFAMGIGWVRQPSGKGLEWLAHIWWDGEKYNNPVLKSRLTISKDTSKNQVFLRIANVDTPDTATYYCARISSIYYIIDNWGQGTSVTVSS

SEQ ID NO: 3 - protein, 7 aa, artificial sequence
  antibody sequence of fragment
TFAMGIG

SEQ ID NO: 4 - protein, 16 aa, artificial sequence
  antibody sequence of fragment
HIWWDGEKYNNPVLKS

SEQ ID NO: 5 - protein, 10 aa, artificial sequence
  antibody sequence of fragment
ISSIYYIIDN

SEQ ID NO: 6 - protein, 127 aa, artificial sequence
  antibody sequence of fragment
MVFTPQILGLMLFWISASRGDIVLTQSPATLSVTPGHSVSLSCRASQSISNNLHWYQKKSHESPRLLIKYVSQSISGIPSRFSGSGSGTDFTLSINSVETEDFGMYFCQQSYNWPLTFGAGTKLELK

SEQ ID NO: 7 - protein, 107 aa, artificial sequence
  antibody sequence of fragment
DIVLTQSPATLSVTPGHSVSLSCRASQSISNNLHWYQKKSHESPRLLIKYVSQSISGIPSRFSGSGSGTDFTLSINSVETEDFGMYFCQQSYNWPLTFGAGTKLELK

SEQ ID NO: 8 - protein, 11 aa, artificial sequence
  antibody sequence of fragment
RASQSISNNLH

SEQ ID NO: 9 - protein, 7 aa, artificial sequence
  antibody sequence of fragment
YVSQSIS

SEQ ID NO: 10 - protein, 9 aa, artificial sequence
  antibody sequence of fragment
QQSYNWPLT

SEQ ID NO: 11 - protein, 142 aa, artificial sequence
  antibody sequence of fragment
MGRLTSSFLLLIVPAYVLSQVTLKESGPGILQPSQTLSLTCSFSGFSLSTFGMGVGWIRQPSGKGLEWLAHIWWDDEKYYNSALKSRLTISKATSKNQAFLKIANVDTADTATYYCARISNYGSNYWYFNVWGTGTTVTVSS

SEQ ID NO: 12 - protein, 123 aa, artificial sequence
  antibody sequence of fragment
QVTLKESGPGILQPSQTLSLTCSFSGFSLSTFGMGVGWIRQPSGKGLEWLAHIWWDDEKYYNSALKSRLTISKATSKNQAFLKIANVDTADTATYYCARISNYGSNYWYFNVWGTGTTVTVSS

SEQ ID NO: 13 - protein, 7 aa, artificial sequence
  antibody sequence of fragment
TFGMGVG

SEQ ID NO: 14 - protein, 16 aa, artificial sequence
  antibody sequence of fragment
HIWWDDEKYYNSALKS

SEQ ID NO: 15 - protein, 13 aa, artificial sequence
  antibody sequence of fragment
ISNYGSNYWYFNV

SEQ ID NO: 16 - protein, 128 aa, artificial sequence
  antibody sequence of fragment
MDFQVQIFSFLLISASVILSRGQVVLTQSPALMSASPGEKVTMTCSAGSSVSCMYWYQQKPGSSPRVLIYDTSNLASGVPARFTGSGSGTSYSLTISRMEAEDAASYYCQQWNSYPLTFGAGTKLELK

SEQ ID NO: 17 - protein, 106 aa, artificial sequence
  antibody sequence of fragment
QVVLTQSPALMSASPGEKVTMTCSAGSSVSCMYWYQQKPGSSPRVLIYDTSNLASGVPARFTGSGSGTSYSLTISRMEAEDAASYYCQQWNSYPLTFGAGTKLELK

SEQ ID NO: 18 - protein, 10 aa, artificial sequence
  antibody sequence of fragment
SAGSSVSCMY

SEQ ID NO: 19 - protein, 7 aa, artificial sequence
  antibody sequence of fragment
DTSNLAS

SEQ ID NO: 20 - protein, 9 aa, artificial sequence
  antibody sequence of fragment
QQWNSYPLT

SEQ ID NO: 21 - protein, 139 aa, artificial sequence
  antibody sequence of fragment
MGRLTSSFLLLIVPAYVLSQVTLKESGPGILQPSQTLSLTCSFSGFSLSTFALGVGWIRQPSGRGLEWLAHIWWDDDKYYNPALKSRLTISKDTSKNHVFLKIANVDTADTATYYCARITTYYYGMDYWGQGTSVTVSS

SEQ ID NO: 22 - protein, 120 aa, artificial sequence
  antibody sequence of fragment
QVTLKESGPGILQPSQTLSLTCSFSGFSLSTFALGVGWIRQPSGRGLEWLAHIWWDDDKYYNPALKSRLTISKDTSKNHVFLKIANVDTADTATYYCARITTYYYGMDYWGQGTSVTVSS

SEQ ID NO: 23 - protein, 7 aa, artificial sequence
  antibody sequence of fragment
TFALGVG

SEQ ID NO: 24 - protein, 16 aa, artificial sequence
  antibody sequence of fragment
HIWWDDDKYYNPALKS

SEQ ID NO: 25 - protein, 10 aa, artificial sequence
  antibody sequence of fragment
ITTYYYGMDY

SEQ ID NO: 26 - protein, 127 aa, artificial sequence
  antibody sequence of fragment
MVFTPQILGLMLFWISASRGDIVLSQSPATLSVTPGDSVSLFCRASQSIGNHLHWYQQESHASPRLLIKYASQSISGIPSRFSGSGSGTDFTLSINSVETEDFGMYFCQQSYNWPLTFGAGTKLELK

SEQ ID NO: 27 - protein, 107 aa, artificial sequence
  antibody sequence of fragment
DIVLSQSPATLSVTPGDSVSLFCRASQSIGNHLHWYQQESHASPRLLIKYASQSISGIPSRFSGSGSGTDFTLSINSVETEDFGMYFCQQSYNWPLTFGAGTKLELK

SEQ ID NO: 28 - protein, 11 aa, artificial sequence
  antibody sequence of fragment
RASQSIGNHLH

SEQ ID NO: 29 - protein, 7 aa, artificial sequence
  antibody sequence of fragment
YASQSIS

SEQ ID NO: 30 - protein, 9 aa, artificial sequence
  antibody sequence of fragment
QQSYNWPLT

SEQ ID NO: 31 - protein, 142 aa, artificial sequence
  antibody sequence of fragment
MGRLTSSFLLLIVPAYVLSQVTLKESGPGILQPSQTLSLTCSFSGFSLSTFGMGVGWIRQPSGKGLEWLAHIWWDDEKYYNSALKSRLTISKATSKNQVFLKIANVDTADTATYYCARISNYGSNYWYFNVWGTGTTVTVSS

SEQ ID NO: 32 - protein, 123 aa, artificial sequence
  antibody sequence of fragment
QVTLKESGPGILQPSQTLSLTCSFSGFSLSTFGMGVGWIRQPSGKGLEWLAHIWWDDEKYYNSALKSRLTISKATSKNQVFLKIANVDTADTATYYCARISNYGSNYWYFNVWGTGTTVTVSS

SEQ ID NO: 33 - protein, 7 aa, artificial sequence
  antibody sequence of fragment
TFGMGVG

SEQ ID NO: 34 - protein, 16 aa, artificial sequence
  antibody sequence of fragment
HIWWDDEKYYNSALKS

SEQ ID NO: 35 - protein, 13 aa, artificial sequence
  antibody sequence of fragment
ISNYGSNYWYFNV

SEQ ID NO: 36 - protein, 128 aa, artificial sequence
  antibody sequence of fragment
MDFQVQIFSFLLISASVILSRGQVVLTQSPALMSASPGEKVTMTCSAGSSVSYMYWYQQKPGSSPRVLIYDTSNLASGVPARFTGSGSGTSYSLTISRMEAEDAATYYCQQWNSYPLTFGAGTKLELK

SEQ ID NO: 37 - protein, 106 aa, artificial sequence
  antibody sequence of fragment
QVVLTQSPALMSASPGEKVTMTCSAGSSVSYMYWYQQKPGSSPRVLIYDTSNLASGVPARFTGSGSGTSYSLTISRMEAEDAATYYCQQWNSYPLTFGAGTKLELK

SEQ ID NO: 38 - protein, 10 aa, artificial sequence
  antibody sequence of fragment
SAGSSVSYMY

SEQ ID NO: 39 - protein, 7 aa, artificial sequence
  antibody sequence of fragment
DTSNLAS

SEQ ID NO: 40 - protein, 9 aa, artificial sequence
  antibody sequence of fragment
QQWNSYPLT

SEQ ID NO: 41 - protein, 141 aa, artificial sequence
  antibody sequence of fragment
MGRLTSSFLLLIVPAYVLSQVTLKESGPGILQPSQTLSLTCSFSGFSLSTFSLGVGWIRQSSGKGLEWLAHIWWDDEKYYNPALKSRLTISKDTSKNQVFLKIANVDAADTATYYCARAYYSKSYYALDYWGQGTSVTVSS

SEQ ID NO: 42 - protein, 122 aa, artificial sequence
  antibody sequence of fragment
QVTLKESGPGILQPSQTLSLTCSFSGFSLSTFSLGVGWIRQSSGKGLEWLAHIWWDDEKYYNPALKSRLTISKDTSKNQVFLKIANVDAADTATYYCARAYYSKSYYALDYWGQGTSVTVSS

SEQ ID NO: 43 - protein, 7 aa, artificial sequence
  antibody sequence of fragment
TFSLGVG

SEQ ID NO: 44 - protein, 16 aa, artificial sequence
  antibody sequence of fragment
HIWWDDEKYYNPALKS

SEQ ID NO: 45 - protein, 12 aa, artificial sequence
  antibody sequence of fragment
AYYSKSYYALDY

SEQ ID NO: 46 - protein, 127 aa, artificial sequence
  antibody sequence of fragment
MRVPAHVFGFLLLWFPGTRCDIQMTQSPSSLSASLGERVSLTCRASQEISGYLSWLQQKPDGTIKRLIYAASTLDSGVPKRFSGSRSGSDYSLAISSLESEDFADYYCLQYANFPFTFGSGTKLEIK

SEQ ID NO: 47 - protein, 107 aa, artificial sequence
  antibody sequence of fragment
DIQMTQSPSSLSASLGERVSLTCRASQEISGYLSWLQQKPDGTIKRLIYAASTLDSGVPKRFSGSRSGSDYSLAISSLESEDFADYYCLQYANFPFTFGSGTKLEIK

SEQ ID NO: 48 - protein, 11 aa, artificial sequence
  antibody sequence of fragment
RASQEISGYLS

SEQ ID NO: 49 - protein, 7 aa, artificial sequence
  antibody sequence of fragment
AASTLDS

SEQ ID NO: 50 - protein, 9 aa, artificial sequence
  antibody sequence of fragment
LQYANFPFT

SEQ ID NO: 51 - protein, 142 aa, artificial sequence
  antibody sequence of fragment
MGRLTSSFLLLIVPAYVLSQVTLKESGPGILQPSQTLSLTCSLSGFSLSTFGRGVGWIRQPSGKGLEWLAHIWWDDEKYYNPALKSRLTISKDTSKNQVFLRIANVDTADTATYYCARIQNYGSNYWYFDVWGTGTTVTVSS

SEQ ID NO: 52 - protein, 123 aa, artificial sequence
  antibody sequence of fragment
QVTLKESGPGILQPSQTLSLTCSLSGFSLSTFGRGVGWIRQPSGKGLEWLAHIWWDDEKYYNPALKSRLTISKDTSKNQVFLRIANVDTADTATYYCARIQNYGSNYWYFDVWGTGTTVTVSS

SEQ ID NO: 53 - protein, 7 aa, artificial sequence
  antibody sequence of fragment
TFGRGVG

SEQ ID NO: 54 - protein, 16 aa, artificial sequence
  antibody sequence of fragment
HIWWDDEKYYNPALKS

SEQ ID NO: 55 - protein, 13 aa, artificial sequence
  antibody sequence of fragment
IQNYGSNYWYFDV

SEQ ID NO: 56 - protein, 128 aa, artificial sequence
  antibody sequence of fragment
MDFQVQIFSFLLISASVILSRGLIVLTQSPAIMSASPGEKVTMTCGATSRISYMFWYQQKPGSSPRVLIYDTSNLASGVPVRFSGSGSGTSYSLTISRVEAEDVATYYCQQWNSYPLTFGAGTKLELK

SEQ ID NO: 57 - protein, 106 aa, artificial sequence
  antibody sequence of fragment
LIVLTQSPAIMSASPGEKVTMTCGATSRISYMFWYQQKPGSSPRVLIYDTSNLASGVPVRFSGSGSGTSYSLTISRVEAEDVATYYCQQWNSYPLTFGAGTKLELK

SEQ ID NO: 58 - protein, 10 aa, artificial sequence
  antibody sequence of fragment
GATSRISYMF

SEQ ID NO: 59 - protein, 7 aa, artificial sequence
  antibody sequence of fragment
DTSNLAS

SEQ ID NO: 60 - protein, 9 aa, artificial sequence
  antibody sequence of fragment
QQWNSYPLT

SEQ ID NO: 61 - protein, 142 aa, artificial sequence
  antibody sequence of fragment
MGRLTSSFLLLIVPAYVLSQVALKESGPGILQPSQTLSLTCSFSGFSLSTFGMGVGWIRQSSGKGLEWLANIWWDDDKYYNPALKSRLTISKDASKNQAFLKIANVDTADTATYYCARIGNYGSNYWYFDVWGTGTTVTVSS

SEQ ID NO: 62 - protein, 123 aa, artificial sequence
  antibody sequence of fragment
QVALKESGPGILQPSQTLSLTCSFSGFSLSTFGMGVGWIRQSSGKGLEWLANIWWDDDKYYNPALKSRLTISKDASKNQAFLKIANVDTADTATYYCARIGNYGSNYWYFDVWGTGTTVTVSS

SEQ ID NO: 63 - protein, 7 aa, artificial sequence
  antibody sequence of fragment
TFGMGVG

SEQ ID NO: 64 - protein, 16 aa, artificial sequence
  antibody sequence of fragment
NIWWDDDKYYNPALKS

SEQ ID NO: 65 - protein, 13 aa, artificial sequence
  antibody sequence of fragment
IGNYGSNYWYFDV

SEQ ID NO: 66 - protein, 128 aa, artificial sequence
  antibody sequence of fragment
MDFQVQIFSFLLISASVILSRGQIVLTQSPAIMSASPGERVTMTCSANSRISYMYWYQQKPGSSPRLLIYDTSNLASGVPVRFSGSGSGTSYSLTISRMEAEDAATYNCQQWSSYPLTFGAGTKLELK

SEQ ID NO: 67 - protein, 106 aa, artificial sequence
  antibody sequence of fragment
QIVLTQSPAIMSASPGERVTMTCSANSRISYMYWYQQKPGSSPRLLIYDTSNLASGVPVRFSGSGSGTSYSLTISRMEAEDAATYNCQQWSSYPLTFGAGTKLELK

SEQ ID NO: 68 - protein, 10 aa, artificial sequence
  antibody sequence of fragment
SANSRISYMY

SEQ ID NO: 69 - protein, 7 aa, artificial sequence
  antibody sequence of fragment
DTSNLAS

SEQ ID NO: 70 - protein, 9 aa, artificial sequence
  antibody sequence of fragment
QQWSSYPLT

SEQ ID NO: 71 - protein, 142 aa, artificial sequence
  antibody sequence of fragment
MGRLTSSFLLLIVPAYVLSQVTLKESGPGILQPSQTLSLTCSFSGFSLSTFGMGVGWIRQPSEKGLEWLAHIWWDDDKYYNPALKSRLTISKDTSKNQIFLKITNVDTAETATYYCSRIGNYGSNYWYFDVWGTGTTVTVSS

SEQ ID NO: 72 - protein, 123 aa, artificial sequence
  antibody sequence of fragment
QVTLKESGPGILQPSQTLSLTCSFSGFSLSTFGMGVGWIRQPSEKGLEWLAHIWWDDDKYYNPALKSRLTISKDTSKNQIFLKITNVDTAETATYYCSRIGNYGSNYWYFDVWGTGTTVTVSS

SEQ ID NO: 73 - protein, 7 aa, artificial sequence
  antibody sequence of fragment
TFGMGVG

SEQ ID NO: 74 - protein, 16 aa, artificial sequence
  antibody sequence of fragment
HIWWDDDKYYNPALKS

SEQ ID NO: 75 - protein, 13 aa, artificial sequence
  antibody sequence of fragment
IGNYGSNYWYFDV

SEQ ID NO: 76 - protein, 128 aa, artificial sequence
  antibody sequence of fragment
MDFQVQIFSFLLISASVIVSRGQIVLTQSPAVMSASPGEKVTMTCTASSSVYYMYWYQQTPGSSPRLLIYDTSNLASGVPVRFSGSGSGTSYSLTISRMEAEDAATYYCQQWNTYPLTFGAGTKLELK

SEQ ID NO: 77 - protein, 106 aa, artificial sequence
  antibody sequence of fragment
QIVLTQSPAVMSASPGEKVTMTCTASSSVYYMYWYQQTPGSSPRLLIYDTSNLASGVPVRFSGSGSGTSYSLTISRMEAEDAATYYCQQWNTYPLTFGAGTKLELK

SEQ ID NO: 78 - protein, 10 aa, artificial sequence
  antibody sequence of fragment
TASSSVYYMY

SEQ ID NO: 79 - protein, 7 aa, artificial sequence
  antibody sequence of fragment
DTSNLAS

SEQ ID NO: 80 - protein, 9 aa, artificial sequence
  antibody sequence of fragment
QQWNTYPLT

SEQ ID NO: 81 - protein, 143 aa, artificial sequence
  antibody sequence of fragment
MGRLTSSFLLLIVPAYVLSQVTLKESGPGILQPSQTLSLTCSFSGFSLSTFGMGVGWIRQPSGKGLEWLAHIWWDDDNYYNQALKSRLTISKDNTKNQVFLNIANVDTADTATYYCARIRSYDYDVRYAMDYWGQGTSVTVSS

SEQ ID NO: 82 - protein, 124 aa, artificial sequence
  antibody sequence of fragment
QVTLKESGPGILQPSQTLSLTCSFSGFSLSTFGMGVGWIRQPSGKGLEWLAHIWWDDDNYYNQALKSRLTISKDNTKNQVFLNIANVDTADTATYYCARIRSYDYDVRYAMDYWGQGTSVTVSS

SEQ ID NO: 83 - protein, 7 aa, artificial sequence
  antibody sequence of fragment
TFGMGVG

SEQ ID NO: 84 - protein, 16 aa, artificial sequence
  antibody sequence of fragment
HIWWDDDNYYNQALKS

SEQ ID NO: 85 - protein, 14 aa, artificial sequence
  antibody sequence of fragment
IRSYDYDVRYAMDY

SEQ ID NO: 86 - protein, 127 aa, artificial sequence
  antibody sequence of fragment
MRTPAQFLGILLLWFPGIKCDIKMTQSPSSMYASLRERVTITCKASQDINSYLSWFQQKPGKSPKTLIYRANRLVDGVPSRFSGSGSGQDYSLTISSLEYEDLGIYYCQQYYEFPLTFGAGTRLELK

SEQ ID NO: 87 - protein, 107 aa, artificial sequence
  antibody sequence of fragment
DIKMTQSPSSMYASLRERVTITCKASQDINSYLSWFQQKPGKSPKTLIYRANRLVDGVPSRFSGSGSGQDYSLTISSLEYEDLGIYYCQQYYEFPLTFGAGTRLELK

SEQ ID NO: 88 - protein, 11 aa, artificial sequence
  antibody sequence of fragment
KASQDINSYLS

SEQ ID NO: 89 - protein, 7 aa, artificial sequence
  antibody sequence of fragment
RANRLVD

SEQ ID NO: 90 - protein, 9 aa, artificial sequence
  antibody sequence of fragment
QQYYEFPLT

SEQ ID NO: 91 - protein, 136 aa, artificial sequence
  antibody sequence of fragment
MEWSRVFIFLLSVTAGVHSQVQLQQSGAELVRPGTSVKVSCKASGYAFTNYLIEWVKQRPGQGLEWIGVINPGSGFTNYNEKFKGKAILNADKSSSTAYMQLISLTSEDSAVYFCARDLKRAMDHWGQGTSVTVSS

SEQ ID NO: 92 - protein, 117 aa, artificial sequence
  antibody sequence of fragment
QVQLQQSGAELVRPGTSVKVSCKASGYAFTNYLIEWVKQRPGQGLEWIGVINPGSGFTNYNEKFKGKAILNADKSSSTAYMQLISLTSEDSAVYFCARDLKRAMDHWGQGTSVTVSS

SEQ ID NO: 93 - protein, 5 aa, artificial sequence
  antibody sequence of fragment
NYLIE

SEQ ID NO: 94 - protein, 17 aa, artificial sequence
  antibody sequence of fragment
VINPGSGFTNYNEKFKG

SEQ ID NO: 95 - protein, 8 aa, artificial sequence
  antibody sequence of fragment
DLKRAMDH

SEQ ID NO: 96 - protein, 125 aa, artificial sequence
  antibody sequence of fragment
MLTQLLGLLLLWFAGGKCDIQMTQSPASQSASLGESVTITCLASQTIGTWLAWYQQKPGKSPQLLIHAATSLADGVPSRFSGSGSGTKFSFKISSLQAEDFVSYYCQQIYSSPYTFGGGTKLEIK

SEQ ID NO: 97 - protein, 107 aa, artificial sequence
  antibody sequence of fragment
DIQMTQSPASQSASLGESVTITCLASQTIGTWLAWYQQKPGKSPQLLIHAATSLADGVPSRFSGSGSGTKFSFKISSLQAEDFVSYYCQQIYSSPYTFGGGTKLEIK

SEQ ID NO: 98 - protein, 11 aa, artificial sequence
  antibody sequence of fragment
LASQTIGTWLA

SEQ ID NO: 99 - protein, 7 aa, artificial sequence
  antibody sequence of fragment
AATSLAD

SEQ ID NO: 100 - protein, 9 aa, artificial sequence
  antibody sequence of fragment
QQIYSSPYT

SEQ ID NO: 101 - protein, 136 aa, artificial sequence
  antibody sequence of fragment
MEWSRVFIFLLSVTAGVHSQVQLQQSGTELVRPGTSVKVSCKASGYAFTNYLIEWVKQRPGQGLEWIGVINPGSGFTKYNEKFKGKATLTADKSSSTVYMHLSSLTSEDSAVYFCARGLYRAMDYWGQGTSVTVSS

SEQ ID NO: 102 - protein, 117 aa, artificial sequence
  antibody sequence of fragment
QVQLQQSGTELVRPGTSVKVSCKASGYAFTNYLIEWVKQRPGQGLEWIGVINPGSGFTKYNEKFKGKATLTADKSSSTVYMHLSSLTSEDSAVYFCARGLYRAMDYWGQGTSVTVSS

SEQ ID NO: 103 - protein, 5 aa, artificial sequence
  antibody sequence of fragment
NYLIE

SEQ ID NO: 104 - protein, 17 aa, artificial sequence
  antibody sequence of fragment
VINPGSGFTKYNEKFKG

SEQ ID NO: 105 - protein, 8 aa, artificial sequence
  antibody sequence of fragment
GLYRAMDY

SEQ ID NO: 106 - protein, 125 aa, artificial sequence
  antibody sequence of fragment
MLTQLLGLLLLWFAGGKCDIQMTQSPASQSASLGESVTITCLASQTIATWLAWYQQKPGKSPQLLIYGATTLADGVPSRFSGGGSGTKFSFKISSLQAEDFVSYYCQQLYSTPYTFGGGTKLEIK

SEQ ID NO: 107 - protein, 107 aa, artificial sequence
  antibody sequence of fragment
DIQMTQSPASQSASLGESVTITCLASQTIATWLAWYQQKPGKSPQLLIYGATTLADGVPSRFSGGGSGTKFSFKISSLQAEDFVSYYCQQLYSTPYTFGGGTKLEIK

SEQ ID NO: 108 - protein, 11 aa, artificial sequence
  antibody sequence of fragment
LASQTIATWLA

SEQ ID NO: 109 - protein, 7 aa, artificial sequence
  antibody sequence of fragment
GATTLAD

SEQ ID NO: 110 - protein, 9 aa, artificial sequence
  antibody sequence of fragment
QQLYSTPYT

SEQ ID NO: 111 - protein, 142 aa, artificial sequence
  antibody sequence of fragment
MGRLTSSFLLLIVPAYVLSQVTLKESGPGILQPSQTLSLTCSFSGFSLSTFGMGVGWIRQPSGKGLEWLAHIWWDDDKYYNPALQSRLTVSKDTAKNQVFLKIANVDTADTAIYYCARVGNYGSNYWYFAVWGTGTTVTVSS

SEQ ID NO: 112 - protein, 123 aa, artificial sequence
  antibody sequence of fragment
QVTLKESGPGILQPSQTLSLTCSFSGFSLSTFGMGVGWIRQPSGKGLEWLAHIWWDDDKYYNPALQSRLTVSKDTAKNQVFLKIANVDTADTAIYYCARVGNYGSNYWYFAVWGTGTTVTVSS

SEQ ID NO: 113 - protein, 7 aa, artificial sequence
  antibody sequence of fragment
TFGMGVG

SEQ ID NO: 114 - protein, 16 aa, artificial sequence
  antibody sequence of fragment
HIWWDDDKYYNPALQS

SEQ ID NO: 115 - protein, 13 aa, artificial sequence
  antibody sequence of fragment
VGNYGSNYWYFAV

SEQ ID NO: 116 - protein, 128 aa, artificial sequence
  antibody sequence of fragment
MDFQVQIFSFLLISASVILSRGQIVLTQSPAIMSASPGEKVTMTCSASSSVSYMYWYQQKPGSSPRLLIYDTSNLASGVPVRFSGSGSGTSYSLTISRMEAEDAATYYCQQWNSYPLTFGAGTKLELK

SEQ ID NO: 117 - protein, 106 aa, artificial sequence
  antibody sequence of fragment
QIVLTQSPAIMSASPGEKVTMTCSASSSVSYMYWYQQKPGSSPRLLIYDTSNLASGVPVRFSGSGSGTSYSLTISRMEAEDAATYYCQQWNSYPLTFGAGTKLELK

SEQ ID NO: 118 - protein, 10 aa, artificial sequence
  antibody sequence of fragment
SASSSVSYMY

SEQ ID NO: 119 - protein, 7 aa, artificial sequence
  antibody sequence of fragment
DTSNLAS

SEQ ID NO: 120 - protein, 9 aa, artificial sequence
  antibody sequence of fragment
QQWNSYPLT

SEQ ID NO: 121 - protein, 136 aa, artificial sequence
  antibody sequence of fragment
MEWSRVFIFLLSVTAGVHSQVQLQQSGAELVRPGASVKVSCKASGYAFSNYLIEWVKQRPGQGLEWIGVINPGSGFTKYNEKFKGKATLTADKSSSTAYMQLSSLTSEDSAVYFCARGLYRAMDYWGQGTSVTVSS

SEQ ID NO: 122 - protein, 117 aa, artificial sequence
  antibody sequence of fragment
QVQLQQSGAELVRPGASVKVSCKASGYAFSNYLIEWVKQRPGQGLEWIGVINPGSGFTKYNEKFKGKATLTADKSSSTAYMQLSSLTSEDSAVYFCARGLYRAMDYWGQGTSVTVSS

SEQ ID NO: 123 - protein, 5 aa, artificial sequence
  antibody sequence of fragment
NYLIE

SEQ ID NO: 124 - protein, 17 aa, artificial sequence
  antibody sequence of fragment
VINPGSGFTKYNEKFKG

SEQ ID NO: 125 - protein, 8 aa, artificial sequence
  antibody sequence of fragment
GLYRAMDY

SEQ ID NO: 126 - protein, 125 aa, artificial sequence
  antibody sequence of fragment
MLTQLLGLLLLWFAGGKCDIQMTQSPASQSASLGESVTITCLASQTIGTWLAWYQQKPGKSPQLLIYAAASLADGVPSRFSGGGSGTKFSFKISSLQAEDFVNYYCQQIYSTPYTFGGGTKLEIK

SEQ ID NO: 127 - protein, 107 aa, artificial sequence
  antibody sequence of fragment
DIQMTQSPASQSASLGESVTITCLASQTIGTWLAWYQQKPGKSPQLLIYAAASLADGVPSRFSGGGSGTKFSFKISSLQAEDFVNYYCQQIYSTPYTFGGGTKLEIK

SEQ ID NO: 128 - protein, 11 aa, artificial sequence
  antibody sequence of fragment
LASQTIGTWLA

SEQ ID NO: 129 - protein, 7 aa, artificial sequence
  antibody sequence of fragment
AAASLAD

SEQ ID NO: 130 - protein, 9 aa, artificial sequence
  antibody sequence of fragment
QQIYSTPYT

SEQ ID NO: 131 - protein, 135 aa, artificial sequence
  antibody sequence of fragment
MEWPLIFLFLLSGTAGVQSQVQLQQSGAELVKPGASVKISCKASGYTFSNYWMNWVKQRPGKGLEWIGQIYPGDGDTKYNGKFKNKATLTADKSSSTAYMQFSSLTSEDSAVYFCARGDLVFVYWGLGTLVTVSA

SEQ ID NO: 132 - protein, 116 aa, artificial sequence
  antibody sequence of fragment
QVQLQQSGAELVKPGASVKISCKASGYTFSNYWMNWVKQRPGKGLEWIGQIYPGDGDTKYNGKFKNKATLTADKSSSTAYMQFSSLTSEDSAVYFCARGDLVFVYWGLGTLVTVSA

SEQ ID NO: 133 - protein, 5 aa, artificial sequence
  antibody sequence of fragment
NYWMN

SEQ ID NO: 134 - protein, 17 aa, artificial sequence
  antibody sequence of fragment
QIYPGDGDTKYNGKFKN

SEQ ID NO: 135 - protein, 7 aa, artificial sequence
  antibody sequence of fragment
GDLVFVY

SEQ ID NO: 136 - protein, 127 aa, artificial sequence
  antibody sequence of fragment
MRFQVQVLGLLLLWISGAQCDVQITQSPSYLAVSPGETITINCRASKNIRKYLAWYQEKPGKTNKLLIYSGSTSQSGVPSRFSGSGSGTDFTLTISSLEPEDFAMYYCQQHNEYPYTFGGGTKLEIK

SEQ ID NO: 137 - protein, 107 aa, artificial sequence
  antibody sequence of fragment
DVQITQSPSYLAVSPGETITINCRASKNIRKYLAWYQEKPGKTNKLLIYSGSTSQSGVPSRFSGSGSGTDFTLTISSLEPEDFAMYYCQQHNEYPYTFGGGTKLEIK

SEQ ID NO: 138 - protein, 11 aa, artificial sequence
  antibody sequence of fragment
RASKNIRKYLA

SEQ ID NO: 139 - protein, 7 aa, artificial sequence
  antibody sequence of fragment
SGSTSQS

SEQ ID NO: 140 - protein, 9 aa, artificial sequence
  antibody sequence of fragment
QQHNEYPYT

SEQ ID NO: 141 - protein, 136 aa, artificial sequence
  antibody sequence of fragment
MEWSRVFIFLLSVTAGAHSQVQLQQSGAELVRPGTSVKVSCKASGYAFTNYLIEWVKQRPGQGLEWIGVFNPESGYINYNEKLKGKATLTADKSSSTAYMQLSSLTSEDSAVYFCARTSRRGFDYWGHGTTLTVSS

SEQ ID NO: 142 - protein, 117 aa, artificial sequence
  antibody sequence of fragment
QVQLQQSGAELVRPGTSVKVSCKASGYAFTNYLIEWVKQRPGQGLEWIGVFNPESGYINYNEKLKGKATLTADKSSSTAYMQLSSLTSEDSAVYFCARTSRRGFDYWGHGTTLTVSS

SEQ ID NO: 143 - protein, 5 aa, artificial sequence
  antibody sequence of fragment
NYLIE

SEQ ID NO: 144 - protein, 17 aa, artificial sequence
  antibody sequence of fragment
VFNPESGYINYNEKLKG

SEQ ID NO: 145 - protein, 8 aa, artificial sequence
  antibody sequence of fragment
TSRRGFDY

SEQ ID NO: 146 - protein, 127 aa, artificial sequence
  antibody sequence of fragment
MNMLTQLLGLLLLWFAGGKCDIQMTQSPASQSASLGESVTITCLASQTIGTWLAWYQQKPGKSPQLLIYAATSLADGVPSRFSGSGSGTKFSFKISSLQAEDFVSYYCQQLYSTPRTFGGGTKLEIK

SEQ ID NO: 147 - protein, 107 aa, artificial sequence
  antibody sequence of fragment
DIQMTQSPASQSASLGESVTITCLASQTIGTWLAWYQQKPGKSPQLLIYAATSLADGVPSRFSGSGSGTKFSFKISSLQAEDFVSYYCQQLYSTPRTFGGGTKLEIK

SEQ ID NO: 148 - protein, 11 aa, artificial sequence
  antibody sequence of fragment
LASQTIGTWLA

SEQ ID NO: 149 - protein, 7 aa, artificial sequence
  antibody sequence of fragment
AATSLAD

SEQ ID NO: 150 - protein, 9 aa, artificial sequence
  antibody sequence of fragment
QQLYSTPRT

SEQ ID NO: 151 - protein, 142 aa, artificial sequence
  antibody sequence of fragment
MGRLTSSFLLLIVPAYVLSQVTLKESGPGILQPSQTLSLTCSFSGFSLSTFGLGVGWIRQPSGKGLECLAHIWWDDDKYSNPALKSRLTISKDTSKNQVFLKIANVDSADTATYFCARILNYGSNYWYFDVWGTGTTVTVSS

SEQ ID NO: 152 - protein, 123 aa, artificial sequence
  antibody sequence of fragment
QVTLKESGPGILQPSQTLSLTCSFSGFSLSTFGLGVGWIRQPSGKGLECLAHIWWDDDKYSNPALKSRLTISKDTSKNQVFLKIANVDSADTATYFCARILNYGSNYWYFDVWGTGTTVTVSS

SEQ ID NO: 153 - protein, 7 aa, artificial sequence
  antibody sequence of fragment
TFGLGVG

SEQ ID NO: 154 - protein, 16 aa, artificial sequence
  antibody sequence of fragment
HIWWDDDKYSNPALKS

SEQ ID NO: 155 - protein, 13 aa, artificial sequence
  antibody sequence of fragment
ILNYGSNYWYFDV

SEQ ID NO: 156 - protein, 128 aa, artificial sequence
  antibody sequence of fragment
MDFQVQIFSFLLISASVILSRGQIVLTQSPALMSASPGEKVTMTCSASSSISYMYWYQQKPGSSPRLLIYDTSNLASGVPVRFSGSGSGTSFSLTVSRMEAEDAATYYCQQWNSYPLTFGAGTKLELK

SEQ ID NO: 157 - protein, 106 aa, artificial sequence
  antibody sequence of fragment
QIVLTQSPALMSASPGEKVTMTCSASSSISYMYWYQQKPGSSPRLLIYDTSNLASGVPVRFSGSGSGTSFSLTVSRMEAEDAATYYCQQWNSYPLTFGAGTKLELK

SEQ ID NO: 158 - protein, 10 aa, artificial sequence
  antibody sequence of fragment
SASSSISYMY

SEQ ID NO: 159 - protein, 7 aa, artificial sequence
  antibody sequence of fragment
DTSNLAS

SEQ ID NO: 160 - protein, 9 aa, artificial sequence
  antibody sequence of fragment
QQWNSYPLT

SEQ ID NO: 161 - protein, 142 aa, artificial sequence
  antibody sequence of fragment
MGRLTSSFLLLIVPAYVLSQVTLKESGPGILQPSQTLSLTCSFSGFSLSTFGMGVGWIRQPSGKGLEWLTNIWWDDDKYYNSVLKSRLTISKDTSKNQVFLKIANVDTADTATYYCARIAAYGSNYWYFDVWGTGTTVTVSS

SEQ ID NO: 162 - protein, 123 aa, artificial sequence
  antibody sequence of fragment
QVTLKESGPGILQPSQTLSLTCSFSGFSLSTFGMGVGWIRQPSGKGLEWLTNIWWDDDKYYNSVLKSRLTISKDTSKNQVFLKIANVDTADTATYYCARIAAYGSNYWYFDVWGTGTTVTVSS

SEQ ID NO: 163 - protein, 7 aa, artificial sequence
  antibody sequence of fragment
TFGMGVG

SEQ ID NO: 164 - protein, 16 aa, artificial sequence
  antibody sequence of fragment
NIWWDDDKYYNSVLKS

SEQ ID NO: 165 - protein, 13 aa, artificial sequence
  antibody sequence of fragment
IAAYGSNYWYFDV

SEQ ID NO: 166 - protein, 128 aa, artificial sequence
  antibody sequence of fragment
MDFQVQIFSFLLISASVKLSRGQIVLTQSPAIMSASPGEKVTMTCSASSSVSYMYWYQQKPGSSPRVLIYDTSNLSSGVPVRFSGSGSGTSYSLTISRMEAEDAATYYCQQWSSYPLTFGAGTKLELK

SEQ ID NO: 167 - protein, 106 aa, artificial sequence
  antibody sequence of fragment
QIVLTQSPAIMSASPGEKVTMTCSASSSVSYMYWYQQKPGSSPRVLIYDTSNLSSGVPVRFSGSGSGTSYSLTISRMEAEDAATYYCQQWSSYPLTFGAGTKLELK

SEQ ID NO: 168 - protein, 10 aa, artificial sequence
  antibody sequence of fragment
SASSSVSYMY

SEQ ID NO: 169 - protein, 7 aa, artificial sequence
  antibody sequence of fragment
DTSNLSS

SEQ ID NO: 170 - protein, 9 aa, artificial sequence
  antibody sequence of fragment
QQWSSYPLT

SEQ ID NO: 171 - protein, 142 aa, artificial sequence
  antibody sequence of fragment
MGRLTSSLLLLIVPAYVLSQVTLKESGPGILQPSQTLSLTCSFSGFSLSTFGMGVGWIRQPSGKGLEWLAHIWWDDDKYYNPALKSRLTISKDTSKNQIFLKIANVDTADSATFYCARIENYGSNYWYFDVWGTGTTVTVSS

SEQ ID NO: 172 - protein, 123 aa, artificial sequence
  antibody sequence of fragment
QVTLKESGPGILQPSQTLSLTCSFSGFSLSTFGMGVGWIRQPSGKGLEWLAHIWWDDDKYYNPALKSRLTISKDTSKNQIFLKIANVDTADSATFYCARIENYGSNYWYFDVWGTGTTVTVSS

SEQ ID NO: 173 - protein, 7 aa, artificial sequence
  antibody sequence of fragment
TFGMGVG

SEQ ID NO: 174 - protein, 16 aa, artificial sequence
  antibody sequence of fragment
HIWWDDDKYYNPALKS

SEQ ID NO: 175 - protein, 13 aa, artificial sequence
  antibody sequence of fragment
IENYGSNYWYFDV

SEQ ID NO: 176 - protein, 128 aa, artificial sequence
  antibody sequence of fragment
MDFQVQIFSFLLISASVRLSRGQIVLTQSPAIMSASPGEKVTMTCSASSSVSYMYWYQQKPGSSPRVLIYDTSNLASGVPVRFSGSGSGTSYSLTVSRMEAEDAATYYCQQWNSYPLTFGAGTKLELK

SEQ ID NO: 177 - protein, 106 aa, artificial sequence
  antibody sequence of fragment
QIVLTQSPAIMSASPGEKVTMTCSASSSVSYMYWYQQKPGSSPRVLIYDTSNLASGVPVRFSGSGSGTSYSLTVSRMEAEDAATYYCQQWNSYPLTFGAGTKLELK

SEQ ID NO: 178 - protein, 10 aa, artificial sequence
  antibody sequence of fragment
SASSSVSYMY

SEQ ID NO: 179 - protein, 7 aa, artificial sequence
  antibody sequence of fragment
DTSNLAS

SEQ ID NO: 180 - protein, 9 aa, artificial sequence
  antibody sequence of fragment
QQWNSYPLT

SEQ ID NO: 181 - protein, 856 aa, Homo sapiens
MASADKNGGSVSSVSSSRLQSRKPPNLSITIPPPEKETQAPGEQDSMLPEGFQNRRLKKSQPRTWAAHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHAPHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 182 - protein, 855 aa, Homo sapiens
MSEARRDSTSSLQRKKPPWLKLDIPSAVPLTAEEPSFLQPLRRQAFLRSVSMPAETAHISSPHHELRRPVLQRQTSITQTIRRGTADWFGVSKDSDSTQKWQRKSIRHCSQRYGKLKPQVLRELDLPSQDNVSLTSTETPPPLYVGPCQLGMQKIIDPLARGRAFRVADDTAEGLSAPHTPVTPGAASLCSFSSSRSGFHRLPRRRKRESVAKMSFRAAAALMKGRSVRDGTFRRAQRRSFTPASFLEEDTTDFPDELDTSFFAREGILHEELSTYPDEVFESPSEAALKDWEKAPEQADLTGGALDRSELERSHLMLPLERGWRKQKEGAAAPQPKVRLRQEVVSTAGPRRGQRIAVPVRKLFAREKRPYGLGMVGRLTNRTYRKRIDSFVKRQIEDMDDHRPFFTYWLTFVHSLVTILAVCIYGIAPVGFSQHETVDSVLRNRGVYENVKYVQQENFWIGPSSEALIHLGAKFSPCMRQDPQVHSFIRSAREREKHSACCVRNDRSGCVQTSEEECSSTLAVWVKWPIHPSAPELAGHKRQFGSVCHQDPRVCDEPSSEDPHEWPEDITKWPICTKNSAGNHTNHPHMDCVITGRPCCIGTKGRCEITSREYCDFMRGYFHEEATLCSQVHCMDDVCGLLPFLNPEVPDQFYRLWLSLFLHAGILHCLVSICFQMTVLRDLEKLAGWHRIAIIYLLSGVTGNLASAIFLPYRAEVGPAGSQFGILACLFVELFQSWQILARPWRAFFKLLAVVLFLFTFGLLPWIDNFAHISGFISGLFLSFAFLPYISFGKFDLYRKRCQIIIFQVVFLGLLAGLVVLFYVYPVRCEWCEFLTCIPFTDKFCEKYELDAQLH

SEQ ID NO: 183 - protein, 827 aa, Mus musculus
MASADKNGSNLPSVSGSRLQSRKPPNLSITIPPPESQAPGEQDSMLPERRKNPAYLKSVSLQEPRGRWQEGAEKRPGFRRQASLSQSIRKSTAQWFGVSGDWEGKRQNWHRRSLHHCSVHYGRLKASCQRELELPSQEVPSFQGTESPKPCKMPKIVDPLARGRAFRHPDEVDRPHAAHPPLTPGVLSLTSFTSVRSGYSHLPRRKRISVAHMSFQAAAALLKGRSVLDATGQRCRHVKRSFAYPSFLEEDAVDGADTFDSSFFSKEEMSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLH

SEQ ID NO: 184 - protein, 856 aa, Artificial Sequence
  Synthetic Construct
MASADKNGGSVSSVSSSRLQSRKPPNLSITIPPPEKETQAPGEQDSMLPEGFQNRRLKKSQPRTWAAHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKTVDPLARGRAFRHPEEMDRPHAPHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 185 - protein, 614 aa, Artificial Sequence
  Synthetic Construct
MSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLH

SEQ ID NO: 186 - protein, 827 aa, Artificial Sequence
  Synthetic Construct
MASADKNGSNLPSVSGSRLQSRKPPNLSITIPPPESQAPGEQDSMLPERRKNPAYLKSVSLQEPRGRWQEGAEKRPGFRRQASLSQSIRKSTAQWFGVSGDWEGKRQNWHRRSLHHCSVHYGRLKASCQRELELPSQEVPSFQGTESPKPCKMPKTVDPLARGRAFRHPDEVDRPHAAHPPLTPGVLSLTSFTSVRSGYSHLPRRKRISVAHMSFQAAAALLKGRSVLDATGQRCRHVKRSFAYPSFLEEDAVDGADTFDSSFFSKEEMSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLH

SEQ ID NO: 187 - protein, 615 aa, Artificial Sequence
  Synthetic Construct
MSFQAAAALLKGRSVLDATGQRCRHVKRSFAYPSFLEEDAVDGADTFDSSFFSKEEMSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLH

SEQ ID NO: 188 - protein, 559 aa, Artificial Sequence
  Synthetic Construct
MSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLH

SEQ ID NO: 189 - protein, 888 aa, Artificial Sequence
  Synthetic Construct
MSEARRDSTSSLQRKKPPWLKLDIPSAVPLTAEEPSFLQPLRRQAFLRSVSMPAETAHISSPHHELRRPVLQRQTSITQTIRRGTADWFGVSKDSDSTQKWQRKSIRHCSQRYGKLKPQVLRELDLPSQDNVSLTSTETPPPLYVGPCQLGMQKIIDPLARGRAFRVADDTAEGLSAPHTPVTPGAASLCSFSSSRSGFHRLPRRRKRESVAKMSFRAAAALMKGRSVRDGTFRRAQRRSFTPASFLEEDTTDFPDELDTSFFAREGILHEELSTYPDEVFESPSEAALKDWEKAPEQADLTGGALDRSELERSHLMLPLERGWRKQKEGAAAPQPKVRLRQEVVSTAGPRRGQRIAVPVRKLFAREKRPYGLGMVGRLTNRTYRKRIDSFVKRQIEDMDDHRPFFTYWLTFVHSLVTILAVCIYGIAPVGFSQHETVDSVLRNRGVYENVKYVQQENFWIGPSSEALIHLGAKFSPCMRQDPQVHSFIRSAREREKHSACCVRNDRSGCVQTSEEECSSTLAVWVKWPIHPSAPELAGHKRQFGSVCHQDPRVCDEPSSEDPHEWPEDITKWPICTKNSAGNHTNHPHMDCVITGRPCCIGTKGRCEITSREYCDFMRGYFHEEATLCSQVHCMDDVCGLLPFLNPEVPDQFYRLWLSLFLHAGILHCLVSICFQMTVLRDLEKLAGWHRIAIIYLLSGVTGNLASAIFLPYRAEVGPAGSQFGILACLFVELFQSWQILARPWRAFFKLLAVVLFLFTFGLLPWIDNFAHISGFISGLFLSFAFLPYISFGKFDLYRKRCQIIIFQVVFLGLLAGLVVLFYVYPVRCEWCEFLTCIPFTDKFCEKYELDAQLHMASMTGGQQMGMASMTGGQQMGMASMTGGQQMG

SEQ ID NO: 190 - protein, 889 aa, Artificial Sequence
  Synthetic Construct
MASADKNGGSVSSVSSSRLQSRKPPNLSITIPPPEKETQAPGEQDSMLPEGFQNRRLKKSQPRTWAAHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHAPHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLHMASMTGGQQMGMASMTGGQQMGMASMTGGQQMG

SEQ ID NO: 191 - protein, 647 aa, Artificial Sequence
  Synthetic Construct
MSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLHMASMTGGQQMGMASMTGGQQMGMASMTGGQQMG

SEQ ID NO: 192 - protein, 889 aa, Artificial Sequence
  Synthetic Construct
MSEARRDSTSSLQRKKPPWLKLDIPAAVPPAAEEPSFLQPLRRQAFLRSVSMPAETARVPSPHHEPRRLVLQRQTSITQTIRRGTADWFGVSKDSDSTQKWQRKSIRHCSQRYGKLKPQVIRELDLPSQDNVSLTSTETPPPLYVGPCQLGMQKIIDPLARGRAFRMADDTADGLSAPHTPVTPGAASLCSFSSSRSGFNRLPRRRKRESVAKMSFRAAAALVKGRSIRDGTLRRGQRRSFTPASFLEEDMVDFPDELDTSFFAREGVLHEEMSTYPDEVFESPSEAALKDWEKAPDQADLTGGALDRSELERSHLMLPLERGWRKQKEGGPLAPQPKVRLRQEVVSAAGPRRGQRIAVPVRKLFAREKRPYGLGMVGRLTNRTYRKRIDSYVKRQIEDMDDHRPFFTYWLTFVHSLVTILAVCIYGIAPVGFSQHETVDSVLRKRGVYENVKYVQQENFWIGPSSEALIHLGAKFSPCMRQDPQVHSFILAAREREKHSACCVRNDRSGCVQTSKEECSSTLAVWVKWPVHPSAPDLAGNKRQFGSVCHQDPRVCDEPSSEDPHEWPEDITKWPICTKSSAGNHTNHPHMDCVITGRPCCIGTKGRCEITSREYCDFMRGYFHEEATLCSQVHCMDDVCGLLPFLNPEVPDQFYRLWLSLFLHAGILHCLVSVCFQMTVLRDLEKLAGWHRIAIIYLLSGITGNLASAIFLPYRAEVGPAGSQFGILACLFVELFQSWQILARPWRAFFKLLAVVLFLFAFGLLPWIDNFAHISGFVSGLFLSFAFLPYISFGKFDLYRKRCQIIIFQVVFLGLLAGLVVLFYFYPVRCEWCEFLTCIPFTDKFCEKYELDAQLHMASMTGGQQMGMASMTGGQQMGMASMTGGQQMG

SEQ ID NO: 193 - protein, 860 aa, Artificial Sequence
  Synthetic Construct
MASADKNGSNLPSVSGSRLQSRKPPNLSITIPPPESQAPGEQDSMLPERRKNPAYLKSVSLQEPRGRWQEGAEKRPGFRRQASLSQSIRKSTAQWFGVSGDWEGKRQNWHRRSLHHCSVHYGRLKASCQRELELPSQEVPSFQGTESPKPCKMPKIVDPLARGRAFRHPDEVDRPHAAHPPLTPGVLSLTSFTSVRSGYSHLPRRKRISVAHMSFQAAAALLKGRSVLDATGQRCRHVKRSFAYPSFLEEDAVDGADTFDSSFFSKEEMSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLHMASMTGGQQMGMASMTGGQQMGMASMTGGQQMG

SEQ ID NO: 194 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 195 - DNA, 22 bp, artificial sequence
  mRhbdf2.3 fwd  primer
tcaatgagctctttatggggca

SEQ ID NO: 196 - DNA, 22 bp, artificial sequence
  mRhbdf2.3 rev primer
aaggtctccatcccctcaggtc

SEQ ID NO: 197 - DNA, 20 bp, artificial sequence
  mRhbdf2.3 IVT gRNA
aagcatgctatcctgctcgc

SEQ ID NO: 198 - protein, 878 aa, Artificial Sequence
  Synthetic Construct
MASADKNGGSVSSVSSSRLQSRKPPNLSITIPPPEKETQAPGEQDSMLPEGFQNRRLKKSQPRTWAAHTTACPPSFLPKRKNPAYLKSVSLQEPRSRWQESSEKRPGFRRQASLSQSIRKGAAQWFGVSGDWEGQRQQWQRRSLHHCSMRYGRLKASCQRDLELPSQEAPSFQGTESPKPCKMPKIVDPLARGRAFRHPEEMDRPHAPHPPLTPGVLSLTSFTSVRSGYSHLPRRKRMSVAHMSLQAAAALLKGRSVLDATGQRCRVVKRSFAFPSFLEEDVVDGADTFDSSFFSKEEMSSMPDDVFESPPLSASYFRGIPHSASPVSPDGVQIPLKEYGRAPVPGPRRGKRIASKVKHFAFDRKKRHYGLGVVGNWLNRSYRRSISSTVQRQLESFDSHRPYFTYWLTFVHVIITLLVICTYGIAPVGFAQHVTTQLVLRNKGVYESVKYIQQENFWVGPSSIDLIHLGAKFSPCIRKDGQIEQLVLRERDLERDSGCCVQNDHSGCIQTQRKDCSETLATFVKWQDDTGPPMDKSDLGQKRTSGAVCHQDPRTCEEPASSGAHIWPDDITKWPICTEQARSNHTGFLHMDCEIKGRPCCIGTKGSCEITTREYCEFMHGYFHEEATLCSQVHCLDKVCGLLPFLNPEVPDQFYRLWLSLFLHAGVVHCLVSVVFQMTILRDLEKLAGWHRIAIIFILSGITGNLASAIFLPYRAEVGPAGSQFGLLACLFVELFQSWPLLERPWKAFLNLSAIVLFLFICGLLPWIDNIAHIFGFLSGLLLAFAFLPYITFGTSDKYRKRALILVSLLAFAGLFAALVLWLYIYPINWPWIEHLTCFPFTSRFCEKYELDQVLHDYKDHDGDYKDHDIDYKDDDDK

SEQ ID NO: 199 - protein, 849 aa, Artificial Sequence
  Synthetic Construct
MASADKNGSNLPSVSGSRLQSRKPPNLSITIPPPESQAPGEQDSMLPERRKNPAYLKSVSLQEPRGRWQEGAEKRPGFRRQASLSQSIRKSTAQWFGVSGDWEGKRQNWHRRSLHHCSVHYGRLKASCQRELELPSQEVPSFQGTESPKPCKMPKIVDPLARGRAFRHPDEVDRPHAAHPPLTPGVLSLTSFTSVRSGYSHLPRRKRISVAHMSFQAAAALLKGRSVLDATGQRCRHVKRSFAYPSFLEEDAVDGADTFDSSFFSKEEMSSMPDDVFESPPLSASYFRGVPHSASPVSPDGVHIPLKEYSGGRALGPGTQRGKRIASKVKHFAFDRKKRHYGLGVVGNWLNRSYRRSISSTVQRQLESFDSHRPYFTYWLTFVHIIITLLVICTYGIAPVGFAQHVTTQLVLKNRGVYESVKYIQQENFWIGPSSIDLIHLGAKFSPCIRKDQQIEQLVRRERDIERTSGCCVQNDRSGCIQTLKKDCSETLATFVKWQNDTGPSDKSDLSQKQPSAVVCHQDPRTCEEPASSGAHIWPDDITKWPICTEQAQSNHTGLLHIDCKIKGRPCCIGTKGSCEITTREYCEFMHGYFHEDATLCSQVHCLDKVCGLLPFLNPEVPDQFYRIWLSLFLHAGIVHCLVSVVFQMTILRDLEKLAGWHRISIIFILSGITGNLASAIFLPYRAEVGPAGSQFGLLACLFVELFQSWQLLERPWKAFFNLSAIVLFLFICGLLPWIDNIAHIFGFLSGMLLAFAFLPYITFGTSDKYRKRALILVSLLVFAGLFASLVLWLYIYPINWPWIEYLTCFPFTSRFCEKYELDQVLHDYKDHDGDYKDHDIDYKDDDDK